SEQUENCE LISTING (195 sequences)

SEQ ID NO: 1 - DNA, 402 bp, Mus musculus
atgagagtgctgattcttttgtggctgttcacagcctttcctggtgtcctgtctgatgtgcagcttcaggagtcgggacctagcctggtgaaaccttctcagtctctgtccctcacctgcactgtcactggctactcaatcaccagtgattttgcctggaactggatccggcagtttccaggaaacaagctggagtggatgggctacataagttatagtggtaacactaggtacaacccatctctcaaaagtcgaatctctatcactcgagacacatccaagaaccaattcttcctgcagttgaattctgtgactattgaggacacagccacatattactgtgtaacggcgggacgcgggtttccttattggggccaagggactctggtcactgtctctgca

SEQ ID NO: 2 - protein, 134 aa, Mus musculus
MRVLILLWLFTAFPGVLSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 3 - DNA, 384 bp, Mus musculus
atggtgtccacagctcagttccttgcattcttgttgctttggtttccaggtgcaagatgtgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgt

SEQ ID NO: 4 - protein, 128 aa, Mus musculus
MVSTAQFLAFLLLWFPGARCDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 5 - protein, 13 aa, Artificial Sequence
  synthetic construct
LEEKKGNYVVTDH

SEQ ID NO: 6 - protein, 13 aa, Artificial Sequence
  synthetic construct
LEEKKGNYVVTDH

SEQ ID NO: 7 - DNA, 6149 bp, Artificial Sequence
  synthetic vector
ctcgagagcgggcagtgagcgcaacgcaattaatgtgagttagctcactcattaggcaccccaggctttacactttatgctcccggctcgtatgttgtgtggagattgtgagcggataacaatttcacacagaattcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtacgcgtctcgggaagctttagtttaaacgccgccaccatggtgtccacagctcagttccttgcattcttgttgctttggtttccaggtgcaagatgtgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagcatgctcagtttccgtggacgttcggtggaggcaccaagctggaaatcaaacgggtgagtggatccatctgggataagcatgctgttttctgtctgtccctaacatgccctgtgattatgcgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagctagaactaactaactaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgataataccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgatcgacctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgcctttgacgttggagtccacgttctttaatagtggactcttgttccaaactggaacaacactcaaccctatctcggtctatttataagggattttgccgatttcggcctattggttaaaaaatgagctgatttaacaaaatttaacgcgaattttaacaaaatattaacgcttacaatttaggtggcacttttcggggaaatgtgcgcggaacccctatatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttactgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatattacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatgttcttgagatcctttttttctgcacgtaatctgctgcttgcaaacaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggtatcacgaggccctttcgtcttcac

SEQ ID NO: 8 - DNA, 6625 bp, Artificial Sequence
  synthetic vector
ctcgagagcgggcagtgagcgcaacgcaattaatgtgagttagctcactcattaggcaccccaggctttacactttatgctcccggctcgtatgttgtgtggagattgtgagcggataacaatttcacacagaattcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtacgcgtctcgggaagctttagtttaaacgccgccaccatgagagtgctgattcttttgtggctgttcacagcctttcctggtgtcctgtctgatgtgcagcttcaggagtcgggacctagcctggtgaaaccttctcagactctgtccctcacctgcactgtcactggctactcaatcaccagtgattttgcctggaactggatccggcagtttccaggaaacaagctggagtggatgggctacataagttatagtggtaacactaggtacaacccatctctcaaaagtcgaatctctatcactcgagacacatccaagaaccaattcttcctgcagttgaattctgtgactattgaggacacagccacatattactgtgtaacggcgggacgcgggtttccttattggggccaagggactctggtcactgtctctgcacagtgagtggatcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataacgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagaaactaactaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgataataccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatggttcgatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggtcgatcgacctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgcctttgacgttggagtccacgttctttaatagtggactcttgttccaaactggaacaacactcaaccctatctcggtctatttataagggattttgccgatttcggcctattggttaaaaaatgagctgatttaacaaaatttaacgcgaattttaacaaaatattaacgcttacaatttaggtggcacttttcggggaaatgtgcgcggaacccctatatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttactgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatattacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatgttcttgagatcctttttttctgcacgtaatctgctgcttgcaaacaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggtatcacgaggccctttcgtcttcac

SEQ ID NO: 9 - protein, 234 aa, Artificial Sequence
  synthetic vector
MVSTAQFLAFLLLWFPGARCDILMTQSPSSMSVSLGDTVSITCHSSQDINSNIGWLQQRPGKSFKGLIYHGTNLDDEVPSRFSGSGSGADYSLTISSLESEDFADYYCVQHAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 463 aa, Artificial Sequence
  synthetic vector
MRVLILLWLFTAFPGVLSDVQLQESGPSLVKPSQTLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSASTKGPSVFPLAPSSKSTSGGTAALGCLVKDYFPEPVTVSWNSGALTSGVHTFPAVLQSSGLYSLSSVYSVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 116 aa, Mus musculus
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 12 - protein, 108 aa, Mus musculus
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 13 - protein, 13 aa, Artificial Sequence
  synthetic construct
LEEKKGNYVVTDH

SEQ ID NO: 14 - protein, 16 aa, Artificial Sequence
  synthetic construct
CGADSYEMEEDGVRKC

SEQ ID NO: 15 - protein, 6 aa, Mus musculus
SDFAWN

SEQ ID NO: 16 - protein, 16 aa, Mus musculus
YISYSGNTRYNPSLKS

SEQ ID NO: 17 - protein, 9 aa, Mus musculus
VTAGRGFPY

SEQ ID NO: 18 - protein, 11 aa, Mus musculus
HSSQDINSNIG

SEQ ID NO: 19 - protein, 7 aa, Mus musculus
HGTNLDD

SEQ ID NO: 20 - protein, 9 aa, Mus musculus
VQYAQFPWT

SEQ ID NO: 21 - DNA, 348 bp, Mus musculus
gatgtgcagcttcaggagtcgggacctagcctggtgaaaccttctcagtctctgtccctcacctgcactgtcactggctactcaatcaccagtgactatgcctggaactggatccggcagtttccaggaaacaaactggagtggatgggctacataagttacagtgctaacactaggtacaacccatctctcaaaagtcgaatctctatcactcgagacacatccaagaaccaattcttcctgcagttgaattctgtgactactgaggacacagccacatattactgtgcaacggcgggacgcgggtttccttactggggccaagggactctggtcactgtctctgca

SEQ ID NO: 22 - protein, 116 aa, Mus musculus
DVQLQESGPSLVKPSQSLSLTCTVTGYSITSDYAWNWIRQFPGNKLEWMGYISYSANTRYNPSLKSRISITRDTSKNQFFLQLNSVTTEDTATYYCATAGRGFPYWGQGTLVTVSA

SEQ ID NO: 23 - protein, 6 aa, Mus musculus
SDYAWN

SEQ ID NO: 24 - protein, 16 aa, Mus musculus
YISYSANTRYNPSLKS

SEQ ID NO: 25 - protein, 7 aa, Mus musculus
AGRGFPY

SEQ ID NO: 26 - DNA, 324 bp, Mus musculus
gacatcctgatgacccaatctccatcctccatgtctctatctctgggagacacagtcagtatcacttgccattcaagtcaggacattaacagtaatatagggtggttgcagcagaaaccagggaaatcatttaagggcctgatctatcatggaaccaacttggacgatggagttccatcaaggttcagtggcagtggatctggagccgattattctctcaccatcagcagcctggaatctgaagattttgtagactattactgtgtacagtatggtcagtttccgtggacgttcggtggaggcaccaagctggaaatcaaacgg

SEQ ID NO: 27 - protein, 108 aa, Mus musculus
DILMTQSPSSMSLSLGDTVSITCHSSQDINSNIGWLQQKPGKSFKGLIYHGTNLDDGVPSRFSGSGSGADYSLTISSLESEDFVDYYCVQYGQFPWTFGGGTKLEIKR

SEQ ID NO: 28 - protein, 11 aa, Mus musculus
HSSQDINSNIG

SEQ ID NO: 29 - protein, 7 aa, Mus musculus
HGTNLDD

SEQ ID NO: 30 - protein, 9 aa, Mus musculus
VQYGQFPWT

SEQ ID NO: 31 - DNA, 348 bp, Mus musculus
gatgtgcagcttcaggggtcgggacctagcctggtgaaaccttctcagtctctgtccctcacctgcactgtcactggctactcaatcaccagtgattatgcctggaactggatccggcagtttccaggaaacaaactggagtggatgggctacataagctacagtggtaacactagatacaacccatctctcagaagtcgaatctctatcactcgagacacatccaagaaccaattcttcctgcagttgaattctgtgactactgaggacacagccacatattactgtgcaacggcgggacgcggatttccttactggggccaagggactctggtcactgtctctgca

SEQ ID NO: 32 - protein, 116 aa, Mus musculus
DVQLQGSGPSLVKPSQSLSLTCTVTGYSITSDYAWNWIRQFPGNKLEWMGYISYSGNTRYNPSLRSRISITRDTSKNQFFLQLNSVTTEDTATYYCATAGRGFPYWGQGTLVTVSA

SEQ ID NO: 33 - protein, 6 aa, Mus musculus
SDYAWN

SEQ ID NO: 34 - protein, 16 aa, Mus musculus
YISYSGNTRYNPSLRS

SEQ ID NO: 35 - protein, 9 aa, Mus musculus
ATAGRGFPY

SEQ ID NO: 36 - DNA, 322 bp, Mus musculus
gacatcctgatgacccaatctccatcctccatgtctgtgtctctgggagacacagtcaacatcacttgccattcaagtcaggacattaacagtaatatagggtggttgcagcagaaaccagggaaatcatttaagggcctgatctatcatggaaccaacttggacgatggagttccatcaaggttcagtggcagtggatctggagccgattattctctcaccatcagcagcctggaatctgaggattttgcagactattactgtgtacagtatggtcagtttccgtggacgttcggtggaggcaccaagctggaaatcaaac

SEQ ID NO: 37 - protein, 108 aa, Mus musculus
DILMTQSPSSMSVSLGDTVNITCHSSQDINSNIGWLQQKPGKSFKGLIYHGTNLDDGVPSRFSGSGSGADYSLTISSLESEDFADYYCVQYGQFPWTFGGGTKLEIKR

SEQ ID NO: 38 - protein, 11 aa, Mus musculus
HSSQDINSNIG

SEQ ID NO: 39 - protein, 7 aa, Artificial Sequence
  synthetic construct
HGTNLDD

SEQ ID NO: 40 - protein, 9 aa, Mus musculus
VQYGQFPWT

SEQ ID NO: 41 - DNA, 11891 bp, Artificial Sequence
  synthetic vector
aagcttgccgccaccatggattggacctggcgcattctctttctggtagcagccgccacaggtaaggggctgccaaatcccagtgaggaggaagggatcgaaggtcaccatcgaagccagtcacccagtgaagggggcttccatccactcctgtgtcttctctacaggtgtccacagccaggtgcagctccaagagagtggacctgggcttgtcaagccgagtcaaactttgtccctaacatgtactgtgtccggatactctatctcatcagattttgcgtggaattggataaggcagccaccagggaaaggtttagaatggatgggctacatatcatactctgggaacaccagatatcaaccttctctgaaaagccggatcacaatctcaagggacacgtcgaagaatcagttcttcctgaaactgaactccgttacagccgcagacacagcaacatattactgcgtaaccgctggcagaggcttcccctattggggacagggcaccctagtgacagtgagcagcggtaagatggcacaccgtggccggcctctgcgcctgggcccagctctgtcccacaccgcggtcacatggcaccttttctcttccagcctccaccaagggccccagcgtgttccccctggcccccagcagcaagagcaccagcggcggcacagccgccctgggctgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaattggtatgtggacggcgtggaggtgcacaacgccaagaccaagcccagagaagagcagtacaacagcacctacagggtggtgtccgtgctgaccgtgctgcaccaggactggctgaacggcaaggaatacaaatgcaaggtctccaacaaggccctgccagcccccatcgaaaagaccatcagcaaggccaagggccagccacgggagccccaggtgtacaccctgcccccctcccgggacgagtgcaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcccccggcaagtgatgacgacgcggccgtgcggacgaccgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggcggccgccgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggattggacttggagaatactgtttcttgtagcagccgcaacaggtaaggggctgccaaatcccagtgaggaggaagggatcgaaggtgaccatcgaagccagtcaagggggcggaccgcttccatccactcctgtgtcttctctacaggtgttcacagtgatattcagatgactcagagtccatccagcatgtcagtctccgtgggagatagggtgacgataacctgtcattcaagccaagacatcaactccaatattggatggctccaacagaagcctggtaagtccttcaaaggactaatctatcacggaacaaacttggacgacggcgtgccatcgagattttcagggtctggcagcgggaccgactatacactgaccatctctagcttacaaccagaggactttgccacatactactgcgtccagtacgctcagttcccctggacattcggcggcggcacaaaactggaaatcaaacgtgagtagcggtccgttaattaaagatccttctaaactctgagggggtcggatgacgtggccattgttacttaaacaccatcctgtttgcttctttcctcaggaaccgtcgcagctccctccgtgttcatcttccccccatccgacgagcaactgaagtcaggcacagcctccgtggtgtgcctccttaataacttttacccaagagaggccaaagtccagtggaaagtggacaacgcactacagagcgggaactctcaggaaagcgtgacagagcaggactcaaaagattcaacatacagcctatcttctaccctgacactgtcaaaagctgattatgaaaagcacaaagtatatgcctgtgaagtaactcatcagggactcagcagccctgtcactaaaagttttaatagaggcgaatgctgataagcggccgtgcggacgaccgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaactccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtggtctgactgggttccacgaaacgtccaacatcaacgacttttctgctggtgtcgccaatcgcagtgccagcatccgcattccccggactgtcggccaggagaagaaaggttactttgaagaccgcggcccctctgccaattgtgacccctttgcagtgacagaagccatcgtccgcacatgccttctcaatgagactggcgacgagcccttccaatacaaaaactaattagactttgagtgatcttgagcctttcctagttcatcccaccccgccccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagcttcgtgtcaaggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacg

SEQ ID NO: 42 - protein, 135 aa, Artificial Sequence
  synthetic construct
MDWTWRILFLVAAATGVHSQVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 43 - protein, 330 aa, Artificial Sequence
  synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDECTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 6 aa, Artificial Sequence
  synthetic construct
SDFAWN

SEQ ID NO: 45 - protein, 16 aa, Artificial Sequence
  synthetic construct
YISYSGNTRYQPSLKS

SEQ ID NO: 46 - protein, 9 aa, Artificial Sequence
  synthetic construct
VTAGRGFPY

SEQ ID NO: 47 - protein, 127 aa, Artificial Sequence
  synthetic construct
MDWTWRILFLVAAATGVHSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKR

SEQ ID NO: 48 - protein, 106 aa, Artificial Sequence
  synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 11 aa, Artificial Sequence
  synthetic construct
HSSQDINSNIG

SEQ ID NO: 50 - protein, 7 aa, Artificial Sequence
  synthetic construct
HGTNLDD

SEQ ID NO: 51 - protein, 9 aa, Artificial Sequence
  synthetic construct
VQYAQFPWT

SEQ ID NO: 52 - DNA, 40 bp, Artificial Sequence
  synthetic oligonucleotide
gagaagcttgccgccaccatggattggacctggcgcattc

SEQ ID NO: 53 - DNA, 79 bp, Artificial Sequence
  synthetic oligonucleotide
cccttcctcctcactgggatttggcagccccttacctgtggcggctgctaccagaaagagaatgcgccaggtccaatcc

SEQ ID NO: 54 - DNA, 79 bp, Artificial Sequence
  synthetic oligonucleotide
cccagtgaggaggaagggatcgaaggtcaccatcgaagccagtcaagggggcttccatccactcctgtgtcttctctac

SEQ ID NO: 55 - DNA, 80 bp, Artificial Sequence
  synthetic oligonucleotide
gactcggcttgacaagcccaggtccactctcttggagctgcacctggctgtggacacctgtagagaagacacaggagtgg

SEQ ID NO: 56 - DNA, 84 bp, Artificial Sequence
  synthetic oligonucleotide
gggcttgtcaagccgagtcaaactttgtccctaacatgtactgtgtccggatactctatctcatcagattttgcgtggaattgg

SEQ ID NO: 57 - DNA, 80 bp, Artificial Sequence
  synthetic oligonucleotide
cccagagtatgatatgtagcccatccattctaaacctttccctggtggctgccttatccaattccacgcaaaatctgatg

SEQ ID NO: 58 - DNA, 79 bp, Artificial Sequence
  synthetic oligonucleotide
gggctacatatcatactctgggaacaccagatatcaaccctctctgaaaagccggatcacaatcactagggacacgtcg

SEQ ID NO: 59 - DNA, 83 bp, Artificial Sequence
  synthetic oligonucleotide
gcagtaatatgttgctgtgtctggggctgtaacggagttcagctgcaggaagaactggctcttcgacgtgtccctagtgattg

SEQ ID NO: 60 - DNA, 81 bp, Artificial Sequence
  synthetic oligonucleotide
ccagacacagcaacatattactgcgtaaccgctggcagaggcttcccctattggggacagggcaccctagtgacagtgagc

SEQ ID NO: 61 - DNA, 39 bp, Artificial Sequence
  synthetic oligonucleotide
cacggatccatcttaccgctgctcactgtcactagggtg

SEQ ID NO: 62 - DNA, 26 bp, Artificial Sequence
  synthetic oligonucleotide
gagaagcttgccgccaccatggattg

SEQ ID NO: 63 - DNA, 80 bp, Artificial Sequence
  synthetic oligonucleotide
ctgggatttggcagccccttacctgttgcggctgctacaagaaacagtattctccaagtccaatccatggtggcggcaag

SEQ ID NO: 64 - DNA, 78 bp, Artificial Sequence
  synthetic oligonucleotide
ggggctgccaaatcccagtgaggaggaagggatcgaaggtgaccatcgaagccagtcaagggggcttccatccactcc

SEQ ID NO: 65 - DNA, 77 bp, Artificial Sequence
  synthetic oligonucleotide
catgctggatggactctgagtcatctgaatatcactgtgaacacctgtagagaagacacaggagtggatggaagccc

SEQ ID NO: 66 - DNA, 80 bp, Artificial Sequence
  synthetic oligonucleotide
ctcagagtccatccagcatgtcagtctccgtgggagatagggtgacgataacctgtcattcaagccaagacatcaactcc

SEQ ID NO: 67 - DNA, 82 bp, Artificial Sequence
  synthetic oligonucleotide
gttccgtgatagattagtcctttgaaggacttaccaggcttctgttggagccatccaatattggagttgatgtcttggcttg

SEQ ID NO: 68 - DNA, 84 bp, Artificial Sequence
  synthetic oligonucleotide
caaaggactaatctatcacggaacaaacttggacgacggcgtgccatcgagattttcagggtctggcagcgggaccgactatac

SEQ ID NO: 69 - DNA, 76 bp, Artificial Sequence
  synthetic oligonucleotide
gtgctggacgcagtagtatgtggcaaagtcttctggctctaagctagagatggtcagtgtatagtcggtcccgctg

SEQ ID NO: 70 - DNA, 79 bp, Artificial Sequence
  synthetic oligonucleotide
catactactgcgtccagcacgctcagttcccctggacattcggcggcggcacaaaactggaaatcaaacgtgagtaggg

SEQ ID NO: 71 - DNA, 28 bp, Artificial Sequence
  synthetic oligonucleotide
ctcggatccctactcacgtttgatttcc

SEQ ID NO: 72 - DNA, 37 bp, Artificial Sequence
  synthetic oligonucleotide
gacggatccttctaaactctgagggggtcggatgacg

SEQ ID NO: 73 - DNA, 78 bp, Artificial Sequence
  synthetic oligonucleotide
ggagctgcgacggttcctgaggaaagaagcaaacaggatggtgtttaagtaacaatggccacgtcatccgaccccctc

SEQ ID NO: 74 - DNA, 78 bp, Artificial Sequence
  synthetic oligonucleotide
ggaaccgtcgcagctccctccgtgttcatcttccccccatccgacgagcaactgaagtcaggcacagcctccgtggtg

SEQ ID NO: 75 - DNA, 78 bp, Artificial Sequence
  synthetic oligonucleotide
gtgcgttgtccactttccactggactttggcctctcttgggtaaaagttattaaggaggcacaccacggaggctgtgc

SEQ ID NO: 76 - DNA, 83 bp, Artificial Sequence
  synthetic oligonucleotide
gtggaaagtggacaacgcactacagagcgggaactctcaggaaagcgtgacagagcaggactcaaaagattcaacatacagcc

SEQ ID NO: 77 - DNA, 88 bp, Artificial Sequence
  synthetic oligonucleotide
cttcacaggcatataccttgtgcttttcataatcagcttttgacagtgtcagggtagaagataggctgtatgttgaatcttttgagtc

SEQ ID NO: 78 - DNA, 71 bp, Artificial Sequence
  synthetic oligonucleotide
gcacaaggtatatgcctgtgaagtaactcatcagggactcagcagccctgtcactaaaagttttaatagag

SEQ ID NO: 79 - DNA, 51 bp, Artificial Sequence
  synthetic oligonucleotide
cctgcggccgcttatcagcattcgcctctattaaaacttttggtgagaggg

SEQ ID NO: 80 - DNA, 1128 bp, Artificial Sequence
  synthetic construct
aagatggcacaccgtggccggcctctgcgcctgggcccagctctgtcccacaccgcggtcacatggcaccttttctcttccagcctccaccaagggccccagcgtgttccccctggcccccagcagcaagagcaccagcggcggcacagccgccctgggctgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaattggtatgtggacggcgtggaggtgcacaacgccaagaccaagcccagagaagagcagtacaacagcacctacagggtggtgtccgtgctgaccgtgctgcaccaggactggctgaacggcaaggaatacaaatgcaaggtctccaacaaggccctgccagcccccatcgaaaagaccatcagcaaggccaagggccagccacgggagccccaggtgtacaccctgcccccctcccgggacgagtgcaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcccccggcaagtgatgacgacgcggccgtgcggacgaccgaattcattgatcataatcagccatacc

SEQ ID NO: 81 - protein, 465 aa, Artificial Sequence
  synthetic construct
MDWTWRILFLVAAATGVHSQVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDECTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 209 aa, Artificial Sequence
  synthetic construct
MDWTWRILFLVAAATGVHSDIQMTQSPSSMSVSVGDRVTITCHSSQDINSNIGWLQQKPGKSFKGLIYHGTNLDDGVPSRFSGSGSGTDYTLTISSLEPEDFATYYCVQHAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHK

SEQ ID NO: 83 - protein, 233 aa, Artificial Sequence
  synthetic construct
MDWTWRILFLVAAATGVHSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 7 aa, Mus musculus
CVQHAQF

SEQ ID NO: 85 - protein, 7 aa, Mus musculus
CVQYAQF

SEQ ID NO: 86 - DNA, 40 bp, Artificial Sequence
  synthetic primer
ccacatactactgcgtccagtacgctcagttcccctggac

SEQ ID NO: 87 - DNA, 20 bp, Artificial Sequence
  synthetic primer
ctggacgcagtagtatgtgg

SEQ ID NO: 88 - DNA, 26 bp, Artificial Sequence
  synthetic primer
gagaagcttgccgccaccatggattg

SEQ ID NO: 89 - DNA, 28 bp, Artificial Sequence
  synthetic primer
cactgggtgactggcttcgatggtgacc

SEQ ID NO: 90 - DNA, 49 bp, Artificial Sequence
  synthetic primer
ggtcaccatcgaagccagtcacccagtgaagggggcttccatccactcc

SEQ ID NO: 91 - DNA, 44 bp, Artificial Sequence
  synthetic primer
ccaagatctggccggccacggtgtgccatcttaccgctgctcac

SEQ ID NO: 92 - DNA, 22 bp, Artificial Sequence
  synthetic primer
gagaagcttgccgccaccatgg

SEQ ID NO: 93 - DNA, 25 bp, Artificial Sequence
  synthetic primer
cggtccgcccccttgactggcttcg

SEQ ID NO: 94 - DNA, 45 bp, Artificial Sequence
  synthetic primer
cgaagccagtcaagggggcggaccgcttccatccactcctgtgtc

SEQ ID NO: 95 - DNA, 50 bp, Artificial Sequence
  synthetic primer
ccaagatctttaattaacggaccgctactcacgtttgatttccagttttg

SEQ ID NO: 96 - protein, 7 aa, Mus musculus
SVTIEDT

SEQ ID NO: 97 - protein, 7 aa, Artificial Sequence
  synthetic construct
SVTAPDT

SEQ ID NO: 98 - protein, 7 aa, Artificial Sequence
  synthetic construct
SVTAADT

SEQ ID NO: 99 - DNA, 49 bp, Artificial Sequence
  synthetic primer
ctgcagctgaactccgttacagccgcagacacagcaacatattactgcg

SEQ ID NO: 100 - DNA, 49 bp, Artificial Sequence
  synthetic primer
cgcagtaatatgttgctgtgtctgcggctgtaacggagttcagctgcag

SEQ ID NO: 101 - protein, 12 aa, Artificial Sequence
  synthetic construct
TRDTSKSQFFLQ

SEQ ID NO: 102 - protein, 12 aa, Artificial Sequence
  synthetic construct
SRDTSKNQFFLK

SEQ ID NO: 103 - DNA, 49 bp, Artificial Sequence; identical to SEQ ID NO: 90
  synthetic primer

SEQ ID NO: 104 - DNA, 45 bp, Artificial Sequence
  synthetic primer
gattcttcgacgtgtcccttgagattgtgatccggcttttcagag

SEQ ID NO: 105 - DNA, 55 bp, Artificial Sequence
  synthetic primer
caagggacacgtcgaagaatcagttcttcctgaaactgaactccgttacagccgc

SEQ ID NO: 106 - DNA, 44 bp, Artificial Sequence; identical to SEQ ID NO: 91
  synthetic primer

SEQ ID NO: 107 - protein, 6 aa, Artificial Sequence
  synthetic construct
SSLEPE

SEQ ID NO: 108 - protein, 6 aa, Artificial Sequence
  synthetic construct
SSLQPE

SEQ ID NO: 109 - DNA, 45 bp, Artificial Sequence; identical to SEQ ID NO: 94
  synthetic primer

SEQ ID NO: 110 - DNA, 34 bp, Artificial Sequence
  synthetic primer
ctctggttgtaagctagagatggtcagtgtatag

SEQ ID NO: 111 - DNA, 45 bp, Artificial Sequence
  synthetic primer
ccatctctagcttacaaccagaggactttgccacatactactgcg

SEQ ID NO: 112 - DNA, 50 bp, Artificial Sequence; identical to SEQ ID NO: 95
  synthetic primer

SEQ ID NO: 113 - protein, 8 aa, Artificial Sequence
  synthetic construct
VYACEVTH

SEQ ID NO: 114 - DNA, 21 bp, Artificial Sequence
  synthetic primer
ggcggcacaaaactggaaatc

SEQ ID NO: 115 - DNA, 59 bp, Artificial Sequence
  synthetic primer
gatgagttacttcacaggcatatactttgtgcttttcataatcagcttttgacagtgtc

SEQ ID NO: 116 - DNA, 26 bp, Artificial Sequence
  synthetic primer
agtatatgcctgtgaagtaactcatc

SEQ ID NO: 117 - DNA, 17 bp, Artificial Sequence
  synthetic primer
gccacgatgcgtccggc

SEQ ID NO: 118 - DNA, 23 bp, Artificial Sequence
  synthetic primer
gcacttgatgtaattctccttgg

SEQ ID NO: 119 - DNA, 19 bp, Artificial Sequence
  synthetic primer
gaagtagtccttgaccagg

SEQ ID NO: 120 - DNA, 23 bp, Artificial Sequence
  synthetic primer
gaagatgaagacagatggtgcag

SEQ ID NO: 121 - DNA, 20 bp, Artificial Sequence
  synthetic primer
cggtggagggcagtgtagtc

SEQ ID NO: 122 - DNA, 21 bp, Artificial Sequence
  synthetic primer
gtgatgctattgctttatttg

SEQ ID NO: 123 - DNA, 21 bp, Artificial Sequence
  synthetic primer
catacctaccagttctgcgcc

SEQ ID NO: 124 - DNA, 21 bp, Artificial Sequence
  synthetic primer
ccatcctgtttgcttctttcc

SEQ ID NO: 125 - DNA, 17 bp, Artificial Sequence
  synthetic primer
gacagggctgctgagtc

SEQ ID NO: 126 - DNA, 22 bp, Artificial Sequence
  synthetic primer
gtgcagctccaagagagtggac

SEQ ID NO: 127 - DNA, 20 bp, Artificial Sequence
  synthetic primer
cagagtccatccagcatgtc

SEQ ID NO: 128 - DNA, 363 bp, Mus musculus
ttagtcaagctgcaggagtctggacctagcctggtgaaaccttctcagtctctgtccctcacctgcactgtcactggctactcaatcaccagtgactatgcctggaactggatccggcagtttccaggaaacaaactggagtggatgggctacataagttacagtgctaacactaggtacaacccatctctcaaaagtcgaatctctatcactcgagacacatccaagaaccaattcttcctgcagttgaattctgtgactactgaggacacagccacatattactgtgcaacggcgggacgcgggtttccttactggggccaagggactctggtcactgtctctgcagccaaaacgacaccc

SEQ ID NO: 129 - protein, 116 aa, Mus musculus
LVKLQESGPSLVKPSQSLSLTCTVTGYSITSDYAWNWIRQFPGNKLEWMGYISYSANTRYNPSLKSRISITRDTSKNQFFLQLNSVTTEDTATYYCATAGRGFPYWGQGTLVTVSA

SEQ ID NO: 130 - protein, 6 aa, Mus musculus
SDYAWN

SEQ ID NO: 131 - protein, 16 aa, Mus musculus
YISYSANTRYNPSLKS

SEQ ID NO: 132 - protein, 9 aa, Mus musculus
ATAGRGFPY

SEQ ID NO: 133 - DNA, 324 bp, Mus musculus
gacattgtgctgacccagtctccatcctccatgtctctatctctgggagacacagtcagtatcacttgccattcaagtcaggacattaacagtaatatagggtggttgcagcagaaaccagggaaatcatttaagggcctgatctatcatggaaccaacttggacgatggagttccatcaaggttcagtggcagtggatctggagccgattattctctcaccatcagcagcctggaatctgaagattttgtagactattactgtgtacagtatggtcagtttccgtggacgttcggtggaggcaccaagctggaaatcaaacgg

SEQ ID NO: 134 - protein, 107 aa, Mus musculus
DIVLTQSPSSMSLSLGDTVSITCHSSQDISNIGWLQQKPGKSFKGLIYHGTNLEDGVPSRFSGSGSGADYSLTISSLESEDFVDYYCVQYGQFPWTFGGGTKLEIKR

SEQ ID NO: 135 - protein, 11 aa, Mus musculus
HSSQDISSNIG

SEQ ID NO: 136 - protein, 7 aa, Mus musculus
HGTNLED

SEQ ID NO: 137 - protein, 10 aa, Mus musculus
CVQYGQFPWT

SEQ ID NO: 138 - protein, 16 aa, Artificial Sequence
  synthetic construct
CGADSYEMEEDGVRKC

SEQ ID NO: 139 - protein, 7 aa, Mus musculus
HGTNLDD

SEQ ID NO: 140 - protein, 7 aa, Mus musculus
HGTNLDD

SEQ ID NO: 141 - protein, 7 aa, Mus musculus
HGTNLED

SEQ ID NO: 142 - protein, 7 aa, Mus musculus
HGTNLDD

SEQ ID NO: 143 - protein, 11 aa, Mus musculus
GYSITSDFAWN

SEQ ID NO: 144 - protein, 17 aa, Mus musculus
GYISYSGNTRYNPSLKS

SEQ ID NO: 145 - protein, 11 aa, Mus musculus
GYSITSDYAWN

SEQ ID NO: 146 - protein, 17 aa, Artificial Sequence
  synthetic construct
GYISYSANTRYNPSLKS

SEQ ID NO: 147 - protein, 11 aa, Mus musculus
GYSITSDYAWN

SEQ ID NO: 148 - protein, 17 aa, Mus musculus
GYISYSANTRYNPSLKS

SEQ ID NO: 149 - protein, 11 aa, Mus musculus
GYSITSDYAWN

SEQ ID NO: 150 - protein, 17 aa, Mus musculus
GYISYSGNTRYNPSLRS

SEQ ID NO: 151 - protein, 13 aa, Artificial Sequence
  synthetic construct
HSSQDIXAASNIG

SEQ ID NO: 152 - protein, 9 aa, Artificial Sequence
  synthetic construct
HGTNLXAAD

SEQ ID NO: 153 - protein, 11 aa, Artificial Sequence
  synthetic construct
VQYXAAQFPWT

SEQ ID NO: 154 - protein, 8 aa, Artificial Sequence
  synthetic construct
SDXAAAWN

SEQ ID NO: 155 - protein, 18 aa, Artificial Sequence
  synthetic construct
YISYSGNTRYXAAPSLKS

SEQ ID NO: 156 - protein, 18 aa, Artificial Sequence
  synthetic construct
YISYSXAANTRYNPSLKS

SEQ ID NO: 157 - protein, 18 aa, Artificial Sequence
  synthetic construct
YISYSGNTRYNPSLXAAS

SEQ ID NO: 158 - protein, 11 aa, Artificial Sequence
  synthetic construct
XAATAGRGFPY

SEQ ID NO: 159 - protein, 18 aa, Artificial Sequence
  synthetic construct
YISYSGNTRYXAAPSLKS

SEQ ID NO: 160 - protein, 128 aa, Artificial Sequence
  synthetic vector
MVSTAQFLAFLLLWFPGARCDILMTQSPSSMSVSLGDTVSITCHSSQDINSNIGWLQQRPGKSFKGLIYHGTNLDDEVPSRFSGSGSGADYSLTISSLESEDFADYYCVQHAQFPWTFGGGTKLEIKR

SEQ ID NO: 161 - protein, 134 aa, Artificial Sequence
  synthetic vector
MRVLILLWLFTAFPGVLSDVQLQESGPSLVKPSQTLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 162 - DNA, 11891 bp, Artificial Sequence
  plasmid
ttcgaacggcggtggtacctaacctggaccgcgtaagagaaagaccatcgtcggcggtgtccattccccgacggtttagggtcactcctccttccctagcttccagtggtagcttcggtcagtgggtcacttcccccgaaggtaggtgaggacacagaagagatgtccacaggtgtcggtccacgtcgaggttctctcacctggacccgaacagttcggctcagtttgaaacagggattgtacatgacacaggcctatgagatagagtagtctaaaacgcaccttaacctattccgtcggtggtccctttccaaatcttacctacccgatgtatagtatgagacccttgtggtctatagttggaagagacttttcggcctagtgttagagttccctgtgcagcttcttagtcaagaaggactttgacttgaggcaatgtcggcgtctgtgtcgttgtataatgacgcattggcgaccgtctccgaaggggataacccctgtcccgtgggatcactgtcactcgtcgccattctaccgtgtggcaccggccggagacgcggacccgggtcgagacagggtgtggcgccagtgtaccgtggaaaagagaaggtcggaggtggttcccggggtcgcacaagggggaccgggggtcgtcgttctcgtggtcgccgccgtgtcggcgggacccgacggaccacttcctgatgaaggggctcgggcactggcactcgaccttgtcgcctcgggactggaggccgcacgtgtggaaggggcggcacgacgtctcgtcgccggacatgtcggactcgtcgcaccactggcacgggtcgtcgtcggacccgtgggtctggatgtagacgttgcacttggtgttcgggtcgttgtggttccacctgttcttccacctcgggttctcgacgctgttctgggtgtggacgggggggacgggtcggggtctcgacgacccgcctgggaggcacaaggacaagggggggttcgggttcctgtgggactactagtcgtcctgggggctccactggacgcaccaccacctgcactcggtgctcctgggtctccacttcaagttaaccatacacctgccgcacctccacgtgttgcggttctggttcgggtctcttctcgtcatgttgtcgtggatgtcccaccacaggcacgactggcacgacgtggtcctgaccgacttgccgttccttatgtttacgttccagaggttgttccgggacggtcgggggtagcttttctggtagtcgttccggttcccggtcggtgccctcggggtccacatgtgggacggggggagggccctgctcacgtggttcttggtccacagggactggacagaccacttcccgaagatggggtcgctgtagcggcacctcaccctctcgttgccggtcgggctcttgttgatgttctggtgggggggtcacgacctgtcgctgccgtcgaagaaggacatgtcgttcgactggcacctgttctcgtccaccgtcgtcccgttgcacaagtcgacgtcgcactacgtgctccgggacgtgttggtgatgtgggtcttctcggactcggacagggggccgttcactactgctgcgccggcacgcctgctggcttaagtaactagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaagtgacgtaagatcaacaccaaacaggtttgagtagttacatagaatagtacagaccgccggcggctataaacttttataccgtataacttttacagcggctacactcaaagacacattgactatagcggtaaaaaggttttcactaaaaacccgtatgcgctatagaccgctatcgcgaatatagcaaatgccccctaccgctatctgctgaaaccactgaacccgctaagacacacagcgtttatagcgtcaaagctatatccactgtctgctatactccgatatagcggctatctccgctgtagttcgaccgtgtaccggttacgtatagctagatatgtaacttagttataaccggtaatcggtataataagtaaccaatatatcgtatttagttataaccgataaccggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactgcattcatggcggatatctcagatatccgggtgggggaaccgaagaatacgtacgatatgacaaaaaccgaaccccagatatgtgggggcgaaggagtacaatatccactaccatatcgaatcggatatccacacccaataactggtaataactggtgaggggataaccactgctatgaaaggtaatgattaggtattgtaccgagaaacggtgttgagagaaataaccgatatacggttatgtgacaggaagtctctgactgtgcctgagacataaaaatgtcctaccccagagtaaataataaatgtttaagtgtatatgttgtggtggcaggggtcacgggcgtcaaaaataatttgtattgcaccctagaggtgcgcttagagcccatgcacaaggcctgtacccgagaagaggccatcgccgcctcgaagatgtaggctcgggacgagggtacggaggtcgctgagtaccagcgagccgtcgaggaacgaggattgtcacctccggtctgaatccgtgtcgtgctacgggtggtggtggtcacacggcgtgttccggcaccgccatcccatacacagacttttactcgagcccctcgcccgaacgtggcgactgcgtaaaccttctgaattccgtcgccgtcttcttctacgtccgtcgactcaacaacacaagactattctcagtctccattgagggcaacgccacgacaattgccacctcccgtcacatcagactcgtcatgagcaacgacggcgcgcgcggtggtctgtattatcgactgtctgattgtctgacaaggaaaggtacccagaaaagacgtcagtggcaggaactgtgcttcgaacggcggtggtacctaacctgaacctcttatgacaaagaacatcgtcggcgttgtccattccccgacggtttagggtcactcctccttccctagcttccactggtagcttcggtcagttcccccgcctggcgaaggtaggtgaggacacagaagagatgtccacaagtgtcactataagtctactgagtctcaggtaggtcgtacagtcagaggcaccctctatcccactgctattggacagtaagttcggttctgtagttgaggttataacctaccgaggttgtcttcggaccattcaggaagtttcctgattagatagtgccttgtttgaacctgctgccgcacggtagctctaaaagtcccagaccgtcgccctggctgatatgtgactggtagagatcgaatgttggtctcctgaaacggtgtatgatgacgcaggtcatgcgagtcaaggggacctgtaagccgccgccgtgttttgacctttagtttgcactcatcgccaggcaattaatttctaggaagatttgagactcccccagcctactgcaccggtaacaatgaatttgtggtaggacaaacgaagaaaggagtccttggcagcgtcgagggaggcacaagtagaaggggggtaggctgctcgttgacttcagtccgtgtcggaggcaccacacggaggaattattgaaaatgggttctctccggtttcaggtcacctttcacctgttgcgtgatgtctcgcccttgagagtcctttcgcactgtctcgtcctgagttttctaagttgtatgtcggatagaagatgggactgtgacagttttcgactaatacttttcgtgtttcatatacggacacttcattgagtagtccctgagtcgtcgggacagtgattttcaaaattatctccgcttacgactattcgccggcacgcctgctggcttaagtaactagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaagtgacgtaagatcaacaccaaacaggtttgagtagttacatagaatagtacagacctaggagatgcggcctgcgtagcaccggccgtagtggccgcggtgtccacgccaacgaccgcggatatagcggctgtagtggctaccccttctagcccgagcggtgaagcccgagtactcgcgaacaaagccgcacccataccaccgtccggggcaccggccccctgacaacccgcggtagaggaacgtacgtggtaaggaacgccgccgccacgagttgccggagttggatgatgacccgacgaaggattacgtcctcagcgtattccctctcgcagctggagcccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggactaccgagaaacgccgtgggtagcaagcattacaaggcaccgtggctcctgttgggagttctcttttacattagtgtgaccgagtggaagcccacccggaaagacgcaaatattcctctgtgaaatacaaattcttccaaccatttaaggaacgccgaaaccgtcggttcgatctaggccgacaccttacacacagtcaatcccacacctttcaggggtccgaggggtcgtccgtcttcatacgtttcgtacgtagagttaatcagtcgttggtccacacctttcaggggtccgaggggtcgtccgtcttcatacgtttcgtacgtagagttaatcagtcgttggtatcagggcggggattgaggcgggtagggcggggattgaggcgggtcaaggcgggtaagaggcggggtaccgactgattaaaaaaaataaatacgtctccggctccggcggagccggagactcgataaggtcttcatcactcctccgaaaaaacctccggatccgaaaacgtttttcgatcgaaccccggtggcgagtctcgtggaaggtggtaccggtggagtcgttcaagggtgaacttgtttttgtagttcgtttacatgaacacggacggggtcccactctttcaggttcggtacatatagacccaactaccatgacctcttcctgacgcgacgttttgggcgtgggacctgacactcgggttcacacatcttctcaatggactcaccttaaaactaccgagatcatggaaagtcagactcccgaggttgtcactgtacatagagtcgggacaacggtacaaagccctggggaaggcgtctctagggttgttcgaccacaagacacttcaaaagttcatgttggccttcggacgtctctggttaaattccgtgagcacatttgcctattacctgtaccactcgttggtcgtggggaccaaaccttaccttgtcctcatatgagactacccttgtctacccgtgggaaaaccaaccggaaggttaccgaaaggacccggggttccaggcataatgacaccacacccgcgtctgtttcggataccgtccctatagcacctccgagtgatggcgcggacgaacatacgaccccagttctaatgtccttgtttacgactccagtacggacgggtcacccttgaggtttatcctgggacacttccttaggcgtaccctctagtagagacccaccgggcaaagtagaacgtagctcatacacttctgaaaccccattatcgttggaaactggggttcgggtaaggacccttgaccttaccacgtccgacggtatggttgaaatcgtggttccggtacgccctcctcttaccagacttcgtgtagctcctccggtagctctttgattcgttcgccgtggccatggtgtaagctcggatgctagggttccccccggacctgttacgggcaccagactgacccaaggtgctttgcaggttgtagttgctgaaaagacgaccacagcggttagcgtcacggtcgtaggcgtaaggggcctgacagccggtcctcttctttccaatgaaacttctggcgccggggagacggttaacactggggaaacgtcactgtcttcggtagcaggcgtgtacggaagagttactctgaccgctgctcgggaaggttatgtttttgattaatctgaaactcactagaactcggaaaggatcaagtagggtggggcggggtctctctagaaacacttccttggaatgaagacaccacactgtattaacctgtttgatggatgtctctaaatttcgagattccatttatattttaaaaattcacatattacacaatttgatgactaagattaacaaacacataaaatctaaggttggataccttgactacttaccctcgtcaccaccttacggaaattactccttttggacaaaacgagtcttctttacggtagatcactactactccgatgacgactgagagttgtaagatgaggaggttttttcttctctttccatcttctggggttcctgaaaggaagtcttaacgattcaaaaaactcagtacgacacaaatcattatcttgagaacgaacgaaacgataaatgtggtgtttcctttttcgacgtgacgatatgttcttttaatacctttttataagacattggaaatattcatccgtattgtcaatattagtattgtatgacaaaaaagaatgaggtgtgtccgtatctcacagacgataattattgatacgagtttttaacacatggaaatcgaaaaattaaacatttccccaattattccttataaactacatatcacggaactgatctctagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaagtgacgtaagatcaacaccaaacaggtttgagtagttacatagaatagtacagacctagatcgaagcacagttcctgccactgacgtcacttattattttacacacaaacaggctttatgcgcaaaactctaaagacagcggctgatttaagtacagcgcgctatcaccacaaatagcggctatctctaccgctataacctttttagctataaacttttataccgtataacttttacagcggctacactcaaagacacattgactatagcggtaaaaaggttttcactaaaaacccgtatgcgctatagaccgctatcgcgaatatagcaaatgccccctaccgctatctgctgaaaccactgaacccgctaagacacacagcgtttatagcgtcaaagctatatccactgtctgctatactccgatatagcggctatctccgctgtagttcgaccgtgtaccggttacgtatagctagatatgtaacttagttataaccggtaatcggtataataagtaaccaatatatcgtatttagttataaccgataaccggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactgcattcatggcggatatctcagatatccgggtgggggaaccgaagaatacgtacgatatgacaaaaaccgaaccccagatatgtgggggcgaaggagtacaatatccactaccatatcgaatcggatatccacacccaataactggtaataactggtgaggggataaccactgctatgaaaggtaatgattaggtattgtaccgagaaacggtgttgagagaaataaccgatatacggttatgtgacaggaagtctctgactgtgcctgagacataaaaatgtcctaccccagagtaaataataaatgtttaagtgtatatgttgtggtggcaggggtcacgggcgtcaaaaataatttgtattgcaccctagaggtgcgcttagagcccatgcacaaggcctgtacccgagaagaggccatcgccgcctcgaagatgtaggctcgggacgagggtacggaggtcgctgagtaccagcgagccgtcgaggaacgaggattgtcacctccggtctgaatccgtgtcgtgctacgggtggtggtggtcacacggcgtgttccggcaccgccatcccatacacagacttttactcgagcccctcgcccgaacgtggcgactgcgtaaaccttctgaattccgtcgccgtcttcttctacgtccgtcgactcaacaacacaagactattctcagtctccattgagggcaacgccacgacaattgccacctcccgtcacatcagactcgtcatgagcaacgacggcgcgcgcggtggtctgtattatcgactgtctgattgtctgacaaggaaaggtacccagaaaagacgtcagtggcaggaactgtgc

SEQ ID NO: 163 - protein, 19 aa, Artificial Sequence
  synthetic construct
MDWTWRILFLVAAATGVHS

SEQ ID NO: 164 - protein, 116 aa, Artificial Sequence
  synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 165 - protein, 19 aa, Artificial Sequence
  synthetic construct
MDWTWRILFLVAAATGVHS

SEQ ID NO: 166 - protein, 108 aa, Artificial Sequence
  synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKR

SEQ ID NO: 167 - protein, 116 aa, Mus musculus
DVQLQESGPSLVKPSQTLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 168 - protein, 116 aa, Artificial Sequence
  synthetic construct
QVQLQESGPGLVKPSQTLSLTCTVSGYSISSDFAWNWIRQPPGKGLEWMGYISYSGNTRYQPSLKSRITITRDTSKSQFFLQLNSVTAPDTATYYCVTAGRGFPYWGQGTLVTVSS

SEQ ID NO: 169 - DNA, 80 bp, Artificial Sequence
  synthetic oligonucleotide
acctaacctggaccgcgtaagagaaagaccatcgtcggcggtgtccattccccgacggtttagggtcactcctccttccc

SEQ ID NO: 170 - DNA, 81 bp, Artificial Sequence
  synthetic oligonucleotide
atcccagtgaggaggaagggatcgaaggtcaccatcgaagccagtcaagggggcttccatccactcctgtgtcttctctac

SEQ ID NO: 171 - DNA, 80 bp, Artificial Sequence
  synthetic oligonucleotide
ggtgaggacacagaagagatgtccacaggtgtcggtccacgtcgaggttctctcacctggacccgaacagttcggctcag

SEQ ID NO: 172 - DNA, 85 bp, Artificial Sequence
  synthetic oligonucleotide
tgggcttgtcaagccgagtcaaactttgtccctaacatgtactgtgtccggatactctatctcatcagattttgcgtggaattgg

SEQ ID NO: 173 - DNA, 82 bp, Artificial Sequence
  synthetic oligonucleotide
gagtagtctaaaacgcaccttaacctattccgtcggtggtccctttccaaatcttacctacccgatgtatagtatgagaccc

SEQ ID NO: 174 - DNA, 80 bp, Artificial Sequence
  synthetic oligonucleotide
gggctacatatcatactctgggaacaccagatatcaaccctctctgaaaagccggatcacaatcactagggacacgtcga

SEQ ID NO: 175 - DNA, 83 bp, Artificial Sequence
  synthetic oligonucleotide
gttagtgatccctgtgcagcttctcggtcaagaaggacgtcgacttgaggcaatgtcggggtctgtgtcgttgtataatgacg

SEQ ID NO: 176 - DNA, 82 bp, Artificial Sequence
  synthetic oligonucleotide
ccagacacagcaacatattactgcgtaaccgctggcagaggcttcccctattggggacagggcaccctagtgacagtgagca

SEQ ID NO: 177 - DNA, 39 bp, Artificial Sequence
  synthetic oligonucleotide
gtgggatcactgtcactcgtcgccattctacctaggcac

SEQ ID NO: 178 - DNA, 1128 bp, Artificial Sequence
  synthetic construct
ttctaccgtgtggcaccggccggagacgcggacccgggtcgagacagggtgtggcgccagtgtaccgtggaaaagagaaggtcggaggtggttcccggggtcgcacaagggggaccgggggtcgtcgttctcgtggtcgccgccgtgtcggcgggacccgacggaccacttcctgatgaaggggctcgggcactggcactcgaccttgtcgcctcgggactggaggccgcacgtgtggaaggggcggcacgacgtctcgtcgccggacatgtcggactcgtcgcaccactggcacgggtcgtcgtcggacccgtgggtctggatgtagacgttgcacttggtgttcgggtcgttgtggttccacctgttcttccacctcgggttctcgacgctgttctgggtgtggacgggggggacgggtcggggtctcgacgacccgcctgggaggcacaaggacaagggggggttcgggttcctgtgggactactagtcgtcctgggggctccactggacgcaccaccacctgcactcggtgctcctgggtctccacttcaagttaaccatacacctgccgcacctccacgtgttgcggttctggttcgggtctcttctcgtcatgttgtcgtggatgtcccaccacaggcacgactggcacgacgtggtcctgaccgacttgccgttccttatgtttacgttccagaggttgttccgggacggtcgggggtagcttttctggtagtcgttccggttcccggtcggtgccctcggggtccacatgtgggacggggggagggccctgctcacgtggttcttggtccacagggactggacagaccacttcccgaagatggggtcgctgtagcggcacctcaccctctcgttgccggtcgggctcttgttgatgttctggtgggggggtcacgacctgtcgctgccgtcgaagaaggacatgtcgttcgactggcacctgttctcgtccaccgtcgtcccgttgcacaagtcgacgtcgcactacgtgctccgggacgtgttggtgatgtgggtcttctcggactcggacagggggccgttcactactgctgcgccggcacgcctgctggcttaagtaactagtattagtcggtatgg

SEQ ID NO: 179 - protein, 107 aa, Mus musculus
DILMTQSPSSMSVSLGDTVSITCHSSQDINSNIGWLQQRPGKSFKGLIYHGTNLDDEVPSRFSGSGSGADYSLTISSLESEDFADYYCVQHAQFPWTFGGGTKLEIK

SEQ ID NO: 180 - protein, 107 aa, Artificial Sequence
  synthetic construct
DILMTQSPSSMSVSLGDTVSITCHSSQDINSNIGWLQQRPGKSFKGLIYHGTNLDDEVPSRFSGSGSGTDYTLTISSLEPEDFATYYCVQYAQFPWTFGGGTKLEIK

SEQ ID NO: 181 - DNA, 80 bp, Artificial Sequence
  synthetic oligonucleotide
gaacggcggtggtacctaacctgaacctcttatgacaaagaacatcgtcggcgttgtccattccccgacggtttagggtc

SEQ ID NO: 182 - DNA, 80 bp, Artificial Sequence
  synthetic oligonucleotide
aaggggctgccaaatcccagtgaggaggaagggatcgaaggtgaccatcgaagccagtcaagggggcttccatccactcc

SEQ ID NO: 183 - DNA, 80 bp, Artificial Sequence
  synthetic oligonucleotide
tcccccgaaggtaggtgaggacacagaagagatgtccacaagtgtcactataagtctactgagtctcaggtaggtcgtac

SEQ ID NO: 184 - DNA, 82 bp, Artificial Sequence
  synthetic oligonucleotide
gttcggttctgtagttgaggttataacctaccgaggttgtcttcggaccattcaggaagtttcctgattagatagtgccttg

SEQ ID NO: 185 - DNA, 80 bp, Artificial Sequence
  synthetic oligonucleotide
gaccgtcgccctggctgatatgtgactggtagagatcgaatctcggtcttctgaaacggtgtatgatgacgcaggtcgtg

SEQ ID NO: 186 - DNA, 80 bp, Artificial Sequence
  synthetic oligonucleotide
catactactgcgtccagcacgctcagttcccctggacattcggcggcggcacaaaactggaaatcaaacgtgagtaggga

SEQ ID NO: 187 - DNA, 28 bp, Artificial Sequence
  synthetic oligonucleotide
cctttagtttgcactcatccctaggctc

SEQ ID NO: 188 - protein, 233 aa, Artificial Sequence
  synthetic construct
MDWTWRILFLVAAATGVHSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNVYPREAKVQWKVDNALQSGNSQESVTEQDSKDSTYSLSSTLTLSKADYEKGKVYACEVTHQHLSSPVTKSFNRGEC

SEQ ID NO: 189 - DNA, 704 bp, Artificial Sequence
  synthetic construct
atggtgtccacagctcagttccttgcattcttgttgctttggtttccaggtgcaagatgtgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttg

SEQ ID NO: 190 - DNA, 702 bp, Artificial Sequence
  synthetic construct
atggattggacttggagaatactgtttcttgtagcagccgcaacaggtgttcacagtgatattcagatgactcagagtccatccagcatgtcagtctccgtgggagatagggtgacgataacctgtcattcaagccaagacatcaactccaatattggatggctccaacagaagcctggtaagtccttcaaaggactaatctatcacggaacaaacttggacgacggcgtgccatcgagattttcagggtctggcagcgggaccgactatacactgaccatctctagcttacaaccagaggactttgccacatactactgcgtccagtacgctcagttcccctggacattcggcggcggcacaaaactggaaatcaaacgaaccgtcgcagctccctccgtgttcatcttccccccatccgacgagcaactgaagtcaggcacagcctccgtggtgtgcctccttaataacttttacccaagagaggccaaagtccagtggaaagtggacaacgcactacagagcgggaactctcaggaaagcgtgacagagcaggactcaaaagattcaacatacagcctatcttctaccctgacactgtcaaaagctgattatgaaaagcacaaagtatatgcctgtgaagtaactcatcagggactcagcagccctgtcactaaaagttttaatagaggcgaatgctga

SEQ ID NO: 191 - DNA, 408 bp, Artificial Sequence
  synthetic construct
gccaccatgagagtgctgattcttttgtggctgttcacagcctttcctggtgtcctgtctgatgtgcagcttcaggagtcgggacctagcctggtgaaaccttctcagactctgtccctcacctgcactgtcactggctactcaatcaccagtgattttgcctggaactggatccggcagtttccaggaaacaagctggagtggatgggctacataagttatagtggtaacactaggtacaacccatctctcaaaagtcgaatctctatcactcgagacacatccaagaaccaattcttcctgcagttgaattctgtgactattgaggacacagccacatattactgtgtaacggcgggacgcgggtttccttattggggccaagggactctggtcactgtctctgca

SEQ ID NO: 192 - DNA, 405 bp, Artificial Sequence
  synthetic construct
atggattggacctggcgcattctctttctggtagcagccgccacaggtgtccacagccaggtgcagctccaagagagtggacctgggcttgtcaagccgagtcaaactttgtccctaacatgtactgtgtccggatactctatctcatcagattttgcgtggaattggataaggcagccaccagggaaaggtttagaatggatgggctacatatcatactctgggaacaccagatatcaaccttctctgaaaagccggatcacaatctcaagggacacgtcgaagaatcagttcttcctgaaactgaactccgttacagccgcagacacagcaacatattactgcgtaaccgctggcagaggcttcccctattggggacagggcaccctagtgacagtgagcagc

SEQ ID NO: 193 - protein, 8 aa, Mus musculus
YHGTNLDD

SEQ ID NO: 194 - protein, 8 aa, Mus musculus
YHGTNLED

SEQ ID NO: 195 - protein, 9 aa, Mus musculus
VQYAQFPWT

(19) United States
(12) Patent Application Publication
(10) Pub. No.: US 2011/0076232 A1
(43) Pub. Date: Mar. 31, 2011

(54) SPECIFIC BINDING PROTEINS AND USES THEREOF

(75) Inventors: Lloyd J. Old, New York, NY (US); Terrance Grant Johns, Clayton (AU); Con Panousis, Melbourne (AU); Andrew Mark Scott, Kew (AU); Christoph Renner, Homburg/Saar (DE); Achim Jungbluth, New York, NY (US); Elizabeth Stockert, Wien (AT); Vincent Peter Collins, Cambridge (GB); Webster K. Cavenee, Del Mar, CA (US); Huei-Jen Su Huang, Rancho Santa Fe, CA (US); Antony Wilks Burgess, Camberwell (AU); Edouard Collins Nice, St. Kilda (AU); Anne Murray, New York, NY (US); George Mark, New York, NY (US); Gerd Ritter, New York, NY (US)

(73) Assignee: LUDWIG INSTITUTE FOR CANCER RESEARCH, Abbott Park, IL (US)

(21) Appl. No.: 12/890,029

(22) Filed: Sep. 24, 2010

Related U.S. Application Data
(60) Provisional application No. 61/397,697, filed on Sep. 29, 2009.

(51) Int. Cl. A61K 51/10; C07K 16/28; A61K 39/395; A61P 35/00

(52) U.S. Cl. 424/1.49; 530/387.9; 530/387.3; 424/139.1; 424/178.1; 424/179.1; 530/389.1

(57) ABSTRACT

The present invention relates to specific binding members, particularly antibodies and fragments thereof, which bind to amplified epidermal growth factor receptor (EGFR) and to the de2-7 EGFR truncation of the EGFR. In particular, the epitope recognized by the specific binding members, particularly antibodies and fragments thereof, is enhanced or evident upon aberrant post-translational modification. These specific binding members are useful in the diagnosis and treatment of cancer. The binding members of the present invention may also be used in therapy in combination with chemotherapeutics or anti-cancer agents and/or with other antibodies or fragments thereof.

### RELATED APPLICATION DATA

This application hereby claims the benefit of U.S. patent application Ser. No. ______, filed Sep. 29, 2009, the disclosure of which is hereby incorporated by reference in its entirety.

### FIELD OF THE INVENTION

The present invention relates to specific binding members, particularly antibodies and fragments thereof, which bind to amplified epidermal growth factor receptor (EGFR) and to the in-frame deletion of exons 2 to 7 of EGFR, resulting in a truncated EGFR receptor missing 267 amino acids from the extracellular domain (de2-7 EGFR). In particular, the epitope recognized by the specific binding members, particularly antibodies and fragments thereof, is enhanced or evident upon aberrant post-translational modification. These specific binding members are useful in the diagnosis and treatment of cancer. The binding members of the present invention may also be used in therapy in combination with chemotherapeutics or anti-cancer agents and/or with other antibodies or fragments thereof.

### BACKGROUND OF RELATED TECHNOLOGY

The treatment of proliferative disease, particularly cancer, by chemotherapeutic means often relies upon exploiting differences in target proliferating cells and other normal cells in the human or animal body. For example, many chemical agents are designed to be taken up by rapidly replicating DNA so that the process of DNA replication and cell division is disrupted. Another approach is to identify antigens on the surface of tumor cells or other abnormal cells which are not normally expressed in developed human tissue, such as tumor antigens or embryonic antigens. Such antigens can be targeted with binding proteins such as antibodies which can block or neutralize the antigen. In addition, the binding proteins, including antibodies and fragments thereof, may deliver a toxic agent or other substance which is capable of directly or indirectly activating a toxic agent at the site of a tumor.

The EGFR is an attractive target for tumor-targeted antibody therapy because it is over-expressed in many types of epithelial tumors (Voldborg et al. (1997). Epidermal growth factor receptor (EGFR) and EGFR mutations, function and possible role in clinical trials. Ann Oncol. 8, 1197-206; den Eynde, B. and Scott, A. M. Tumor Antigens. In: P. J. Delves and I. M. Roitt (eds.), Encyclopedia of Immunology, Second Edition, pp. 2424-31. London: Academic Press (1998)). Moreover, expression of the EGFR is associated with poor prognosis in a number of tumor types including stomach, colon, urinary bladder, breast, prostate, endometrium, kidney and brain (e.g., glioma). Consequently, a number of EGFR antibodies have been reported in the literature with several undergoing clinical evaluation (Baselga et al. (2000) Phase I Studies of Anti-Epidermal Growth Factor Receptor Chimeric Antibody C225 Alone and in Combination With Cisplatin. J. Clin. Oncol. 18, 904; Fullot et al. (1996): A phase I study of an anti-epidermal growth factor receptor monoclonal antibody for the treatment of malignant gliomas. Neurosurgery. 39, 478-83; Seymour, L. (1999) Novel anti-cancer agents in development: exciting prospects and new challenges. Cancer Treat. Rev. 25, 301-12)).

Results from studies using EGFR mAbs in patients with head and neck cancer, squamous cell lung cancer, brain gliomas and malignant astrocytomas have been encouraging. The antitumor activity of most EGFR antibodies is enhanced by their ability to block ligand binding (Sturgis et al. (1994) Effects of antiepidermal growth factor receptor antibody 528 on the proliferation and differentiation of head and neck cancer. Otolaryngol. Head Neck. Surg. 111, 633-43; Goldstein et al. (1995) Biological efficacy of a chimeric antibody to the epidermal growth factor receptor in a human tumor xenograft model. Clin. Cancer Res. 1, 1311-8). Such antibodies may mediate their efficacy through both modulation of cellular proliferation and antibody dependent immune functions (e.g. complement activation). The use of these antibodies, however, may be limited by uptake in organs that have high endogenous levels of EGFR such as the liver and skin (Baselga et al., 2000; Faillot et al., 1996).

A significant proportion of tumors containing amplifications of the EGFR gene (i.e., multiple copies of the EGFR gene) also co-express a truncated version of the receptor (Wikstrand et al. (1998) The class III variant of the epidermal growth factor receptor (EGFR): characterization and utilization as an immunotherapeutic target. J. Neurovirol. 4, 148-158) known as de2-7 EGFR, ΔEGFR, or Δ2-7 (terms used interchangeably herein) (Olapade-Olaopa et al. (2000) Evidence for the differential expression of a variant EGF receptor protein in human prostate cancer. Br. J. Cancer. 82, 186-94). The rearrangement seen in the de2-7 EGFR results in an in-frame mature mRNA lacking 801 nucleotides spanning exons 2-7 (Wong et al. (1992) Structural alterations of the epidermal growth factor receptor gene in human gliomas. Proc. Natl. Acad. Sci. U.S.A. 89, 2965-9; Yamazaki et al. (1990) A deletion mutation within the ligand binding domain is responsible for activation of epidermal growth factor receptor gene in human brain tumors. Jpn. J. Cancer Res. 81, 773-9; Yamazaki et al. (1988) Amplification of the structurally and functionally altered epidermal growth factor receptor gene (c-erbB) in human brain tumors. Mol. Cell. Biol. 8, 1816-20; Sugawa et al. (1990) Identical splicing of aberrant epidermal growth factor receptor transcripts from amplified rearranged genes in human glioblastomas. Proc. Natl. Acad. Sci. U.S.A. 87, 8602-6). The corresponding EGFR protein has a 267 amino acid deletion comprising residues 6-273 of the extracellular domain and a novel glycine residue at the fusion junction (Sugawa et al., 1990). This deletion, together with the insertion of a glycine residue, produces a unique junctional peptide at the deletion interface (Sugawa et al., 1990).

The de2-7 EGFR has been reported in a number of tumor types including glioma, breast, lung, ovarian and prostate (Wikstrand et al. (1997) Cell surface localization and density of the tumor-associated variant of the epidermal growth factor receptor, EGFRvIII. Cancer Res. 57, 4130-40; Olapade-Olaopa et al. (2000) Evidence for the differential expression of a variant EGF receptor protein in human prostate cancer. Br. J. Cancer. 82, 186-94; Wikstrand, et al. (1995) Monoclonal antibodies against EGFRvIII in are tumor specific and react with breast and lung carcinomas and malignant gliomas. Cancer Res. 55, 3140-8; Garcia de Palazzo et al. (1993) Expression of mutated epidermal growth factor receptor by non-small cell lung carcinomas. Cancer Res. 53, 3217-20). While this truncated receptor does not bind ligand, it possesses low constitutive activity and imparts a significant growth advantage to glioma cells grown as tumor xenografts in nude mice (Nishikawa et al. (1994) A mutant epidermal growth factor receptor common in human glioma confers enhanced tumorigenicity. Proc. Natl. Acad. Sci. U.S.A. 91, 7727-31) and is able to transform NIH3T3 cells (Batra et al. (1995) Epidermal growth factor ligand independent, unregulated, cell-transforming potential of a naturally occurring human mutant EGFRvIII gene. Cell Growth Differ. 6, 1251-9) and MCF-7 cells. The cellular mechanisms utilized by the de2-7 EGFR in glioma cells are not fully defined but are reported to include a decrease in apoptosis (Nagane et al. (1996) A common mutant epidermal growth factor receptor confers enhanced tumorigenicity on human glioblastoma cells by increasing proliferation and reducing apoptosis. Cancer Res. 56, 5079-86) and a small enhancement of proliferation (Nagane et al., 1996).

As expression of this truncated receptor is restricted to tumor cells it represents a highly specific target for antibody therapy. Accordingly, a number of laboratories have reported the generation of both polyclonal (Humphrey et al. (1990) Anti-synthetic peptide antibody reacting at the fusion junction of deletion mutant epidermal growth factor receptors in human glioblastoma. Proc. Natl. Acad. Sci. U.S.A. 87, 4207-11) and monoclonal (Wikstrand et al. (1995) Monoclonal antibodies against EGFRvIII are tumor specific and react with breast and lung carcinomas and malignant gliomas; Okamoto et al. (1996) Monoclonal antibody against the fusion junction of a deletion-mutant epidermal growth factor receptor. Br. J. Cancer. 73, 1366-72; Hills et al. (1995) Specific targeting of a mutant, activated EGF receptor found in glioblastoma using a monoclonal antibody. Int. J. Cancer. 63, 537-43) antibodies specific to the unique peptide of de2-7 EGFR. A series of mouse mAbs, isolated following immunization with the unique de2-7 peptide, all showed selectivity and specificity for the truncated receptor and targeted de2-7 EGFR positive xenografts grown in nude mice (Wikstrand et al. (1995); Reist et al. (1997) Improved targeting of an anti-epidermal growth factor receptor variant III monoclonal antibody in tumor xenografts after labeling using N-succinimidyl 5-iodo-3-pyridinecarboxylate. Cancer Res. 57, 1510-5; Reist et al. (1995) Tumor-specific anti-epidermal growth factor receptor variant III monoclonal antibodies: use of the tyramine-cellobiose radioiodination method enhances cellular retention and uptake in tumor xenografts. Cancer Res. 55, 4375-82).

However, one potential shortcoming of de2-7 EGFR antibodies is that only a proportion of tumors exhibiting amplification of the EGFR gene also express the de2-7 EGFR (Ekstrand et al. (1992) Amplified and rearranged epidermal growth factor receptor genes in human glioblastomas reveal deletions of sequences encoding portions of the N- and/or C-terminal tails. Proc. Natl. Acad. Sci. U.S.A. 89, 4309-13). The exact percentage of tumors containing the de2-7 EGFR is not completely established, because the use of different techniques (i.e. PCR versus immunohistochemistry) and various antibodies, has produced a wide range of reported values for the frequency of its presence. Published data indicates that approximately 25-30% of gliomas express de2-7 EGFR with expression being lowest in anaplastic astrocytomas and highest in glioblastoma multiforme (Wong et al. (1992); Wikstrand et al. (1998) The class III variant of the epidermal growth factor receptor (EGFR): characterization and utilization as an immunotherapeutic target. J. Neurovirol. 4, 148-58; Moscatello et al. (1995) Frequent expression of a mutant epidermal growth factor receptor in multiple human tumors. Cancer Res. 55, 5536-9). The proportion of positive cells within de2-7 EGFR expressing gliomas has been reported to range from 37-86% (Wikstrand et al. (1997)). 27% of breast carcinomas and 17% of lung cancers were found to be positive for the de2-7 EGFR (Wikstrand et al. (1997); Wikstrand et al. (1995); Wikstrand et al. (1998); and Hills et al., 1995). Thus, de2-7 EGFR specific antibodies would be expected to be useful in only a percentage of EGFR positive tumors.

Thus, while the extant evidence of activity of EGFR antibodies is encouraging, the observed limitations on range of applicability and efficacy reflected above remain. Accordingly, it would be desirable to develop antibodies and like agents that demonstrate efficacy with a broad range of tumors, and it is toward the achievement of that objective that the present invention is directed.

The citation of references herein shall not be construed as an admission that such is prior art to the present invention.

### SUMMARY OF THE INVENTION

The present invention provides isolated specific binding members, particularly antibodies or fragment thereof, which recognizes an EGFR epitope which does not demonstrate any amino acid sequence alterations or substitutions from wild-type EGFR and which is found in tumorigenic, hyperproliferative or abnormal cells and is not generally detectable in normal or wild type cells (the term “wild-type cell” as used herein contemplates a cell that expresses endogenous EGFR but not the de 2-7EGFR and the term specifically excludes a cell that over-expresses the EGFR gene; the term “wild-type” refers to a genotype or phenotype or other characteristic present in a normal cell rather than in an abnormal or tumorigenic cell). In a further aspect, the present invention provides specific binding members, particularly antibodies or fragments thereof, which recognizes an EGFR epitope which is found in tumorigenic, hyperproliferative or abnormal cells and is not generally detectable in normal or wild type cells, wherein the epitope is enhanced or evident upon aberrant post translational modification or aberrant expression. In a particular non-limiting exemplification provided herein, the EGFR epitope is enhanced or evident wherein post-translational modification is not complete or full to the extent seen with normal expression of EGFR in wild type cells. In one aspect, the EGFR epitope is enhanced or evident upon initial or simple carbohydrate modification or early glycosylation, particularly high mannose modification, and is reduced or not evident in the presence of complex carbohydrate modification.

The specific binding members, which may be antibodies or fragments thereof, such as immunogenic fragments thereof, do not substantially bind to or recognize normal or wild type cells containing normal or wild type EGFR epitope in the absence of aberrant expression and in the presence of normal EGFR post-translational modification.

More particularly, the specific binding member of the invention, may be antibodies or fragments thereof, which recognizes an EGFR epitope which is present in cells overexpressing EGFR (e.g., EGFR gene is amplified) or expressing the de2-7 EGFR, particularly in the presence of aberrant post-translational modification, and that is not generally detectable in cells expressing EGFR under normal conditions, particularly in the presence of normal post-translational modification.

The present inventors have discovered novel monoclonal antibodies, exemplified herein by the antibodies designated mAb806, ch806, hu806, mAb175, mAb124, and mAb1133, which specifically recognize aberrantly expressed EGFR. In particular, the antibodies of the present invention recognize an EGFR epitope which is found in tumorigenic, hyperproliferative or abnormal cells and is not generally detectable in normal or wild type cells, wherein the epitope is enhanced or evident upon aberrant post-translational modification. The novel antibodies of the invention also recognize amplified wild type EGFR and the de2-7 EGFR, yet bind to an epitope distinct from the unique junctional peptide of the de2-7 EGFR mutation. The antibodies of the present invention specifically recognize aberrantly expressed EGFR, including amplified EGFR and mutant EGFR (exemplified herein by the de2-7 mutation), particularly upon aberrant post-translational modification. Additionally, while these antibodies do not recognize the EGFR when expressed on the cell surface of a glioma cell line expressing normal amounts of EGFR, they do bind to the extracellular domain of the EGFR (sEGFR) immobilized on the surface of ELISA plates, indicating the recognition of a conformational epitope. These antibodies bind to the surface of A431 cells, which have an amplification of the EGFR gene but do not express the de2-7 EGFR Importantly, these antibodies did not bind significantly to normal tissues such as liver and skin, which express levels of endogenous, wild type (wt) EGFR that are higher than in most other normal tissues, but wherein EGFR is not aberrantly expressed or amplified.

The antibodies of the present invention can specifically categorize the nature of EGFR tumors or tumorigenic cells, by staining or otherwise recognizing those tumors or cells wherein aberrant EGFR expression, including EGFR amplification and/or EGFR mutation, particularly de2-7EGFR, is present. Further, the antibodies of the present invention demonstrate significant in vivo anti-tumor activity against tumors containing amplified EGFR and against de2-7 EGFR positive xenografts.

The unique specificity of these antibodies to bind to the de2-7 EGFR and amplified EGFR, but not to the normal, wild type EGFR, provides diagnostic and therapeutic uses to identify, characterize and target a number of tumor types, for example, head and neck, breast, or prostate tumors and glioma, without the problems associated with normal tissue uptake that may be seen with previously known EGFR antibodies.

Accordingly, the invention provides specific binding proteins, such as antibodies, which bind to the de2-7 EGFR at an epitope which is distinct from the junctional peptide but which do not substantially bind to EGFR on normal cells in the absence of amplification of the EGFR gene. By amplification, it is meant to include that the cell comprises multiple copies of the EGFR gene.

Preferably the epitope recognized by the inventive antibodies is located within the region comprising residues 273-501 of the mature normal or wild type EGFR sequence, and preferably comprises residues 287-302 (SEQ ID NO:14) of the mature normal or wild type EGFR sequence. Therefore, also provided are specific binding proteins, such as antibodies, which bind to the de2-7 EGFR at an epitope located within the region comprising residues 273-501 and/or 287-302 (SEQ ID NO:14) of the EGFR sequence. The epitope may be determined by any conventional epitope mapping techniques known to the person skilled in the art. Alternatively, the DNA sequence encoding residues 273-501 and/or 287-302 (SEQ ID NO:14) could be digested, and the resultant fragments expressed in a suitable host. Antibody binding could be determined as mentioned above.

In a preferred aspect, the antibodies are ones which have the characteristics of the antibodies which the inventors have identified and characterized, in particular recognizing aberrantly expressed EGFR, as found in amplified EGFR and de2-7EGFR.

In another aspect, the invention provides antibodies capable of competing with the inventive antibodies, under conditions in which at least 10% of an antibody having the VH and VL chain sequences of the inventive antibodies are blocked from binding to de2-7EGFR by competition with such an antibody in an ELISA assay. In particular, anti-idiotype antibodies are contemplated and are exemplified herein. The anti-idiotype antibodies LMH-11, LMH-12 and LMH-13 are provided herein.

The binding of an antibody to its target antigen is mediated through the complementarity-determining regions (CDRs) of its heavy and light chains, with the role of CDR3 being of particular importance. Accordingly, specific binding members based on the CDR3 regions of the heavy or light chain, and preferably both, of the inventive antibodies will be useful specific binding members for in vivo therapy.

Accordingly, specific binding proteins such as antibodies which are based on the CDRs of the inventive antibodies identified, particularly the CDR3 regions, will be useful for targeting tumors with amplified EGFR regardless of their de2-7 EGFR status. As the inventive antibodies do not bind significantly to normal, wild type receptor, there would be no significant uptake in normal tissue, a limitation of EGFR antibodies currently being developed.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody does not bind to the de2-7 EGFR junctional peptide consisting of the amino acid sequence of SEQ ID NO:13, wherein the antibody binds to an epitope within the sequence of residues 287-302 (SEQ ID NO:14) of human wild-type EGFR, and wherein the antibody does not comprise a heavy chain variable region sequence having the amino acid sequence set forth in SEQ ID NO:2 and does not comprise a light chain variable region sequence having the amino acid sequence set forth in SEQ ID NO:4.

In another aspect, there is provided an isolated antibody wherein the antibody comprises a heavy chain and a light chain, the heavy chain having the amino acid sequence set forth in SEQ ID NO:42, and the light chain having the amino acid sequence set forth in SEQ ID NO:47.

In another aspect, there is provided an isolated antibody wherein the antibody comprises a heavy chain and a light chain, the heavy chain having the amino acid sequence set forth in SEQ ID NO:129, and the light chain having the amino acid sequence set forth in SEQ ID NO:134.

In another aspect, there is provided an isolated antibody, wherein the antibody comprises a heavy chain and a light chain, the heavy chain having the amino acid sequence set forth in SEQ ID NO:22, and the light chain having the amino acid sequence set forth in SEQ ID NO:27.

In another aspect, there is provided an isolated antibody, wherein the antibody comprises a heavy chain and a light chain, the heavy chain having the amino acid sequence set forth in SEQ ID NO:32, and the light chain having the amino acid sequence set forth in SEQ ID NO:37.

In another aspect, there is provided an isolated antibody, wherein the antibody comprises a heavy chain and a light chain, wherein the variable region of the heavy chain comprises polypeptide binding domain regions having amino acid sequences highly homologous to the amino acid sequences set forth in SEQ ID NOS:44, 45, and 46.

In another aspect, there is provided an isolated antibody, wherein the antibody comprises a heavy chain and a light chain, wherein the variable region of the light chain comprises polypeptide binding domain regions having amino acid sequences highly homologous to the amino acid sequences set forth in SEQ ID NOS:49, 50, and 51.

In another aspect, there is provided an isolated antibody, wherein the antibody comprises a heavy chain and a light chain, wherein the variable region of the heavy chain comprises polypeptide binding domain regions having amino acid sequences highly homologous to the amino acid sequences set forth in SEQ ID NOS:130, 131, and 132.

In another aspect, there is provided an isolated antibody, wherein the antibody comprises a heavy chain and a light chain, wherein the variable region of the light chain comprises polypeptide binding domain regions having amino acid sequences highly homologous to the amino acid sequences set forth in SEQ ID NOS:135, 136, and 137.

In another aspect, there is provided an isolated antibody, wherein the antibody comprises a heavy chain and a light chain, wherein the variable region of the heavy chain comprises polypeptide binding domain regions having amino acid sequences highly homologous to the amino acid sequences set forth in SEQ ID NOS:23, 24, and 25.

In another aspect, there is provided an isolated antibody, wherein the antibody comprises a heavy chain and a light chain, wherein the variable region of the light chain comprises polypeptide binding domain regions having amino acid sequences highly homologous to the amino acid sequences set forth in SEQ ID NOS:28, 29, and 30.

In another aspect, there is provided an isolated antibody, wherein the antibody comprises a heavy chain and a light chain, wherein the variable region of the heavy chain comprises polypeptide binding domain regions having amino acid sequences highly homologous to the amino acid sequences set forth in SEQ ID NOS:33, 34, and 35.

In another aspect, there is provided an isolated antibody, wherein the antibody comprises a heavy chain and a light chain, wherein the variable region of the light chain comprises polypeptide binding domain regions having amino acid sequences highly homologous to the amino acid sequences set forth in SEQ ID NOS:38, 39, and 40.

In another aspect, there is provided an isolated antibody, wherein the isolated antibody is the form of an antibody F(ab′)2, scFv fragment, diabody, triabody or tetrabody.

In another aspect, there is provided an isolated antibody further comprising a detectable or functional label.

In another aspect, the detectable or functional label is a covalently attached drug.

In another aspect, the label is a radiolabel.

In another aspect, there is provided an isolated antibody, wherein the isolated antibody is peglyated.

In another aspect, there is provided an isolated nucleic acid which comprises a sequence encoding an isolated antibody recited herein.

In another aspect, there is provided a method of preparing an isolated antibody, comprising expressing a nucleic acid as recited above and herein under conditions to bring about expression of the antibody, and recovering the antibody.

In another aspect, there is provided a method of treatment of a tumor in a human patient which comprises administering to the patient an effective amount of an isolated antibody recited herein.

In another aspect, there is provided a kit for the diagnosis of a tumor in which EGFR is aberrantly expressed or in which EGFR is expressed in the form of a truncated protein, comprising an isolated antibody recited herein.

In another aspect, the kit further comprises reagents and/or instructions for use.

In another aspect, there is provided a pharmaceutical composition comprising an isolated antibody as recited herein.

In another aspect, the pharmaceutical composition further comprises a pharmaceutically acceptable vehicle, carrier or diluent.

In another aspect, the pharmaceutical composition further comprises an anti-cancer agent selected from the group consisting of chemotherapeutic agents, anti-EGFR antibodies, radioimmunotherapeutic agents, chemical ablation agents, toxins, immunomodulators, cytokines, cytotoxic agents, drugs and combinations thereof.

In another aspect, the chemotherapeutic agents are selected from the group consisting of tyrosine kinase inhibitors, phosphorylation cascade inhibitors, post-translational modulators, cell growth or division inhibitors (e.g. anti-mitotics), signal transduction inhibitors, and combinations thereof.

In another aspect, the tyrosine kinase inhibitors are selected from the group consisting of AG1478, ZD1839, STI571, OSI-774, SU-6668, and combinations thereof.

In another aspect, the anti-EGFR antibodies are selected from the group consisting of the anti-EGFR antibodies 528,225, SC-03, DR8. 3, L8A4, Y10, ICR62, ABX-EGF, and combinations thereof.

In another aspect, the anti-cancer agent is selected from the group consisting: 4-desacetylvinblastine-3-carbohydiazide; 5-fluoro-T-deoxyuridine; 5-fluorouracil; 5-fluorouracil decarbonizes; 6-mercaptopurine; 6-thioguanine; abrin; abrin A chain; actinomycin D; actinomycin D, 1-dehydrotestosterone; adriamycin; alkylating agents; alkylphosphocholines; aminopterin; angiogenin; angiostatin; anthracyclines; anthramycin; anti-angiogenics; anti-folates; anti-metabolites; anti-mitotics; antibiotics; ara-C; auristatin derivatives; auristatin E; auristatin E valeryl benzylhydrazone; auristatin F phenylene diamine; auristatins; auromycins; bis-iodo-phenol mustard; bismuth; bleomycin; busulfan; calicheamicin; carboplatin; caminomycin; carmustine; cc-1065 compounds; chlorambucil; cis-dichlorodiamine platinum (cisplatin); colchicin (colchicine); combrestatin; crotin; curicin; cyclothosphamide; cytarabine; cytochalasin B; cytosine arabinoside; cytoxin; dacarbazine; dactinomycin (actinomycin); daunorubicin (daunomycin); dibromomannitol; dihydroxy anthracin dione; diphtheria toxin; dolastatin-10; doxetaxel; doxorubicin; doxorubicin hydrazides; duocarmycins; emetine; endostatin; enediyenes; enomycin; epirubicin; esperamicin compounds; ethidium bromide; etoposide; gelonin; glucocorticoids; gramicidin D; granulocyte colony stimulating factor; granulocyte macrophage colony stimulating factor; idarubicin; intercalating agents; interleukin-1; interleukin-2; interleukin-6; lidocaine; lomustine; lymphokine; maytansinols; mechlorethamine; melphalan (and other related nitrogen mustards); methotrexate; minor groove-binders; mithramycin; mitogellin; mitomycin C; mitomycins; mitoxantrone; MMAF-dimethylaminoethylamine; MMAF-N-t-butyl; MMAF-tetraethylene glycol; modeccin A chain; mono-methyl auristatin E (MMAE); mono-methyl auristatin F (MMAF); morpholinodoxorubicin; N2′-deacetyl-N2′-α-mercapto-1 oxopropyl)-maytansine (DM1); N2′-deacetyl-N2′-(4-mercapto-4-methyl-1-oxopentyl)-maytansine (DM4); neocarzinostatin; nerve growth factor (and other growth factors); onapristone; paclitaxel; PE40; phenomycin; platelet derived growth factor; prednisone; procaine; propranolol; Pseudomonas exotoxin A; puromycin; radioactive isotopes (such as, for example and without limitation, At211, Bi212, Bi213, Cf252, 1125, 1131, In111, Ir192, Lu177, P32, Re186, Re188, Sm153, Y90, and W188); retstrictocin; ricin A; ricins; Sapaonaria officinalis inhibitor; saporin; streptozotocin; suramin; tamoxifen; taxanes; taxoids; taxol; tenoposide; tetracaine; thioepa chlorambucil; thiotepa; thrombotic agents; tissue plasminogen activator; topoisomerase I inhibitors; topoisomerase II inhibitors; toxotere; tumor necrosis factor; vinblastine; vinca alkaloids; vincas; vincristine; vindesine; vinorelbine; yttrium; α-interferon; α-sarcin; and β-interferon.

In another aspect, the anti-cancer agent is conjugated to an isolated antibody as recited herein, and may be conjugated using one or more linker, spacer and stretcher compounds.

In another aspect, the one or more linker, space and stretcher compounds are selected from the group consisting of: valine-citrulline; maleimidocaproyl; amino benzoic acids; p-aminobenzylcarbamoyl (PAB); lysosomal enzyme-cleavable linkers; maleimidocaproyl-polyethylene glycol (MC(PEG)6-OH); N-methyl-valine citrulline; N-succinimidyl 4-(N-maleimidomethyl)cyclohexane-1-carboxylate (SMCC); N-Succinimidyl 4-(2-pyridyldithio)butanoate (SPDB); and N-Succinimidyl 4-(2-pyridylthio)pentanoate (SPP).

In another aspect, there is provided a method of preventing and/or treating cancer in mammals, comprising administering to a mammal a therapeutically effective amount of a pharmaceutical composition as recited herein.

In another aspect, there is provided a method for the treatment of brain-resident cancers that produce aberrantly expressed EGFR in mammals, comprising administering to a mammal a therapeutically effective amount of a pharmaceutical composition as recited herein.

In another aspect, the brain-resident cancers are selected from the group consisting of glioblastomas, medulloblastomas, meningiomas, neoplastic astrocytomas and neoplastic arteriovenous malformations.

In another aspect, there is provided a unicellular host transformed with a recombinant DNA molecule which encodes an isolated antibody recited herein.

In another aspect, the unicellular host is selected from the group consisting of E. coli, Pseudomonas, Bacillus, Streptomyces, yeasts, CHO, YB/20, NSO, SP2/0, R1.1, B-W, L-M, COS 1, COS 7, BSC1, BSC40, and BMT10 cells, plant cells, insect cells, and human cells in tissue culture.

In another aspect, there is provided a method for detecting the presence of amplified EGFR, de2-7EGFR or EGFR with high mannose glycosylation wherein the EGFR is measured by: (a) contacting a biological sample from a mammal in which the presence of amplified EGFR, de2-7EGFR or EGFR with high mannose glycosylation is suspected with an isolated antibody of claim 1 under conditions that allow binding of the EGFR to the isolated antibody to occur; and (b) detecting whether binding has occurred between the EGFR from the sample and the isolated antibody; wherein the detection of binding indicates that presence or activity of the EGFR in the sample.

In another aspect of the method of detecting the presence of amplified EGFR, de2-7EGFR or EGFR with high mannose glycosylation, the detection of the presence of the EGFR indicates the existence of a tumor or cancer in the mammal.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, the heavy chain having an amino acid sequence that is substantially homologous to the amino acid sequence set forth in SEQ ID NO:42, and the light chain having an amino acid sequence that is substantially homologous to the amino acid sequence set forth in SEQ ID NO:47.

In another aspect, the heavy chain of the antibody comprises the amino acid sequence set forth in SEQ ID NO:42, and wherein the light chain of the antibody comprises the amino acid sequence set forth in SEQ ID NO:47.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, wherein the variable region of the heavy chain comprises polypeptide binding domain regions having amino acid sequences highly homologous to the amino acid sequences set forth in SEQ ID NOS:44, 45, and 46, and wherein the variable region of the light chain comprises polypeptide binding domain regions having amino acid sequences highly homologous to the amino acid sequences set forth in SEQ ID NOS:49, 50, and 51.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, the heavy chain having an amino acid sequence that is substantially homologous to the amino acid sequence set forth in SEQ ID NO:129, and the light chain having an amino acid sequence that is substantially homologous to the amino acid sequence set forth in SEQ ID NO:134.

In another aspect, the heavy chain of the antibody comprises the amino acid sequence set forth in SEQ ID NO:129, and wherein the light chain of the antibody comprises the amino acid sequence set forth in SEQ ID NO:134.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, wherein the variable region of the heavy chain comprises polypeptide binding domain regions having amino acid sequences highly homologous to the amino acid sequences set forth in SEQ ID NOS:130, 131, and 132, and wherein the variable region of the light chain comprises polypeptide binding domain regions having amino acid sequences highly homologous to the amino acid sequences set forth in SEQ ID NOS:135, 136, and 137.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, the heavy chain having an amino acid sequence that is substantially homologous to the amino acid sequence set forth in SEQ ID NO:22, and the light chain having an amino acid sequence that is substantially homologous to the amino acid sequence set forth in SEQ ID NO:27.

In another aspect, the heavy chain of the antibody comprises the amino acid sequence set forth in SEQ ID NO:22, and wherein the light chain of the antibody comprises the amino acid sequence set forth in SEQ ID NO:27.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, wherein the variable region of the heavy chain comprises polypeptide binding domain regions having amino acid sequences highly homologous to the amino acid sequences set forth in SEQ ID NOS:23, 24, and 25, and wherein the variable region of the light chain comprises polypeptide binding domain regions having amino acid sequences highly homologous to the amino acid sequences set forth in SEQ ID NOS:28, 29, and 30.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, the heavy chain having an amino acid sequence that is substantially homologous to the amino acid sequence set forth in SEQ ID NO:32, and the light chain having an amino acid sequence that is substantially homologous to the amino acid sequence set forth in SEQ ID NO:37.

In another aspect, the heavy chain of the antibody comprises the amino acid sequence set forth in SEQ ID NO:32, and wherein the light chain of the antibody comprises the amino acid sequence set forth in SEQ ID NO:37.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, wherein the variable region of the heavy chain comprises polypeptide binding domain regions having amino acid sequences highly homologous to the amino acid sequences set forth in SEQ ID NOS:33, 34, and 35, and wherein the variable region of the light chain comprises polypeptide binding domain regions having amino acid sequences highly homologous to the amino acid sequences set forth in SEQ ID NOS:38, 39, and 40.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody does not bind to the de2-7 EGFR junctional peptide consisting of the amino acid sequence of SEQ ID NO:13, wherein the antibody binds to an epitope within the sequence of residues 287-302 of human wild-type EGFR,

the antibody comprising a light chain and a heavy chain, wherein the variable region of the light chain comprises a first polypeptide binding domain region having an amino acid sequence corresponding to the amino acid sequence set forth in Formula I:

HSSQDIXₐₐ₁SNIG (I),

wherein Xaa₁ is an amino acid residue having an uncharged polar R group (SEQ ID NO:151);

a second polypeptide binding domain region having an amino acid sequence corresponding to the amino acid sequence set forth in Formula II:

HGTNLXₐₐ₂D (II),

wherein Xaa₂ is an amino acid residue having a charged polar R group (SEQ ID NO:152);

and a third polypeptide binding domain region having an amino acid sequence corresponding to the amino acid sequence set forth in Formula III:

VQYXₐₐ₃QFPWT (III),

wherein Xaa₃ is selected from the group consisting of A, G, and an amino acid residue which is conservatively substituted for A or G (SEQ ID NO:153); and

wherein the variable region of the heavy chain comprises a first polypeptide binding domain region having an amino acid sequence corresponding to the amino acid sequence set forth in Formula IV:

SDXₐₐ₄AWN (IV),

wherein Xaa₄ is selected from the group consisting of F, Y, and an amino acid residue which is conservatively substituted for F or Y (SEQ ID NO:154);

a second polypeptide binding domain region having an amino acid sequence corresponding to the amino acid sequence set forth in Formula V, Formula VI, or Formula VII:

YISYSGNTRYXₐₐ₅PSLKS (V),

wherein Xaa₅ is an amino acid residue having an uncharged polar R group (SEQ ID NO:155),

YISYSXₐₐ₆NTRYNPSLKS (VI),

wherein Xaa₆ is selected from the group consisting of G, A, and an amino acid residue which is conservatively substituted for G or A (SEQ ID NO:156),

YISYSGNTRYNPSLXₐₐ₇S (VII),

and Xaa₇ is a basic amino acid residue (SEQ ID NO:157); and

a third polypeptide binding domain region having an amino acid sequence corresponding to the amino acid sequence set forth in Formula VIII:

Xₐₐ₈TAGRGFPY (VIII),

wherein Xaa_{g} is selected from the group consisting of V, A, and an amino acid residue which is conservatively substituted for V or A (SEQ ID NO:158),

and wherein the antibody does not comprise a heavy chain variable region sequence having the amino acid sequence set forth in SEQ ID NO:2 and does not comprise a light chain variable region sequence having the amino acid sequence set forth in SEQ ID NO:4.

In another aspect, Xₐₐ₁ is N; Xₐₐ₂ is D; Xₐₐ₃ is A; Xₐₐ₄ is F; Xₐₐ₅ is an amino acid residue having an uncharged polar R group; Xₐₐ₆ is G; Xₐₐ₇ is K; and Xₐₐ₈ is V.

In another aspect, Xₐₐ₅ is N or Q.

In another aspect, Xₐₐ₁ is N or S.

In another aspect, Xₐₐ₂ is D or E.

In another aspect, Xₐₐ₃ is A or G.

In another aspect, Xₐₐ₄ is F or Y.

In another aspect, Xₐₐ₅ is N or Q.

In another aspect, Xₐₐ₆ is G or A, and Xₐₐ₇ is independently K or R.

In another aspect, Xₐₐ₈ is V or A.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody does not bind to the de2-7 EGFR junctional peptide consisting of the amino acid sequence of SEQ ID NO:13, wherein the antibody binds to an epitope within the sequence of residues 273-501 of human wild-type EGFR,

the antibody comprising a light chain and a heavy chain, wherein the variable region of the light chain comprises a first polypeptide binding domain region having the amino acid sequence HSSQDINSNIG (SEQ ID NO:18); a second polypeptide binding domain region having the amino acid sequence HGTNLDD (SEQ ID NO:19); and a third polypeptide binding domain region having the amino acid sequence VQYAQFPWT (SEQ ID NO:20),

wherein the variable region of the heavy chain comprises a first polypeptide binding domain region having the amino acid sequence SDFAWN (SEQ ID NO:15); a second polypeptide binding domain region having an amino acid sequence corresponding to the amino acid sequence set forth in Formula IX:

YISYSGNTRYXₐₐ₉PSLKS (IX),

wherein Xₐₐ₉ is an amino acid residue having an uncharged polar R group; and

a third polypeptide binding domain region having the amino acid sequence VTAGRGFPY (SEQ ID NO:17).

In another aspect, the antibody binds to an epitope within the sequence of residues 287-302 (SEQ ID NO:14) of human wild-type EGFR.

In another aspect, Xₐₐ₉ is N or Q.

In another aspect, the binding domain regions are carried by a human antibody framework.

In another aspect, the human antibody framework is a human IgG1 antibody framework.

In another aspect, there is provided an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, the heavy chain having an amino acid sequence that is substantially homologous to the amino acid sequence set forth in SEQ ID NO:2, and the light chain having an amino acid sequence that is substantially homologous to the amino acid sequence set forth in SEQ ID NO:4.

In another aspect, the heavy chain of the antibody comprises the amino acid sequence set forth in SEQ ID NO:2, and wherein the light chain of the antibody comprises the amino acid sequence set forth in SEQ ID NO:4.

In another aspect, there is provided, an isolated antibody capable of binding EGFR on tumors containing amplifications of the EGFR gene, wherein cells of the tumors contain multiple copies of the EGFR gene, and on tumors that express the truncated version of the EGFR receptor de2-7, wherein the antibody comprises a heavy chain and a light chain, wherein the variable region of the heavy chain comprises polypeptide binding domain regions having amino acid sequences highly homologous to the amino acid sequences set forth in SEQ ID NOS:15, 16, and 17, and wherein the variable region of the light chain comprises polypeptide binding domain regions having amino acid sequences highly homologous to the amino acid sequences set forth in SEQ ID NOS:18, 19, and 20.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1 presents the results of flow cytometric analysis of glioma cell lines. U87MG (light gray histograms) and U87MG.Δ2-7 (dark gray histograms) cells were stained with either an irrelevant IgG2b antibody (open histograms), DH8.3 (specific for de2-7 EGFR), mAb806 or 528 (binds both wild type and de2-7 EGFR) as indicated.

FIGS. 2A-D present the results of ELISA of mAb806, mAbDH8.3 and mAb528. (A) binding of increasing concentrations of mAb806 (▴) DH8.3 () or 528 (▪) antibody to sEGFR coated ELISA plates. (B) inhibition of mAb806 and mAb528 binding to sEGFR coated ELISA plates by increasing concentrations of soluble EGFR (sEGFR) in solution. (C) binding of increasing concentrations of DH8.3 to the de2-7 junctional peptide illustrates binding curves for mAb806 and mAb528 to immobilized wild-type sEGFR (D).

FIGS. 2E and 2F graphically present the results of BIAcore binding studies using C-terminal biotinylated peptide and including a monoclonal antibody of the invention, along with other known antibodies, among them the L8A4 antibody which recognizes the junction peptide of the de2-7 EGFR mutant, and controls.

FIG. 3 depicts the internalization of mAb806 and the DH8.3 antibody. U87MG.Δ2-7 cells were pre-incubated with mAb806 (▴) or DH8.3 () at 4° C., transferred to 37° C. and internalization determined by FACS. Data represents mean internalization at each time point±SE of 3 (DH8.3) or 4 (mAb806) separate experiments.

FIGS. 4A and 4B illustrate biodistribution (% ID/g tumor tissue) of radiolabeled (a) ¹²⁵I-mAb806 and (b) ¹³¹I-DH8.3 in nude mice bearing U87MG and U87MG.Δ2-7 xenografts. Each point represents the mean of 5 mice±SE except for 1 hr where n=4.

FIGS. 5A and 5B illustrate biodistribution of radiolabeled ¹²⁵I-mAb806 (open bar) and ¹³¹I-DH8.3 (filled bar) antibodies expressed as (a) tumor:blood or (b) tumor:liver ratios in nude mice bearing U87MG.Δ2-7 xenografts. Each bar represents the mean of 5 mice±SE except for 1 hr where n=4

FIGS. 6A-C illustrate flow cytometric analysis of cell lines containing amplification of the EGFR gene. A431 cells were stained with either mAb806, DH8.3 or 528 (black histograms) and compared to an irrelevant IgG2b antibody (open histogram).

FIGS. 7A and 7B illustrate biodistribution (% ID/g tumor tissue) of radiolabeled (a) 1251-mAb806 and (b) ¹³¹I-528 in nude mice bearing U87MG.Δ2-7 and A431 xenografts.

FIGS. 8A-D illustrate biodistribution of radiolabeled ¹²⁵I-mAb806 (open bar) and ¹³¹I-528 (filled bar) and antibodies expressed as (A, B) tumor:blood or (C, D) tumor:liver ratios in nude mice bearing (A, C) U87MG.Δ2-7 and (B, D) A431 xenografts.

FIGS. 9A and 9B illustrate anti-tumor effect of mAb806 on (A) U87MG and (B) U87MG.Δ2-7 xenograft growth rates in a preventative model. 3×10⁶ U87MG or U87MG.Δ2-7 cells were injected s.c. into both flanks of 4-6 week old BALB/c nude mice, (n=5) at day 0. Mice were injected i.p. with either 1 mg of mAb806 (); 0.1 mg of mAb806 (▴); or vehicle (∘) starting one day prior to tumor cell inoculation. Injections were given three times per week for two weeks as indicated by the arrows. Data are expressed as mean tumor volume±S.E.

FIGS. 10A, 10B, and 10C illustrate the anti-tumor effect of mAb806 on (A) U87MG, (B) U87MG.Δ2-7 and (C) U87MG.wtEGFR xenografts in an established model. 3×10⁶ U87MG, U87MG.Δ2-7, or U87MG.wtEGFR cells, were injected s.c. into both flanks of 4-6 week old BALB/c nude mice, (n=5). Mice were injected i.p. with either 1 mg doses of mAb806 (); 0.1 mg doses of mAb806 (A); or vehicle (O) starting when tumors had reached a mean tumor volume of 65-80 mm³. Injections were given three times per week for two weeks as indicated by the arrows. Data are expressed as mean tumor volume±S.E.

FIGS. 11A and 11B illustrate anti-tumor effect of mAb806 on A431 xenografts in (A) preventative and (B) established models. 3×10⁶ A431 cells were injected s.c. into both flanks of 4-6 week old BALB/c nude mice (n=5). Mice were injected i.p. with either 1 mg doses of mAb806 (); or vehicle (∘), starting one day prior to tumor cell inoculation in the preventative model, or when tumors had reached a mean tumor volume of 200 mm³. Injections were given three times per week for two weeks as indicated by the arrows. Data are expressed as mean tumor volume±S.E.

FIG. 12 illustrates the anti-tumor effect of treatment with mAb806 combined with treatment with AG1478 on A431 xenografts in a preventative model. Data are expressed as mean tumor volume±S.E.

FIG. 13 depicts mAb806 binding to A431 cells in the presence of increasing concentrations of AG1478 (0.5 μM and 5 μM).

FIGS. 14A and 14B illustrate the (A) nucleic acid sequence and the (B) amino acid translation thereof of the 806 VH chain gene (SEQ ID NO:1 and SEQ ID NO:2, respectively).

FIGS. 15A and 15B illustrate the (A) nucleic acid sequence and the (B) amino acid translation thereof of the 806 VL chain gene (SEQ ID NO:3 and SEQ ID NO:4, respectively).

FIG. 16 shows the VH chain sequence (SEQ ID NO:2) numbered according to Kabat, with the CDRs (SEQ ID NOS:15, 16 and 17) underlined. Key residues of the VH chain sequence (SEQ ID NO:2) are 24, 37, 48, 67 and 78.

FIG. 17 shows the VL chain sequence (SEQ ID NO:4) numbered according to Kabat, with the CDRs (SEQ ID NOS:18, 19 and 20) underlined. Key residues of the VL chain sequence (SEQ ID NO:4) are 36, 46, 57 and 71.

FIGS. 18A-18D show the results of in vivo studies designed to determine the therapeutic effect of combination antibody therapy, particularly mAb806 and the 528 antibody. Mice received inoculations of U87MG.D2-7 (A and B), U87MG.DK (C), or A431 (D) cells.

FIGS. 19 A-D show analysis of internalization by electron microscopy. U87MG.Δ2-7 cells were pre-incubated with mAb806 or DH8.3 followed by gold conjugated anti-mouse IgG at 4° C., transferred to 37° C. and internalization examined at various time points by electron microscopy. (A) localization of the DH8.3 antibody to a coated pit (arrow) after 5 min; (B) internalization of mAb806 by macropinocytosis (arrow) after 2 min; (C) localization of DH8.3 to lysosomes (arrow) after 20 min; (D) localization of mAb806 to lysosomes (arrow) after 30 min Original magnification for all images is X30,000.

FIG. 20 shows autoradiography of a U87MG.Δ2-7 xenograft section collected 8 hr after injection of ¹²⁵I-mAb806.

FIG. 21 shows flow cytometric analysis of cell lines containing amplification of the EGFR gene. HN5 and MDA-468 cells were stained with an irrelevant IgG2b antibody (open histogram with dashed line), mAb806 (black histogram) or 528 (open histogram with closed lines). The DH8.3 antibody was completely negative on both cell lines (data not shown).

FIG. 22 shows immunoprecipitation of EGFR from cell lines. The EGFR was immunoprecipitated from ³⁵S-labeled U87MG.Δ2-7 or A431 cells with mAb806, sc-03 antibody or a IgG2b isotype control. Arrows at the side indicate the position of the de2-7 and wt EGFR. Identical banding patterns were obtained in 3 independent experiments.

FIG. 23 shows autoradiography of an A431 xenograft section collected 24 hr after injection of ¹²⁵I-mAb806, areas of localization to viable tissue are indicated (arrows).

FIGS. 24A and 24B show extended survival of nude mice bearing intracranial U87MG.ΔEGFR (A) and LN-Z308.ΔEGFR (B) xenografts with systemic mAb806 treatment. U87MG.EGFR cells (1×10⁵) or LN-Z308.ΔEGFR cells (5×10⁵) were implanted into nude mice brains, and the animals were treated with either mAb806, PBS, or isotype IgG from post-implantation days 0 through 14.

FIGS. 24C and 24D show growth inhibition of intracranial tumors by mAb806 treatment. Nude mice (five per group), treated with either mAb806 or the isotype IgG control, were euthanized on day 9 for U87MG.EGFR(C) and on day 15 for LN-Z308.ΔEGFR (D), and their brains were harvested, fixed, and sectioned. Data were calculated by taking the tumor volume of control as 100%. Values are mean±SD. ***, P<0.001; control versus mAb806. Arrowheads, tumor tissue.

FIG. 24E shows extended survival of nude mice bearing intracranial U87MG.ΔEGFR xenografts with intratumoral mAb806 treatment. U87MG.ΔEGFR cells were implanted as described. 10 mg of mAb806 or isotype IgG control in a volume of 5 μl were injected at the tumor-injection site every other day starting at day 1 for five times.

FIGS. 25A, 25B, and 25C show that mAb806 extends survival of mice with U87MG.wtEGFR brain tumors but not with U87MG.DK. or U87MG brain tumors. U87MG (A), U87MG.DK (B), or U87MG.wtEGFR(C) cells (5×10⁵) were implanted into nude mice brains, and the animals were treated with mAb806 from post-implantation days 0 through 14 followed by observation after discontinuation of therapy.

FIG. 26A shows FACS analysis of mAb806 reactivity with U87MG cell lines. U87MG, U87MG.ΔEGFR, U87MG.DK, and U87MG.wtEGFR cells were stained with anti-EGFR mAbs 528, EGFR.1, and anti-ΔEGFR antibody, mAb806. Monoclonal EGFR. 1 antibody recognized wtEGFR exclusively and monoclonal 528 antibody reacted with both wtEGFR and ΔEGFR. mAb806 reacted intensively with U87MG.ΔEGFR and U87MG.DK and weakly with U87MG.wtEGFR. Bars on the abscissa, maximum staining of cells in the absence of primary antibody. Results were reproduced in three independent experiments.

FIG. 26B shows mAb806 immunoprecipitation of EGFR forms. Mutant and wtEGFR were immunoisolated with anti-EGFR antibodies, 528, EGFR. 1, or anti-ΔEGFR antibody, mAb806, from (Lane 1) U87MG, (Lane 2) U87Δ.EGFR, (Lane 3) U87MG.DK, and (Lane 4) U87MG.wtEGFR cells, and were then detected by Western blotting with anti-pan EGFR antibody, C13.

FIGS. 27A and 27B show that systemic treatment with mAb806 decreases the phosphorylation of ΔEGFR and Bel-XL expression in U87MG.ΔEGFR brain tumors. U87MG.ΔEGFR tumors were resected at day 9 of mAb806 treatment, immediately frozen in liquid nitrogen and stored at −80° C. before tumor lysate preparation.

(A) Western blot analysis of expression and the degree of autophosphorylation of ΔEGFR. Thirty μg of tumor lysates were subjected to SDS-polyacrylamide gels, transferred to nitrocellulose membranes, and probed with anti-phosphotyrosine mAb, then were stripped and re-probed with anti-EGFR antibody, C13.

(B) Western blotting of Bcl-XL by using the same tumor lysates as in (A). Membranes were probed with anti-human Bcl-X polyclonal antibody. Lanes 1 and 2, U87MG.ΔEGFR brain tumors treated with isotype control; Lanes 3 and 4, U87MG.ΔEGFR brain tumors treated with mAb806.

FIG. 28 shows mAb806 treatment leads to a decrease in growth and vasculogenesis and to increases in apoptosis and accumulating macrophages in U87MG.ΔEGFR tumors. Tumor sections were stained for Ki-67. Cell proliferative index was assessed by the percentage of total cells that were Ki-67 positive from four randomly selected high power fields (X400) in intracranial tumors from four mice of each group. Data are the mean±SE. Apoptotic cells were detected by TUNEL assay. Apoptotic index was assessed by the ratio of TUNEL-positive cells: total number of cells from four randomly selected high-power fields (X400) in intracranial tumors from four mice of each group. Data are the mean±SE. Tumor sections were immunostained with anti-CD31 antibody. MVAs were analyzed by computerized image analysis from four randomly selected fields (X200) from intracranial tumors from four mice of each group. Peritumoral infiltrates of macrophages in mAb806-treated U87MG.ΔEGFR tumors. Tumor sections were stained with anti-F4/80 antibody.

FIG. 29 shows flow cytometric analysis of parental and transfected U87MG glioma cell lines. Cells were stained with either an irrelevant IgG2b antibody (open histograms) or the 528 antibody or mAb806 (filled histograms) as indicated.

FIG. 30 shows immunoprecipitation of EGFR from cell lines. The EGFR was immunoprecipitated from ³⁵S-labeled U87MG.wtEGFR, U87MG.Δ2-7, and A431 cells with mAb806 (806), sc-03 antibody (c-term), or a IgG2b isotype control (con). Arrows, position of the de2-7 and wt EGFR.

FIG. 31 shows representative H&E-stained paraffin sections of U87MG.Δ2-7 and U87MG.wtEGFR xenografts. U87MG.Δ2-7 (collected 24 days after tumor inoculation) and U87MG.wtEGFR (collected 42 days after tumor inoculation) xenografts were excised from mice treated as described in FIG. 10 above, and stained with H&E. Vehicle-treated U87MG.Δ2-7 (collected 18 days after tumor inoculation) and U87MG.wtEGFR (collected 37 days after tumor inoculation) xenografts showed very few areas of necrosis (left panel), whereas extensive necrosis (arrows) was observed in both U87MG.Δ2-7 and U87MG.wtEGFR xenografts treated with mAb806 (right panel).

FIG. 32 shows immunohistochemical analysis of EGFR expression in frozen sections derived from U87MG, U87MG.Δ2-7, and U87MG.wtEGFR xenografts. Sections were collected at the time points described in FIG. 31 above. Xenograft sections were immunostained with the 528 antibody (left panel) and mAb806 (right panel). No decreased immunoreactivity to either wtEGFR, amplified EGFR, or de2-7 EGFR was observed in xenografts treated with mAb806. Consistent with the in vitro data, parental U87MG xenografts were positive for 528 antibody but were negative for mAb806 staining.

FIG. 33 shows a schematic representation of generated bicistronic expression constructs. Transcription of the chimeric antibody chains is initiated by Elongation Factor-1 promoter and terminated by a strong artificial termination sequence. IRES sequences were introduced between coding regions of light chain and NeoR and heavy chain and dhfr gene.

FIGS. 34A and 34B show biodistribution analysis of the ch806 radiolabeled with either (A) ¹²⁵I or (B) ¹¹¹In was performed in BALB/c nude mice bearing U87MG-de2-7 xenograft tumors. Mice were injected with 5 m of radiolabeled antibody and in groups of 4 mice per time point, sacrificed at either 8, 28, 48 or 74 hours. Organs were collected, weighed and radioactivity measured in a gamma counter.

FIGS. 35A and 35B depict (A) the % ID gram tumor tissue and (B) the tumor to blood ratio. Indium-111 antibody shows approximately 30% ID/gram tissue and a tumor to blood ratio of 4.0.

FIG. 36 depicts the therapeutic efficacy of chimeric antibody ch806 in an established tumor model. 3×10⁶ U87MG.Δ2-7 cells in 100 μl of PBS were inoculated s.c. into both flanks of 4-6 week old female nude mice. mAb806 was included as a positive control. Treatment was started when tumors had reached a mean volume of 50 mm³ and consisted of 1 mg of ch806 or mAb806 given i.p. for a total of 5 injections on the days indicated. Data was expressed as mean tumor volume±S.E. for each treatment group.

FIG. 37 shows CDC Activity on Target (A) U87MG.de2-7 and (B) A431 cells for anti-EGFR chimeric IgG1 antibodies ch806 and control cG250. Mean (bars; ±SD) percent cytotoxicity of triplicate determinations are presented.

FIG. 38 shows ADCC on target (A) U87MG.de2-7 and (B) A431 cells at Effector:Target cell ratio of 50:1 mediated by ch806 and isotype control cG250 (0-10 μg/ml). Results are expressed as mean (bars; ±SD) percent cytotoxicity of triplicate determinations.

FIG. 39 shows ADCC mediated by 1 μg/ml parental mAb806 and ch806 on target U87MG.de2-7 cells over a range of Effector:Target ratios. Mean (bars; ±SD) of triplicate determinations are presented.

FIG. 40 shows twenty-five hybridomas producing antibodies that bound ch806 but not huIgG were initially selected. Four of these anti-ch806 hybridomas with high affinity binding (clones 3E3, 5B8, 9D6 and 4D8) were subsequently pursued for clonal expansion from single cells by limiting dilution and designated Ludwig Institute for Cancer Research Melbourne Hybridoma (LMH)-11, -12, -13 and -14, respectively. In addition, two hybridomas that produced mAbs specific for huIgG were also cloned and characterized further: clones 2C10 (LMH-15) and 2B8 (LMH-16).

FIGS. 41A, 41B, and 41C show that after clonal expansion, the hybridoma culture supernatants were examined in triplicate by ELISA for the ability to neutralize ch806 or mAb806 antigen binding activity with sEGFR621. Mean (±SD) results demonstrated the antagonist activity of anti-idiotype mAbs LMH-11, -12, -13 and -14 with the blocking in solution of both ch806 and murine mAb806 binding to plates coated with sEGFR (LMH-14 not shown).

FIGS. 42A, 42B, and 42C show microtitre plates that were coated with 10 μg/ml purified (A) LMH-11, (B) LMH-12 and (C) LMH-13. The three purified clones were compared for their ability to capture ch806 or mAb806 in sera or 1% FCS/Media and then detect bound ch806 or mAb806. Isotype control antibodies hu3S193 and m3S193 in serum and 1% FCS/Media were included in addition to controls for secondary conjugate avidin-HRP and ABTS substrate. Results are presented as mean (±SD) of triplicate samples using biotinylated-LMH-12 (10 μg/ml) for detection and indicate LMH-12 used for capture and detection had the highest sensitivity for ch806 in serum (3 ng/ml) with negligible background binding.

FIG. 43 shows validation of the optimal pharmacokinetic ELISA conditions using 1 μg/ml anti-idiotype LMH-12 and 1 μg/ml biotinylated LMH-12 for capture and detection, respectively. Three separate ELISAs were performed in quadruplicate to measure ch806 in donor serum () from three healthy donors or 1% BSA/media (▪) with isotype control hu3S193 in serum (▴) or 1% BSA/media (▾). Controls for secondary conjugate avidin-HRP (♦) and ABTS substrate (hexagon) alone were also included with each ELISA. Mean (±SD) results demonstrate highly reproducible binding curves for measuring ch806 (2 μg/ml-1.6 ng/ml) in sera with a 3 ng/ml limit of detection. (n=12; 1-100 ng/ml, Coefficient of Variation <25%; 100 ng/ml-5 μg/ml, Coefficient of Variation <15%). No background binding was evident with any of the three sera tested and negligible binding was observed with isotype control hu3S193.

FIG. 44 depicts an immunoblot of recombinant sEGFR expressed in CHO cells, blotted with mAb806. Recombinant sEGFR was treated with PNGaseF to remove N-linked glycosylation (deglycosylated), or untreated (untreated), the protein was run on SDS-PAGE, transferred to membrane and immunoblotted with mAb806.

FIG. 45 depicts immunoprecipitation of EGFR from ³⁵S-labelled cell lines (U87MG.Δ2-7, U87MG-wtEGFR, and A431) with different antibodies (SC-03, 806 and 528 antibodies).

FIG. 46 depicts immunoprecipitation of EGFR from different cells (A431 and U87MG.Δ2-7) at different time points (time 0 to 240 minutes) after pulse-labeling with ³⁵S methionine/cysteine. Antibodies 528 and 806 are used for immunoprecipitation.

FIG. 47 depicts immunoprecipitation of EGFR from various cell lines (U87MGA2-7, U87MG-wtEGFR and A431) with various antibodies (SC-03, 806 and 528) in the absence of (−) and after Endo H digestion (+) to remove high mannose type carbohydrates.

FIG. 48 depicts cell surface iodination of the A431 and U87MG.Δ2-7 cell lines followed by immunoprecipitation with the 806 antibody, and with or without Endo H digestion, confirming that the EGFR bound by mAb806 on the cell surface of A431 cells is an EndoH sensitive form.

FIG. 49 shows the pREN ch806 LC Neo Vector (SEQ ID NO:7).

FIG. 50 shows the pREN ch806 HC DHFR Vector (SEQ ID NO:8).

FIGS. 51A-D shows the mAb124 VH and VL chain nucleic acid sequences (SEQ ID NOS:21 and 26, respectively) and amino acid sequences (SEQ ID NOS:22 and 27, respectively).

FIGS. 52A-D shows the mAb1133 VH and VL chain nucleic acid sequences (SEQ ID NO:31 and 36, respectively) and amino acid sequences (SEQ ID NOS:32 and 37, respectively).

FIG. 53 shows a DNA plasmid graphic of the combined, double gene Lonza plasmid including pEE12.4 containing the hu806H (VH+CH) expression cartridge, and pEE6.4 containing the hu806L (VL+CL) expression cartridge.

FIG. 54 shows the DNA sequence (SEQ ID NO:41; complement SEQ ID NO:162) of the combined Lonza plasmid described in FIG. 53. This sequence also shows all translations (SEQ ID NOS:42-51 and 163-166) relevant to the hu806 antibody. The plasmid has been sequence-verified, and the coding sequence and translation checked. Sections of the sequence have been shaded to identify regions of interest; the shaded regions correspond to actual splice junctions. The color code is as follows:

(gray): signal region, initial coding sequences found at both the heavy and light-chain variable regions;

(lavender): hu806 VH chain, veneered heavy-chain variable region;

(pink): hu806 CH chain, codon-optimized heavy-chain constant region;

(green): hu806 VL chain, veneered light-chain variable region; and

(yellow): hu806 CL chain, codon-optimized light-chain constant region.

FIGS. 55A and 55B show the hu806 translated amino acid sequences (VH and VL chains of SEQ ID NOS:164 and 166 and their respective signal peptides of SEQ ID NOS:163 and 165; CH and CL chains of SEQ ID NOS:43 and 48), and give the Kabat numbers for the VH and VL chains (SEQ ID NOS:164 and 165, respectively), with CDRs (SEQ ID NOS:44-46 and 49-51) underlined.

FIGS. 56A, 56B, 56C, 57A, 57B, and 57C show the initial step in veneering design, the grading of amino acid residues in the mAb806 sequence (VH chain of SEQ ID NO:167 and VL chain of SEQ ID NO:12) for surface exposure. Grades are given in the number of asterisks (*) above each residue, with the most exposed residues having three asterisks. These figures include a design indicating how the initial oligonucleotides (VH chain: FIG. 56C and SEQ ID NOS:52 and 169-177; VL chain: FIG. 57C and SEQ ID NOS: 62, 66, 68 and 181-187) overlapped to form the first veneered product (VH chain of SEQ ID NO:168 and VL chain of SEQ ID NO:180).

FIG. 58 shows a map of codon optimized huIgG1 heavy chain DNA sequence (SEQ ID NO:80; complement SEQ ID NO:178) and amino acid translation (SEQ ID NO:43).

FIG. 59 shows the protein alignment comparing the hu806 VH+CH amino acid sequence (8C65AAG hu806 VH+CH; SEQ ID NO:81) to the original reference file for the mAb806 VH chain (SEQ ID NO:167). Highlighted regions indicate conserved amino acid sequences in the VH chain. The CDRs are underlined. Asterisks reflect changes that were planned and carried out in the initial veneering process. The numbered sites are references to later modifications.

FIG. 60 shows the corresponding alignment for the hu806 VL+CL amino acid sequence (8C65AAG hu806 signal+VL+CL; SEQ ID NO:83) to the original reference file for the mAb806 VL chain (SEQ ID NO:179). It contains an additional file (r2vk1 hu806 signal+VL+CL; SEQ ID NO:82), a precursor construct, which was included to illustrate the change made at modification #7.

FIG. 61 shows a nucleotide and amino acid alignment of the hu806 signal+VL and CL sequences (8C65AAG hu806 Vl+Cl; SEQ ID NOS:190 and 188) with the corresponding ch806 sequences (pREN ch806 LC Neo; LICR; SEQ ID NO:189). It has been modified and annotated as described in FIG. 62.

FIG. 62 shows the nucleotide alignment of the hu806 signal+VH sequence (8C65AAG hu806 VH chain; SEQ ID NO:192) with the corresponding mAb806 sequence [mAb806 VH chain before codon change (cc) and veneering (ven); SEQ ID NO:191]. The nucleotide changes behind the amino acid changes of FIGS. 59 and 60 are illustrated, as well as showing conservative nucleic acid changes that led to no change in amino acid. The intron between the signal and the VH chain in hu806 has been removed for easier viewing. The signal sequence and CDRs are underlined. The corresponding amino acid sequence (SEQ ID NO:42) has been superimposed on the alignment.

FIG. 63. shows binding of purified hu806 antibody obtained from transient transfectant 293 cells to recombinant EGFR-ECD as determined by Biacore. No binding to the EGFR-ECD was observed with purified control human IgG1 antibody.

FIG. 64 shows the GenBank formatted text document of the sequence (SEQ ID NO:41) and annotations of plasmid 8C65AAG encoding the IgG1 hu806.

FIG. 65 shows the alignment of amino acid sequences for CDRs from mAb806 (SEQ ID NOS:15-18, 20 and 193) and mAb175 (SEQ ID NOS:130-132, 135 and 194-195). Sequence differences between the two antibodies are bolded.

FIGS. 66A and 66B show immunohistochemical staining of cell lines and normal human liver with mAb175. (A) Biotinylated mAb175 was used to stain sections prepared from blocks containing A431 cells (over-express the wtEGFR), U87MG.Δ2-7 cells (express the Δ2-7EGFR) and U87MG cells (express the wtEGFR at modest levels). (B) Staining of normal human liver (400×) with mAb175 (left panel), isotype control (centre panel) and secondary antibody control (right panel). No specific sinusoidal or hepatocyte staining was observed.

FIGS. 67A, 67B, and 67C show the reactivity of mAb806 and mAb175 with fragments of the EGFR displayed on yeast. (A) Representative flow cytometry histograms depicting the mean fluorescence signal of mAb175 and mAb806-labeling of yeast-displayed EGFR fragments. With yeast display a percentage of cells do not express protein on their surface resulting in 2 histogram peaks. The 9E10 antibody is used as a positive control as all fragments contain a linear C-terminal c-myc tag. (B) Summary of antibody binding to various EGFR fragments. (C) The EGFR fragments were denatured by heating yeast pellets to 800° C. for 30 min The c-myc tag was still recognized by the 9E10 anti-myc antibody in all cases, demonstrating that heat treatment does not compromise the yeast surface displayed protein. The conformation sensitive EGFR antibody mAb225 was used to confirm denaturation.

FIGS. 68A, 68B, 68C, and 68D show the antitumor effects of mAb175 on brain and prostate cancer xenografts. (A) Mice (n=5) bearing U87MG.Δ2-7 xenografts were injected i.p. with PBS, 1 mg of mAb175 or mAb806 (positive control), three times weekly for two weeks on days 6, 8, 10, 13, 15 and 17 when the starting tumor volume was 100 mm³. Data are expressed as mean tumor volume±SE. (B) Cells were stained with two irrelevant antibodies (blue, solid and green, hollow), mAb 528 for total EGFR (pink, solid), mAb806 (light blue, hollow) and mAb175 (orange, hollow) and then analyzed by FACS. (C) DU145 cells were lysed, subjected to IP with mAb 528, mAb806, mAb175 or two independent irrelevant antibodies and then immunoblotted for EGFR. (D) Mice (n=5) bearing DU145 xenografts were injected i.p. with PBS, 1 mg of mAb175 or mAb806, daily on days 18-22, 25-29 and 39-43 when the starting tumor volume was 85 mm³. Data are expressed as mean tumor volume±SE.

FIGS. 69A, 69B, 69C, 69D, 69E, and 69F show the crystal structures of EGFR peptide 287-302 bound to the Fab fragments (A) Cartoon of Fab 806, with the light chain, red; heavy chain, blue; bound peptide, yellow; and the superposed EGFR₂₈₇₋₃₀₂ from EGFR, purple. (B) Cartoon of Fab 175 with the light chain, yellow; heavy chain, green; bound peptide, lilac; and EGFR₂₈₇₋₃₀₂ from EGFR(DI-3), purple. (C) Detail from (B) showing the similarity of EGFR₂₈₇₋₃₀₂ in the receptor to the peptide bound to FAb 175. Peptides backbones are shown as Cα traces and the interacting side chains as sticks. O atoms are colored red; N, blue; S, orange and C, as for the main chain. (D) Superposition of EGFR with the Fab175:peptide complex showing spacial overlap. Coloring as in (C) with the surface of EGFR187-286 colored turquoise. (E) Orthogonal view to (D) with EGFR187-286 shown in opaque blue and the surface of the light (orange) and heavy (green) chains transparent. (F) Detailed stereoview of 175 Fab complex looking into the antigen-binding site. Coloring as in (C) and side chain hydrogen bonds dotted in black. Water molecules buried upon complex formation are shown as red spheres.

FIGS. 70A, 70B, 70C, and 70D show the influence of the 271-283 cysteine bond on mAb806 binding to the EGFR. (A) Cells transfected with wtEGFR, EGFR-C271A, EGFR—C283A or the C271A/C283A mutant were stained with mAb528 (solid pink histogram), mAb806 (blue line) or only the secondary antibody (purple) and then analyzed by FACS. The gain was set up using a class-matched irrelevant antibody. (B) BaF3 cells expressing the EGFR-C271A or C271/283A EGFR were examined for their response to EGF in an MTT assay as described. EC_{50S} were derived using the Bolzman fit of the data points. Data represent mean and sd of triplicate measurements. (C) BaF3 cells expressing the wild-type or the EGFR-C271A/C283A were IL-3 and serum starved, then exposed to EGF or vehicle control. Whole cell lysates were separated by SDS-PAGE and immunoblotted with anti-phosphotyrosine antibody (top panel) or anti-EGFR antibody (bottom panel). (D) BaF3 cells expressing the wild-type (left panel) or the C271A/C283A (right panel) EGFR were stimulated with increasing concentrations of EGF in the presence of no antibody (open symbols), mAb 528 (grey circles) or mAb806 (black triangles), both at 10 μg/ml. Data are expressed as mean and sd of triplicate measurements.

FIGS. 71A, 71B, and 71C show: (A) Whole body gamma camera image of the biodistribution of ¹¹¹In ch806 in a patient with metastatic squamous cell carcinoma of the vocal cord, showing quantitative high uptake in tumor in the right neck (arrow). Blood pool activity, and minor catabolism of free ¹¹¹In in liver, is also seen. (B) Single Photon Computed Tomography (SPECT) image of the neck of this patient, showing uptake of ¹¹¹In-ch806 in viable tumor (arrow), with reduced central uptake indicating necrosis. (C) Corresponding CT scan of the neck demonstrating a large right neck tumor mass (arrow) with central necrosis.

FIGS. 72A and 72B show a stereo model of the structure of the untethered EGFR1-621. The receptor backbone is traced in blue and the ligand TGF-α in red. The mAb806/175 epitope is drawn in turquoise and the disulfide bonds in yellow. The atoms of the disulfide bond which ties the epitope back into the receptor are shown in space-filling format. The model was constructed by docking the EGFR-ECD CR2 domain from the tethered conformation onto the structure of an untethered EGFR monomer in the presence of its ligand.

FIG. 73 shows the reactivity of mAb806 with fragments of the EGFR. Lysates from 293T cells transfected with vectors expressing the soluble 1-501 EGFR fragment or GH/EGFR fragment fusion proteins (GH-274-501, GH-282-501, GH-290-501 and GH-298-501) were resolved by SDS-PAGE, transferred to membrane and immunoblotted with mAb806 (left panel) or the anti-myc antibody 9B11 (right panel).

FIGS. 74A and 74B show the mAb175 VH chain nucleic acid sequence (SEQ ID NO:128) and amino acid sequence (SEQ ID NO:129), respectively.

FIGS. 75A and 75B show the mAb175 VL chain nucleic acid sequence (SEQ ID NO:133) and amino acid sequence (SEQ ID NO:134), respectively.

FIGS. 76A, 76B, and 76C show: (A) Volumetric product concentration and (B) viable cell concentration of GS-CHO (14D8, 15B2 and 40A10) and GS-NSO (36) hu806 transfectants in small scale (100 mL) shake flasks cultures. Product concentration was estimated by ELISA using the 806 anti-idiotype as coating antibody and ch806 Clinical Lot: J06024 as standard; (C) GS-CHO 40A10 transfectant cell growth and volumetric production in a 15 L stiffed tank bioreactor. Viable cell density (♦×10⁵ cell/mL), cell viability (▪) and production ( [symbol] mg/L).

FIGS. 77A, 77B, 77C, 77D, and 77E show Size Exclusion Chromatography (Biosep SEC-S3000) Analysis of Protein-A purified hu806 antibody constructs produced by small scale culture and control ch806 and mAb 806. Chromatograms at A214 nm are presented in the upper panels and at A280 nm in the lower panel of each Figure.

FIG. 78 shows Size Exclusion Chromatography (Biosep SEC-S3000) Analysis of Protein-A purified hu806 antibody construct 40A10 following large scale production and Protein-A purification. Chromatogram at A214 nm is presented indicating 98.8% purity with 1.2% aggregate present.

FIG. 79 shows that precast 4-20% Tris/Glycine Gels from Novex, USA were used under standard SDS-PAGE conditions to analyze purified transfectant hu806 preparations (5 μg) GS CHO (14D8, 15B2 and 40A10) and GS-NS0 (36) hu806 under reduced conditions. Proteins detected by Coomassie Blue Stain.

FIG. 80 shows that precast 4-20% Tris/Glycine Gels were used under standard SDS-PAGE conditions to analyze purified transfectant hu806 preparations (5 μg) GS CHO (14D8, 15B2 and 40A10) and GS-NS0 (36) under non-reduced conditions. Proteins detected by Coomassie Blue Stain.

FIG. 81 shows that precast 4-20% Tris/Glycine Gels were used under standard SDS-PAGE conditions to analyze purified transfectant hu806 GS CHO 40A10 (5 μg) following large scale production. Proteins detected by Coomassie Blue Stain.

FIG. 82 shows Isoelectric Focusing gel analysis of purified transfectant hu806 GS CHO 40A10 (5 μg) following 15 L production. Proteins detected by Coomassie Blue Stain. Lane 1, pI markers; Lane 2, hu806 (three isoforms, pI 8.66 to 8.82); Lane 3, pI markers.

FIG. 83 shows binding to A431 cells: Flow Cytometry analysis of Protein-A purified hu806 antibody preparations (20 μg/ml), and isotype control huA33 (20 μg/ml). Controls include secondary antibody alone (green) and ch806 (red). Hu806 constructs were produced by small scale culture.

FIG. 84 shows binding to A431 cells: Flow Cytometry analysis of purified mAb806, ch806 and hu806 40A10 antibody preparations (20 μg/ml) that bind ˜10% of wild type EGFR on cell surface, 528 (binds both wild type and de2-7 EGFR) and irrelevant control antibody (20 μg/ml) as indicated.

FIG. 85 shows binding to U87MG.de2-7 glioma cells. Flow Cytometry analysis of purified mAb806, ch806 and hu806 40A10 antibody preparations (20 μg/ml) and 528 anti-EGFR and irrelevant control antibody (20 μg/ml).

FIG. 86 shows specific binding of ¹²⁵I-radiolabelled 806 antibody constructs to: (A) U87MG.de2-7 glioma cells and (B) A431 carcinoma cells.

FIG. 87 shows Scatchard Analyses: ¹²⁵I-radiolabelled (A) ch806 and (B) hu806 antibody constructs binding to U87MG.de2-7 cells.

FIG. 88 shows Scatchard Analyses: ¹²⁵I-radiolabelled (A) ch806 and (B) hu806 antibody constructs binding to A431 cells.

FIG. 89 shows BIAcore analysis of binding to 287-302 EGFR 806 peptide epitope by (A) hu806 and (B) ch806 passing over the immobilized peptide in increasing concentrations of 50 nM, 100 nM, 150 nM, 200 nM, 250 nM and 300 nM.

FIG. 90 shows ch806- and hu806-mediated Antibody Dependant Cellular Cytotoxicity on target A431 cells determined at (A) 1 μg/ml each antibody over a range of effector to target cell ratios (E:T=0.78:1 to 100:1); (B) at E:T=50:1 over a concentration range of each antibody (3.15 ng/ml-10 μg/ml).a on target A431.

FIG. 91 shows treatment of established A431 xenografts in BALB/c nude mice. Groups of 5 mice received 6×1 mg dose over 2 weeks antibody therapy as indicated (arrows). Mean±SEM tumor volume is presented until study termination.

FIG. 92 shows treatment of established U87MG.de2-7 xenografts in BALB/c nude mice. Groups of 5 mice received 6×1 mg dose over 2 weeks antibody therapy as indicated (arrows). Mean±SEM tumor volume is presented until study termination.

FIG. 93 shows deviations from random coil chemical shift values for the mAb806 peptide (A) N, (B) HN and (C) HA. Peptide was prepared in H₂O solution containing 5%₂H₂O, 70 mM NaCl and 50 mM NaPO₄ at pH 6.8. All spectra used for sequential assignments were acquired at 298K on a Bruker Avance500.

FIGS. 94A, 94B, 94C, 94D, 94E, and 94F show whole body gamma camera images of Patient 7 A) Anterior, and B) Posterior, Day 5 post infusion of ¹¹¹In-ch806. High uptake of ¹¹¹In-ch806 in metastatic lesions in the lungs (arrows) is evident. C) and D) show metastatic lesions (arrows) on CT scan. E) 3D SPECT images of the chest, and F) co-registered transaxial images of SPECT and CT showing specific uptake of ¹¹¹In-ch806 in metastatic lesions.

FIGS. 95A, 95B, 95C, 95D, 95E, and 95F show planar images of the head and neck of Patient 8 obtained A) Day 0, B) Day 3 and C) Day 7 post infusion of ¹¹¹In-ch806. Initial blood pool activity is seen on Day 0, and uptake of ¹¹¹In-ch806 in an anaplastic astrocytoma in the right frontal lobe is evident by Day 3 (arrow), and increases by Day 7. Specific uptake of ¹¹¹In-ch806 is confirmed in D) SPECT image of the brain (arrow), at the site of tumor (arrow) evident in E) ¹⁸F-FDG PET, and F) MRI.

FIGS. 96A, 96B, 96C, and 96D show similar uptake of 111In-ch806 in tumor is evident in Patient 3 compared to Patient 4, despite differences in 806 antigen expression in screened tumor samples. A) ¹¹¹In-ch806 localization in lung metastasis (arrow) on SPECT transaxial image in Patient 4, with cardiac blood pool activity (B) evident. B) corresponding CT scan. Archived tumor was shown to have <10% positivity for 806 expression. C) ¹¹¹In-ch806 localization in lung metastasis (arrow) in Patient 3, with cardiac blood pool activity (B) evident. D) corresponding CT scan. Archived tumor was shown to have 50-75% positivity for 806 expression.

FIG. 97 shows pooled population pharmacokinetics of ch806 protein measured by ELISA. Observed and predicted ch806 (% ID/L) vs. time post infusion (hrs).

FIGS. 98A and 98B show individual patient results for A) Normalised Whole Body Clearance and B) Hepatic Clearance of ¹¹¹In-ch806 at the 5 mg/m² (▪), 10 mg/m² (Δ), 20 mg/m² (∇), and 40 mg/m² (♦) dose levels. Linear regression for data sets indicated in each panel [A) r²=0.9595; B) r²=0.9415].

### DETAILED DESCRIPTION

In accordance with the present invention there may be employed conventional molecular biology, microbiology, and recombinant DNA techniques within the skill of the art. Such techniques are explained fully in the literature. See, for example, Sambrook et al., “Molecular Cloning: A Laboratory Manual” (1989); “Current Protocols in Molecular Biology” Volumes I-E [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

As used herein, the following terms are deemed to have, without limitation, the provided definitions.

The term “specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. This application is concerned with antigen-antibody type reactions.

The term “aberrant expression” in its various grammatical forms may mean and include any heightened or altered expression or overexpression of a protein in a tissue, e.g. an increase in the amount of a protein, caused by any means including enhanced expression or translation, modulation of the promoter or a regulator of the protein, amplification of a gene for a protein, or enhanced half-life or stability, such that more of the protein exists or can be detected at any one time, in contrast to a nonoverexpressed state. Aberrant expression includes and contemplates any scenario or alteration wherein the protein expression or post-translational modification machinery in a cell is taxed or otherwise disrupted due to enhanced expression or increased levels or amounts of a protein, including wherein an altered protein, as in mutated protein or variant due to sequence alteration, deletion or insertion, or altered folding is expressed.

It is important to appreciate that the term “aberrant expression” has been specifically chosen herein to encompass the state where abnormal (usually increased) quantities/levels of the protein are present, irrespective of the efficient cause of that abnormal quantity or level. Thus, abnormal quantities of protein may result from overexpression of the protein in the absence of gene amplification, which is the case e.g. in many cellular/tissue samples taken from the head and neck of subjects with cancer, while other samples exhibit abnormal protein levels attributable to gene amplification.

In this latter connection, certain of the work of the inventors that is presented herein to illustrate the invention includes the analysis of samples certain of which exhibit abnormal protein levels resulting from amplification of EFGR. This therefore accounts for the presentation herein of experimental findings where reference is made to amplification and for the use of the terms “amplification/amplified” and the like in describing abnormal levels of EFGR. However, it is the observation of abnormal quantities or levels of the protein that defines the environment or circumstance where clinical intervention as by resort to the binding members of the invention is contemplated, and for this reason, the present specification considers that the term “aberrant expression” more broadly captures the causal environment that yields the corresponding abnormality in EFGR levels.

Accordingly, while the terms “overexpression” and “amplification” in their various grammatical forms are understood to have distinct technical meanings, they are to be considered equivalent to each other, insofar as they represent the state where abnormal EFGR protein levels are present in the context of the present invention. Consequently, the term “aberrant expression” has been chosen as it is believed to subsume the terms “overexpression” and “amplification” within its scope for the purposes herein, so that all terms may be considered equivalent to each other as used herein.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al. (1989) Nature 341, 544-546) which consists of a VH domain; (v) isolated CDR regions; (vi) F (ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science. 242, 423-426; Huston et al. (1988) PNAS USA. 85, 5879-5883); (viii) multivalent antibody fragments (scFv dimers, trimers and/or tetramers (Power and Hudson (2000) J. Immunol. Methods 242, 193-204) (ix) bispecific single chain Fv dimers (PCT/US92/09965) and (x) “diabodies”, multivalent or multispecific fragments constructed by gene fusion (WO94/13804; P. Holliger et al. (1993) Proc. Natl. Acad. Sci. USA 90, 6444-6448).

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F (ab′) Z and F (v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct anti-EGFR antibody, for instance antibody 528 (U.S. Pat. No. 4,943,533), the chimeric and humanized 225 antibody (U.S. Pat. No. 4,943,533 and WO/9640210), an anti-de2-7 antibody such as DH8.3 (Hills, D. et al (1995) Int. J. Cancer. 63(4), 537-543), antibody L8A4 and Y10 (Reist, C J et al. (1995) Cancer Res. 55 (19):4375-4382; Foulon C F et al. (2000) Cancer Res. 60 (16):44534460), ICR62 (Modjtahedi H et al. (1993) Cell Biophys. Jan-Jun; 22 (1-3):129-46; Modjtahedi et al. (2002) P.A.A.C.R. 55 (14):3140-3148, or the antibody of Wikstrand et al (Wikstrand C. et al (1995) Cancer Res. 55 (14):3140-3148). The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF), and particularly, the TNF bispecific modality demonstrated in U.S. Ser. No. 60/355,838 filed Feb. 13, 2002, incorporated herein in its entirety) or a toxin (e.g., ricin) or anti-mitotic or apoptotic agent or factor.

Fab and F(ab′)₂ portions of antibody molecules may be prepared by the proteolytic reaction of papain and pepsin, respectively, on substantially intact antibody molecules by methods that are well-known. See, for example, U.S. Pat. No. 4,342,566 to Theofilopolous et al. Fab′ antibody molecule portions are also well-known and are produced from F (ab′) 2 portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

“Post-translational modification” may encompass any one of or combination of modification (s), including covalent modification, which a protein undergoes after translation is complete and after being released from the ribosome or on the nascent polypeptide co-translationally. Post-translational modification includes but is not limited to phosphorylation, myristylation, ubiquitination, glycosylation, coenzyme attachment, methylation and acetylation. Post-translational modification can modulate or influence the activity of a protein, its intracellular or extracellular destination, its stability or half-life, and/or its recognition by ligands, receptors or other proteins. Post-translational modification can occur in cell organelles, in the nucleus or cytoplasm or extracellularly.

The term “specific” may be used to refer to the situation in which one member of a specific binding pair will not show any significant binding to molecules other than its specific binding partner (s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus

The term “isolated” refers to the state in which specific binding members of the invention, or nucleic acid encoding such binding members will be, in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practiced in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Specific binding members may be glycosylated, either naturally or by systems of heterologous eukaryotic cells, or they may be (for example if produced by expression in a prokaryotic cell) unglycosylated.

Also, as used herein, the terms “glycosylation” and “glycosylated” includes and encompasses the post-translational modification of proteins, termed glycoproteins, by addition of oligosaccarides. Oligosaccharides are added at glycosylation sites in glycoproteins, particularly including N-linked oligosaccharides and O-linked oligosaccharides. N-linked oligosaccharides are added to an Asn residue, particularly wherein the Asn residue is in the sequence N-X-S/T, where X cannot be Pro or Asp, and are the most common ones found in glycoproteins. In the biosynthesis of N-linked glycoproteins, a high mannose type oligosaccharide (generally comprised of dolichol, N-Acetylglucosamine, mannose and glucose is first formed in the endoplasmic reticulum (ER). The high mannose type glycoproteins are then transported from the ER to the Golgi, where further processing and modification of the oligosaccharides occurs. 0-linked oligosaccharides are added to the hydroxyl group of Ser or Thr residues. In O-linked oligosaccharides, N-Acetylglucosamine is first transferred to the Ser or Thr residue by N-Acetylglucosaminyltransferase in the ER. The protein then moves to the Golgi where further modification and chain elongation occurs. O-linked modifications can occur with the simple addition of the OGlcNAc monosaccharide alone at those Ser or Thr sites which can also under different conditions be phosphorylated rather than glycosylated.

As used herein, “pg” means picogram, “ng” means nanogram, “ug” or “ug” mean microgram, “mg” means milligram, “ul” or “μl” mean microliter, “ml” means milliliter, “1” means liter.

The terms “806 antibody”, “mAb806”, “ch806”, and any variants not specifically listed, may be used herein interchangeably, and as used throughout the present application and claims refer to proteinaceous material including single or multiple proteins, and extends to those proteins having the amino acid sequence data described herein and presented in SEQ ID NO:2 and SEQ ID NO:4, and the chimeric antibody ch806 which is incorporated in and forms a part of SEQ ID NOS:7 and 8, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “806 antibody”, “mAb806” and “ch806” are intended to include within their scope proteins specifically recited herein as well as all substantially homologous analogs and allelic variations.

The terms “humanized 806 antibody”, “hu806”, and “veneered 806 antibody” and any variants not specifically listed, may be used herein interchangeably, and as used throughout the present application and claims refer to proteinaceous material including single or multiple proteins, and extends to those proteins having the amino acid sequence data described herein and presented in SEQ ID NO:42 and SEQ ID NO:47, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “humanized 806 antibody”, “hu806”, and “veneered 806 antibody” are intended to include within their scope proteins specifically recited herein as well as all substantially homologous analogs and allelic variations.

The terms “175 antibody” and “mAb175”, and any variants not specifically listed, may be used herein interchangeably, and as used throughout the present application and claims refer to proteinaceous material including single or multiple proteins, and extends to those proteins having the amino acid sequence data described herein and presented in SEQ ID NO:129 and SEQ ID NO:134, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “175 antibody” and “mAb175” are intended to include within their scope proteins specifically recited herein as well as all substantially homologous analogs and allelic variations.

The terms “124 antibody” and “mAb124”, and any variants not specifically listed, may be used herein interchangeably, and as used throughout the present application and claims refer to proteinaceous material including single or multiple proteins, and extends to those proteins having the amino acid sequence data described herein and presented in SEQ ID NO:22 and SEQ ID NO:27, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “124 antibody” and “mAb124” are intended to include within their scope proteins specifically recited herein as well as all substantially homologous analogs and allelic variations.

The terms “1133 antibody” and “mAb1133”, and any variants not specifically listed, may be used herein interchangeably, and as used throughout the present application and claims refer to proteinaceous material including single or multiple proteins, and extends to those proteins having the amino acid sequence data described herein and presented in SEQ ID NO:32 and SEQ ID NO:37, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “11133 antibody” and “mAb1133” are intended to include within their scope proteins specifically recited herein as well as all substantially homologous analogs and allelic variations.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

Table of Correspondence

Symbol

| 1-Letter | 3-Letter | Amino Acid |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | aspargine |
| C | Cys | cysteine |

It should be noted that all amino-acid residue sequences are represented herein by formulae whose left and right orientation is in the conventional direction of amino terminus to carboxy-terminus Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding specific binding members (antibodies) of the invention which code for antibodies having the disclosed sequences but which are degenerate to such sequences. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA |
| | or CUG |
| Isoleucine (He or I) | AUU or AUC or AUA |
| Methionine (Met or M) | AUG |
| Valine (Valor V) | GUU or GUC of GUA or GUG |
| Serine (Ser or S) | UCU or UCC or UCA or UCG or AGU |
| | or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA |
| | or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in, for example, the disclosed sequences of antibodies of the present invention, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at Ph 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gin for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces. (3-turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, preferably by at least 50 percent, preferably by at least 70 percent, preferably by at least 80 percent, preferably by at least 90%, a clinically significant change in the growth or progression or mitotic activity of a target cellular mass, group of cancer cells or tumor, or other feature of pathology. For example, the degree of EGFR activation or activity or amount or number of EGFR positive cells, particularly of antibody or binding member reactive or positive cells may be reduced.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tm with washes of higher stringency, if desired.

The present invention provides a novel specific binding member, particularly an antibody or fragment thereof, including immunogenic fragments, which recognizes an EGFR epitope which is found in tumorigenic, hyperproliferative or abnormal cells wherein the epitope is enhanced or evident upon aberrant post-translational modification and not detectable in normal or wild-type cells. In a particular but nonlimiting embodiment, the binding member, such as the antibody, recognizes an EGFR epitope which is enhanced or evident upon simple carbohydrate modification or early glycosylation and is reduced or not evident in the presence of complex carbohydrate modification or glycosylation. The specific binding member, such as the antibody or fragment thereof, does not bind to or recognize normal or wild-type cells containing normal or wild-type EGFR epitope in the absence of overexpression and in the presence of normal EGFR post-translational modification.

The present invention further provides novel antibodies 806, 175, 124, 1133, ch806, and hu806 and fragment thereof, including immunogenic fragments, which recognizes an EGFR epitope, particularly the EGFR peptide (₂₈₇CGADSYEMEEDGVRKC₃₀₂ (SEQ ID NO:14)), which is exposed in tumorigenic, hyperproliferative or abnormal cells wherein the epitope is enhanced,

revealed, or evident and not detectable in normal or wild-type cells. In a particular but non-limiting embodiment, the antibody recognizes an EGFR epitope which is enhanced or evident upon simple carbohydrate modification or early glycosylation and is reduced or not evident in the presence of complex carbohydrate modification or glycosylation. The antibody or fragment thereof does not bind to or recognize normal or wild-type cells containing normal or wild-type EGFR epitope in the absence of overexpression, amplification, or a tumorigenic event.

In a particular aspect of the invention and as stated above, the present inventors have discovered the novel monoclonal antibodies 806, 175, 124, 1133, ch806, and hu806 which specifically recognize amplified wild-type EGFR and the de2-7 EGFR, yet bind to an epitope distinct from the unique junctional peptide of the de2-7 EGFR mutation. Additionally, while mAb806, mAb175, mAb124, mAb1133, and hu806 do not recognize the normal, wild-type EGFR expressed on the cell surface of glioma cells, they do bind to the extracellular domain of the EGFR immobilized on the surface of ELISA plates, indicating a conformational epitope with a polypeptide aspect.

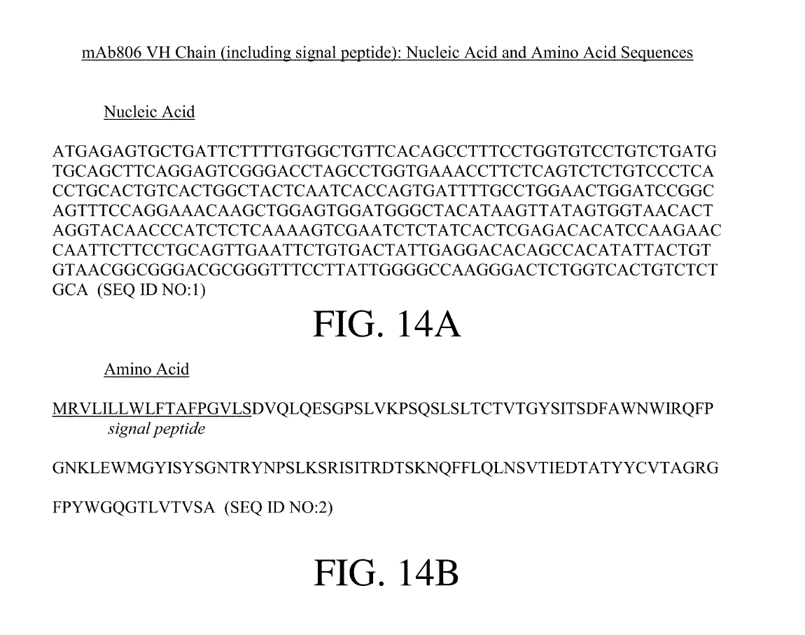

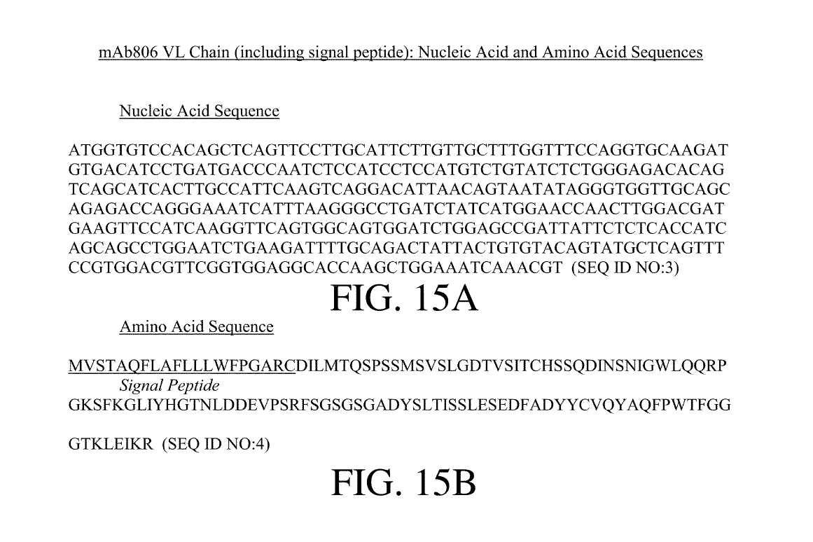

Importantly, mAb806, mAb175, mAb124, mAb1133, ch806, and hu806 do not bind significantly to normal tissues such as liver and skin, which express levels of endogenous wtEGFR that are higher than in most other normal tissues, but wherein EGFR is not overexpressed or amplified. Thus, mAb806, mAb175, mAb124, mAb1133, and hu806 demonstrate novel and useful specificity, recognizing de2-7 EGFR and amplified EGFR, while not recognizing normal, wild-type EGFR or the unique junctional peptide which is characteristic of de2-7 EGFR. In a preferred aspect mAb806, mAb175, mAb124, mAb1133, and hu806 of the present invention comprises the VH and VL chain CDR domain amino acid sequences depicted in FIGS. 14B and 15B; 74B and 75B; 51B and 51D; 52B and 52D; and 55A and 55B, respectively (SEQ ID NOS:2 and 4; 129 and 134; 22 and 27; 32 and 37; and 42 and 47, respectively; SEQ ID NO:42 including the hu806 VH chain signal peptide and VH chain sequences of SEQ ID NOS:163 and 164, respectively, and SEQ ID NO:47 including the hu806 VL chain signal peptide and VL chain sequences of SEQ ID NOS: 165 and 166, respectively).

In another aspect, the invention provides an antibody capable of competing with the 175 antibody, under conditions in which at least 10% of an antibody having the VH and VL chain sequences of the 175 antibody (SEQ ID NOS:129 and 134, respectively) is blocked from binding to de2-7EGFR by competition with such an antibody in an ELISA assay. As set forth above, anti-idiotype antibodies are contemplated herein.

The present invention relates to specific binding members, particularly antibodies or fragments thereof, which recognizes an EGFR epitope which is present in cells expressing amplified EGFR or expressing the de2-7 EGFR and not detectable in cells expressing normal or wild-type EGFR, particularly in the presence of normal posttranslational modification.

It is further noted and herein demonstrated that an additional non-limiting observation or characteristic of the antibodies of the present invention is their recognition of their epitope in the presence of high mannose groups, which is a characteristic of early glycosylation or simple carbohydrate modification. Thus, altered or aberrant glycosylation facilitates the presence and/or recognition of the antibody epitope or comprises a portion of the antibody epitope.

Glycosylation includes and encompasses the post-translational modification of proteins, termed glycoproteins, by addition of oligosaccarides. Oligosaccharides are added at glycosylation sites in glycoproteins, particularly including N-linked oligosaccharides and O-linked oligosaccharides. N-linked oligosaccharides are added to an Asn residue, particularly wherein the Asn residue is in the sequence N-X-S/T, where X cannot be Pro or Asp, and are the most common ones found in glycoproteins. In the biosynthesis of N-linked glycoproteins, a high mannose type oligosaccharide (generally comprised of dolichol, N-Acetylglucosamine, mannose and glucose is first formed in the endoplasmic reticulum (ER). The high mannose type glycoproteins are then transported from the ER to the Golgi, where further processing and modification of the oligosaccharides normally occurs. O-linked oligosaccharides are added to the hydroxyl group of Ser or Thr residues. In O-linked oligosaccharides, N Acetylglucosamine is first transferred to the Ser or Thr residue by N Acetylglucosaminyltransferase in the ER. The protein then moves to the Golgi where further modification and chain elongation occurs.

In a particular aspect of the invention and as stated above, the present inventors have discovered novel monoclonal antibodies, exemplified herein by the antibodies designated mAb806 (and its chimeric ch806), mAb175, mAb124, mAb1133, and hu806 which specifically recognize amplified wild-type EGFR and the de2-7 EGFR, yet bind to an epitope distinct from the unique junctional peptide of the de2-7 EGFR mutation. The antibodies of the present invention specifically recognize overexpressed EGFR, including amplified EGFR and mutant EGFR (exemplified herein by the de2-7 mutation), particularly upon aberrant post-translational modification. Additionally, while these antibodies do not recognize the normal, wild-type EGFR expressed on the cell surface of glioma cells, they do bind to the extracellular domain of the EGFR immobilized on the surface of ELISA plates, indicating a conformational epitope with a polypeptide aspect. Importantly, these antibodies do not bind significantly to normal tissues such as liver and skin, which express levels of endogenous wtEGFR that are higher than in most other normal tissues, but wherein EGFR is not overexpressed or amplified. Thus, these antibodies demonstrate novel and useful specificity, recognizing de2-7 EGFR and amplified EGFR, while not recognizing normal, wild-type EGFR or the unique junctional peptide which is characteristic of de2-7 EGFR.

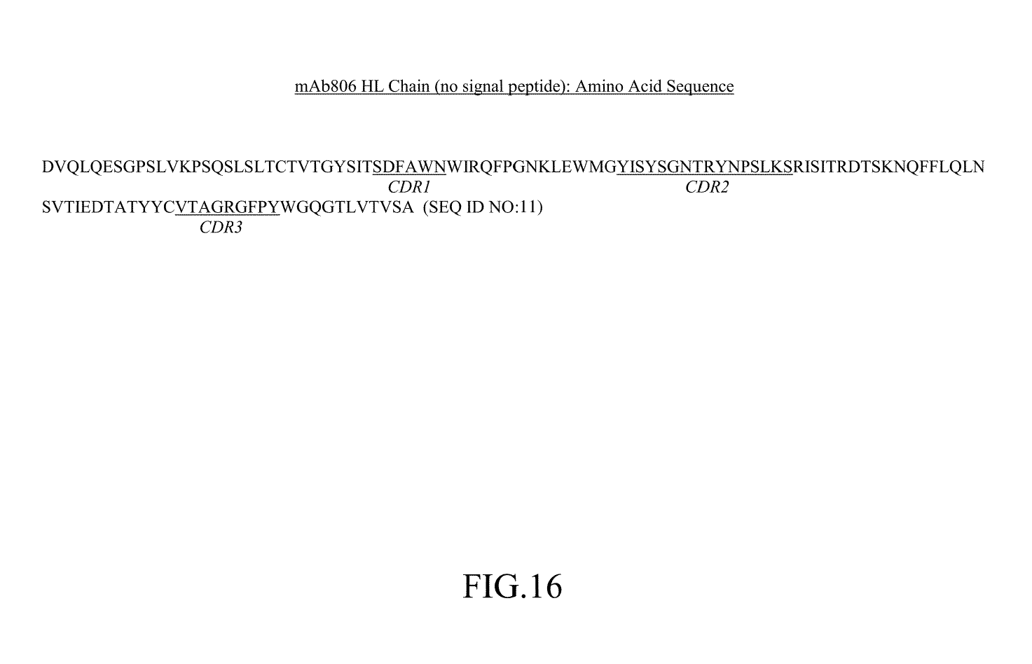

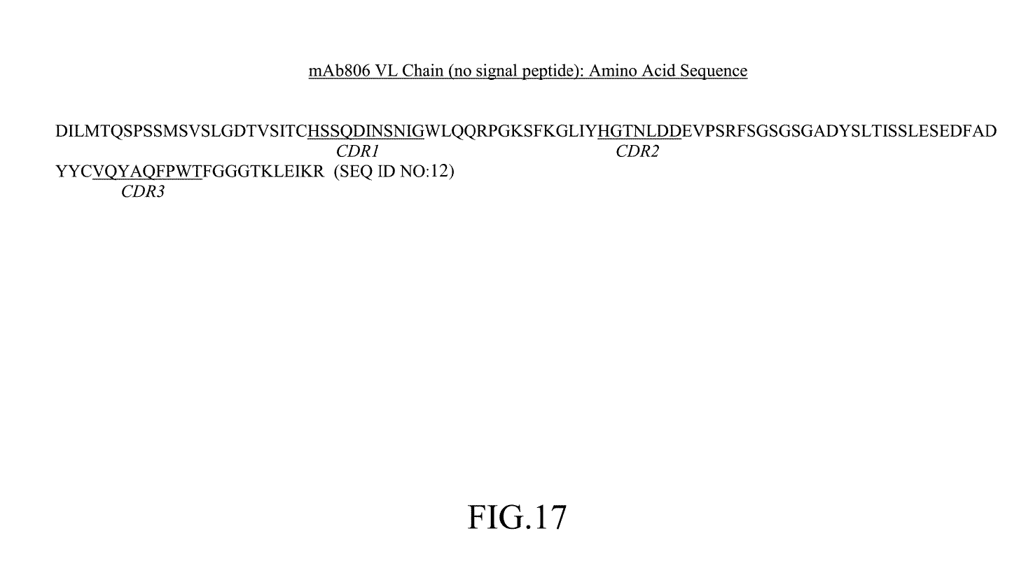

In a preferred aspect, the antibodies are ones which have the characteristics of the antibodies which the inventors have identified and characterized, in particular recognizing amplified EGFR and de2-7EGFR. In particularly preferred aspects, the antibodies are mAb806, mAb175, mAb124, mAb1133, and hu806 or active fragments thereof. In a further preferred aspect the antibody of the present invention comprises the VH and VL chain amino acid sequences depicted FIGS. 16 and 17; 74B and 75B; 51B and 51D; 52B and 52D; and 55A and 55B, respectively.

Preferably the epitope of the specific binding member or antibody is located within the region comprising residues 273-501 of the mature normal or wild-type EGFR sequence, and preferably the epitope comprises residues 287-302 of the mature normal or wild-type EGFR sequence (SEQ ID NO:14). Therefore, also provided are specific binding proteins, such as antibodies, which bind to the de2-7 EGFR at an epitope located within the region comprising residues 273-501 of the EGFR sequence, and comprising residues 287-302 of the EGFR sequence (SEQ ID NO:14). The epitope may be determined by any conventional epitope mapping techniques known to the person skilled in the art. Alternatively, the DNA sequences encoding residues 273-501 and 287-302 (SEQ ID NO:14) could be digested, and the resultant fragments expressed in a suitable host. Antibody binding could be determined as mentioned above.

In particular, the member will bind to an epitope comprising residues 273-501, and more specifically comprising residues 287-302 (SEQ ID NO:14), of the mature normal or wild-type EGFR. However other antibodies which show the same or a substantially similar pattern of reactivity also form an aspect of the invention. This may be determined by comparing such members with an antibody comprising the VH and VL chain domains shown in SEQ ID NOS:2 and 4; 129 and 134; 22 and 27; 32 and 37; and 42 and 47, respectively. The comparison will typically be made using a Western blot in which binding members are bound to duplicate blots prepared from a nuclear preparation of cells so that the pattern of binding can be directly compared.

In another aspect, the invention provides an antibody capable of competing with mAb806 under conditions in which at least 10% of an antibody having the VH and VL chain sequences of one of such antibodies is blocked from binding to de2-7EGFR by competition with such an antibody in an ELISA assay. As set forth above, anti-idiotype antibodies are contemplated and are illustrated herein.

In another aspect, the invention provides an antibody capable of competing with mAb175, mAb124, and/or mAb1133 under conditions in which at least 10% of an antibody having the VH and VL chain sequences of one of such antibodies is blocked from binding to de2-7EGFR by competition with such an antibody in an ELISA assay. As set forth above, anti-idiotype antibodies are contemplated and are illustrated herein.

In another aspect, the invention provides an antibody capable of competing with mAb806, mAb175, mAb124, mAb1133 and/or hu806, under conditions in which at least 10% of an antibody having the VH and VL chain sequences of one of such antibodies is blocked from binding to de2-7EGFR by competition with such an antibody in an ELISA assay. As set forth above, anti-idiotype antibodies are contemplated and are illustrated herein.

An isolated polypeptide consisting essentially of the epitope comprising residues 273-501 and more specifically comprising residues 287-302 (SEQ ID NO:14) of the mature wild-type EGFR forms another aspect of the present invention. The peptide of the invention is particularly useful in diagnostic assays or kits and therapeutically or prophylactically, including as an anti-tumor or anti-cancer vaccine. Thus compositions of the peptide of the present invention include pharmaceutical composition and immunogenic compositions.

### Diagnostic and Therapeutic Uses

The unique specificity of the specific binding members, particularly antibodies or fragments thereof, of the present invention, whereby the binding member (s) recognize an EGFR epitope which is found in tumorigenic, hyperproliferative or abnormal cells and not detectable in normal or wild-type cells and wherein the epitope is enhanced or evident upon aberrant post-translational modification and wherein the member (s) bind to the de2-7 EGFR and amplified EGFR but not the wtEGFR, provides diagnostic and therapeutic uses to identify, characterize, target and treat, reduce or eliminate a number of tumorigenic cell types and tumor types, for example head and neck, breast, lung, bladder or prostate tumors and glioma, without the problems associated with normal tissue uptake that may be seen with previously known EGFR antibodies. Thus, cells overexpressing EGFR (e.g. by amplification or expression of a mutant or variant EGFR), particularly those demonstrating aberrant post-translational modification may be recognized, isolated, characterized, targeted and treated or eliminated utilizing the binding member (s), particularly antibody (ies) or fragments thereof of the present invention.

In a further aspect of the invention, there is provided a method of treatment of a tumor, a cancerous condition, a precancerous condition, and any condition related to or resulting from hyperproliferative cell growth comprising administration of mAb806, mAb175, mAb124, mAb1133, and/or hu806.

The antibodies of the present invention can thus specifically categorize the nature of EGFR tumors or tumorigenic cells, by staining or otherwise recognizing those tumors or cells wherein EGFR overexpression, particularly amplification and/or EGFR mutation, particularly de2-7EGFR, is present. Further, the antibodies of the present invention, as exemplified by mAb806 (and chimeric antibody ch806), mAb175, mAb124, mAb1133, and hu806, demonstrate significant in vivo anti-tumor activity against tumors containing amplified EGFR and against de2-7 EGFR positive xenografts.

As outlined above, the inventors have found that the specific binding member of the invention recognizes tumor-associated forms of the EGFR (de2-7 EGFR and amplified EGFR) but not the normal, wild-type receptor when expressed in normal cells. It is believed that antibody recognition is dependent upon an aberrant posttranslational modification (e.g., a unique glycosylation, acetylation or phosphorylation variant) of the EGFR expressed in cells exhibiting overexpression of the EGFR gene.

As described below, antibodies of the present invention have been used in therapeutic studies and shown to inhibit growth of overexpressing (e.g. amplified) EGFR xenografts and human de2-7 EGFR expressing xenografts of human tumors and to induce significant necrosis within such tumors.

Moreover, the antibodies of the present invention inhibit the growth of intracranial tumors in a preventative model. This model involves injecting glioma cells expressing de2-7 EGFR into nude mice and then injecting the antibody intracranially either on the same day or within 1 to 3 days, optionally with repeated doses. The doses of antibody are suitably about 10 μg. Mice injected with antibody are compared to controls, and it has been found that survival of the treated mice is significantly increased.

Therefore, in a further aspect of the invention, there is provided a method of treatment of a tumor, a cancerous condition, a precancerous condition, and any condition related to or resulting from hyperproliferative cell growth comprising administration of a specific binding member of the invention.

Antibodies of the present invention are designed to be used in methods of diagnosis and treatment of tumors in human or animal subjects, particularly epithelial tumors. These tumors may be primary or secondary solid tumors of any type including, but not limited to, glioma, breast, lung, prostate, head or neck tumors.

### Binding Member and Antibody Generation

The general methodology for making monoclonal antibodies by hybridomas is well known Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammering et al., “Monoclonal Antibodies And T cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; and 4,493,890.

Panels of monoclonal antibodies produced against EFGR can be screened for various properties; i.e., isotype, epitope, affinity, etc. Of particular interest are monoclonal antibodies that mimic the activity of EFGR or its subunits. Such monoclonals can be readily identified in specific binding member activity assays. High affinity antibodies are also useful when immunoaffinity purification of native or recombinant specific binding member is possible.

Methods for producing polyclonal anti-EFGR antibodies are well-known in the art. See U.S. Pat. No. 4,493,795 to Nestor et al. A monoclonal antibody, typically containing Fab and/or F (ab′) 2 portions of useful antibody molecules, can be prepared using the hybridoma technology described in Antibodies-A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an appropriate EGFR.

Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the present antibody or binding member and their ability to inhibit specified tumorigenic or hyperproliferative activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

Methods for producing monoclonal anti-EGFR antibodies are also well-known in the art. See Niman et al., Proc. Natl. Acad. Sci. USA, 80:4949-4953 (1983). Typically, the EGFR or a peptide analog is used either alone or conjugated to an immunogenic carrier, as the immunogen in the before described procedure for producing anti-EGFR monoclonal antibodies. The hybridomas are screened for the ability to produce an antibody that immunoreacts with the EGFR present in tumorigenic, abnormal or hyperproliferative cells. Other anti-EGFR antibodies include but are not limited to the HuMAX-EGFr antibody from Genmab/Medarex, the 108 antibody (ATCC HB9764) and U.S. Pat. No. 6,217,866, and antibody 14E1 from Schering A G (U.S. Pat. No. 5,942,602).

### Recombinant Binding Members, Chimerics, Bispecifics and Fragments

In general, the CDR1 regions, comprising amino acid sequences substantially as set out as the CDR1 regions of SEQ ID NOS:2 and 4; 129 and 134; 22 and 27; 32 and 37; and 42 and 47, respectively, will be carried in a structure which allows for binding of the CDR1 regions to an tumor antigen. In the case of the CDR1 region of SEQ ID NO:4, for example, this is preferably carried by the VL chain region of SEQ ID NO:4 (and similarly for the other recited sequences).

In general, the CDR2 regions, comprising amino acid sequences substantially as set out as the CDR2 regions of SEQ ID NOS:2 and 4; 129 and 134; 22 and 27; 32 and 37; and 42 and 47, respectively, will be carried in a structure which allows for binding of the CDR2 regions to an tumor antigen. In the case of the CDR2 region of SEQ ID NO:4, for example, this is preferably carried by the VL chain region of SEQ ID NO:4 (and similarly for the other recited sequences).

In general, the CDR3 regions, comprising amino acid sequences substantially as set out as the CDR3 regions of SEQ ID NOS:2 and 4; 129 and 134; 22 and 27; 32 and 37; and 42 and 47, respectively, will be carried in a structure which allows for binding of the CDR3 regions to an tumor antigen. In the case of the CDR3 region of SEQ ID NO:4, for example, this is preferably carried by the VL chain region of SEQ ID NO:4 (and similarly for the other recited sequences).

By “substantially as set out” it is meant that that CDR regions, for example CDR3 regions, of the invention will be either identical or highly homologous to the specified regions of SEQ ID NOS:2 and 4; 129 and 134; 22 and 27; 32 and 37; and 42 and 47, respectively. By “highly homologous” it is contemplated that only a few substitutions, preferably from 1 to 8, preferably from 1 to 5, preferably from 1 to 4, or from 1 to 3 or 1 or 2 substitutions may be made in one or more of the CDRs. It is also contemplated that such terms include truncations to the CDRs, so long as the resulting antibody exhibits the unique properties of the class of antibodies discussed herein, as exhibited by mAb806, mAb175, mAb124, mAb1133 and hu806.

The structure for carrying the CDRs of the invention, in particular CDR3, will generally be of an antibody heavy or light chain sequence or substantial portion thereof in which the CDR regions are located at locations corresponding to the CDR region of naturally occurring VH and VL chain antibody variable domains encoded by rearranged immunoglobulin genes. The structures and locations of immunoglobulin variable domains may be determined by reference to Kabat, E. A. et al, Sequences of Proteins of Immunological Interest. 4th Edition. US Department of Health and Human Services. 1987, and updates thereof, now available on the Internet (http://immuno.bme.nwu.edu)). Moreover, as is known to those of skill in the art, CDR determinations can be made in various ways. For example, Kabat, Chothia and combined domain determination analyses may be used. In this regard, see for example http://www.bioinf.org.uk/abs/#cdrid.

Preferably, the amino acid sequences substantially as set out as the VH chain CDR residues in the inventive antibodies are in a human heavy chain variable domain or a substantial portion thereof, and the amino acid sequences substantially as set out as the VL chain CDR residues in the inventive antibodies are in a human light chain variable domain or a substantial portion thereof.

The variable domains may be derived from any germline or rearranged human variable domain, or may be a synthetic variable domain based on consensus sequences of known human variable domains. The CDR3-derived sequences of the invention, for example, as defined in the preceding paragraph, may be introduced into a repertoire of variable domains lacking CDR3 regions, using recombinant DNA technology.

For example, Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide specific binding members of the invention. The repertoire may then be displayed in a suitable host system such as the phage display system of WO92/01047 so that suitable specific binding members may be selected. A repertoire may consist of from anything from 10⁴ individual members upwards, for example from 10⁶ to 10⁸ or 10¹⁰ members.

Analogous shuffling or combinatorial techniques are also disclosed by Stemmer (Nature, 1994, 370:389-391), who describes the technique in relation to a p-lactamase gene but observes that the approach may be used for the generation of antibodies.

A further alternative is to generate novel VH or VL regions carrying the CDR3 derived sequences of the invention using random mutagenesis of, for example, the mAb806 VH or VL genes to generate mutations within the entire variable domain. Such a technique is described by Gram et al (1992, Proc. Natl. Acad. Sci., USA, 89:3576-3580), who used error-prone PCR.

Another method which may be used is to direct mutagenesis to CDR regions of VH or VL genes. Such techniques are disclosed by Barbas et al, (1994, Proc. Natl. Acad. Sci., USA, 91:3809-3813) and Schier et al. (1996, J. Mol. Biol. 263:551-567).

All the above described techniques are known as such in the art and in themselves do not form part of the present invention. The skilled person will be able to use such techniques to provide specific binding members of the invention using routine methodology in the art.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as discussed in more detail below.

Although in a preferred aspect of the invention specific binding members comprising a pair of binding domains based on sequences substantially set out in SEQ ID NOS:2 and 4; 129 and 134; 22 and 27; 32 and 37; and 42 and 47, respectively, are preferred, single binding domains based on these sequences form further aspects of the invention. In the case of the binding domains based on the sequence substantially set out in VH chains, such binding domains may be used as targeting agents for tumor antigens since it is known that immunoglobulin VH domains are capable of binding target antigens in a specific manner.

In the case of either of the single chain specific binding domains, these domains may be used to screen for complementary domains capable of forming a two-domain specific binding member which has in vivo properties as good as or equal to the mAb806, ch806, mAb175, mAb124, mAb1133 and hu806 antibodies disclosed herein.

This may be achieved by phage display screening methods using the so-called hierarchical dual combinatorial approach as disclosed in U.S. Pat. No. 5,969,108 in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al, ibid.

Specific binding members of the present invention may further comprise antibody constant regions or parts thereof. For example, specific binding members based on VL chain sequences may be attached at their C-terminal end to antibody light chain constant domains including human Ck of Cλ chains, preferably Cλ chains. Similarly, specific binding members based on VH chain sequences may be attached at their C-terminal end to all or part of an immunoglobulin heavy chain derived from any antibody isotype, e.g. IgG, IgA, IgE, IgD and IgM and any of the isotype sub-classes, particularly IgG1, IgG2b, and IgG4. IgG1 is preferred.

The advent of monoclonal antibody (mAb) technology 25 years ago has provide an enormous repertoire of useful research reagents and created the opportunity to use antibodies as approved pharmaceutical reagents in cancer therapy, autoimmune disorders, transplant rejection, antiviral prophylaxis and as anti-thrombotics (Glennie and Johnson, 2000). The application of molecular engineering to convert murine mAbs into chimeric mAbs (mouse V-region, human C-region) and humanized reagents where only the mAb complementarity-determining regions (CDR) are of murine origin has been critical to the clinical success of mAb therapy. The engineered mAbs have markedly reduced or absent immunogenicity, increased serum half-life and the human Fc portion of the mAb increases the potential to recruit the immune effectors of complement and cytotoxic cells (Clark 2000). Investigations into the biodistribution, pharmacokinetics and any induction of an immune response to clinically administered mAbs requires the development of analyses to discriminate between the pharmaceutical and endogenous proteins.

The antibodies, or any fragments thereof, may also be conjugated or recombinantly fused to any cellular toxin, bacterial or other, e.g. pseudomonas exotoxin, ricin, or diphtheria toxin. The part of the toxin used can be the whole toxin, or any particular domain of the toxin. Such antibody-toxin molecules have successfully been used for targeting and therapy of different kinds of cancers, see e.g. Pastan, Biochim Biophys Acta. 1997 Oct. 24; 1333 (2):C₁₋₆; Kreitman et al., N. Engl. 1 Med. 2001 Jul. 26; 345 (4):241-7; Schnell et al., Leukemia. 2000 Jan.; 14 (1):129-35; Ghetie et al., Mol. Biotechnol. 2001 Jul.; 18 (3):251-68.

Bi- and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J. Immunol. Methods. 2001 Feb. 1; 248 (1-2):47-66; Tomlinson et al., Methods Enzymol. 2000; 326:461-79; McCall et al., J. Immunol. 2001 May 15; 166 (10):6112-7.

Fully human antibodies can be prepared by immunizing transgenic mice carrying large portions of the human immunoglobulin heavy and light chains. These mice, examples of such mice are the Xenomouse™ (Abgenix, Inc.) (U.S. Pat. Nos. 6,075,181 and 6,150,584), the HuMAb-Mouse™ (Medarex, Inc./GenPharm) (U.S. Pat. Nos. 5,545,806 and 5,569,825), the TransChromo Mouse (Kirin) and the KM Mouse (Medarex/Kirin), are well known within the art.

Antibodies can then be prepared by, e.g. standard hybridoma technique or by phage display. These antibodies will then contain only fully human amino acid sequences.

Fully human antibodies can also be generated using phage display from human libraries. Phage display may be performed using methods well known to the skilled artisan, as in Hoogenboom et al. and Marks et al. (Hoogenboom H R and Winter G. (1992) J. Mol. Biol. 227 (2):381-8; Marks J D et al. (1991) J. Mol. Biol. 222 (3):581-97; and also U.S. Pat. Nos. 5,885,793 and 5,969,108).

### Therapeutic Antibodies and Uses

The in vivo properties, particularly with regard to tumor:blood ratio and rate of clearance, of specific binding members of the invention will be at least comparable to mAb806. Following administration to a human or animal subject such a specific binding member will show a peak tumor to blood ratio of >1:1. Preferably at such a ratio the specific binding member will also have a tumor to organ ratio of greater than 1:1, preferably greater than 2:1, more preferably greater than 5:1. Preferably at such a ratio the specific binding member will also have an organ to blood ratio of <1:1 in organs away from the site of the tumor. These ratios exclude organs of catabolism and secretion of the administered specific binding member. Thus in the case of scFvs and Fabs (as shown in the accompanying examples), the binding members are secreted via the kidneys and there is greater presence here than other organs. In the case of whole IgGs, clearance will be at least in part, via the liver. The peak localization ratio of the intact antibody will normally be achieved between 10 and 200 hours following administration of the specific binding member. More particularly, the ratio may be measured in a tumor xenograft of about 0.2-1.0 g formed subcutaneously in one flank of an athymic nude mouse.

Antibodies of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ²¹¹At, ¹⁹⁸Au, ⁶⁷CU, ²²⁵Ac, ²¹³Bi, ⁹⁹TC and ¹⁸⁶Re, which may be attached to antibodies of the invention using conventional chemistry known in the art of antibody imaging. Labels also include fluorescent labels and labels used conventionally in the art for MRI-CT imagine. They also include enzyme labels such as horseradish peroxidase. Labels further include chemical moieties such as biotin which may be detected via binding to a specific cognate detectable moiety, e.g. labeled avidin.

Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Also, antibodies including both polyclonal and monoclonal antibodies, and drugs that modulate the production or activity of the specific binding members, antibodies and/or their subunits may possess certain diagnostic applications and may for example, be utilized for the purpose of detecting and/or measuring conditions such as cancer, precancerous lesions, conditions related to or resulting from hyperproliferative cell growth or the like. For example, the specific binding members, antibodies or their subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity (ies) of the specific binding members of the invention may be discovered or synthesized, and may be used in diagnostic and/or therapeutic protocols.

The radiolabeled specific binding members, particularly antibodies and fragments thereof, are useful in in vitro diagnostics techniques and in in vivo radioimaging techniques and in radioimmunotherapy. In the instance of in vivo imaging, the specific binding members of the present invention may be conjugated to an imaging agent rather than a radioisotope (s), including but not limited to a magnetic resonance image enhancing agent, wherein for instance an antibody molecule is loaded with a large number of paramagnetic ions through chelating groups. Examples of chelating groups include EDTA, porphyrins, polyamines crown ethers and polyoximes. Examples of paramagnetic ions include gadolinium, iron, manganese, rhenium, europium, lanthanium, holmium and erbium. In a further aspect of the invention, radiolabeled specific binding members, particularly antibodies and fragments thereof, particularly radioimmunoconjugates, are useful in radioimmunotherapy, particularly as radiolabeled antibodies for cancer therapy. In a still further aspect, the radiolabelled specific binding members, particularly antibodies and fragments thereof, are useful in radioimmuno-guided surgery techniques, wherein they can identify and indicate the presence and/or location of cancer cells, precancerous cells, tumor cells, and hyperproliferative cells, prior to, during or following surgery to remove such cells.

The present invention also includes immunoconjugates, wherein specific binding members of the present invention, particularly antibodies and fragments thereof, are conjugated or attached to one or more agents for modifying a biological response (such as, for example and without limitation, inhibiting or preventing the expression activity of cells, causing the destruction of cells, or otherwise effecting the function of cells). Such agents include, for example and without limitation, chemical ablation agents, toxins, immunomodulators, cytokines, cytotoxic agents, chemotherapeutic agents and/or drugs, and include, but are not limited to, the following:
4-desacetylvinblastine-3-carbohydiazide;
5-fluoro-T-deoxyuridine;
5-fluorouracil;
5-fluorouracil decarbonizes;
6-mercaptopurine;
6-thioguanine;
abrin;
abrin A chain;
actinomycin D;
actinomycin D, 1-dehydrotestosterone;
adriamycin;
alkylating agents;
alkylphosphocholines;
aminopterin;
angiogenin;
angiostatin;
anthracyclines;
anthramycin;
anti-angiogenics;
anti-folates;
anti-metabolites;
anti-mitotics;
antibiotics;
ara-C;
auristatin derivatives (see, for example and without limitation, U.S. Patent Publication Nos. 2008/0300192, 2009/0018086, 2009/0018086, and 2009/0111756, each of which is hereby incorporated by reference in its entirety);
auristatin E (see, for example and without limitation, U.S. Pat. No. 5,635,483, hereby incorporated by reference in its entirety);
auristatin E valeryl benzylhydrazone; auristatin F phenylene diamine;
auristatins;
auromycins;
bis-iodo-phenol mustard;
bismuth;
bleomycin;
busulfan;
calicheamicin;
carboplatin;
caminomycin;
carmustine;
cc-1065 compounds (see, for example and without limitation, U.S. Pat. Nos. 5,475,092, 5,585,499, 5,846,545, 6,534,660, 6,586,618, 6,756,397, 7,049,316, 7,329,760, 7,388,026, 7,655,660, and 7,655,661, U.S. Patent Publication. Nos. 2007/0135346, 2008/0260685, and 2009/0281158, and 2009/0318668, and PCT Publication No. WO2009/017394, each of which is hereby incorporated by reference in its entirety);
chlorambucil;
cis-dichlorodiamine platinum (cisplatin);
colchicin (colchicine);
combrestatin;
crotin;
curicin;
cyclothosphamide;
cytarabine;
cytochalasin B;
cytosine arabinoside;
cytoxin;
dacarbazine;
dactinomycin (actinomycin);
daunorubicin (daunomycin);
dibromomannitol;
dihydroxy anthracin dione;
diphtheria toxin;
dolastatin-10;
doxetaxel;
doxorubicin;
doxorubicin hydrazides;
duocarmycins (see, for example and without limitation, U.S. Pat. No. 7,214,685, hereby incorporated by reference in its entirety);
emetine;
endostatin;
enediyenes;
enomycin;
epirubicin;
esperamicin compounds (see, for example and without limitation, U.S. Pat. No. 4,675,187, hereby incorporated by reference in its entirety);
ethidium bromide;
etoposide;
gelonin;
glucocorticoids;
gramicidin D; granulocyte colony stimulating factor;
granulocyte macrophage colony stimulating factor;
idarubicin;
intercalating agents;
interleukin-1;
interleukin-2;
interleukin-6;
lidocaine;
lomustine;
lymphokine;
maytansinols (see, for example and without limitation, U.S. Pat. Nos. 4,137,230, 4,151,042, 4,162,940, 4,190,580, 4,225,494, 4,228,239, 4,248,870, 4,256,746, 4,260,608, 4,263,294, 4,264,596, 4,265,814, 4,294,757, 4,307,016, 4,308,268, 4,308,269, 4,309,428, 4,317,821, 4,320,200, 4,322,348, 4,331,598, 4,360,462, 4,361,650, 4,362,663, 4,364,866, 4,371,533, 4,424,219, 4,450,234, 5,141,736, and 5,217,713, each of which is hereby incorporated by reference in its entirety);
mechlorethamine;
melphalan (and other related nitrogen mustards);
methotrexate;
minor groove-binders;
mithramycin;
mitogellin;
mitomycin C;
mitomycins;
mitoxantrone;
MMAF-dimethylaminoethylamine;
MMAF-N-t-butyl;
MMAF-tetraethylene glycol;
modeccin A chain;
mono-methyl auristatin E (MMAE) (see, for example and without limitation, U.S. Pat. Nos. 6,884,869, 7,098,308, 7,256,257, and 7,423,116, and U.S. Patent Publication Nos. 2003/0083263, 2004/0157782, 2005/0009751, 2005/0113308, and 2006/0229253, each of which is hereby incorporated by reference in its entirety);
mono-methyl auristatin F (MMAF) (see, for example and without limitation, U.S. Pat. No. 7,498,298, and U.S. Patent Publication Nos. 2008/0226657, 2008/0248051, 2008/0248053, and 2009/0047296, each of which is hereby incorporated by reference in its entirety);
morpholinodoxorubicin;
N2′-deacetyl-N2′-(c-mercapto-1 oxopropyl)-maytansine (DM1) (see, for example and without limitation, U.S. Pat. No. 5,208,020, hereby incorporated by reference in its entirety);
N2′-deacetyl-N2′-(4-mercapto-4-methyl-1-oxopentyl)-maytansine (DM4) (see, for example and without limitation, U.S. Pat. No. 7,276,497, hereby incorporated by reference in its entirety);
neocarzinostatin;
nerve growth factor (and other growth factors);
onapristone;
paclitaxel;
PE40;
phenomycin;
platelet derived growth factor;
prednisone;
procaine;
propranolol;
Pseudomonas exotoxin A;
puromycin;
radioactive isotopes (such as, for example and without limitation, At²¹¹, Bi²¹², Bi²¹³, Cf²⁵², I¹²⁵, I¹³¹, In¹¹¹, Ir¹⁹², Lu¹⁷⁷, P³², Re¹⁸⁶, Re¹⁸⁸, Sm¹⁵³, Y⁹⁰, and W¹⁸⁸);
retstrictocin;
ricin A;
ricins;
Sapaonaria officinalis inhibitor;
saporin;
streptozotocin;
suramin;
tamoxifen;
taxanes;
taxoids;
taxol;
tenoposide;
tetracaine;
thioepa chlorambucil;
thiotepa;
thrombotic agents;
tissue plasminogen activator;
topoisomerase I inhibitors;
topoisomerase II inhibitors;
toxotere;
tumor necrosis factor;
vinblastine;
vinca alkaloids;
vincas;
vincristine;
vindesine;
vinorelbine;
yttrium;
α-interferon;
α-sarcin; and
β-interferon,
   as well as analogs, homologs, fragments, variants, and derivatives thereof (see also Garnett (2001) Advanced drug Delivery Reviews 53:171-216, hereby incorporated by reference in its entirety).

As will be understood by those of skill in the art, the agents set forth above, as well as other suitable agents, may be conjugated or attached to specific binding members of the present invention, particularly antibodies and fragments thereof, in any suitable manner to produce immunoconjugates of the present invention. For example and without limitation, in various embodiments of the present invention the binding member(s) and agent(s) may be covalently attached and/or may be conjugated using linker, spacer and/or stretcher compounds, which in various embodiments of the present invention are cleavable, are non-cleavable, and result in the therapeutic agent(s) being internalized by the target cell.

For example, such linker, spacer and/or stretcher compounds include, but are not limited to, the following: amino benzoic acid spacers (see, for example and without limitation, U.S. Pat. Nos. 7,091,186 and 7,553,816, each of which is hereby incorporated by reference in its entirety); maleimidocaproyl; p-aminobenzylcarbamoyl (PAB); lysosomal enzyme-cleavable linkers (see, for example and without limitation, U.S. Pat. No. 6,214,345, hereby incorporated by reference in its entirety); maleimidocaproyl-polyethylene glycol (MC(PEG)6-OH); N-methyl-valine citrulline; N-succinimidyl 4-(N-maleimidomethyl)cyclohexane-1-carboxylate (SMCC) (see, for example and without limitation, Yoshitake et al. (1979) Eur. J. Biochem., 101, 395-399, hereby incorporated by reference in its entirety); N— succinimidyl 4-(2-pyridyldithio)butanoate (SPDB) (see, for example and without limitation, U.S. Pat. No. 4,563,304, hereby incorporated by reference in its entirety); N-Succinimidyl 4-(2-pyridylthio)pentanoate (SPP); valine-citrulline; and other linker, spacer, and/or stretcher compounds (see, for example and without limitation, U.S. Pat. Nos. 7,090,843, 7,223,837, and 7,659,241, and U.S. Patent Publication Nos. 2004/0018194, 2004/0121940, 2006/0116422, 2007/0258987, 2008/0213289, 2008/0241128, 2008/0311136, 2008/0317747, and 2009/0010945, each of which is hereby incorporated by reference in its entirety).

Generally speaking, techniques for attaching and/or conjugating the agents set forth above, as well as other agents, to specific binding members of the present invention, particularly antibodies and fragments thereof, are known in the art. See, for example and without limitation, Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982), each of which is hereby incorporated by reference in its entirety.

Furthermore, specific binding members of the present invention, particularly antibodies and fragments thereof, may be conjugated to a second antibody to form an antibody heteroconjugate (see, for example and without limitation, U.S. Pat. No. 4,676,980, hereby incorporated by reference in its entirety), may be administered (either with or without an agent attached or conjugated thereto) alone or in combination with another agent (for example and without limitation, an agent set forth above), and/or may be conjugated to an anti-cancer pro-drug activating enzyme capable of converting the pro-drug to its active form.

Radioimmunotherapy (RAIT) has entered the clinic and demonstrated efficacy using various antibody immunoconjugates. ¹³¹1 labeled humanized anti-carcinoembryonic antigen (anti-CEA) antibody hMN-14 has been evaluated in colorectal cancer (Behr T M et al (2002) Cancer 94 (4Suppl):1373-81) and the same antibody with 90Y label has been assessed in medullary thyroid carcinoma (Stein R et al (2002) Cancer 94 (1):51-61). Radioimmunotherapy using monoclonal antibodies has also been assessed and reported for non-Hodgkin's lymphoma and pancreatic cancer (Goldenberg D M (2001) Crit. Rev. Oncol. Hematol. 39 (1-2):195-201; Gold D V et al. (2001) Crit. Rev. Oncol. Hematol. 39 (1-2) 147-54). Radioimmunotherapy methods with particular antibodies are also described in U.S. Pat. Nos. 6,306,393 and 6,331,175. Radioimmunoguided surgery (RIGS) has also entered the clinic and demonstrated efficacy and usefulness, including using anti-CEA antibodies and antibodies directed against tumor-associated antigens (Kim J C et al (2002) Jut. J. Cancer 97(4):542-7; Schneebaum, S. et al. (2001) World J. Surg. 25(12):1495-8; Avital, S. et al. (2000) Cancer 89(8):1692-8; McIntosh D G et al (1997) Cancer Biother. Radiopharm. 12 (4):287-94).

Antibodies of the present invention may be administered to a patient in need of treatment via any suitable route, usually by injection into the bloodstream or CSF, or directly into the site of the tumor. The precise dose will depend upon a number of factors, including whether the antibody is for diagnosis or for treatment, the size and location of the tumor, the precise nature of the antibody (whether whole antibody, fragment, diabody, etc), and the nature of the detectable or functional label attached to the antibody. Where a radioisotope is used for therapy, a suitable maximum single dose is about 45 mCi/m², to a maximum of about 250 mCi/m². Preferable dosage is in the range of 15 to 40 mCi, with a further preferred dosage range of 20 to 30 mCi, or to 30 mCi, depending on the isotope utilized. Such therapy may require bone marrow or stem cell replacement. A typical antibody dose for either tumor imaging or radioisotope-conjugated tumor treatment will be in the range of from 0.5 to 1000 mg. Naked antibodies are preferably administered in doses of 20 to 1000 mg protein per dose, or 20 to 500 mg protein per dose, or 20 to 100 mg protein per dose. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician.

These formulations may include a second binding protein, such as the EGPR binding proteins described supra. In an especially preferred form, this second binding protein is a monoclonal antibody such as 528 or 225, discussed infra.

### Pharmaceutical and Therapeutic Compositions

Specific binding members of the present invention will usually be administered in the form of a pharmaceutical composition, which may comprise at least one component in addition to the specific binding member.

Thus pharmaceutical compositions according to the present invention, and for use in accordance with the present invention, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A composition may be administered alone or in combination with other treatments, therapeutics or agents, either simultaneously or sequentially dependent upon the condition to be treated. In addition, the present invention contemplates and includes compositions comprising the binding member, particularly antibody or fragment thereof, herein described and other agents or therapeutics such as anti-cancer agents or therapeutics, hormones, anti-EGFR agents or antibodies, or immune modulators. More generally these anti-cancer agents may be tyrosine kinase inhibitors or phosphorylation cascade inhibitors, post-translational modulators, cell growth or division inhibitors (e.g. anti-mitotics), or signal transduction inhibitors. Other treatments or therapeutics may include the administration of suitable doses of pain relief drugs such as non-steroidal anti-inflammatory drugs (e.g., aspirin, paracetamol, ibuprofen or ketoprofen) or opiates such as morphine, or anti-emetics. The composition can be administered in combination (either sequentially (i.e. before or after) or simultaneously) with tyrosine kinase inhibitors (including, but not limited to AG1478 and ZD1839, STI571, OSI-774, SU-6668), doxorubicin, temozolomide, cisplatin, carboplatin, nitrosoureas, procarbazine, vincristine, hydroxyurea, 5-fluoruracil, cytosine arabinoside, cyclophosphamide, epipodophyllotoxin, carmustine, lomustine, and/or other chemotherapeutic agents. Thus, these agents may be anti-EGFR specific agents, or tyrosine kinase inhibitors such as AG1478, ZD1839, STI571, OSI-774, or SU-6668 or may be more general anti-cancer and anti-neoplastic agents such as doxorubicin, cisplatin, temozolomide, nitrosoureas, procarbazine, vincristine, hydroxyurea, 5-fluoruracil, cytosine arabinoside, cyclophosphamide, epipodophyllotoxin, carmustine, or lomustine. In addition, the composition may be administered with hormones such as dexamethasone, immune modulators, such as interleukins, tumor necrosis factor (TNF) or other growth factors or cytokines which stimulate the immune response and reduction or elimination of cancer cells or tumors.

An immune modulator such as TNF may be combined together with a member of the invention in the form of a bispecific antibody recognizing the EGFR epitope recognized by the inventive antibodies, as well as binding to TNF receptors. The composition may also be administered with, or may include combinations along with other anti-EGFR antibodies, including but not limited to the anti-EGFR antibodies 528, 225, SC-03, DR8. 3, L8A4, Y10, ICR62 and ABX-EGF.

Previously the use of agents such as doxorubicin and cisplatin in conjunction with anti-EGFR antibodies have produced enhanced anti-tumor activity (Fan et al, 1993; Baselga et al, 1993). The combination of doxorubicin and mAb 528 resulted in total eradication of established A431 xenografts, whereas treatment with either agent alone caused only temporary in vivo growth inhibition (Baselga et al, 1993). Likewise, the combination of cisplatin and either mAb528 or 225 also led to the eradication of well established A431 xenografts, which was not observed when treatment with either agent was used (Fan et al, 1993).

### Conventional Radiotherapy

In addition, the present invention contemplates and includes therapeutic compositions for the use of the binding member in combination with conventional radiotherapy. It has been indicated that treatment with antibodies targeting EGF receptors can enhance the effects of conventional radiotherapy (Milas et al., Clin. Cancer Res. 2000 Feb.:6 (2):701, Huang et al., Clin. Cancer Res. 2000 Jun.:6 (6):2166).

As demonstrated herein, combinations of the binding member of the present invention, particularly an antibody or fragment thereof, preferably the mAb806, ch806, mAb175, mAb124, mAb1133 or hu806 or a fragment thereof, and anti-cancer therapeutics, particularly anti-EGFR therapeutics, including other anti-EGFR antibodies, demonstrate effective therapy, and particularly synergy, against xenografted tumors. In the Examples, it is demonstrated, for example, that the combination of AG1478 and mAb806 results in significantly enhanced reduction of A431 xenograft tumor volume in comparison with treatment with either agent alone. AG1478 (4-(3-chloroanilino)-6,7-dimethoxyquinazoline) is a potent and selective inhibitor of the EGF receptor kinase and is particularly described in U.S. Pat. No. 5,457,105, incorporated by reference herein in its entirety (see also, Liu, W. et al (1999) J. Cell Sci. 112:2409; Eguchi, S. et al. (1998) J. Biol. Chem. 273:8890; Levitsky, A. and Gazit, A. (1995) Science 267:1782). The Specification Examples further demonstrate therapeutic synergy of antibodies of the present invention with other anti-EGFR antibodies, particularly with the 528 anti-EGFR antibody.

The present invention further contemplates therapeutic compositions useful in practicing the therapeutic methods of this invention. A subject therapeutic composition includes, in admixture, a pharmaceutically acceptable excipient (carrier) and one or more of a specific binding member, polypeptide analog thereof or fragment thereof, as described herein as an active ingredient. In a preferred embodiment, the composition comprises an antigen capable of modulating the specific binding of the present binding member/antibody with a target cell.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of EFGR binding capacity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

### Diagnostic Assays

The present invention also relates to a variety of diagnostic applications, including methods for detecting the presence of stimuli such as aberrantly expressed EGFR, by reference to their ability to be recognized by the present specific binding member. As mentioned earlier, the EGFR can be used to produce antibodies to itself by a variety of known techniques, and such antibodies could then be isolated and utilized as in tests for the presence of particular EGFR activity in suspect target cells.

Diagnostic applications of the specific binding members of the present invention, particularly antibodies and fragments thereof, include in vitro and in vivo applications well known and standard to the skilled artisan and based on the present description. Diagnostic assays and kits for in vitro assessment and evaluation of EGFR status, particularly with regard to aberrant expression of EGFR, may be utilized to diagnose, evaluate and monitor patient samples including those known to have or suspected of having cancer, a precancerous condition, a condition related to hyperproliferative cell growth or from a tumor sample. The assessment and evaluation of EGFR status is also useful in determining the suitability of a patient for a clinical trial of a drug or for the administration of a particular chemotherapeutic agent or specific binding member, particularly an antibody, of the present invention, including combinations thereof, versus a different agent or binding member. This type of diagnostic monitoring and assessment is already in practice utilizing antibodies against the HER2 protein in breast cancer (Hercep Test, Dako Corporation), where the assay is also used to evaluate patients for antibody therapy using Herceptin. In vivo applications include imaging of tumors or assessing cancer status of individuals, including radioimaging.

As suggested previously, the diagnostic method of the present invention comprises examining a cellular sample or medium by means of an assay including an effective amount of an antagonist to an EFGR/protein, such as an anti-EFGR antibody, preferably an affinity-purified polyclonal antibody, and more preferably a mAb. In addition, it is preferable for the anti-EFGR antibody molecules used herein be in the form of Fab, Fab′, F (ab′)₂ or F (v) portions or whole antibody molecules. As previously discussed, patients capable of benefiting from this method include those suffering from cancer, a pre-cancerous lesion, a viral infection, pathologies involving or resulting from hyperproliferative cell growth or other like pathological derangement. Methods for isolating EFGR and inducing anti-EFGR antibodies and for determining and optimizing the ability of anti-EFGR antibodies to assist in the examination of the target cells are all well-known in the art.

Preferably, the anti-EFGR antibody used in the diagnostic methods of this invention is an affinity purified polyclonal antibody. More preferably, the antibody is a monoclonal antibody (mAb). In addition, the anti-EFGR antibody molecules used herein can be in the form of Fab, Fab′, F(ab′)₂ or F(v) portions of whole antibody molecules.

As described in detail above, antibody(ies) to the EGFR can be produced and isolated by standard methods including the well known hybridoma techniques. For convenience, the antibody (ies) to the EGFR will be referred to herein as Ab₁ and antibody (ies) raised in another species as Ab₂.

The presence of EGFR in cells can be ascertained by the usual in vitro or in vivo immunological procedures applicable to such determinations. A number of useful procedures are known. Three such procedures which are especially useful utilize either the EGFR labeled with a detectable label, antibody Ab, labeled with a detectable label, or antibody Ab2 labeled with a detectable label. The procedures may be summarized by the following equations wherein the asterisk indicates that the particle is labeled, and “R” stands for the EGFR:

### A. R*+Ab₁=R*Ab₁,

### B. R+Ab*=RAb₁*

### C. R+Ab₁+Ab₂*=RAb₁Ab₂*

The procedures and their application are all familiar to those skilled in the art and accordingly may be utilized within the scope of the present invention. The “competitive” procedure, Procedure A, is described in U.S. Pat. Nos. 3,654,090 and 3,850,752. Procedure C, the “sandwich” procedure, is described in U.S. Pat. Nos. RE 31,006 and 4,016,043. Still other procedures are known such as the “double antibody,” or “DASP” procedure.

In each instance above, the EGFR forms complexes with one or more antibody (ies) or binding partners and one member of the complex is labeled with a detectable label. The fact that a complex has formed and, if desired, the amount thereof, can be determined by known methods applicable to the detection of labels.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary or anti-EGFR antibody, and Ab₂ will be referred to as a secondary or anti-Ab₁ antibody.

The labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The EGFR or its binding partner (s) such as the present specific binding member, can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ²¹¹At, ¹⁹⁸Au, ⁶⁷Cu ²²⁵Ac, ²¹³Bi, ⁹⁹Tc and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A particular assay system that may be advantageously utilized in accordance with the present invention, is known as a receptor assay. In a receptor assay, the material to be assayed such as the specific binding member, is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the specific binding member may be radiolabeled and combined, for example, with antibodies or other inhibitors thereto, after which binding studies would be carried out. Solutions would then be prepared that contain various quantities of labeled and unlabeled uncombined specific binding member, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay may be performed and utilized, in the instance where the cellular binding ability of the assayed material may serve as a distinguishing characteristic.

An assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and PCT International Publication No. WO 88/03168, for which purpose the artisan is referred.

In a further embodiment of this invention, commercial test kits suitable for use by a medical specialist may be prepared to determine the presence or absence of aberrant expression of EGFR, including but not limited to amplified EGFR and/or an EGFR mutation, in suspected target cells. In accordance with the testing techniques discussed above, one class of such kits will contain at least the labeled EGFR or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for aberrant expression or post-translational modification of EGFR, comprising:

(a) a predetermined amount of at least one labeled immunochemically reactive component obtained by the direct or indirect attachment of the present specific binding member or a specific binding partner thereto, to a detectable label;

(b) other reagents; and

(c) directions for use of said kit.

More specifically, the diagnostic test kit may comprise:

(a) a known amount of the specific binding member as described above (or a binding partner) generally bound to a solid phase to form an immunosorbent, or in the alternative, bound to a suitable tag, or plural such end products, etc. (or their binding partners) one of each;

(b) if necessary, other reagents; and

(c) directions for use of said test kit.

In a further variation, the test kit may be prepared and used for the purposes stated above, which operates according to a predetermined protocol (e.g., “competitive,” “sandwich,” “double antibody,” etc.), and comprises:

(a) a labeled component which has been obtained by coupling the specific binding member to a detectable label;

(b) one or more additional immunochemical reagents of which at least one reagent is a ligand or an immobilized ligand, which ligand is selected from the group consisting of:
(i) a ligand capable of binding with the labeled component (a);
(ii) a ligand capable of binding with a binding partner of the labeled component (a);
(iii) a ligand capable of binding with at least one of the component (s) to be determined; and
(iv) a ligand capable of binding with at least one of the binding partners of at least one of the component (s) to be determined; and

(c) directions for the performance of a protocol for the detection and/or determination of one or more components of an immunochemical reaction between the EFGR, the specific binding member, and a specific binding partner thereto.

In accordance with the above, an assay system for screening potential drugs effective to modulate the activity of the EFGR, the aberrant expression or post-translational modification of the EGFR, and/or the activity or binding of the specific binding member may be prepared. The receptor or the binding member may be introduced into a test system, and the prospective drug may also be introduced into the resulting cell culture, and the culture thereafter examined to observe any changes in the S-phase activity of the cells, due either to the addition of the prospective drug alone, or due to the effect of added quantities of the known agent (s).

### Nucleic Acids

The present invention further provides an isolated nucleic acid encoding a specific binding member of the present invention. Nucleic acid includes DNA and RNA. In a preferred aspect, the present invention provides a nucleic acid which codes for a polypeptide of the invention as defined above, including a polypeptide as set out as the CDR residues of the VH and VL chains of the inventive antibodies.

The present invention also provides constructs in the form of plasmids, vectors, transcription or expression cassettes which comprise at least one polynucleotide as above.

The present invention also provides a recombinant host cell which comprises one or more constructs as above. A nucleic acid encoding any specific binding member as provided itself forms an aspect of the present invention, as does a method of production of the specific binding member which method comprises expression from encoding nucleic acid therefore. Expression may conveniently be achieved by culturing under appropriate conditions recombinant host cells containing the nucleic acid. Following production by expression a specific binding member may be isolated and/or purified using any suitable technique, then used as appropriate.

Specific binding members and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Pluckthun, A. Bio/Technology 9:545-551 (1991). Expression in eukaryotic cells in culture is also available to those skilled in the art as an option for production of a specific binding member, see for recent reviews, for example Raff, M. E. (1993) Curr. Opinion Biotech. 4:573-576; Trill J. J. et al. (1995) Curr. Opinion Biotech 6:553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express a specific binding member or polypeptide as above.

As stated above, the present invention also relates to a recombinant DNA molecule or cloned gene, or a degenerate variant thereof, which encodes a specific binding member, particularly antibody or a fragment thereof, that possesses an amino acid sequence set forth in SEQ ID NOS:2 and 4; 129 and 134; 22 and 27; 32 and 37; and/or 42 and 47, preferably a nucleic acid molecule, in particular a recombinant DNA molecule or cloned gene, encoding the binding member or antibody has a nucleotide sequence or is complementary to a DNA sequence encoding one of such sequences.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS1, COS 7, BSC1, BSC40, and BMT10), insect cells (e.g., SD), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that specific binding member analogs may be prepared from nucleotide sequences of the protein complex/subunit derived within the scope of the present invention. Analogs, such as fragments, may be produced, for example, by pepsin digestion of specific binding member material. Other analogs, such as muteins, can be produced by standard site-directed mutagenesis of specific binding member coding sequences. Analogs exhibiting “specific binding member activity” such as small molecules, whether functioning as promoters or inhibitors, may be identified by known in vivo and/or in vitro assays.

As mentioned above, a DNA sequence encoding a specific binding member can be prepared synthetically rather than cloned. The DNA sequence can be designed with the appropriate codons for the specific binding member amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes which will express specific binding member analogs or “muteins”. Alternatively, DNA encoding muteins can be made by site-directed mutagenesis of native specific binding member genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The present invention extends to the preparation of antisense oligonucleotides and ribozymes that may be used to interfere with the expression of the EGFR at the translational level. This approach utilizes antisense nucleic acid and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule (See Weintraub, 1990; Marcus-Sekura, 1988.). In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into producing cells. Antisense methods have been used to inhibit the expression of many genes in vitro (Marcus-Sekura, 1988; Hambor et al., 1988).

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, 1988.). Because they are sequence-specific, only mRNAs with particular sequences are inactivated.

Investigators have identified two types of ribozymes, Tetrahymena-type and “hammerhead”-type (Hasselhoff and Gerlach, 1988). Tetrahymena-type ribozymes recognize four-base sequences, while “hammerhead”—type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The DNA sequences described herein may thus be used to prepare antisense molecules against, and ribozymes that cleave mRNAs for EFGRs and their ligands.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Generation and Isolation of Antibodies

### Cell Lines

For immunization and specificity analyses, several cell lines, native or transfected with either the normal, wild-type or “wtEGFR” gene or the ΔEGFR gene carrying the Δ2-7 deletion mutation were used: Murine fibroblast cell line NR6, NR6_{ΔEGFR} (transfected with ΔEGFR) and NR6_{wtEGFR} (transfected with wtEGFR), human glioblastoma cell line U87MG (expressing low levels of endogenous wtEGFR), U87MG_{wtEGFR} (transfected with wtEGFR), U87MG_{ΔEGFR} (transfected with ΔEGFR), and human squamous cell carcinoma cell line A431 (expressing high levels of wtEGFR).

For immunization and specificity analyses, several cell lines, native or transfected with either the normal, wild-type or “wtEGFR” gene or the ΔEGFR gene carrying the de2-7 or Δ2-7 deletion mutation were used: Murine fibroblast cell line NR6, NR6_{ΔEGFR} (transfected with ΔEGFR) and NR6_{wtEGFR} (transfected with wtEGFR), human glioblastoma cell line U87MG (expressing low levels of endogenous wtEGFR), U87MG_{WtEGFR} or “U87MG.wtEGFR” (transfected with wtEGFR), U87MG_{ΔEGFR} or “U87MG.Δ2-7” (transfected with ΔEGFR), and human squamous cell carcinoma cell line A431 (expressing high levels of wtEGFR). The NR6, NR6_{ΔEGFR}, and NR6_{wtEGFR} cell lines were previously described (Batra et al. (1995) Epidermal Growth Factor Ligand-independent, Unregulated, Cell-Transforming Potential of a Naturally Occurring Human Mutant EGFRvIII Gene. Cell Growth Differ. 6(10): 1251-1259). The NR6 cell line lacks normal endogenous EGFR. (Batra et al., 1995). U87MG cell lines and transfections were described previously (Nishikawa et al. (1994) A mutant epidermal growth factor receptor common in human glioma confers enhanced tumorigenicity. Proc. Natl. Acad. Sci. U.S.A. 91, 7727-7731).

The U87MG astrocytoma cell line (Ponten, J. and Macintyre, E. H. (1968) Long term culture of normal and neoplastic human glia. Acta. Pathol. Microbiol. Scand. 74, 465-86) which endogenously expresses low levels of the wtEGFR, was infected with a retrovirus containing the de2-7 EGFR to produce the U87MG.Δ2-7 cell line (Nishikawa et al., 1994). The transfected cell line U87MG.wtEGFR was produced as described in Nagane et al. (1996) Cancer Res. 56, 5079-5086. Whereas U87MG cells express approximately 1×10⁵ EGFR, U87MG.wtEGFR cells express approximately 1×10⁶ EGFR, and thus mimic the situation seen with gene amplification. The murine pro-B cell line BaF/3, which does not express any known EGFR related molecules, was also transfected with de2-7 EGFR. resulting in the BaF/3. Δ2-7 cell line (Luwor et al. (2004) The tumor-specific de2-7 epidermal growth factor receptor (EGFR) promotes cells survival and heterodimerizes with the wild-type EGFR, Oncogene 23: 6095-6104). Human squamous carcinoma A431 cells were obtained from ATCC (Rockville, Md.). The epidermoid carcinoma cell line A431 has been described previously (Sato et al. (1987) Derivation and assay of biological effects of monoclonal antibodies to epidermal growth factor receptors. Methods Enzymol. 146, 63-81).

All cell lines were cultured in DMEM/F-12 with GlutaMAX™ (Life Technologies, Inc., Melbourne, Australia and Grand Island, N.Y.) supplemented with 10% FCS(CSL, Melbourne, Australia); 2 mM glutamine (Sigma Chemical Co., St. Louis, Mo.), and penicillin/streptomycin (Life Technologies, Inc., Grand Island, N.Y.). In addition, the U87MG.Δ2-7 and U87MG.wtEGFR cell lines were maintained in 400 mg/ml of geneticin (Life Technologies, Inc., Melbourne, Victoria, Australia). Cell lines were grown at 37° C. in a unmodified atmosphere of 5% CO₂.

### Reagents

The de2-7 EGFR unique junctional peptide has the amino acid sequence: LEEKKGNYVVTDH (SEQ ID NO:13). Biotinylated unique junctional peptides (Biotin-LEEKKGNYVVTDH (SEQ ID NO:5) and LEEKKGNYVVTDH-Biotin (SEQ ID NO:6)) from de2-7 EGFR were synthesized by standard Fmoc chemistry and purity (>96%) determined by reverse phase HPLC and mass spectral analysis (Auspep, Melbourne, Australia).

### Antibodies Used in Studies

In order to compare our findings with other reagents, additional mAbs were included in our studies. These reagents were mAb528 to the wtEGFR (Sato et al. (1983) Mol. Biol. Med. 1(5), 511-529) and DH8.3, which was generated against a synthetic peptide spanning the junctional sequence of the Δ2-7 EGFR deletion mutation. The DH8.3 antibody (IgG1), which is specific for the de2-7 EGFR, has been described previously (Hills et al. (1995) Specific targeting of a mutant, activated EGF receptor found in glioblastoma using a monoclonal antibody. Int. J. Cancer. 63, 537-43, 1995) and was obtained following immunization of mice with the unique junctional peptide found in de2-7 EGFR (Hills et al., 1995).

The 528 antibody, which recognizes both de2-7 and wild-type EGFR, has been described previously (Masui et al. (1984) Growth inhibition of human tumor cells in athymic mice by anti-epidermal growth factor receptor monoclonal antibodies. Cancer Res. 44, 1002-7) and was produced in the Biological Production Facility, Ludwig Institute for Cancer Research (Melbourne, Australia) using a hybridoma (ATCC HB-8509) obtained from the American Type Culture Collection (Rockville, Md.). The polyclonal antibody SC-03 is an affinity purified rabbit polyclonal antibody raised against a carboxy terminal peptide of the EGFR (Santa Cruz Biotechnology Inc.).

### Antibody Generation

The murine fibroblast line NR6_{ΔEGFR} was used as immunogen. Mouse hybridomas were generated by immunizing BALB/c mice five times subcutaneously at 2- to 3-week intervals, with 5×10⁵-2×10⁶ cells in adjuvant. Complete Freund's adjuvant was used for the first injection. Thereafter, incomplete Freund's adjuvant (Difco™, Voigt Global Distribution, Lawrence, Kans.) was used. Spleen cells from immunized mice were fused with mouse myeloma cell line SP2/0 (Shulman et al. (1978) Nature 276:269-270). Supernatants of newly generated clones were screened in hemadsorption assays for reactivity with cell line NR6, NR6_{wtEGFR}, and NR6_{ΔEGFR} and then analyzed by hemadsorption assays with human glioblastoma cell lines U87MG, U87MG_{wtEGFR}, and U87_{ΔEGFR}. Selected hybridoma supernatants were subsequently tested by western blotting and further analyzed by immunohistochemistry. Newly generated mAbs showing the expected reactivity pattern were purified.

Five hybridomas were established and three clones, 124 (IgG2a), 806 (IgG2b), and 1133 (IgG2a) were initially selected for further characterization based on high titer (1:2500) with NR6_{ΔEGFR} and low background on NR6 and NR6_{wtEGFR} cells in the rosette hemagglutination assay. A fourth clone, 175 (IgG2a) was subsequently further characterized and is discussed separately in Example 23, below. In a subsequent hemagglutination analysis, these antibodies showed no reactivity (undiluted supernatant ≦10%) with the native human glioblastoma cell line U87MG and U87MG_{wtEGFR}, but were strongly reactive with U87MG_{ΔEGFR}; less reactivity was seen with A431. By contrast, in FACS analysis, 806 was unreactive with native U87MG and intensively stained U87MG_{ΔEGFR} and to a lesser degree U87MG_{wtEGFR} indicating binding of 806 to both, ΔEGFR and wtEGFR (see below).

In Western blot assays, mAb124, mAb806 and mAb1133 were then analyzed for reactivity with wtEGFR and ΔEGFR. Detergent lysates were extracted from NR6_{ΔEGFR}, U87MG_{ΔEGFR} as well as from A431. All three mAbs showed a similar reactivity pattern with cell lysates staining both the wtEGFR (170 kDa) and ΔEGFR protein (140 kDa). As a reference reagent, mAbR.I. known to be reactive with the wtEGFR (Waterfield et al. (1982) J. Cell Biochem. 20(2), 149-161) was used instead of mAb528, which is known to be non-reactive in western blot analysis. mAbR.I. showed reactivity with wild-type and ΔEGFR. All three newly generated clones showed reactivity with ΔEGFR and less intense with wtEGFR. DH8.3 was solely positive in the lysate of U87MG_{ΔEGFR} and NR6_{ΔEGFR}.

The immunohistochemical analysis of clones 124, 806, and 1133 as well as mAb528 and mAbDH8.3 on xenograft tumors U87MG, U87MG_{ΔEGFR}, and A431 are shown in Table 1. All mAbs showed strong staining of xenograft U87MG_{ΔEGFR}. Only mAb528 showed weak reactivity in the native U87MG xenograft. In A431 xenografts, mAb528 showed strong homogeneous reactivity. mAb124, mAb806, and mAb1133 revealed reactivity with mostly the basally located cells of the squamous cell carcinoma of A431 and did not react with the upper cell layers or the keratinizing component. DH8.3 was negative in A431 xenografts.

TABLE 1
Immunohistochemical Analysis of Antibodies
528, DH8.3, and 124, 806 and 1133

| | xenograft | | xenograft |
|---|---|---|---|
| Antibody | ΔU87MG_{ΔEGFR} | xenograft A431 | U87MG(native) |
| mAb528 | pos. | pos. | pos. (focal staining) |
| mAb124 | pos. | pos. (predominantly | — |
| | | basal cells) | |
| mAb806 | pos. | pos. (predominantly | — |
| | | basal cells) | |
| mAb1133 | pos. | pos. (predominantly | — |
| | | basal cells) | |
| DH8.3 | pos. | — | — |

minor stromal staining due to detection of endogenous mouse antibodies.

### Sequencing

The variable heavy (VH) and variable light (VL) chains of mAb806, mAb124 and mAb1133 were sequenced, and their complementarity determining regions (CDRs) identified, as follows:

mAb806

mAb806 VH chain: nucleic acid sequence (SEQ ID NO:1) and amino acid sequence, with signal peptide (SEQ ID NO:2) are shown in FIGS. 14A and 14B, respectively (signal peptide underlined in FIG. 14B). Complementarity determining regions CDR1, CDR2, and CDR3 (SEQ ID NOS: 15, 16, and 17, respectively) are indicated by underlining in FIG. 16. The mAb806 VH chain amino acid sequence without its signal peptide (SEQ ID NO:11) is shown in FIG. 16.

mAb806 VL chain: nucleic acid sequence (SEQ ID NO:3) and amino acid sequence, with signal peptide (SEQ ID NO:4) are shown in FIGS. 15A and 15B, respectively (signal peptide underlined in FIG. 15B). Complementarity determining regions CDR1, CDR2, and CDR3 (SEQ ID NOS: 18, 19, and 20, respectively) are indicated by underlining in FIG. 17. The mAb806 VL chain amino acid sequence without its signal peptide (SEQ ID NO:12) is shown in FIG. 17.

mAb 124

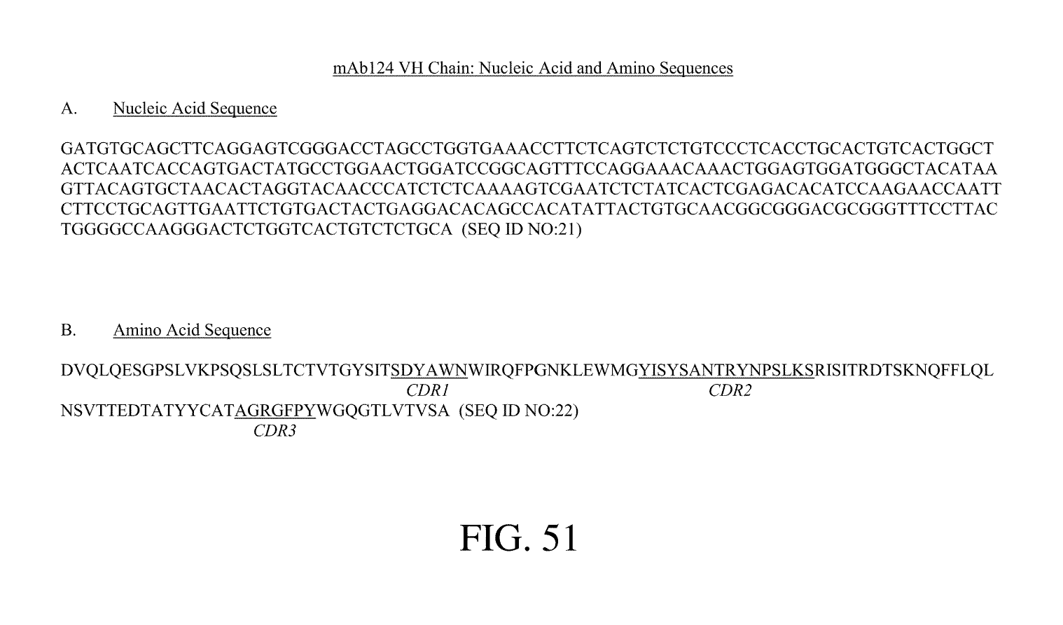

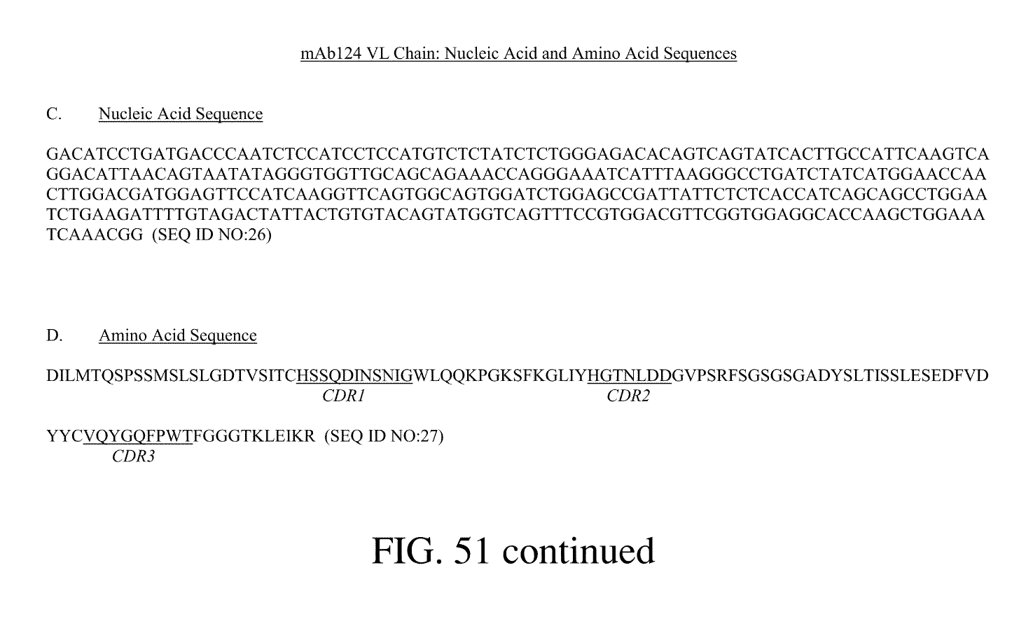

mAb124 VH chain: nucleic acid (SEQ ID NO:21) and amino acid (SEQ ID NO:22) sequences are shown in FIGS. 51A and 51B, respectively. Complementarity determining regions CDR1, CDR2, and CDR3 (SEQ ID NOS: 23, 24, and 25, respectively) are indicated by underlining.

mAb124 VL chain: nucleic acid (SEQ ID NO:26) and amino acid (SEQ ID NO:27) sequences are shown in FIGS. 51C and 51D, respectively. Complementarity determining regions CDR1, CDR2, and CDR3 (SEQ ID NOS: 28, 29, and 30, respectively) are indicated by underlining.

mAb1133

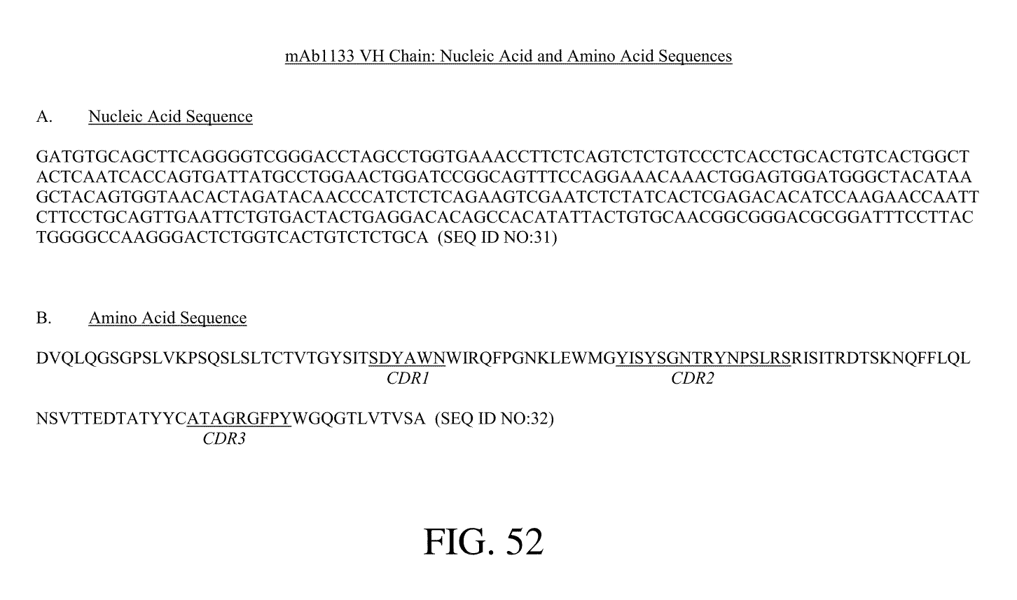

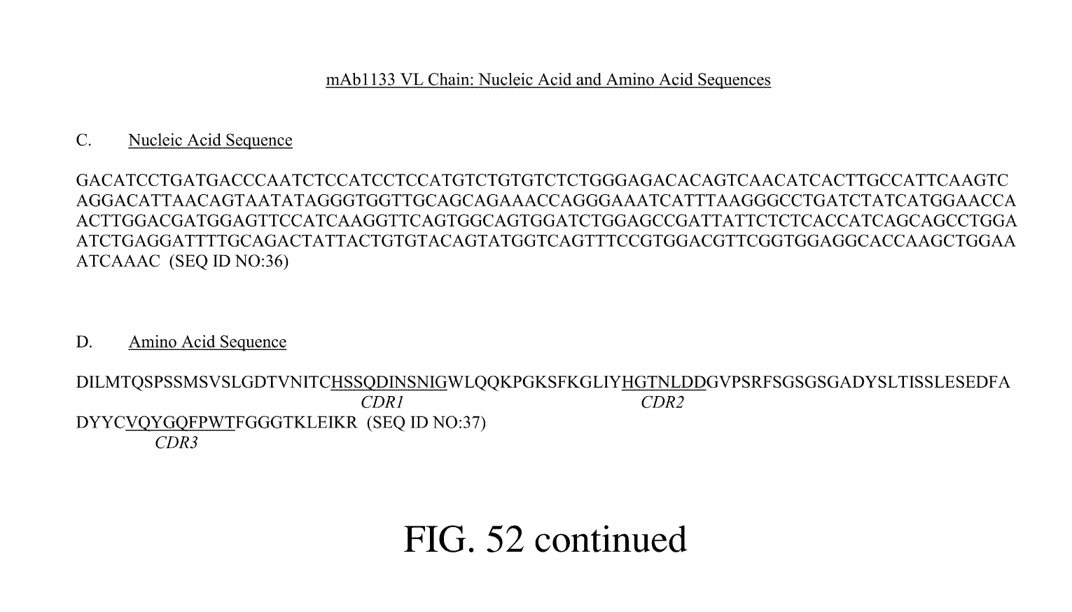

mAb1113 VH chain: nucleic acid (SEQ ID NO:31) and amino acid (SEQ ID NO:32) sequences are shown in FIGS. 52A and 52B, respectively. Complementarity determining regions CDR1, CDR2, and CDR3 (SEQ ID NOS: 33, 34, and 35, respectively) are indicated by underlining.

mAb1133 VL chain: nucleic acid (SEQ ID NO:36) and amino acid (SEQ ID NO:37) sequences are shown in FIGS. 52C and 52D, respectively. Complementarity determining regions CDR1, CDR2, and CDR3 (SEQ ID NOS: 38, 39, and 40, respectively) are indicated by underlining.

### Example 2

### Binding of Antibodies to Cell Lines by FACS

mAb806 was initially selected for further characterization, as set forth herein and in the following Examples. mAb124 and mAb1133 were also selected for further characterization, as discussed in Example 26 below, and found to have properties corresponding to the unique properties of mAb806 discussed herein.

In order to determine the specificity of mAb806, its binding to U87MG, U87MG.Δ2-7 and U87MG.wtEGFR cells was analyzed by flow activated cell sorting (FACS). Briefly, cells were labelled with the relevant antibody (10 μg/ml) followed by fluorescein-conjugated goat anti-mouse IgG (1:100 dilution; Calbiochem San Diego, Calif., USA; Becton-Dickinson PharMingen, San Diego, Calif., US) as described previously (Nishikawa et al., 1994). FACS data was obtained on a Coulter Epics Elite ESP by observing a minimum of 5,000 events and analyzed using EXPO (version 2) for Windows. An irrelevant IgG2b was included as an isotype control for mAb806 and the 528 antibody was included as it recognizes both the de2-7 and wtEGFR.

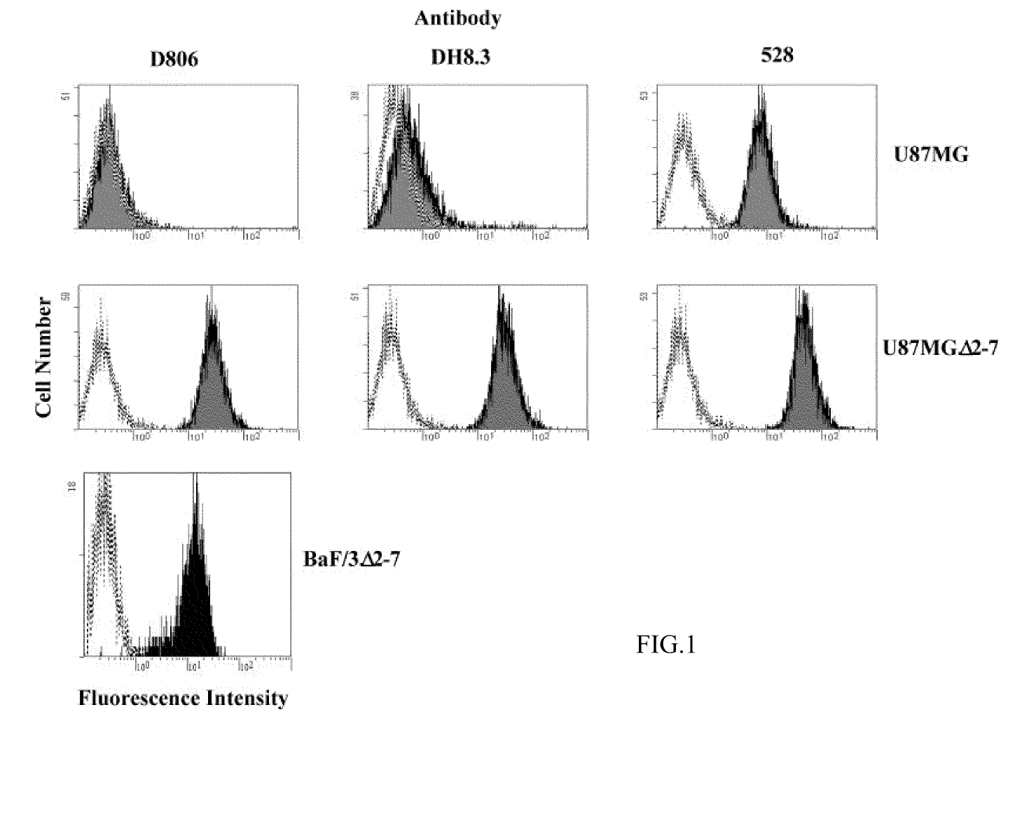

Only the 528 antibody was able to stain the parental U87MG cell line (FIG. 1) consistent with previous reports demonstrating that these cells express the wtEGFR (Nishikawa et al, 1994). mAb806 and DH8.3 had binding levels similar to the control antibody, clearly demonstrating that they are unable to bind the wild-type receptor (FIG. 1). Binding of the isotype control antibody to U87MG.Δ2-7 and U87MG.wtEGFR cells was similar as that observed for the U87MG cells.

mAb806 stained U87MG.Δ2-7 and U87MG.wtEGFR cells, indicating that mAb806 specifically recognizes the de2-7 EGFR and amplified EGFR (FIG. 1). DH8.3 antibody stained U87MG.Δ2-7 cells, confirming that DH8.3 antibody specifically recognizes the de2-7 EGFR (FIG. 1). As expected, the 528 antibody stained both the U87MG.Δ2-7 and U87MG.wtEGFR cell lines (FIG. 1). As expected, the 528 antibody stained U87MG.Δ2-7 with a higher intensity than the parental cell as it binds both the de2-7 and wild-type receptors that are co-expressed in these cells (FIG. 1). Similar results were obtained using a protein A mixed hemadsorption which detects surface bound IgG by appearance of Protein A coated with human red blood cells (group O) to target cells. Monoclonal antibody 806 was reactive with U87MG.Δ2-7 cells but showed no significant reactivity (undiluted supernatant less than 10%) with U87MG expressing wild-type EGFR. Importantly, mAb806 also bound the BaF/3.Δ2-7 cell line, demonstrating that the co-expression of wtEGFR is not a requirement for mAb806 reactivity (FIG. 1).

### Example 3

### Binding of Antibodies in Assays

To further characterize the specificity of mAb806 and the DH8.3 antibody, their binding was examined by ELISA. Two types of ELISA were used to determine the specificity of the antibodies. In the first assay, plates were coated with sEGFR (10 μg/ml in 0.1 M carbonate buffer pH 9.2) for 2 h and then blocked with 2% human serum albumin (HSA) in PBS. sEGFR is the recombinant extracellular domain (amino acids 1-621) of the wild-type EGFR), and was produced as previously described (Domagala et al. (2000) Stoichiometry, kinetic and binding analysis of the interaction between Epidermal Growth Factor (EGF) and the Extracellular Domain of the EGF receptor. Growth Factors. 18, 11-29). Antibodies were added to wells in triplicate at increasing concentration in 2% HSA in phosphate-buffered saline (PBS). Bound antibody was detected by horseradish peroxidase conjugated sheep anti-mouse IgG (Silenus, Melbourne, Australia) using ABTS (Sigma, Sydney, Australia) as a substrate and the absorbance measured at 405 nm

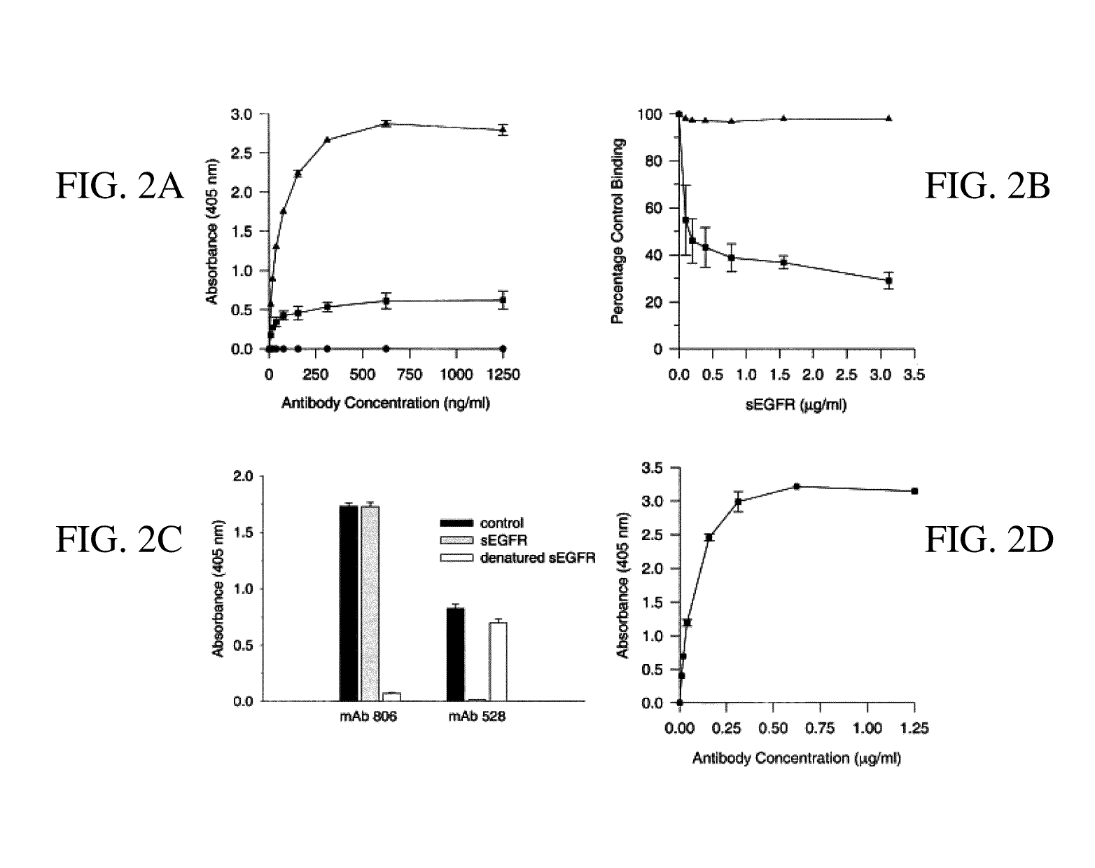

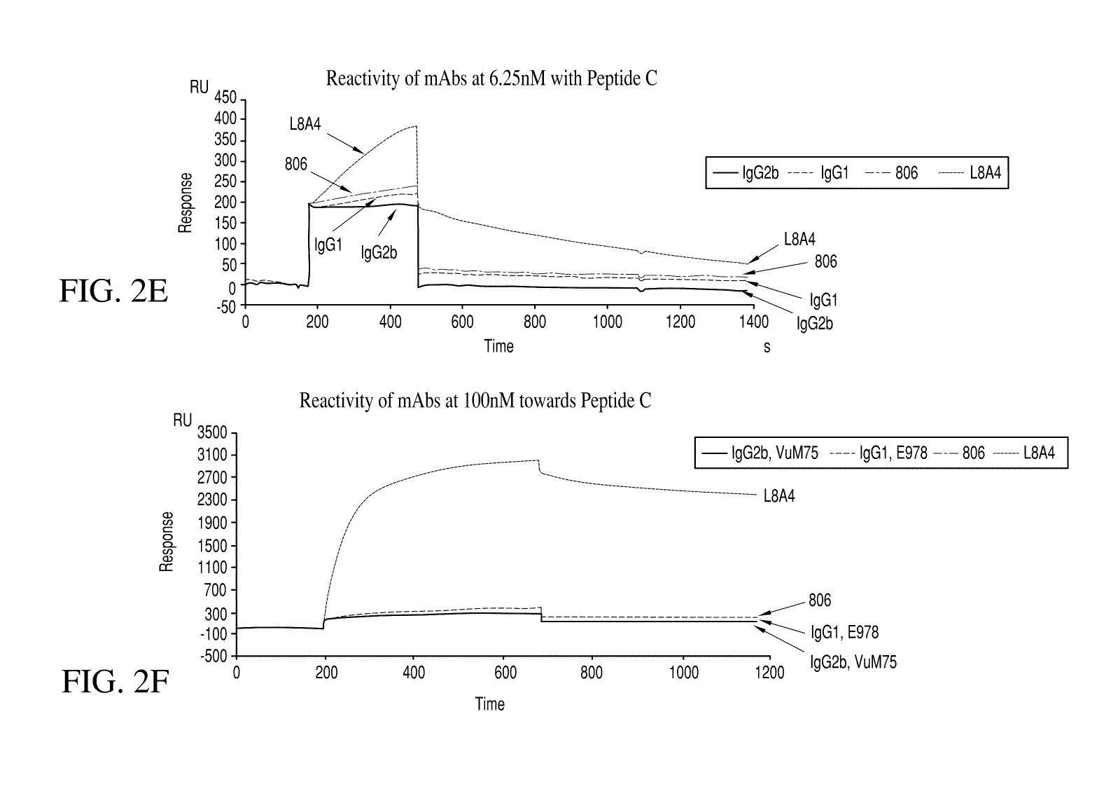

Both mAb806 and the 528 antibody displayed dose-dependent and saturating binding curves to immobilized wild-type sEGFR (FIG. 2A). As the unique junctional peptide found in the de2-7 EGFR is not contained within the sEGFR, mAb806 must be binding to an epitope located within the wild-type EGFR sequence. The binding of the 528 antibody was lower than that observed for mAb806, probably because it recognizes a conformational determinant. As expected, the DH8.3 antibody did not bind the wild-type sEGFR even at concentrations up to 10 μg/ml (FIG. 2A). Although sEGFR in solution inhibited the binding of the 528 antibody to immobilized sEGFR in a dose-dependent fashion, it was unable to inhibit the binding of mAb806 (FIG. 2B). This suggests that mAb806 can only bind wild-type EGFR once immobilized on ELISA plates, a process that may induce conformational changes. Similar results were observed using a BIAcore whereby mAb806 bound immobilized sEGFR but immobilized mAb806 was not able to bind sEGFR in solution (FIG. 2C).

Following denaturation by heating for 10 min at 95° C., sEGFR in solution was able to inhibit the binding of mAb806 to immobilized sEGFR (FIG. 2C), confirming that mAb806 can bind the wild-type EGFR under certain conditions. Interestingly, the denatured sEGFR was unable to inhibit the binding of the 528 antibody (FIG. 2C), demonstrating that this antibody recognizes a conformational epitope. The DH8.3 antibody exhibited dose-dependent and saturable binding to the unique de2-7 EGFR peptide (FIG. 2D). Neither mAb806 or the 528 antibody bound to the peptide, even at concentrations higher than those used to obtain saturation binding of DH8.3, further indicating mAb806 does not recognize an epitope determinant within this peptide.

In the second assay, the biotinylated de2-7 specific peptide (Biotin LEEKKGNYVVTDH (SEQ ID NO:5)) was bound to ELISA plates precoated with streptavidin (Pierce, Rockford, Ill.). Antibodies were bound and detected as in the first assay. Neither mAb806 nor the 528 antibody bound to the peptide, even at concentrations higher than those used to obtain saturation binding of DH8.3, further indicating that mAb806 does not recognize an epitope determinant within this peptide.

To further demonstrate that mAb806 recognizes an epitope distinct from the junction peptide, additional experiments were performed. C-terminal biotinylated de2-7 peptide (LEEKKGNYVVTDH-Biotin (SEQ ID NO:6)) was utilized in studies with mAb806 and mAbL8A4, generated against the de2-7 peptide (Reist et al. (1995) Cancer Res. 55(19), 4375-4382; Foulon et al. (2000) Cancer Res. 60(16), 4453-4460).

### Reagents Used in Peptide Studies

Junction Peptide: LEEKKGNYVVTDH-OH (Biosource, Camarillo, Calif.);
Peptide C: LEEKKGNYVVTDH(K-Biot)-OH (Biosource, Camarillo, Calif.);
sEGFR: CHO-cell-derived recombinant soluble extracellular domain (amino acids 1-621) of the wild-type EGFR (LICR Melbourne);
mAb806: mouse monoclonal antibody, IgG_{2b} (LICR NYB);
mAbL8A4: mouse monoclonal antibody, IgG₁ (Duke University);
IgG₁ isotype control mAb;
IgG_{2b} isotype control mAb.

Peptide C was immobilized on a Streptavidin microsensor chip at a surface density of 350RU (+/−30RU). Serial dilutions of mAbs were tested for reactivity with the peptide. Blocking experiments using non-biotinylated peptide were performed to assess specificity.

mAbL8A4 showed strong reactivity with Peptide C even at low antibody concentrations

(6.25 nM) (FIG. 2E). mAb806 did not show detectable specific reactivity with Peptide C up to antibody concentrations of 100 nM (highest concentration tested) (FIGS. 2E and 2F). It was expected that mAbL8A4 would react with Peptide C because the peptide was used as the immunogen in the generation of mAbL8A4. Addition of the Junction Peptide (non-biotinylated, 50 μg/ml) completely blocks the reactivity of mAbL8A4 with Peptide C, confirming the antibody's specificity for the junction peptide epitope.

In a second set of BIAcore experiments, sEGFR was immobilized on a CM microsensor chip at a surface density of ˜4000RU. Serial dilutions of mAbs were tested for reactivity with sEGFR.

mAb806 was strongly reactive with denaturated sEGFR while mAbL8A4 did not react with denaturated sEGFR. Reactivity of mAb806 with denaturated sEGFR decreases with decreasing antibody concentrations. It was expected that mAbL8A4 does not react with sEGFR because mAbL8A4 was generated using the junction peptide as the immunogen and sEGFR does not contain the junction peptide.

Dot-blot immune stain experiments were also performed. Serial dilutions of peptide were spotted in 0.5 μl onto a PVDF or nitrocellulose membranes. Membranes were blocked with 2% BSA in PBS, and then probed with 806, L8A4, DH8.3 and control antibodies. Antibodies L8A4 and DH8.3 bound to peptide on the membranes (data not shown). mAb806 did not bind peptide at concentrations where L8A4 clearly showed binding (data not shown). Control antibodies were also negative for peptide binding.

mAb806 bound to the wtEGFR in cell lysates following immunoblotting (results not shown). This is different from the results obtained with DH8.3 antibody, which reacted with de2-7 EGFR but not wtEGFR. Thus, mAb806 can recognize the wtEGFR following denaturation but not when the receptor is in its natural state on the cell surface.

### Example 4

### Scatchard Analysis

A Scatchard analysis using U87MG.Δ2-7 cells was performed following correction for immunoreactivity in order to determine the relative affinity of each antibody. Antibodies were labelled with ¹²⁵I (Amrad, Melbourne, Australia) by the Chloramine T method and immunoreactivity determined by Lindmo assay (Lindmo et al. (1984) Determination of the immunoreactive fraction of radiolabeled monoclonal antibodies by linear extrapolation to binding at infinite antigen excess. J. Immunol. Methods. 72, 77-89).

All binding assays were performed in 1% HSA/PBS on 1-2×10⁶ live U87MG.Δ2-7 or A431 cells for 90 min at 4° C. with gentle rotation. A set concentration of 10 ng/ml ¹²⁵I-labeled antibody was used in the presence of increasing concentrations of the appropriate unlabeled antibody. Non-specific binding was determined in the presence of 10.000-fold excess of unlabeled antibody. Neither ¹²⁵I-radiolabeled mAb806 or the DH8.3 antibody bound to parental U87MG cells. After the incubation was completed, cells were washed and counted for bound ¹²⁵I-labeled antibody using a COBRA II gamma counter (Packard Instrument Company, Meriden, Conn., USA).

Both mAb806 and the DH8.3 antibody retained high immunoreactivity when iodinated and was typically greater than 90% for mAb806 and 45-50% for the DH8.3 antibody. mAb806 had an affinity for the de2-7 EGFR receptor of 1.1×10⁹ M⁻¹ whereas the affinity of DH8.3 was some 10-fold lower at 1.0×10⁸ M⁻¹. Neither iodinated antibody bound to U87MG parental cells. mAb806 recognized an average of 2.4×10⁵ binding sites per cell with the DH8.3 antibody binding an average of 5.2×10⁵ sites. Thus, there was not only good agreement in receptor number between the antibodies, but also with a previous report showing 2.5×10⁵ de2-7 receptors per cell as measured by a different de2-7 EGFR specific antibody on the same cell line (Reist et al. (1997) Improved targeting of an anti-epidermal growth factor receptor variant III monoclonal antibody in tumor xenografts after labeling using N-succinimidyl 5-iodo-3-pyridinecarboxylate. Cancer Res. 57, 1510-5).

### Example 5

### Internalization of Antibodies By U87MG.Δ2-7 Cells

The rate of antibody internalization following binding to a target cell influences both its tumor targeting properties and therapeutic options. Consequently, the inventors examined the internalization of mAb806 and the DH8.3 antibody following binding to U87MG.Δ2-7 cells by FACS. U87MG.Δ2-7 cells were incubated with either mAb806 or the DH8.3 antibody (10 μg/ml) for 1 h in DMEM at 4° C. After washing, cells were transferred to DMEM pre-warmed to 37° C. and aliquots taken at various time points following incubation at 37° C. Internalization was stopped by immediately washing aliquots in ice-cold wash buffer (1% HSA/PBS). At the completion of the time course cells were stained by FACS as described above. Percentage internalization was calculated by comparing surface antibody staining at various time points to zero time using the formula: percent antibody internalized=(mean fluorescence at timeₓ-background fluorescence)/(mean fluorescence at time₀−background fluorescence)×100. This method was validated in one assay using an iodinated antibody (mAb806) to measure internalization as previously described (Huang et al. (1997) The enhanced tumorigenic activity of a mutant epidermal growth factor receptor common in human cancers is mediated by threshold levels of constitutive tyrosine phosphorylation and unattenuated signaling. J. Biol. Chem. 272, 2927-35). Differences in internalization rate at different time points were compared using Student's t-test. Throughout this research, data were analyzed for significance by Student's t-test, except for the in vivo survival assays, which were analyzed by Wilcoxon analysis.

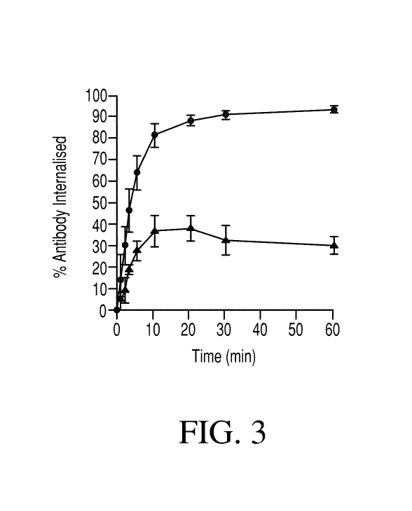

Both antibodies showed relatively rapid internalization reaching steady-state levels at 10 min for mAb806 and 30 min for DH8.3 (FIG. 3). Internalization of DH8.3 was significantly higher both in terms of rate (80.5% of DH8.3 internalized at 10 min compared to 36.8% for mAb806, p<0.01) and total amount internalized at 60 min (93.5% versus 30.4%, p<0.001). mAb806 showed slightly lower levels of internalization at 30 and 60 min compared to 20 min in all 4 assays performed (FIG. 3). This result was also confirmed using an internalization assay based on iodinated mAb806 (data not shown).

### Example 6

### Electron Microscopy Analysis of Antibody Internalization

Given the above noted difference in internalization rates between the antibodies, a detailed analysis of antibody intracellular trafficking was performed using electron microscopy.

U87MG.Δ2-7 cells were grown on gelatin coated chamber slides (Nunc, Naperville, Ill.) to 80% confluence and then washed with ice cold DMEM. Cells were then incubated with mAb806 or the DH8.3 antibody in DMEM for 45 min at 4° C. After washing, cells were incubated for a further 30 min with gold-conjugated (20 nm particles) anti-mouse IgG (BBlnternational, Cardiff, UK) at 4° C. Following a further wash, pre-warmed DMEM/10% PCS was added to the cells, which were incubated at 37° C. for various times from 1-60 min Internalization of the antibody was stopped by ice-cold media and cells fixed with 2.5% glutaraldehyde in PBS/0.1% HSA and then post-fixed in 2.5% osmium tetroxide. After dehydration through a graded series of acetone, samples were embedded in Epon/Araldite resin, cut as ultra-thin sections with a Reichert Ultracut-S microtome (Leica) and collected on nickel grids. The sections were stained with uranyl acetate and lead citrate before being viewed on a Philips CM12 transmission electron microscope at 80 kV. Statistical analysis of gold grains contained within coated pits was performed using a Chi-square test.

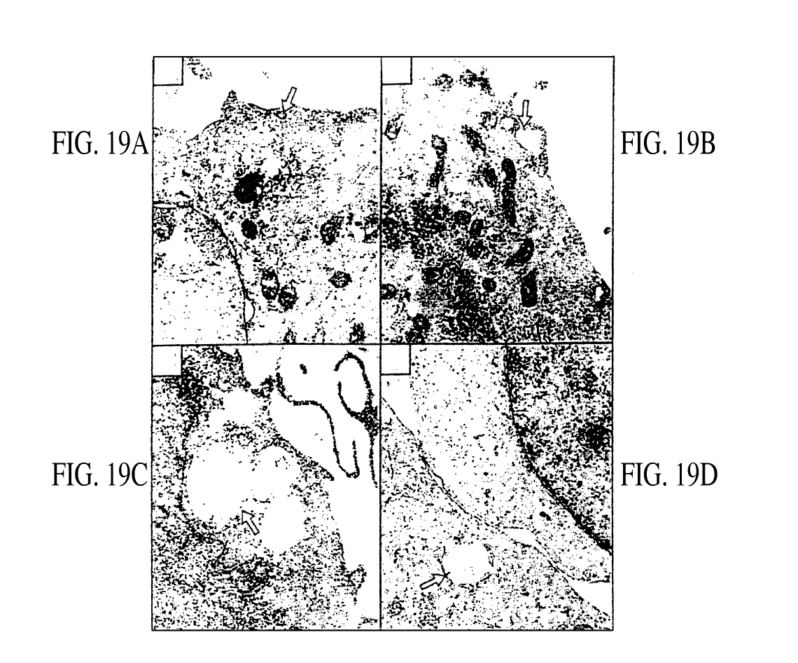

While the DH8.3 antibody was internalized predominantly via coated-pits, mAb806 appeared to be internalized by macropinocytosis (FIG. 19). In fact, a detailed analysis of 32 coated pits formed in cells incubated with mAb806 revealed that none of them contained antibody. In contrast, around 20% of all coated-pits from cells incubated with DH8.3 were positive for antibody, with a number containing multiple gold grains. A statistical analysis of the total number of gold grains contained within coated-pits found that the difference was highly significant (p<0.01). After 20-30 min both antibodies could be seen in structures that morphologically resemble lysosomes (FIG. 19C). The presence of cellular debris within these structures was also consistent with their lysosome nature.

### Example 7

### Biodistribution of Antibodies In Tumor Bearing Nude Mice

The biodistribution of mAb806 and the DH8.3 antibody was compared in nude mice containing U87MG xenografts on one side and U87MG.Δ2-7 xenografts on the other. A relatively short time period was chosen for this study as a previous report demonstrated that the DH8.3 antibody shows peak levels of tumor targeting between 4-24 h (Hills et al. (1995) Specific targeting of a mutant, activated EGF receptor found in glioblastoma using a monoclonal antibody. Int. J. Cancer. 63, 537-43).

Tumor xenografts were established in nude BALB/c mice by s.c. injection of 3×10⁶ U87MG, U87MG.Δ2-7 or A431 cells. de2-7 EGFR expression in U87MG.Δ2-7 xenografts remained stable throughout the period of biodistribution as measured by immunohistochemistry at various time points (data not shown). A431 cells retained their mAb806 reactivity when grown as tumor xenografts as determined by immunohistochemistry. U87MG or A431 cells were injected on one side 7-10 days before U87MG.Δ2-7 cells were injected on the other side because of the faster growth rate observed for de2-7 EGFR expressing xenografts. Antibodies were radiolabeled and assessed for immunoreactivity as described above and were injected into mice by the retro-orbital route when tumors were 100-200 mg in weight. Each mouse received two different antibodies (2 μg per antibody): 2 μCi of ¹²⁵I-labeled mAb806 and 2 μCi of ¹³¹I labelled DH8.3 or 528. Unless indicated, groups of 5 mice were sacrificed at various time points post-injection and blood obtained by cardiac puncture. The tumors, liver, spleen, kidneys and lungs were obtained by dissection. All tissues were weighed and assayed for ¹²⁵I and ¹³¹I activity using a dual-channel counting Window. Data was expressed for each antibody as % ID/g tumor determined by comparison to injected dose standards or converted into tumor to blood/liver ratios (i.e. % ID/g tumor divided by % ID/g blood or liver). Differences between groups were analyzed by Student's t-test. After injection of radiolabeled mAb806, some tumors were fixed in formalin, embedded in paraffin, cut into 5 μm sections and then exposed to X-ray film (AGFA, Mortsel, Belgium) to determine antibody localization by autoradiography.

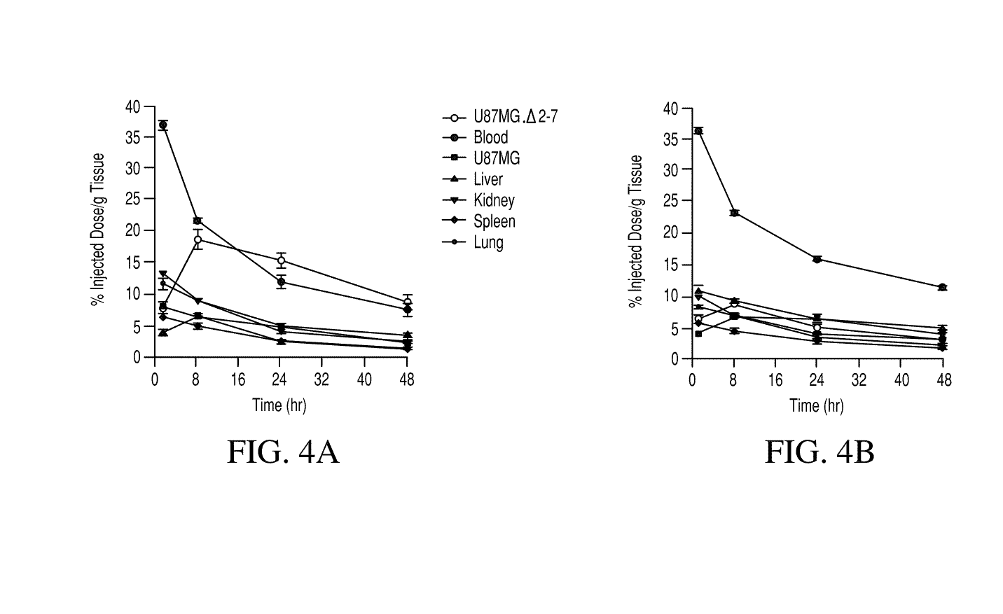

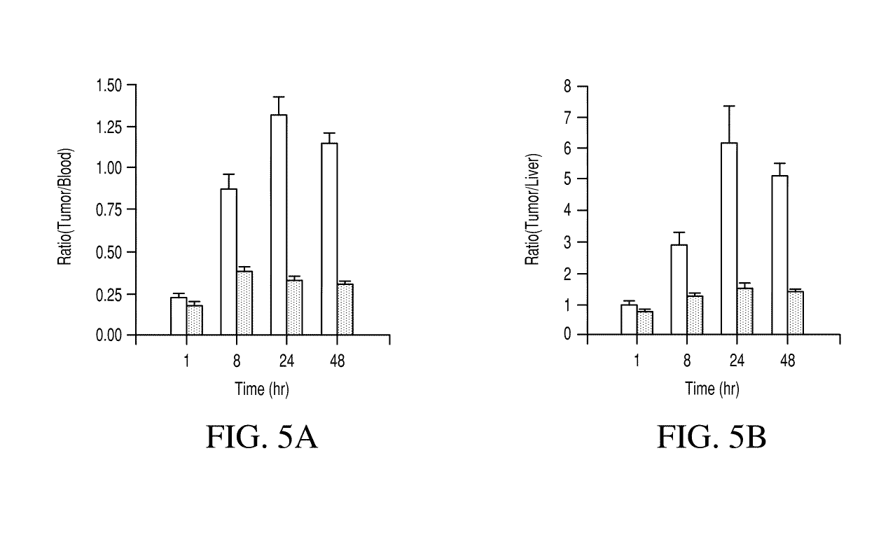

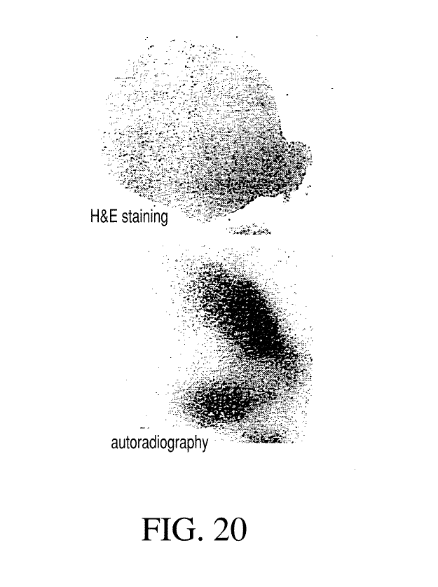

In terms of % ID/g tumor, mAb806 reached its peak level in U87MG.Δ2-7 xenografts of 18.6% m/g tumor at 8 h (FIG. 4A), considerably higher than any other tissue except blood. While DH8.3 also showed peak tumor levels at 8 h, the level was a statistically (p<0.001) lower 8.8% m/g tumor compared to mAb806 (FIG. 4B). Levels of both antibodies slowly declined at 24 and 48 h. Autoradiography of U87MG.Δ2-7 xenograft tissue sections collected 8 hr after injection with ¹²⁵I-labeled mAb806 alone, clearly illustrates localization of antibody to viable tumor (FIG. 20). Neither antibody showed specific targeting of U87MG parental xenografts (FIGS. 4A and 4B). With regards to tumor to blood/liver ratios, mAb806 showed the highest ratio at 24 h for both blood (ratio of 1.3) and liver (ratio of 6.1) (FIGS. 5A and 5B). The DH8.3 antibody had its highest ratio in blood at 8 h (ratio of 0.38) and at 24 h in liver (ratio of 1.5) (FIGS. 5 A and 5B), both of which are considerably lower than the values obtained for mAb806.

As described above, levels of mAb806 in the tumor peaked at 8 hours. While this peak is relatively early compared to many tumor-targeting antibodies, it is completely consistent with other studies using de2-7 EGFR specific antibodies which all show peaks at 4-24 hours post-injection when using a similar dose of antibody (Hills et al., 1995; Reist et al., 1997; Reist et al. (1996) Radioiodination of internalizing monoclonal antibodies using N-succinimidyl 5-iodo-3-pyridinecarboxylate. Cancer Res. 56, 4970-7). Indeed, unlike the earlier reports, the 8 h time point was included on the assumption that antibody targeting would peak rapidly. The % ID/g tumor seen with mAb806 was similar to that reported for other de2-7 EGFR specific antibodies when using standard iodination techniques (Hills et al., 1995; Huang et al., 1997; Reist et al. (1995) Tumor-specific anti-epidermal growth factor receptor variant III monoclonal antibodies: use of the tyramine-cellobiose radioiodination method enhances cellular retention and uptake in tumor xenografts. Cancer Res. 55, 4375-82).

The reason for the early peak is probably two-fold. Firstly, tumors expressing the de2-7 EGFR, including the transfected U87MG cells, grow extremely rapidly as tumor xenografts. Thus, even during the relatively short period of time used in these biodistribution studies, the tumor size increases to such an extent (5-10 fold increase in mass over 4 days) that the % ID/g tumor is reduced compared with slow growing tumors. Secondly, while internalization of mAb806 was relatively slow compared to DH8.3, it is still rapid with respect to many other tumor antibody/antigen systems. Internalized antibodies undergo rapid proteolysis with the degradation products being excreted from the cell (Press et al. (1990) Inhibition of catabolism of radiolabeled antibodies by tumor cells using lysosomotropic amines and carboxylic ionophores. Cancer Res. 50, 1243-50). This process of internalization, degradation and excretion reduces the amount of iodinated antibody retained within the cell. Consequently, internalizing antibodies display lower levels of targeting than their non-internalizing counterparts. The electron microscopy data reported herein demonstrates that internalized mAb806 is rapidly transported to lysosomes where rapid degradation presumably occurs. This observation is consistent with the swift expulsion of iodine from the cell.

The previously described L8A4 monoclonal antibody directed to the unique junctional peptide found in the de2-7 EGFR, behaves in a similar fashion to mAb806 (Reist et al. (1997) In vitro and in vivo behavior of radiolabeled chimeric anti-EGFRvIII monoclonal antibody: comparison with its murine parent. Nucl. Med. Biol. 24, 639-47). Using U87MG cells transfected with the de2-7 EGFR, this antibody had a similar internalization rate (35% at 1 hour compared to 30% at 1 hour for mAb806) and displayed comparable in vivo targeting when using 3T3 fibroblasts transfected with de2-7 EGFR (peak of 24% ID/g tumor at 24 hours compared to 18% ID/g tumor at 8 hours for mAb806) (Reist et al. (1997) Improved targeting of an anti-epidermal growth factor receptor variant III monoclonal antibody in tumor xenografts after labeling using N-succinimidyl 5-iodo-3-pyridinecarboxylate. Cancer Res. 57, 1510-5).

Interestingly, in vivo retention of this antibody in tumor xenografts was enhanced when labeled with N-succinimidyl 5-iodo-3-pyridine carboxylate (Reist et al., 1997). This labeled prosthetic group is positively charged at lysosmal pH and thus has enhanced cellular retention (Reist et al. (1996) Radioiodination of internalizing monoclonal antibodies using N-succinimidyl 5-iodo-3-pyridinecarboxylate. Cancer Res. 56, 4970-7). Enhanced retention is potentially useful when considering an antibody for radioimmunotherapy and this method could be used to improve retention of iodinated mAb806 or its fragments.

### Example 8

### Binding of mAb806 to Cells Containing Amplified EGFR

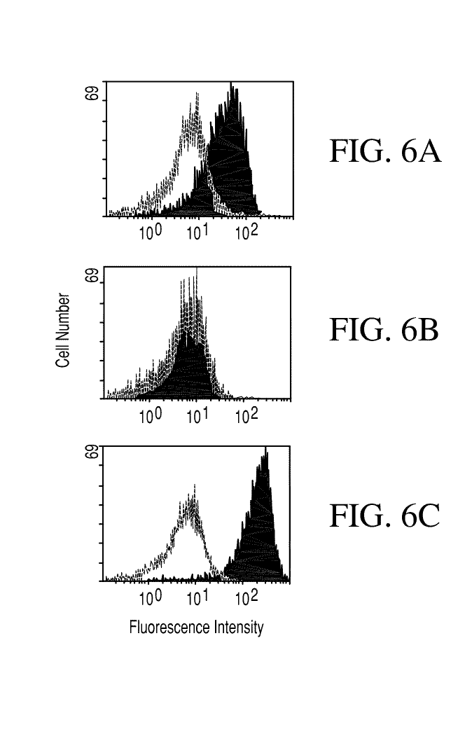

To examine if mAb806 could recognize the EGFR expressed in cells containing an amplified receptor gene, its binding to A431 cells was analyzed. As described previously, A431 cells are human squamous carcinoma cells and express high levels of wtEGFR. Low, but highly reproducible, binding of mAb806 to A431 cells was observed by FACS analysis (FIG. 6). The DH8.3 antibody did not bind A431 cells, indicating that the binding of mAb806 was not the result of low level de2-7 EGFR expression (FIG. 6). As expected, the anti-EGFR 528 antibody showed strong staining of A431 cells (FIG. 6). Given this result, binding of mAb806 to A431 was characterized by Scatchard analysis. While the binding of iodinated mAb806 was comparatively low, it was possible to get consistent data for Scatchard. The average of three such experiments gave a value for affinity of 9.5×10⁷ M⁻¹ with 2.4×10⁵ receptors per cell. Thus, the affinity for this receptor was some 10-fold lower than the affinity for the de2-7 EGFR. Furthermore, mAb806 appears to only recognize a small portion of EGFR found on the surface of A431 cells. The 528 antibody measured approximately 2×10⁶ receptors per cell which is in agreement with numerous other studies (Santon et al. (1986) Effects of epidermal growth factor receptor concentration on tumorigenicity of A431 cells in nude mice. Cancer Res. 46, 4701-5).

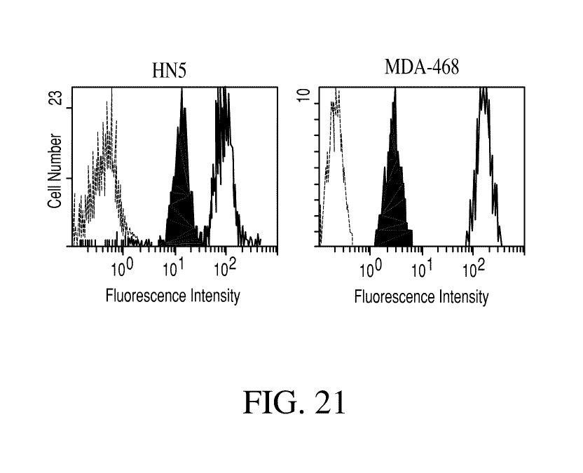

To ensure that these results were not simply restricted to the A431 cell line, mAb806 reactivity was examined in 2 other cells lines exhibiting amplification of the EGFR gene. Both the HN5 head and neck cell line (Kwok T T and Sutherland R M (1991) Differences in EGF related radiosensitisation of human squamous carcinoma cells with high and low numbers of EGF receptors. Br. J. Cancer. 64, 251-4) and the MDA-468 breast cancer cell line (Filmus et al. (1985) MDA-468, a human breast cancer cell line with a high number of epidermal growth factor (EGF) receptors, has an amplified EGF receptor gene and is growth inhibited by EGF. Biochem. Biophys. Res. Commun. 128, 898-905) have been reported to contain multiple copies of the EGFR gene. Consistent with these reports, the 528 antibody displayed intense staining of both cell lines (FIG. 21). As with the A431 cell line, the mAb806 clearly stained both cell lines but at a lower level than that observed with the 528 antibody (FIG. 21). Thus, mAb806 binding is not simply restricted to A431 cells but appears to be a general observation for cells containing amplification of the EGFR gene.

Recognition of the wild-type sEGFR by mAb806 clearly requires some denaturation of the receptor in order to expose the epitope. The extent of denaturation required is only slight as even absorption of the wild-type sEGFR on to a plastic surface induced robust binding of mAb806 in ELISA assays. As mAb806 only bound approximately 10% of the EGFR on the surface of A431 cells, it is tempting to speculate that this subset of receptors may have an altered conformation similar to that induced by the de2-7 EGFR truncation. Indeed, the extremely high expression of the EGFR mediated by gene amplification in A431 cells may cause some receptors to be incorrectly processed leading to altered conformation. Interestingly, semi-quantitative immunoblotting of A431 cell lysates with mAb806 showed that it could recognize most of the A431 EGF receptors following SDS-PAGE and western transfer. This result further supports the argument that mAb806 is binding to a subset of receptors on the surface of A431 cells that have an altered conformation. These observations in A431 cells are consistent with the immunohistochemistry data demonstrating that mAb806 binds gliomas containing amplification of the EGFR gene. As mAb806 binding was completely negative on parental U87MG cells it would appear this phenomenon may be restricted to cells containing amplified EGFR although the level of “denatured” receptor on the surface of U87MG cells may be below the level of detection. However, this would seem unlikely as iodinated mAb806 did not bind to U87MG cell pellets containing up to 1×10⁷ cells.

### Example 9

### In vivo Targeting of A431 Cells By mAb806

A second biodistribution study was performed with mAb806 to determine if it could target A431 tumor xenografts. The study was conducted over a longer time course in order obtain more information regarding the targeting of U87MG.Δ2-7 xenografts by mAb806, which were included in all mice as a positive control. In addition, the anti-EGFR 528 antibody was included as a positive control for the A431 xenografts, since a previous study demonstrated low but significant targeting of this antibody to A431 cells grown in nude mice (Masui et al. (1984) Growth inhibition of human tumor cells in athymic mice by anti-epidermal growth factor receptor monoclonal antibodies. Cancer Res. 44, 1002-7).

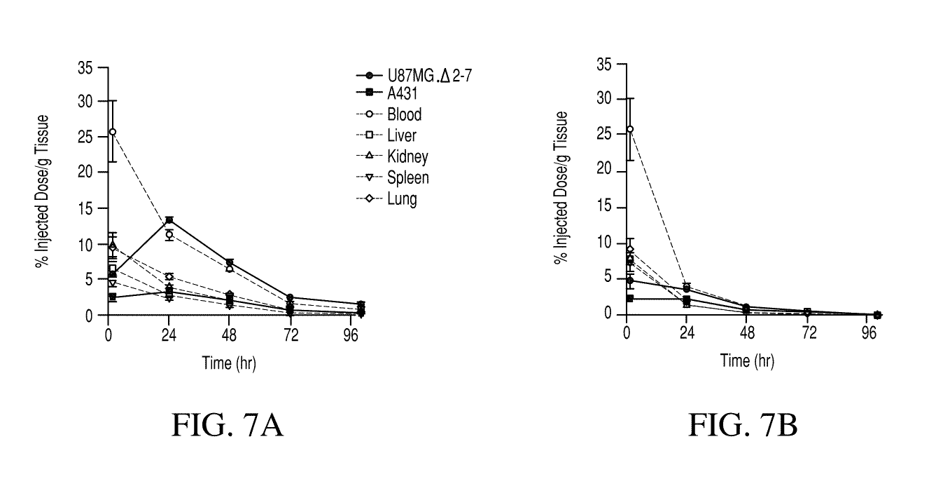

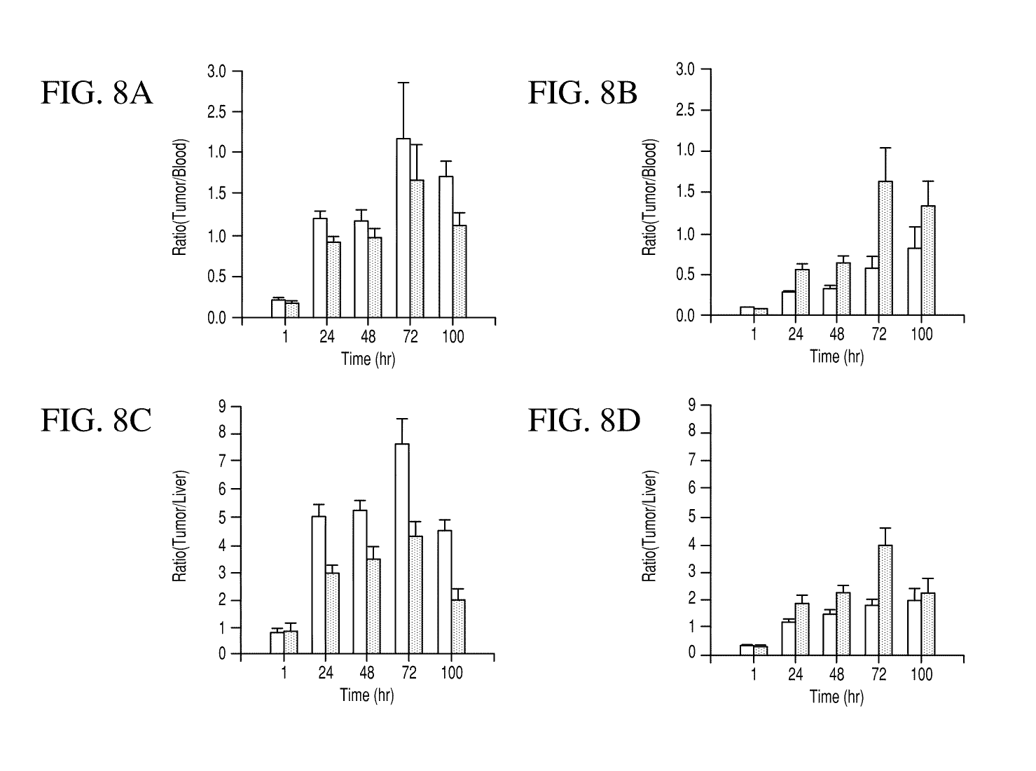

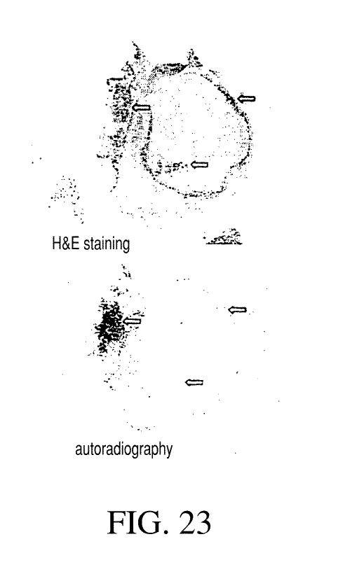

During the first 48 h, mAb806 displayed almost identical targeting properties as those observed in the initial experiments (FIG. 7A compared with FIG. 4A). In terms of % ID/g tumor, levels of mAb806 in U87MG.Δ2-7 xenografts slowly declined after 24 h but always remained higher than levels detected in normal tissue. Uptake in the A431 xenografts was comparatively low, however there was a small increase in % ID/g tumor during the first 24 h not observed in normal tissues such as liver, spleen, kidney and lung (FIG. 7A). Uptake of the 528 antibody was very low in both xenografts when expressed as % ID/g tumor (FIG. 7B) partially due to the faster clearance of this antibody from the blood. Autoradiography of A431 xenograft tissue sections collected 24 hr after injection with ¹²⁵I-labeled mAb806 alone, clearly illustrates localization of antibody to viable tumor around the periphery of the tumor and not central areas of necrosis (FIG. 23). In terms of tumor to blood ratio mAb806 peaked at 72 h for U87MG.Δ2-7 xenografts and 100 h for A431 xenografts (FIGS. 8A, B). While the tumor to blood ratio for mAb806 never surpassed 1.0 with respect to the A431 tumor, it did increase throughout the entire time course (FIG. 8B) and was higher than all other tissues examined (data not shown) indicating low levels of targeting.

The tumor to blood ratio for the 528 antibody showed a similar profile to mAb806 although higher levels were noted in the A431 xenografts (FIGS. 8A, B). mAb806 had a peak tumor to liver ratio in U87MG.Δ2-7 xenografts of 7.6 at 72 h, clearly demonstrating preferential uptake in these tumors compared to normal tissue (FIG. 8C). Other tumor to organ ratios for mAb806 were similar to those observed in the liver (data not shown). The peak tumor to liver ratio for mAb806 in A431 xenografts was 2.0 at 100 h, again indicating a slight preferential uptake in tumor compared with normal tissue (FIG. 8D).

### Example 10

### Therapy Studies

The effects of mAb806 were assessed in two xenograft models of disease—a preventative model and an established tumor model.

### Xenograft Models

Consistent with previous reports (Nishikawa et al., Proc. Natl. Acad. Sci. U.S.A., 91(16), 7727-7731), U87MG cells transfected with de2-7 EGFR grew more rapidly than parental cells and U87MG cells transfected with the wtEGFR. Therefore, it was not possible to grow both cell types in the same mice.

Tumor cells (3×10⁶) in 100 ml of PBS were inoculated subcutaneously into both flanks of 4-6 week old female nude mice (Animal Research Centre, Western Australia, Australia). Therapeutic efficacy of mAb806 was investigated in both preventative and established tumor models. In the preventative model, 5 mice with two xenografts each were treated intraperitoneally with either 1 or 0.1 mg of mAb806 or vehicle (PBS) starting the day before tumor cell inoculation. Treatment was continued for a total of 6 doses, 3 times per week for 2 weeks. In the established model, treatment was started when tumors had reached a mean volume of 65±6.42 mm³ (U87MG.Δ2-7), 84±9.07 mm3 (U87MG), 73±7.5 mm³ (U87MG.wtEGFR) or 201±19.09 mm³ (A431 tumors). Tumor volume in mm³ was determined using the formula (length×width²)/2, where length was the longest axis and width the measurement at right angles to the length (Clark et al. (2000) Therapeutic efficacy of anti-Lewis (y) humanized 3S193 radioimmunotherapy in a breast cancer model: enhanced activity when combined with Taxol chemotherapy. Clin. Cancer Res. 6, 3621-3628). Data was expressed as mean tumor volume±S.E. for each treatment group. Statistical analysis was performed at given time points using Student's t-test. Animals were euthanized when the xenografts reached an approximate volume of 1.5 cm³ and the tumors excised for histological examination. This research project was approved by the Animal Ethics Committee of the Austin and Repatriation Medical Centre.

### Histological Examination of Tumor Xenografts

Xenografts were excised and bisected. One half was fixed in 10% formalin/PBS before being embedded in paraffin. Four micron sections were then cut and stained with haematoxylin and eosin (H&E) for routine histological examination. The other half was embedded in Tissue Tek® OCT compound (Sakura Finetek, Torrance, Calif.), frozen in liquid nitrogen and stored at −80° C. Thin (5 micron) cryostat sections were cut and fixed in ice-cold acetone for 10 min followed by air drying for a further 10 min Sections were blocked in protein blocking reagent (Lipshaw Immunon, Pittsburgh U.S.A.) for 10 min and then incubated with biotinylated primary antibody (1 mg/ml), for 30 min at room temperature (RT). All antibodies were biotinylated using the ECL protein biotinylation module (Amersham, Baulkham Hills, Australia), as per the manufacturer's instructions. After rinsing with PBS, sections were incubated with a streptavidin horseradish peroxidase complex for a further 30 min (Silenus, Melbourne, Australia). Following a final PBS wash the sections were exposed to 3-amino-9-ethylcarbozole (AEC) substrate (0.1 M acetic acid, 0.1 M sodium acetate, 0.02 M AEC (Sigma Chemical Co., St Louis, Mo.)) in the presence of hydrogen peroxide for 30 min Sections were rinsed with water and counterstained with hematoxylin for 5 min and mounted.

Efficacy of mAb806 in Preventative Model

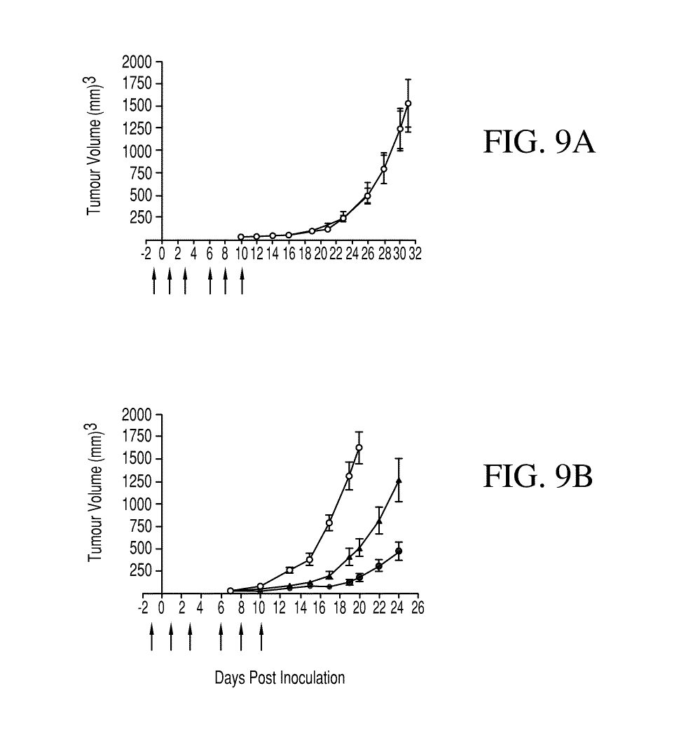

mAb806 was examined for efficacy against U87MG and U87MG.Δ2-7 tumors in a preventative xenograft model. Antibody or vehicle were administered i.p. the day before tumor inoculation and was given 3 times per week for 2 weeks. mAb806 had no effect on the growth of parental U87MG xenografts, which express the wtEGFR, at a dose of 1 mg per injection (FIG. 9A). In contrast, mAb806 significantly inhibited the growth of U87MG.Δ2-7 xenografts in a dose dependent manner (FIG. 9B). At day 20, when control animals were sacrificed, the mean tumor volume was 1637±178.98 mm³ for the control group, a statistically smaller 526±94.74 mm³ for the 0.1 mg per injection group (p<0.0001) and 197±42.06 mm³ for the 1 mg injection group (p<0.0001). Treatment groups were sacrificed at day 24 at which time the mean tumor volumes was 1287±243.03 mm³ for the 0.1 mg treated group and 492±100 8 mm³ for the 1 mg group.

Efficacy of mAb806 in Established Xenograft Model

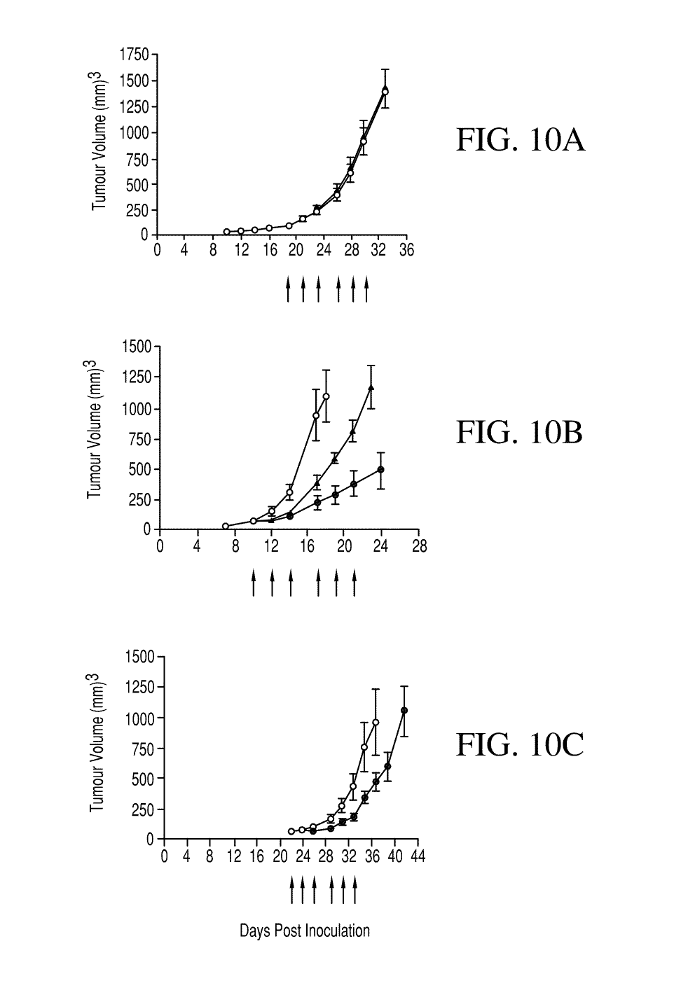

Given the efficacy of mAb806 in the preventative xenograft model, its ability to inhibit the growth of established tumor xenografts was then examined. Antibody treatment was as described in the preventative model except that it commenced when tumors had reached a mean tumor volume of 65±6.42 mm³ for the U87MG.Δ2-7 xenografts and 84±9.07 mm³ for the parental U87MG xenografts. Once again, mAb806 had no effect on the growth of parental U87MG xenografts at a dose of 1 mg per injection (FIG. 10A). In contrast, mAb806 significantly inhibited the growth of U87MG.Δ2-7 xenografts in a dose dependent manner (FIG. 10B). At day 17, one day before control animals were sacrificed, the mean tumor volume was 935±215.04 mm³ for the control group, 386±57.51 mm³ for the 0.1 mg per injection group (p<0.01) and 217±58.17 mm³ for the 1 mg injection group (p<0.002).

To examine whether the growth inhibition observed with mAb806 was restricted to cell expressing de2-7 EGFR, its efficacy against U87MG.wtEGFR tumor xenografts was examined in an established model. These cells serve as a model for tumors containing amplification of the EGFR gene without de2-7 EGFR expression. mAb806 treatment commenced when tumors had reached a mean tumor volume of 73±7.5 mm³ mAb806 significantly inhibited the growth of established U87MG.wtEGFR xenografts when compared to control tumors treated with vehicle (FIG. 10C). On the day control animals were sacrificed, the mean tumor volume was 960±268.9 mm³ for the control group and 468±78.38 mm³ for the group treated with 1 mg injections (p<0.04).

### Histological and Immunohistochemical Analysis of Established Tumors

To evaluate potential histological differences between mAb806-treated and control U87MG.Δ2-7 and U87MG.wtEGFR xenografts (collected at days 24 and 42 respectively), formalin-fixed, paraffin embedded sections were stained with H&E. Areas of necrosis were seen in sections from both U87MG.Δ2-7 (collected 3 days after treatment finished), and U87MG.wtEGFR xenografts (collected 9 days after treatment finished) treated with mAb806. This result was consistently observed in a number of tumor xenografts (n=4). However, analysis of sections from xenografts treated with control did not display the same areas of necrosis seen with mAb806 treatment. Sections from mAb806 or control treated U87MG xenografts were also stained with H&E and revealed no differences in cell viability between the two groups, further supporting the hypothesis that mAb806 binding induces decreased cell viability/necrosis within tumor xenografts.

An immunohistochemical analysis of U87MG, U87MG.Δ2-7 and U87MG.wtEGFR xenograft sections was performed to determine the levels of de2-7 and wtEGFR expression following mAb806 treatment. Sections were collected at days 24 and 42 as above, and were immunostained with the 528 or 806 antibodies. As expected, the 528 antibody stained all xenograft sections with no obvious decrease in intensity between treated and control tumors. Staining of U87MG sections was undetectable with the mAb806, however positive staining of U87MG.Δ2-7 and U87MG.wtEGFR xenograft sections was observed. There was no difference in mAb806 staining density between control and treated U87MG.Δ2-7 and U87MG.wtEGFR xenografts suggesting that antibody treatment does not down regulate de2-7 or wtEGFR expression.

Treatment of A431 Xenografts with mAb806

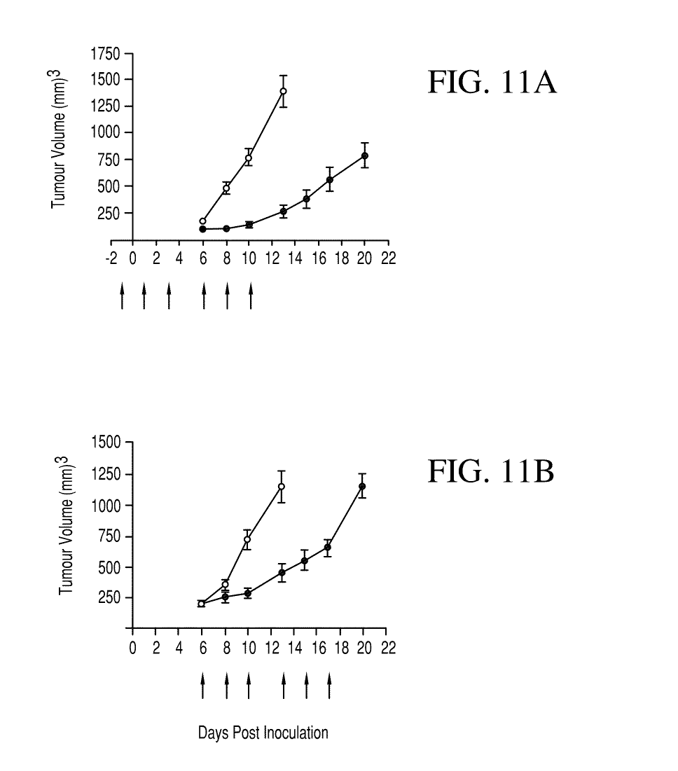

To demonstrate that the anti-tumor effects of mAb806 were not restricted to U87MG cells, the antibody was administered to mice with A431 xenografts. These cells contain an amplified EGFR gene and express approximately 2×10⁶ receptors per cell. As described above, mAb806 binds about 10% of these EGFR and targets A431 xenografts. mAb806 significantly inhibited the growth of A431 xenografts when examined in the previously described preventative xenograft model (FIG. 11A). At day 13, when control animals were sacrificed, the mean tumor volume was 1385±147.54 mm³ in the control group and 260±60.33 mm³ for the 1 mg injection treatment group (p<0.0001).

In a separate experiment, a dose of 0.1 mg mAb also significantly inhibited the growth of A431 xenografts in a preventative model.

Given the efficacy of mAb806 in the preventative A431 xenograft model, its ability to inhibit the growth of established tumor xenografts was examined. Antibody treatment was as described in the preventative model except it was not started until tumors had reached a mean tumor volume of 201±19.09 mm³ mAb806 significantly inhibited the growth of established tumor xenografts (FIG. 11B). At day 13, when control animals were sacrificed, the mean tumor volume was 1142±120.06 mm³ for the control group and 451±65.58 mm³ for the 1 mg injection group (p<0.0001).

In summary, the therapy studies with mAb806 described here clearly demonstrated dose dependent inhibition of U87MG.Δ2-7 xenograft growth. In contrast, no inhibition of parental U87MG xenografts was observed despite the fact they continue to express the wtEGFR in vivo. mAb806 not only significantly reduced xenograft volume, it also induced significant necrosis within the tumor. This is the first report showing the successful therapeutic use of such an antibody in vivo against a human de2-7 EGFR expressing glioma xenografts.

Gene amplification of the EGFR has been reported in a number of different tumors and is observed in approximately 50% of gliomas (Voldberg et al., 1997). It has been proposed that the subsequent EGFR over-expression mediated by receptor gene amplification may confer a growth advantage by increasing intracellular signaling and cell growth (Filmus et al., 1987). The U87MG cell line was transfected with the wtEGFR in order to produce a glioma cell that mimics the process of EGFR gene amplification. Treatment of established U87MG.wtEGFR xenografts with mAb806 resulted in significant growth inhibition. Thus, mAb806 also mediates in vivo antitumor activity against cells containing amplification of the EGFR gene. Interestingly, mAb806 inhibition of U87MG.wtEGFR xenografts appears to be less effective than that observed with U87MG.Δ2-7 tumors. This probably reflects the fact that mAb806 has a lower affinity for the amplified EGFR and only binds a small proportion of receptors expressed on the cell surface. However, it should be noted that despite the small effect on U87MG.wtEGFR xenograft volumes, mAb806 treatment produced large areas of necrosis within these xenografts.

To rule out the possibility that mAb806 only mediates inhibition of the U87MG derived cell lines we tested its efficacy against A431 xenografts. This squamous cell carcinoma derived cell line contains significant EGFR gene amplification which is retained both in vitro and in vivo. Treatment of A431 xenografts with mAb806 produced significant growth inhibition in both a preventative and established model, indicating the anti-tumor effects of mAb806 are not restricted to transfected U87MG cell lines.

### Example 11

### Combination Therapy Treatment of A431 Xenografts with mAb806 and AG1478

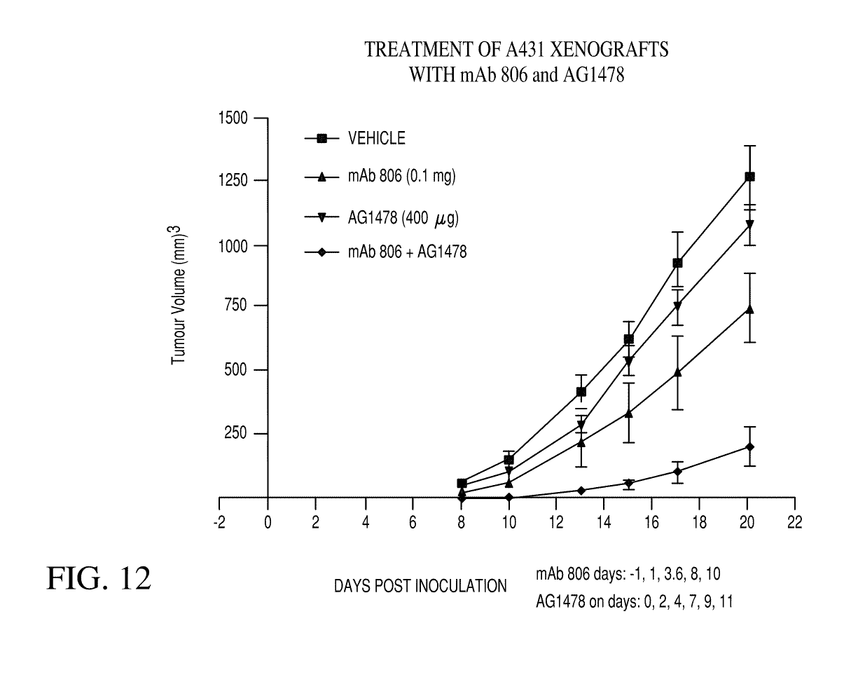

The anti-tumor effects of mAb806 combined with AG1478 was tested in mice with A431 xenografts. AG1478 (4-(3-Chloroanilino)-6,7-dimethoxyquinazoline) is a potent and selective inhibitor of the EGFR kinase versus HER2-neu and platelet-derived growth factor receptor kinase (Calbiochem Cat. No. 658552). Three controls were included: treatment with vehicle only, vehicle+mAb806 only, and vehicle+AG1478 only. The results are illustrated in FIG. 12. 0.1 mg mAb806 was administered at 1 day prior to xenograft and 1, 3, 6, 8 and 10 days post xenograft. 400 μg AG1478 was administered at 0, 2, 4, 7, 9, and 11 days post xenograft.

Both AG1478 and mAb806, when administered alone, produced a significant reduction of tumor volume. However, in combination, the reduction of tumor volume was greatly enhanced.

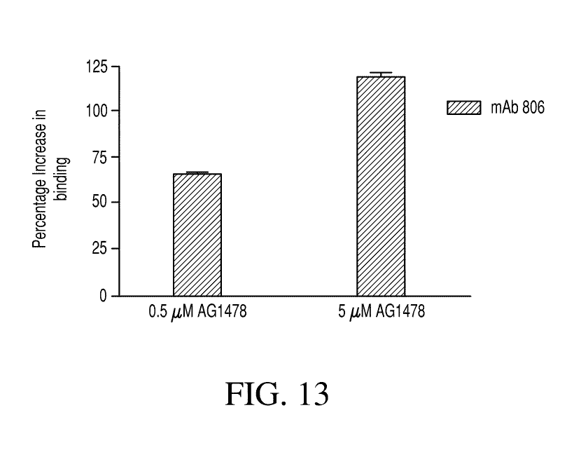

In addition, the binding of mAb806 to EGFR of A431 cells was evaluated in the absence and presence of AG1478. Cells were placed in serum free media overnight, then treated with AG1478 for 10 min at 37° C., washed twice in PBS, then lysed in 1% Triton and lysates prepared by centrifugation for 10 min at 12,000 g. Lysate was then assessed for 806 reactivity by an ELISA in a modified version of an assay described by Schooler and Wiley, Analytical Biochemistry 277, 135-142 (2000). Plates were coated with 10 μg/ml of mAb806 in PBS/EDTA overnight at room temperature and then washed twice. Plates were then blocked with 10% serum albumin/PBS for 2 hours at 37° C. and washed twice. A 1:20 cell lysate was added in 10% serum albumin/PBS for 1 hour at 37° C., then washed four times. Anti-EGFR(SC-03; Santa Cruz Biotechnology Inc.) in 10% serum albumin/PBS was reacted 90 min at room temperature, the plate washed four times, and anti-rabbit-HRP (1:2000 if from Silenus) in 10% serum albumin/PBS was added for 90 min at room temperature, washed four times, and color developed using ABTS as a substrate. It was found that mAb806 binding is significantly increased in the presence of increasing amounts of AG1478 (FIG. 13).

### Example 12

### Immunoreactivity in Human Glioblastomas Pre-Typed For EGFR Status

Given the high incidence of EGFR expression, amplification and mutation in glioblastomas, a detailed immunohistochemical study was performed in order to assess the specificity of 806 in tumors other than xenografts. A panel of 16 glioblastomas was analyzed by immunohistochemistry. This panel of 16 glioblastomas was pre-typed by RT-PCR for the presence of amplified wild-type EGFR and de2-7 EGFR expression. Six of these tumors expressed only the wtEGFR transcript, 10 had wtEGFR gene amplification with 5 of these showing wild-type EGFR transcripts only, and 5 both wild-type EGFR and de2-7 gene transcript.

Immunohistochemical analysis was performed using 5 mm sections of fresh frozen tissue applied to histology slides and fixed for 10 minutes in cold acetone. Bound primary antibody was detected with biotinylated horse anti-mouse antibody followed by an avidin-biotin-complex reaction. Diaminobenzidine tetrahydrochloride (DAB) was used as chromogen. The extent of the immunohistochemical reactivity in tissues was estimated by light microscopy and graded according to the number of immunoreactive cells in 25% increments as follows:

Focal=less than 5%

+=5-25%

++=25-50%

+++=50-75%

++++=>75%

The 528 antibody showed intense reactivity in all tumors, while DH8.3 immunostaining was restricted to those tumors expressing the de2-7 EGFR (Table 2). Consistent with the previous observations in FACS and rosetting assays, mAb806 did not react with the glioblastomas expressing the wtEGFR transcript from nonamplified EGFR genes (Table 2). This pattern of reactivity for mAb806 is similar to that observed in the xenograft studies and again suggests that this antibody recognizes the de2-7 and amplified EGFR but not the wtEGFR when expressed on the cell surface.

TABLE 2
Immunoreactivity of mAbs528, DH8.3 and 806 on glioblastomas
pre-typed for the presence of wild-type EGFR and mutated
de2-7 EGFR and for their amplification status

| | de2-7 EGFR | | | |
|---|---|---|---|---|
| Amplification | Expression | 528 | DH8.3 | 806 |
| | No | ++++ | — | — |
| | No | ++++ | — | —* |
| | No | ++++ | — | — |
| | No | ++ | — | — |
| | No | +++ | — | — |
| | No | ++++ | — | — |
| Yes | No | ++++ | — | ++++ |
| Yes | No | ++++ | — | + |
| Yes | No | ++++ | — | +++ |
| Yes | No | ++++ | — | ++++ |
| Yes | No | ++++ | — | +-++++ |
| Yes | Yes | ++++ | ++++ | ++++ |
| Yes | Yes | ++++ | ++++ | ++++ |
| Yes | Yes | ++++ | ++++ | ++++ |
| Yes | Yes | ++++ | ++++ | ++++ |
| Yes | Yes | ++++ | ++ | ++ |

*focal staining

### Example 13

### EGFR Immunoreactivity In Normal Tissue

In order to determine if the de2-7 EGFR is expressed in normal tissue, an immunohistochemical study with mAb806 and DH8.3 was conducted in a panel of 25 tissues. There was no strong immunoreactivity with either mAb806 or DH8.3 in any tissue tested, suggesting that the de2-7 EGFR is absent in normal tissues (Table 3). There was some variable staining present in tonsils with mAb806 that was restricted to the basal cell layer of the epidermis and mucosal squamous cells of the epithelium. In placenta, occasional immunostaining of the trophoblast epithelium was observed. Interestingly, two tissues that express high endogenous levels of wtEGFR, the liver and skin, failed to show any significant mAb806 reactivity. No reactivity was observed with the liver samples at all, and only weak and inconsistent focal reactivity was detected occasionally (in no more than 10% of all samples studied) in basal keratinocytes in skin samples and in the squamous epithelium of the tonsil mucosa, further demonstrating that this antibody does not bind the wtEGFR expressed on the [0464] surface of cells to any significant extent (Table 3). All tissues were positive for the wtEGFR as evidenced by the universal staining seen with the 528 antibody (Table 3).

TABLE 3
Reactivity of 582, DH8.3 and 806 on normal tissues

| Tissue | 528 | DH8.3 | 806 |
|---|---|---|---|
| Esophagus | pos | — | — |
| Stomach | pos | — | — |
| Duodenum | pos | — | — |
| Small | pos | — | — |
| intestine/duodenum | | | |
| Colon | pos | — | — |
| Liver | pos | — | — |
| Salivary glands | pos | — | — |
| (parotid) | | | |
| Kidney | pos | — | — |
| Urinary Bladder | pos | — | — |
| Prostate | pos | — | — |
| Testis | pos | — | — |
| Uterus (cx/endom) | pos | —* | — |
| Fallopian tube | pos | — | — |
| Ovary | pos | — | — |
| Breast | pos | —* | — |
| Placenta | pos | — | — |
| Peripheral nerve | pos | — | — |
| Skeletal muscle | pos | — | — |
| Thyroid gland | pos | — | — |
| Lymph node | pos | — | — |
| Spleen | pos | — | — |
| Tonsil | pos | — | — occ. weak reactivity |
| | | | of basal layer of |
| | | | squamous epithelium |
| Heart | pos | — | — |
| Lung | pos | — | — |
| Skin | pos | — | — occ. weak reactivity |
| | | | of basal layer of |
| | | | squamous epithelium |

*some stromal staining in various tissue

### Example 14

### EGFR Immunoreactivity in Various Tumors

The extent of de2-7 EGFR in other tumor types was examined using a panel of 12 different malignancies. The 528 antibody showed often homogeneous staining in many tumors analyzed except melanoma and seminoma. When present, DH8.3 immunoreactivity was restricted to the occasional focal tumor cell indicating there is little if any de2-7 EGFR expression in tumors outside the brain using this detection system (Table 4). There was also focal staining of blood vessels and a varying diffuse staining of connective tissue with the DH8.3 antibody in some tumors (Table 4). This staining was strongly dependent on antibody concentration used and was considered nonspecific background reactivity. The mAb806 showed positive staining in 64% of head and neck tumors and 50% of lung carcinomas (Table 4). There was little mAb806 reactivity elsewhere except in urinary tumors that were positive in 30% of cases.

Since the head and neck and lung cancers were negative for the DH8.3 antibody the reactivity seen with the mAb in these tumors maybe associated with EGFR gene amplification.

TABLE 4
Monoclonal antibodies 528, DH8.3 and 806 on tumor panel

| Tumor | 528 | DH8.3 | 806 |
|---|---|---|---|
| Malignant melanoma | 0/10 | 0/10 | 0/10 |
| metastases | | | |
| Urinary bladder (tcc, | 10/10 | 0/10* | 3/10* |
| sqcc, adeno) | (7x++++, 2x++++, 1x+) | | (2x++++, 1x++) |
| Mammary gland | 6/10 | 1/10 | 1/10 |
| | (3x++++, 3x++) | (1x+) | (foc) |
| Head + neck cancer | 11/11 | 0/11* | 7/11 |
| (sqcc) | (1x+++-10x++++) | | (3x++++, 3x+++, 1x+) |
| Lung (sqcc, adeno, | 12/12 | 0/12* | 6/12 |
| neuroend) | (10x++++-1x+++) | | (3x++++ 3x+++) |
| Leiomyosarcoma | 5/5 | 0/5 | 0/5 |
| | (4x++++, 1x+) | | |
| Liposarcoma | 5/5 | 0/5 | 0/5* |
| | (2x + 3x +++) | | |
| Synovial sarcoma | 4/5* | 0/5 | 0/5* |
| | (4x ++++) | | |
| Mfh Malignant | 4/5* | 0/5* | 0/5* |
| fibrous histiocytoma | | | |
| Colonic carcinoma | 10/10 | 0/10* | 0/10 |
| | (9x++++, 1x+) | | |
| Seminoma | 1/10* | 1/10* | 0/10 |
| Ovary (serous- | 4/5 | 0/5* | 0/5 |
| papillary) | (3x++++, 1x+) | | |

*focal staining

### Example 15

### Immunoreactivity In Human Glioblastomas Unselected For EGFR Status

In order to confirm the unique specificity and to evaluate the reactivity of mAb806, it was compared to the 528 and DH8.3 antibodies in a panel of 46 glioblastomas not preselected for their EGFR status. The 528 antibody was strongly and homogeneously positive in all samples except two (Nos. 27 and 29) (44/46, 95.7%). These two cases were also negative for mAb806 and mAbDH8.3. The mAb806 was positive in 27/46 (58.7%) cases, 22 of which displayed homogeneous immunoreactivity in more than 50% of the tumor. The DH8.3 antibody was positive in 15/46 (32.6%) glioblastomas, 9 of which showed homogeneous immunoreactivity. The immunochemical staining of these unselected tumors is tabulated in Table 5.

There was concordance between mAb806 and DH8.3 in every case except one (No. 35). A molecular analysis for the presence of EGFR amplification was done in 44 cases (Table 5). Of these, 30 cases co-typed with the previously established mAb806 immunoreactivity pattern: e.g., 16 mAb806-negative cases revealed no EGFR amplification and 14 EGFR-amplified cases were also mAb806 immunopositive. However, 13 cases, which showed 806 immunoreactivity, were negative for EGFR amplification while one EGFR-amplified case was mAb806 negative. Further analysis of the mutation status of these amplification negative and 806 positive cases is described below and provides explanation for most of the 13 cases which were negative for EGFR amplification and were recognized by 806.

Subsequently, a molecular analysis of the deletion mutation by RT-PCR was performed on 41/46 cases (Table 5). Of these, 34 cases co-typed with DH8.3 specific for the deletion mutation: 12 cases were positive in both RT-PCR and immunohistochemistry and 22 cases were negative/negative. Three cases (#2, #34, and #40) were DH8.3 positive/RT-PCR negative for the deletion mutation and three cases (#12, #18, and #39) were DH8.3 negative/RT-PCR positive. As expected based on our previous specificity analysis, mAb806 immunoreactivity was seen in all DH8.3 positive tissues except in one case (#35).

Case #3 also revealed a mutation (designated A2 in Table 5), which included the sequences of the de2-7 mutation but this did not appear to be the classical de2-7 deletion with loss of the 801 bases (data not shown). This case was negative for DH8.3 reactivity but showed reactivity with 806, indicating that 806 may recognize an additional and possibly unique EGFR mutation.

TABLE 5
Immunohistochemical Analysis of 46 Unselected
Glioblastomas With mAbs 528, 806, and DH8.3

| | | | | EGFR | |
|---|---|---|---|---|---|
| # | 528 | 806 | DH8.3 | Amp.* | 5′ MUT |
| 1 | ++++ | ++++ | ++ | A | 5′ MUT |
| 2 | ++++ | ++++ | ++++ | N | WT |
| 3 | ++++ | ++++ | neg. | N | A2 |
| | | (det.) | | | |
| 4 | ++++ | ++++ | neg. | N | WT |
| 5 | ++++ | ++++ | ++++ | N | 5′ MUT |
| 6 | ++++ | ++++ | neg. | A | WT |
| 7 | ++++ | ++++ | ++++ | N | 5′ MUT |
| 8 | ++++ | ++++ | ++++ | A | 5′ MUT |
| 9 | ++++ | ++++ | neg. | A | WT |
| 10 | ++++ | neg. | neg. | N | WT |
| 11 | ++ | ++ | ++ | A | 5′ MUT |
| 12 | ++++ | ++ | neg. | A | 5′ MUT |
| 13 | ++++ | ++++ | neg. | N | WT |
| 14 | ++ | neg. | neg. | Nd | nd |
| 15 | ++ | ++ | neg. | N | WT |
| 16 | + | neg. | neg. | N | nd |
| 17 | ++++ | neg. | neg. | N | WT |
| 18 | ++++ | ++++ | neg. | A | 5′ MUT |
| 19 | ++++ | ++++ | neg. | N | WT |
| 20 | ++++ | neg. | neg. | N | WT |
| 21 | ++++ | ++++ | neg. | N | WT |
| 22 | +++ | neg. | neg. | N | WT |
| 23 | ++++ | ++++ | ++ | N | 5′ MUT |
| 24 | ++++ | ++++ | neg. | A | WT |
| 25 | ++++ | neg. | neg. | N | WT |
| 26 | ++++ | ++++ | +++ | A | 5′ MUT |
| 27 | neg. | neg. | neg. | N | WT |
| 28 | +++ | neg. | neg. | N | WT |
| 29 | neg. | neg. | neg. | N | WT |
| 30 | ++++ | ++++ | neg. | N | WT |
| 31 | par det | neg. | neg. | N | nd |
| 32 | ++ | +++ | ++ | N | 5′ MUT |
| 33 | +++ | ++++ | ++++ | A | 5′ MUT |
| 34 | ++++ | +++ | ++++ | N | WT |
| 35 | ++++ | neg. | ++++ | A | 5′ MUT |
| 36 | +++ | ++ | +++ | A | 5′ MUT |
| 37 | ++++ | + | + | A | 5′ MUT |
| 38 | ++++ | neg. | neg. | N | WT |
| 39 | ++ | neg. | neg. | N | 5′ MUT |
| 40 | ++++ | ++++ | + | A | WT |
| 41 | ++ | neg. | neg. | N | WT |
| 42 | ++++ | ++++ | neg. | A | WT |
| 43 | ++++ | neg. | neg. | nd | nd |
| 44 | ++++ | neg. | neg. | N | WT |
| 45 | ++++ | neg. | neg. | N | WT |
| 46 | ++++ | neg. | neg. | N | nd |

*N = not amplified, A—amplified,
⁺WT = wild-type, 5′-mut
nd = not done

The 806 antibody reactivity co-typed with amplified or de2-7 mutant EGFR in 19/27 or over 70% of the cases. It is notable that 2 of these 8 cases were also DH8.3 reactive.

### Example 16

### Systemic Treatment and Analysis of Intracranial Glioma Tumors

To test the efficacy of the anti-ΔEGFR monoclonal antibody, mAb806, we treated nude mice bearing intracranial ΔEGFR-overexpressing glioma xenografts with intraperitoneal injections of mAb806, the isotype control IgG or PBS.

Because primary explants of human glioblastomas rapidly lose expression of amplified, rearranged receptors in culture, no existing glioblastoma cell lines exhibit such expression. To force maintenance of expression levels comparable with those seen in human tumors, U87MG, LN-Z308, and A1207 (gift from Dr. S. Aaronson, Mount Sinai Medical Center, New York, N.Y.) cells were infected with ΔEGFR, kinase-deficient ΔEGFR (DK), or wild-type EGFR (wtEGFR) viruses, which also conferred resistance to G418 as described previously (Nishikawa et al. (1994) A mutant epidermal growth factor receptor common in human glioma confers enhanced tumorigenicity. Proc. Natl. Acad. Sci. U.S.A., 91, 7727-7731).

Populations expressing similar levels of the various EGFR alleles (these expression levels correspond approximately to an amplification level of 25 gene copies; human glioblastomas typically have amplification levels from 10 to 50 gene copies of the truncated receptor) were selected by FACS as described previously (Nishikawa et al., 1994) and designated as U87MG.ΔEGFR, U87MG.DK, U87MG.wtEGFR, LN-Z308.ΔEGFR, LN-Z308.DK, LN-Z308.wtEGFR, A1207.ΔEGFR, A1207.DK, and A1207.wtEGFR, respectively. Each was maintained in medium containing G418 (U87MG cell lines, 400 μg/ml; LN-Z308 and A1207 cell lines, 800 μg/ml).

U87MG.ΔEGFR cells (1×10⁵) or 5×10⁵ LN-Z308.ΔEGFR, A1207.ΔEGFR, U87MG, U87MG.DK, and U87MG.wtEGFR cells in 5 μl of PBS were implanted into the right corpus stratum of nude mice brains as described previously (Mishima et al. (2000) A peptide derived from the non-receptor binding region of urokinase plasminogen activator inhibits glioblastoma growth and angiogenesis in vivo in combination with cisplatin. Proc. Natl. Acad. Sci. U.S.A. 97, 8484-8489). Systemic therapy with mAb806, or the IgG2b isotype control, was accomplished by i.p. injection of 1 μg of mAbs in a volume of 100 μl every other day from post-implantation day 0 through 14. For direct therapy of intracerebral U87MG.ΔEGFR tumors, 10 μg of mAb806, or the IgG2b isotype control, in a volume of 5 μl were injected at the tumor-injection site every other day starting at day 1 for 5 days.

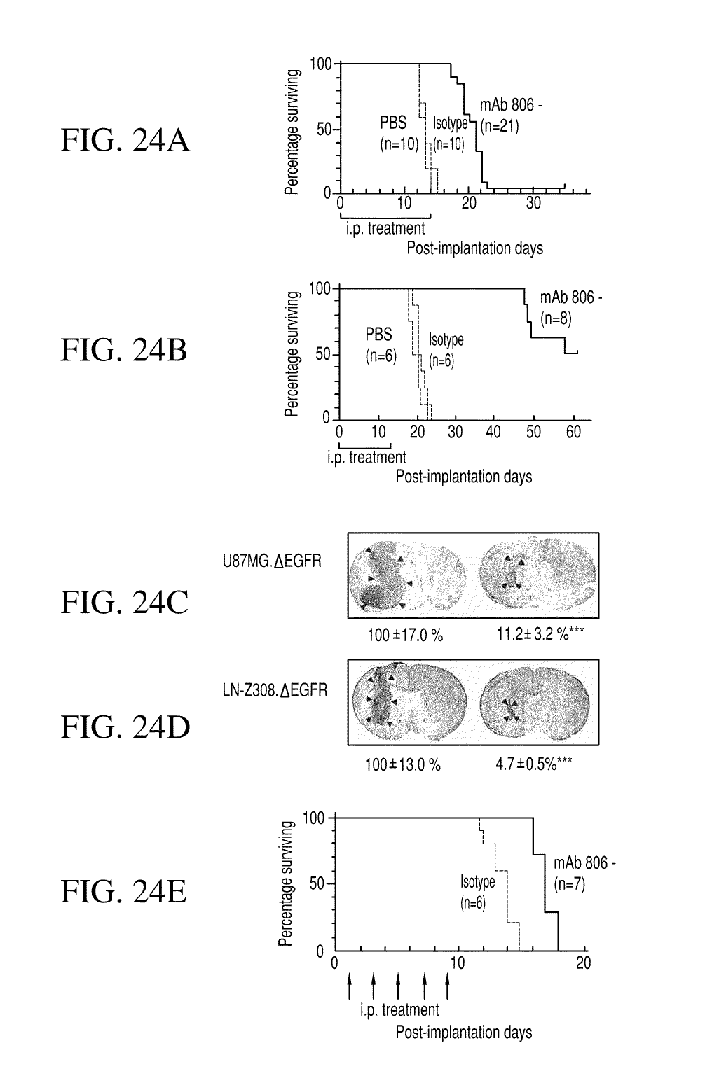

Animals treated with PBS or isotype control IgG had a median survival of 13 days, whereas mice treated with mAb806 had a 61.5% increase in median survival up to 21 days (P<0.001; FIG. 24A).

Treatment of mice 3 days post-implantation, after tumor establishment, also extended the median survival of the mAb806-treated animals by 46.1% (from 13 days to 19 days; P<0.01) compared with that of the control groups (data not shown).

To determine whether these antitumor effects of mAb806 extended beyond U87MG.ΔEGFR xenografts, similar treatments were administered to animals bearing other glioma cell xenografts of LN-Z308.ΔEGFR and A1207.ΔEGFR. The median survival of mAb806-treated mice bearing LN-Z308.ΔEGFR xenografts was extended from 19 days for controls to 58 days (P<0.001; FIG. 24B). Remarkably, four of eight mAb806-treated animals survived beyond 60 days (FIG. 24B). The median survival of animals bearing A1207.ΔEGFR xenografts was also extended from 24 days for controls to 29 days (P<0.01; data not shown).

mAb806 Treatment Inhibits ΔEGFR-overexpressing Brain Tumor Growth

Mice bearing U87MG.ΔEGFR and LN-Z308.ΔEGFR xenografts were euthanized at day 9 and day 15, respectively. Tumor sections were histopathologically analyzed and tumor volumes were determined Consistent with the results observed for animal survival, mAb806 treatment significantly reduced the volumes by about 90% of U87MG.ΔEGFR. (P<0.001; FIG. 24C) and LN-Z308.ΔEGFR by more than 95% (P<0.001; FIG. 24D) xenografts in comparison to that of the control groups. Similar results were obtained for animals bearing A1207.ΔEGFR tumors (65% volume reduction, P<0.01; data not shown).

Intratumoral Treatment with mAb806 Extends Survival of Mice Bearing U87MG.ΔEGFR Brain Tumors

The efficacy of direct intratumoral injection of mAb806 for the treatment of U87MG.ΔEGFR xenografts was also determined Animals were given intratumoral injections of mAb806 or isotype control IgG one day post-implantation. Control animals survived for 15 days, whereas mAb806 treated mice remained alive for 18 days (P<0.01; FIG. 24E). While the intratumoral treatment with mAb806 was somewhat effective, it entailed the difficulties of multiple intracranial injections and increased risk of infection. We therefore focused on systemic treatments for further studies.

mAb806 Treatment Slightly Extends Survival of Mice Bearing U87MG.wtEGFR but not U87MG or U87Mg.Dk Intracranial Xenografts

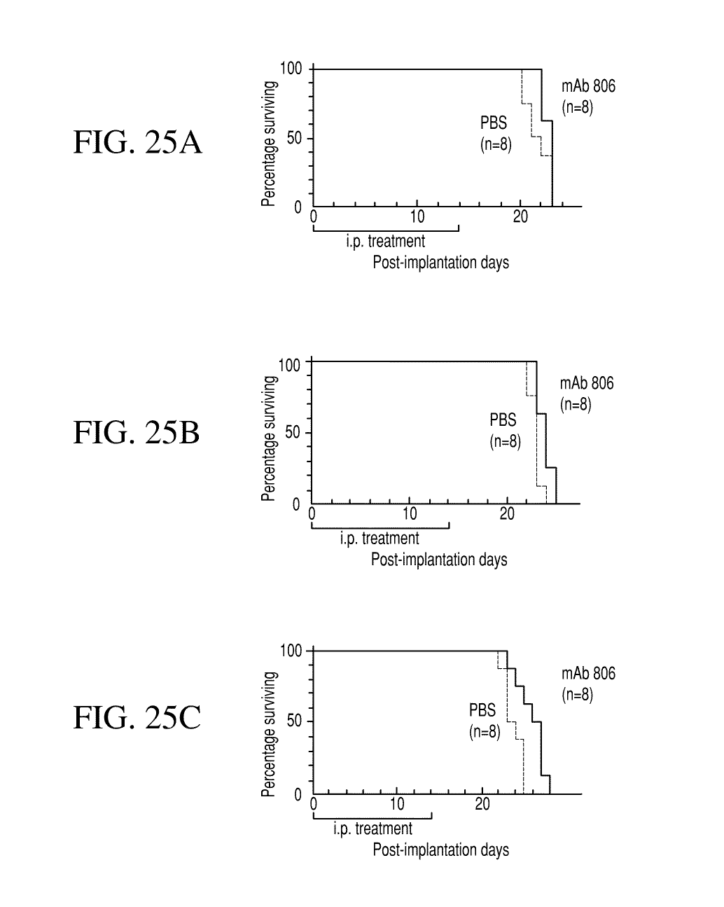

To determine whether the growth inhibition by mAb806 was selective for tumors expressing ΔEGFR, we treated animals bearing U87MG, U87MG.DK (kinase deficient ΔEGFR) and U87MG.wtEGFR brain xenografts. mAb806 treatment did not extend survival of mice implanted with U87MG tumors (FIG. 25A) which expressed a low level of endogenous wild-type EGFR (wtEGFR) (Huang et al. (1997) The enhanced tumorigenic activity of a mutant epidermal growth factor receptor common in human cancers is mediated by threshold levels of constitutive tyrosine phosphorylation and unattenuated signaling. J. Biol. Chem., 272, 2927-2935), or animals bearing U87MG.DK xenografts which overexpressed a kinase-deficient ΔEGFR in addition to a low level of endogenous wtEGFR (FIG. 25B). The mAb806 treatment slightly extended the survival of mice bearing U87MG.wtEGFR tumors (P<0.05, median survival 23 days versus 26 days for the control groups) which overexpressed wtEGFR (FIG. 25C).

mAb806 Reactivity Correlates with In vivo Anti-tumor Efficacy

To understand the differential effect of mAb806 on tumors expressing various levels or different types of EGFR, we determined mAb806 reactivity with various tumor cells by FACS analysis. Stained cells were analyzed with a FACS Calibur using Cell Quest software (Becton-Dickinson PharMingen). For the first antibody, the following mAbs were used: mAb806, anti EGFR mAb clone 528, and clone EGFR. 1. Mouse IgG2a or IgG2b was used as an isotype control.

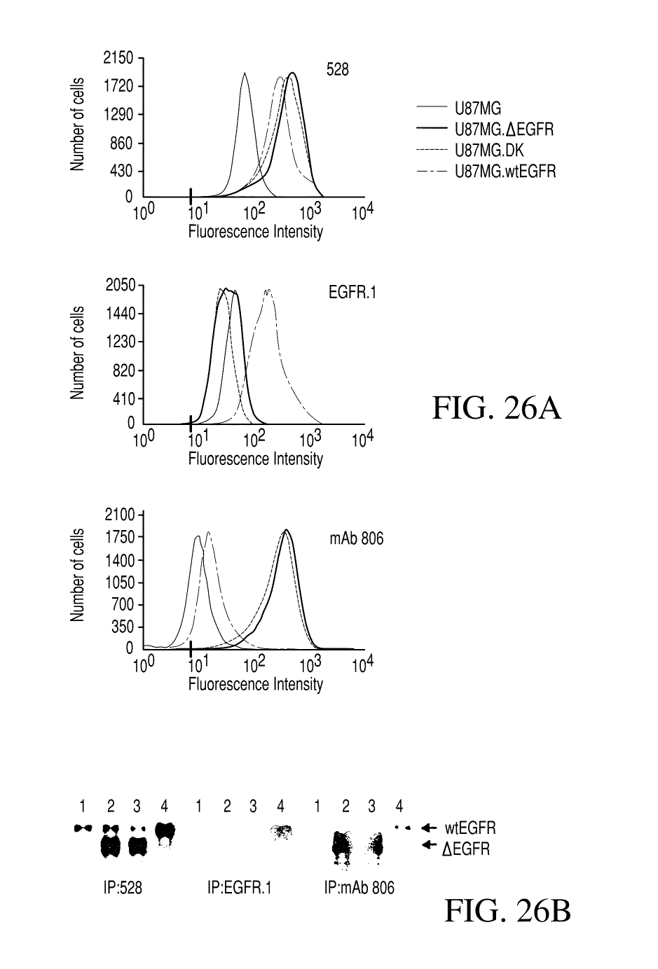

Consistent with previous reports (Nishikawa et al. (1994) A mutant epidermal growth factor receptor common in human glioma confers enhanced tumorigenicity. Proc. Natl. Acad. Sci. U.S.A., 91, 7727-7731), the anti-EGFR mAb528 recognized both ΔEGFR and wtEGFR and demonstrated stronger staining for U87MG.ΔEGFR cells compared with U87MG cells (FIG. 26A, 528).

In contrast, antibody EGFR.1 reacted with wtEGFR but not with ΔEGFR (Nishikawara et al., 1994), because U87MG.ΔEGFR cells were as weakly reactive as U87MG cells (FIG. 26A, panel EGFR.1).

This EGFR.1 antibody reacted with U87MG.wtEGFR more intensively than with U87MG cells, because U87MG.wtEGFR cells overexpressed wtEGFR (FIG. 26A, panel EGFR.1). Although mAb806 reacted intensely with U87MG.ΔEGFR and U87MG.DK cells and not with U87MG cells, it reacted weakly with U87MG.wtEGFR, which indicated that mAb806 is selective for ΔEGFR with a weak cross-activity to overexpressed wtEGFR (FIG. 26A, panel mAb806).

This level of reactivity with U87MG.wtEGFR was quantitatively and qualitatively similar to the extension of survival mediated by the antibody treatment (FIG. 25C).

We further determined mAb806 specificity by immunoprecipitation. EGFRs in various cell lines were immunoprecipitated with antibodies mAb806, anti-EGFR mAb clone 528 (Oncogene Research Products, Boston, Mass.), or clone EGFR.1 (Oncogene Research Products).

Briefly, cells were lysed with lysis buffer containing 50 mM HEPES (pH 7.5), 150 mM NaCl, 10% glycerol, 1% Triton X-100, 2 mM EDTA, 0.1% SDS, 0.5% sodium deoxycholate, 10 mM sodium PPi, 1 mM phenylmethlsulfonyl fluoride, 2 mM Na3 V0₄, 5 μg/ml leupeptin, and 5 μg/ml aprotinin. Antibodies were incubated with cell lysates at 4° C. for 1 h before the addition of protein-A and -G Sepharose Immunoprecipitates were washed twice with lysis buffer and once with HNTG buffer [50 mM HEPES (pH 7.5), 150 mM NaCl, 0.1% Triton X-100, and 10% glycerol], electrophoresed, and transferred to nitrocellulose membranes.

Blots of electrophoretically-separated proteins were probed with the anti-EGFR antibody, C13 (provided by Dr. G. N. Gill, University of California, San Diego, Calif.), used for detection of both wild-type and ΔEGFR on immunoblots (Huang et al., 1997), and proteins were visualized using the ECL chemiluminescent detection system (Amersham Pharmacia Biotech.). Antibodies to Bcl-X (rabbit poly-clonal antibody; Transduction Laboratories, Lexington, Ky.) and phosphotyrosine (4G10, Upstate Biotechnology, Lake Placid, N.Y.) were used for Western blot analysis as described previously (Nagane et al. (1998) Drug resistance of human glioblastoma cells conferred by a tumor-specific mutant epidermal growth factor receptor through modulation of Bcl-XL and caspase-3-like proteases. Proc. Natl. Acad. Sci. U.S.A. 95, 5724-5729).

Consistent with the FACS analysis, antibody 528 recognized wtEGFR and mutant receptors (FIG. 26B-panel IP: 528), whereas antibody EGFR.1 reacted with wtEGFR but not with the mutant species (FIG. 26B, panel IP:EGFR.1). Moreover, the levels of mutant receptors in U87MG.ΔEGFR and U87MG.DK cells are comparable with those of wtEGFR in the U87MG.wtEGFR cells (FIG. 26B, panel IP: 528).

However, antibody mAb806 was able to precipitate only a small amount of the wtEGFR from the U87MG.wtEGFR cell lysates as compared with the larger amount of mutant receptor precipitated from U87MG.ΔEGFR and U87MG.DK cells, and an undetectable amount from the U87MG cells (FIG. 26B, panel IP:mAb806). Collectively, these data suggest that mAb806 recognizes an epitope in ΔEGFR that also exists in a small fraction of wtEGFR only when it is overexpressed on the cell surface (see further discussion of and references to the mAb806 epitope below).

mAb806 Treatment Reduces ΔEGFR Autophosphorylation and Down-regulates Bcl.X_{L} Expression in U87MG.ΔEGFR Brain Tumors

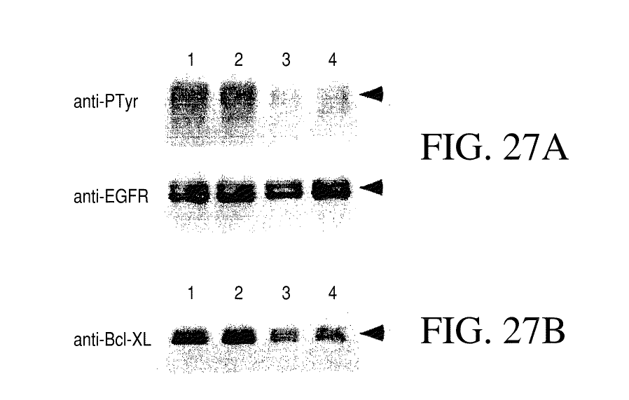

The mechanisms underlying the growth inhibition by mAb806 were next investigated. Since the constitutively active kinase activity and autophosphorylation of the carboxyl terminus of ΔEGFR are essential for its biological functions (Nishikawa et al. (1994) A mutant epidermal growth factor receptor common in human glioma confers enhanced tumorigenicity. Proc. Natl. Acad. Sci. U.S.A. 91, 7727-7731; Huang et al., 1997; Nagane et al. (1996) A common mutant epidermal growth factor receptor confers enhanced tumorigenicity on human glioblastoma cells by increasing proliferation and reducing apoptosis. Cancer Res., 56, 5079-5086; Nagane et al. (2001) Aberrant receptor signaling in human malignant gliomas: mechanisms and therapeutic implications. Cancer Lett. 162 (Suppl.1), S17-S21) ΔEGFR phosphorylation status was determined in tumors from treated and control animals. As shown in FIG. 27A, mAb806 treatment dramatically reduced ΔEGFR autophosphorylation, although receptor levels were only slightly decreased in the mAb806-treated xenografts. We have previously shown that receptor autophosphorylation causes up-regulation of the antiapoptotic gene, Bcl-X_{L}, which plays a key role in reducing apoptosis of ΔEGFR-overexpressing tumors (Nagane et al., 1996; Nagane et al., 2001). Therefore, the effect of mAb806 treatment on Bcl-X_{L} expression was next determined ΔEGFR tumors from mAb806-treated animals did indeed show reduced levels of Bcl-X_{L} (FIG. 27A).

mAb806 Treatment Decreases Growth and Angiogenesis, and Increases Apoptosis in U87MG.ΔEGFR Tumors

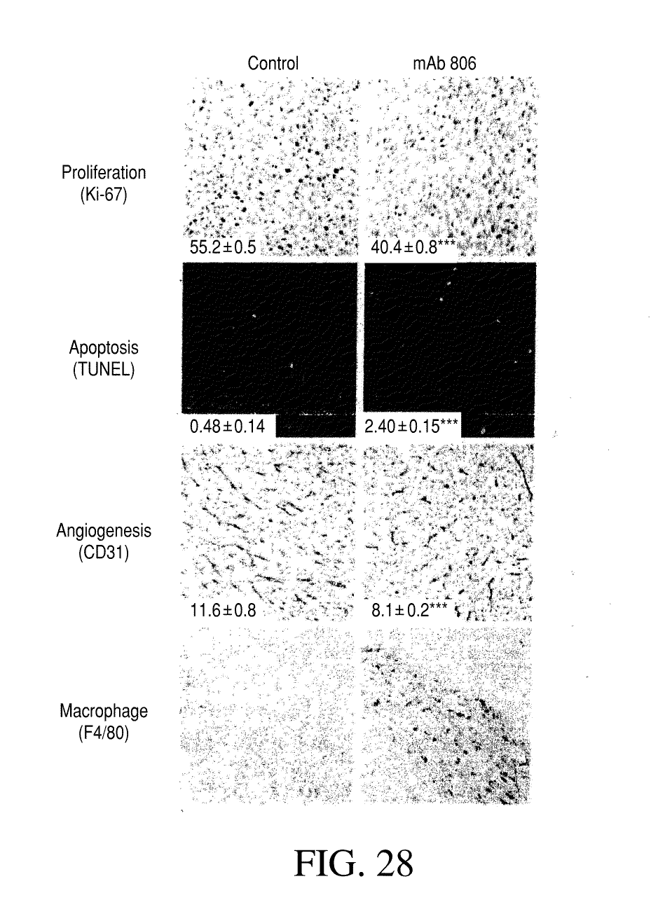

In light of the in vivo suppression caused by mAb806 treatment and its biochemical effects on receptor signaling, we determined the proliferation rate of tumors from control or treated mice. The proliferative index, measured by Ki-67 staining of the mAb806-treated tumors, was significantly lower than that of the control tumors (P<0.001; FIG. 28).

Briefly, to assess angiogenesis in tumors, they were fixed in a solution containing zinc chloride, paraffin embedded, sectioned, and immunostained using a monoclonal rat anti-mouse CD31 antibody (Becton-Dickinson PharMingen; 1:200). Assessment of tumor cell proliferation was performed by Ki-67 immunohistochemistry on formalin-fixed paraffin-embedded tumor tissues. After deparaffinization and rehydration, the tissue sections were incubated with 3% hydrogen peroxide in methanol to quench endogenous peroxidase. The sections were blocked for 30 min with goat serum and incubated overnight with the primary antibody at 4° C. The sections were then washed with PBS and incubated with a biotinylated secondary antibody for 30 min After several washes with PBS, products were visualized using streptavidin horseradish peroxidase with diaminobenzidine as chromogen and hematoxylin as the counterstain. As a measure of proliferation, the Ki-67 labeling index was determined as the ratio of labeled: total nuclei in high-power (3400) fields.

Approximately 2000 nuclei were counted in each case by systematic random sampling. For macrophage and NK cell staining, frozen sections, fixed with buffered 4% paraformaldehyde solution, were immunostained using biotinylated mAbF4/80 (Serotec, Raleigh, N.C.) and polyclonal rabbit anti-asialo GM1 antibody (Dako Chemicals, Richmond, Va.), respectively. Angiogenesis was quantitated as vessel area using computerized analysis. For this purpose, sections were immunostained using anti-CD31 and were analyzed using a computerized image analysis system without counterstain. MVAs were determined by capturing digital images of the sections at 3200 magnification using a CCD color camera as described previously (Mishima et al., 2000). Images were then analyzed using Image Pro Plus version 4.0 software (Media Cybernetics, Silver Spring, Md.) and MVA was determined by measuring the total amount of staining in each section. Four fields were evaluated for each slide. This value was represented as a percentage of the total area in each field. Results were confirmed in each experiment by at least two observers (K. M., H-J. S. H.).

In addition, apoptotic cells in tumor tissue were detected by using the TUNEL method as described previously (Mishima et al., 2000). TUNEL-positive cells were counted at X400. The apoptotic index was calculated as a ratio of apoptotic cell number: total cell number in each field. Analysis of the apoptotic index through TUNEL staining demonstrated a significant increase in the number of apoptotic cells in mAb806 treated tumors as compared with the control tumors (P<0.001; FIG. 28).

The extent of tumor vascularization was also analyzed by immunostaining of tumors from treated and control specimens for CD31. To quantify tumor vascularization, microvascular areas (MVAs) were measured using computerized image analysis. mAb806-treated tumors showed 30% less MVA than did control tumors (P<0.001; FIG. 28).

To understand whether interaction between receptor and antibody may elicit an inflammatory response, we stained tumor sections for the macrophage marker, F4/80, and the NK cell marker, asialo GM1. Macrophages were identified throughout the tumor matrix and especially accumulated around the mAb806-treated-U87MG.ΔEGFR-tumor periphery (FIG. 28). We observed few NK cells infiltrated in and around the tumors and no significant difference between mAb806-treated and isotype-control tumors (data not shown).

### Example 17

### Combination Immunotherapy with mAb806 and mAb528

The experiments set forth herein describe in vivo work designed to determine the efficacy of antibodies in accordance with this invention.

Female nude mice, 4-6 weeks old, were used as the experimental animals. Mice received subscutaneous inoculations of 3×10⁶ tumor cells in each of their flanks.

The animals received either U87MG.D2-7, U87MG.DK, or A431 cells, all of which are described, supra. Therapy began when tumors had grown to a sufficient size.

Mice then received injections of one of (i) phosphate buffered saline, (ii) mAb806 (0.5 mg/injection), (iii) mAb528 (0.5 mg/injection), or (iv) a combination of both mAbs. With respect to “(iv),” different groups of mice received either 0.5 mg/injection of each mAb, or 0.25 mg/injection of each mAb.

The first group of mice examined were those which had received U87MG.D2-7 injections. The treatment protocol began 9 days after inoculation, and continued 3 times per week for 2 weeks (i.e., the animals were inoculated 9, 11, 13, 16, 18 and 20 days after they were injected with the cells). At the start of the treatment protocol, the average tumor diameter was 115 mm³. Each group contained 50 mice, each with two tumors.

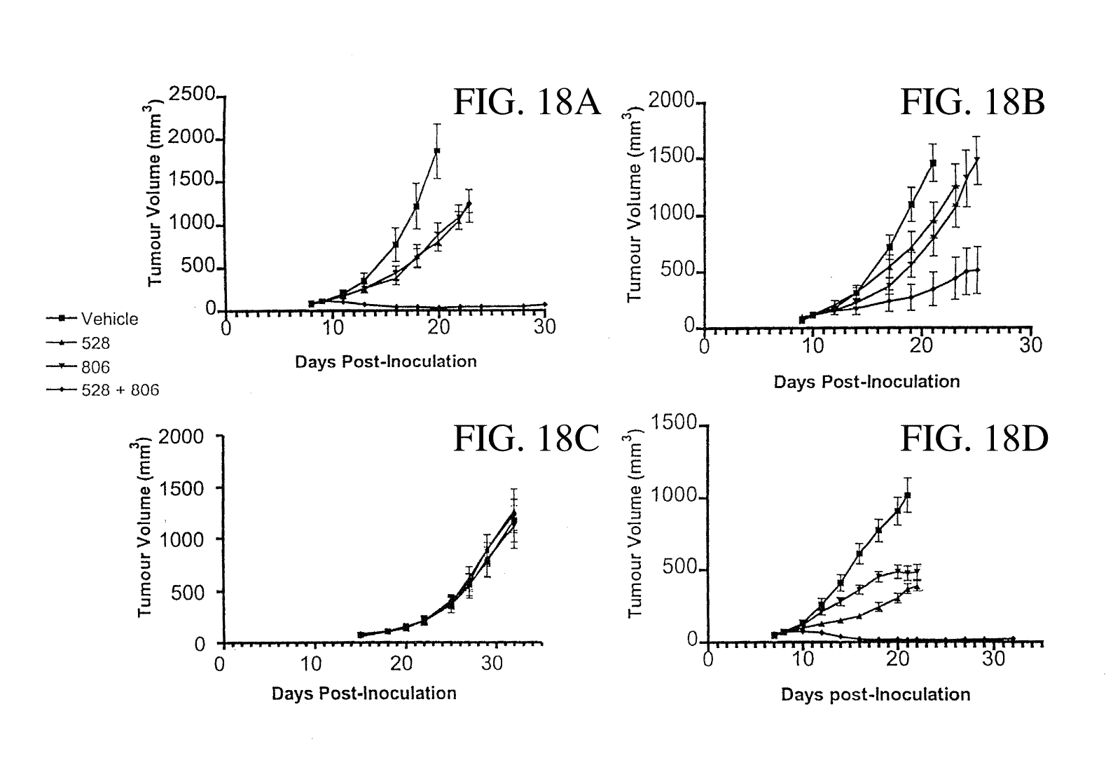

Within the group of mice which received the combination of antibodies (0.5 mg/injection of each), there were three complete regressions. There were no regressions in any of the other groups. FIG. 18A shows the results graphically.

In a second group of mice, the injected materials were the same, except the combination therapy contained 0.25 mg of each antibody per injection. The injections were given 10, 12, 14, 17, 19 and 21 days after inoculation with the cells. At the start of the therapy the average tumor size was 114 mm³. Results are shown in FIG. 18B.

The third group of mice received inoculations of U87MG.DK. Therapeutic injections started 18 days after inoculation with the cells, and continued on days 20, 22, 25, 27 and 29. The average tumor size at the start of the treatment was 107 mm³. FIG. 18C summarizes the results. The therapeutic injections were the same as in the first group.

Finally, the fourth group of mice, which had been inoculated with A431 cells, received injections as in groups I and III, at 8, 10, 12 and 14 days after inoculation. At the start, the average tumor size was 71 mm³. Results are shown in FIG. 18D.

The results indicated that the combination antibody therapy showed a synergistic effect in reducing tumors. See FIG. 18A. A similar effect was seen at a lower dose, as per FIG. 18B, indicating that the effect is not simply due to dosing levels.

The combination therapy did not inhibit the growth of U87MG.DK (FIG. 18C), indicating that antibody immune function was not the cause for the decrease seen in FIGS. 18A and 18B.

It is noted that, as shown in FIG. 18D, the combination therapy also exhibited synergistic efficacy on A431 tumors, with 4 doses leading to a 60% complete response rate. These data suggest that the EGFR molecule recognized by mAb806 is functionally different from that inhibited by 528.

### Example 18

### mAb806 Inhibition of Tumor Xenografts Growth

As discussed herein, and further demonstrated and discussed in this Example, mAb806 has been unexpectedly been found to inhibit the growth of tumor xenographs expressing either de2-7 or amplified EGFR, but not wild-type EGFR

Cell lines and antibodies were prepared as described in Example 1. To determine the specificity of mAb806, its binding to U87MG, U87MG.D2-7, and U87MG.wtEGFR cells was analyzed by FACS. Briefly, cultured parental and transfected U87MG cell lines were analyzed for wild-type and de2-7EGFR expression using the 528, 806, and DH8.3 antibodies. Cells (1 3 10 6) were incubated with 5 μg/ml of the appropriate antibody or an isotype-matched negative control in PBS containing 1% HSA for 30 min at 4° C. After three washes with PBS/1% HSA, cells were incubated an additional 30 min at 4° C. with FTTC-coupled goat anti-mouse antibody (1:100 dilution; Calbiochem, San Diego, Calif.). After three subsequent washes, cells were analyzed on an Epics Elite ESP (Beckman Coulter, Hialeah, Fla.) by observing a minimum of 20,000 events and analyzed using EXPO (version 2) for Windows. An irrelevant IgG2b (mAb 100-310 directed to the human antigen A33) was included as an isotype control for mAb806, and the 528 antibody was included because it recognizes both the de2-7 and wtEGFR.

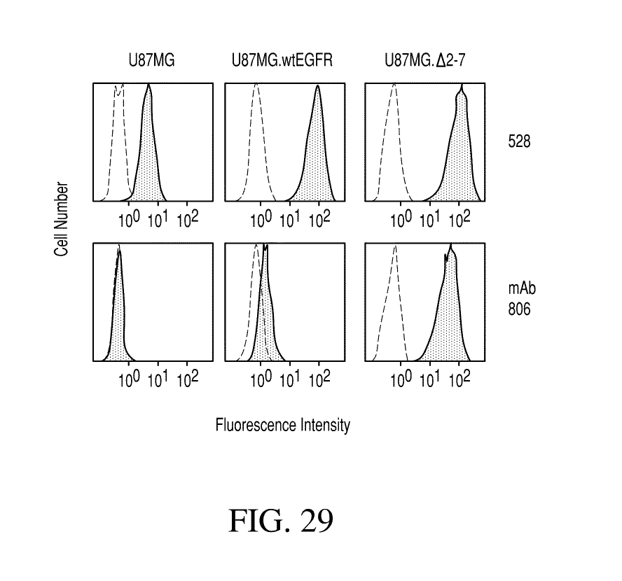

Only the 528 antibody was able to stain the parental U87MG cell line (FIG. 29), consistent with previous reports demonstrating that these cells express the wtEGFR (Nishikawa et al. (1994) A mutant epidermal growth factor receptor common in human glioma confers enhanced tumorigenicity. Proc. Natl. Acad. Sci. U.S.A. 91, 7727-7731). mAb806 had binding levels similar to the control antibody, clearly demonstrating that it is unable to bind the wtEGFR (FIG. 29). Binding of the isotype control antibody to the U87MG.D2-7 and U87MG.wtEGFR cell lines was similar to that observed for the U87MG cells. mAb806 stained U87MG.D2-7 and U87MG.wtEGFR cells, indicating that mAb806 specifically recognized the de2-7 EGFR and a subset of the overexpressed EGFR (FIG. 29). As expected, the 528 antibody stained both the U87MG.D2-7 and U87MG.wtEGFR cell lines (FIG. 29). The intensity of 528 antibody staining on U87MG.wtEGFR cells was much higher than mAb806, suggesting that mAb806 only recognizes a portion of the overexpressed EGFR. The mAb806 reactivity observed with U87MG.wtEGFR cells is similar to that obtained with A431 cells, another cell line that over expresses the wtEGFR.3

A Scatchard analysis was performed using U87MG.D2-7 and A431 cells to determine the relative affinity and binding sites for mAb806 on each cell line. mAb806 had an affinity for the de2-7EGFR receptor of 1.1×10⁹M⁻¹ and recognized an average (three separate experiments) of 2.4×10⁵ binding sites/cell, as noted in Example 4. In contrast, the affinity of mAb806 for the wtEGFR on A431 cells was only 9.5×10⁷M⁻¹, as noted in Example 8. Interestingly, mAb806 recognized 2.3×10⁵ binding sites on the surface of A431, which is some 10-fold lower than the reported number of EGFR found in these cells. To confirm the number of EGFR on the surface of our A431 cells, we performed a Scatchard analysis using ¹²⁵I-labeled 528 antibody. As expected, this antibody bound to approximately 2×10⁶ sites on the surface of A431 cells. Thus, it appears that mAb806 only binds a portion of the EGFR receptors on the surface of A431 cells. Importantly, ¹²⁵I-labeled mAb806 did not bind to the parental U87MG cells at all, even when the number of cells was increased to 1×10⁷.

mAb806 reactivity was further characterized in the various cell lines by immunoprecipitation after ³⁵S-labeling using mAb806, sc-03 (a commercial polyclonal antibody specific for the COOH-terminal domain of the EGFR) and a IgG2b isotype control. Briefly, cells were labeled for 16 h with 100 mCi/ml of Tran ³⁵S-Label (ICN Biomedicals, Irvine, Calif.) in DMEM without methionine/cysteine supplemented with 5% dialyzed FCS. After washing with PBS, cells were placed in lysis buffer (1% Triton X-100, 30 mM HEPES, 150 mM NaCl, 500 μM 4-(2-aminoethyl) benzenesulfonylfluoride (AEBSF), 150 nM aprotinin, 1 μM E-64 protease inhibitor, 0.5 mM EDTA, and 1 μM leupeptin, pH 7.4) for 1 h at 4° C. Lysates were clarified by centrifugation for 10 min at 12,000 g and then incubated with 5 μg of appropriate antibody for 30 min at 4° C. before the addition of Protein A-Sepharose Immunoprecipitates were washed three times with lysis buffer, mixed with SDS sample buffer, separated by gel electrophoresis using a 4-20% Tris/glycine gel that was then dried, and exposed to X-ray film.

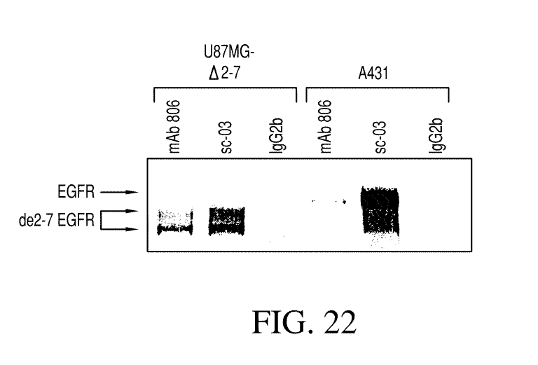

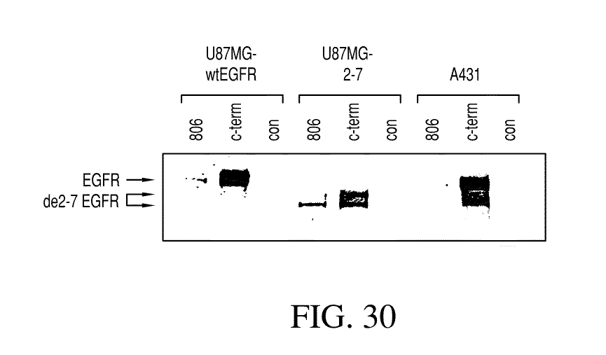

The sc-03 antibody immunoprecipitated three bands from U87MG.Δ2-7 cells; a doublet corresponding to the 2 de2-7 EGFR bands observed in these cells and a higher molecular weight band corresponding to the wtEGFR (FIGS. 22 and 30). In contrast, while mAb806 immunoprecipitated the two de2-7 EGFR bands, the wtEGFR was completely absent. The pattern seen in U87MG.wtEGFR and A431 cells was essentially identical. The sc-03 antibody immunoprecipitated a single band corresponding to the wtEGFR from A431 cells (FIGS. 22 and 30). mAb806 also immunoprecipitated a single band corresponding to the wtEGFR from both U87MG.wtEGFR and A431 cells (FIGS. 22 and 30). Consistent with the FACS and Scatchard data, the amount of EGFR immunoprecipitated by mAb806 was substantially less than the total EGFR present on the cell surface. Given that mAb806 and the sc-03 immunoprecipitated similar amounts of the de2-7 EGFR, this result supports the notion that the mAb806 antibody only recognizes a portion of the EGFR in cells overexpressing the receptor. Comparisons between mAb806 and the 528 antibody showed an identical pattern of reactivity (data not shown). An irrelevant IgG2b (an isotype control for mAb806) did not immunoprecipitate EGFR from either cell line (FIGS. 22 and 30). Using identical conditions, mAb806 did not immunoprecipitate the EGFR from the parental U87MG cells (data not shown).

mAb806 was also examined for efficacy against U87MG and U87MG.Δ2-7 tumors in a preventative xenograft model. Antibody or vehicle was administered i.p. the day before tumor inoculation and was given three times per week for 2 weeks. At a dose of 1 mg/injection, mAb806 had no effect on the growth of parental U87MG xenografts that express the wtEGFR (FIG. 9A). In contrast, mAb806 inhibited significantly the growth of U87MG.Δ2-7 xenografts in a dose-dependent manner (FIG. 9B). Twenty days after tumor inoculation, when control animals were sacrificed, the mean tumor volume was 1600±180 mm³ for the control group, a significantly smaller 500±95 mm³ for the 0.1 mg/injection group (P<0.0001) and 200±42 mm³ for the 1 mg/injection group (P<0.0001). Treatment groups were sacrificed at day 24, at which time the mean tumor volumes were 1300±240 mm³ for the 0.1 mg treated group and 500±100 mm³ for the 1 mg group (P<0.005).

Given the efficacy of mAb806 in the preventative xenograft model, its ability to inhibit the growth of established tumor xenografts was examined. Antibody treatment was as described in the preventative model, except that it commenced when tumors had reached a mean tumor volume of 65 mm³ (10 days after implantation) for the U87MG.Δ2-7 xenografts and 84 mm³ (19 days after implantation) for the parental U87MG xenografts (see Example 10). Once again, mAb806 had no effect on the growth of parental U87MG xenografts, even at a dose of 1 mg/injection (FIG. 10A). In contrast, mAb806 significantly inhibited the growth of U87MG.Δ2-7 xenografts in a dose-dependent manner (FIG. 10B). At day 17, one day before control animals were sacrificed, the mean tumor volume was 900±200 mm³ for the control group, 400±60 mm³ for the 0.1 mg/injection group (P<0.01), and 220±60 mm³ for the 1 mg/injection group (P<0.002). Treatment of U87MG.Δ2-7 xenografts with an IgG2b isotype control had no effect on tumor growth (data not shown).

To examine whether the growth inhibition observed with mAb806 was restricted to cells expressing de2-7 EGFR, its efficacy against the U87MG.wtEGFR xenografts was also examined in an established model. These cells serve as a model for tumors containing amplification of the EGFR gene without de2-7 EGFR expression. mAb806 treatment commenced when tumors had reached a mean tumor volume of 73 mm³ (22 days after implantation). mAb806 significantly inhibited the growth of established U87MG.wtEGFR xenografts when compared with control tumors treated with vehicle (FIG. 10C). On the day control animals were sacrificed, the mean tumor volume was 1000±300 mm³ for the control group and 500±80 mm³ for the group treated with 1 mg/injection (P<0.04).

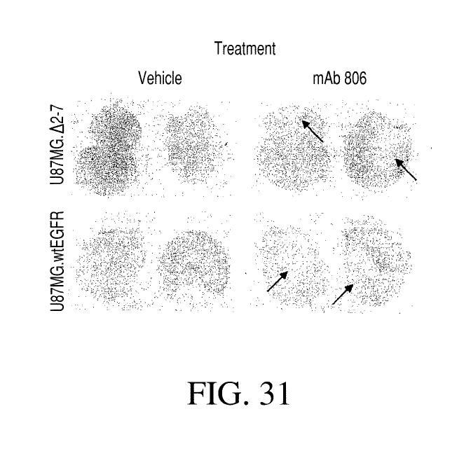

To evaluate potential histological differences between mAb806-treated and control U87MG.Δ2-7 and U87MG.wtEGFR xenografts, formalin-fixed, paraffin-embedded sections were stained with H&E (FIG. 31). Areas of necrosis were seen in sections from mAb806-treated U87MG.Δ2-7 (mAb806-treated xenografts were collected 24 days after tumor inoculation and vehicle treated xenografts at 18 days), and U87MG.wtEGFR xenografts (mAb806 xenografts were collected 42 days after tumor inoculation and vehicle treated xenografts at 37 days; FIG. 31). This result was consistently observed in a number of tumor xenografts (n=4 for each cell line). However, sections from U87MG.Δ2-7 and U87MG.wtEGFR xenografts treated with vehicle (n=5) did not display the same areas of necrosis seen after mAb806 treatment (FIG. 31). Vehicle and mAb806-treated xenografts removed at identical times also showed these differences in tumor necrosis (data not shown). Thus, the increase in necrosis observed was not caused by the longer growth periods used for the mAb806-treated xenografts. Furthermore, sections from mAb806-treated U87MG xenografts were also stained with H&E and did not reveal any areas of necrosis (data not shown), further supporting the hypothesis that mAb806 binding induces decreased cell viability, resulting in increased necrosis within tumor xenografts.

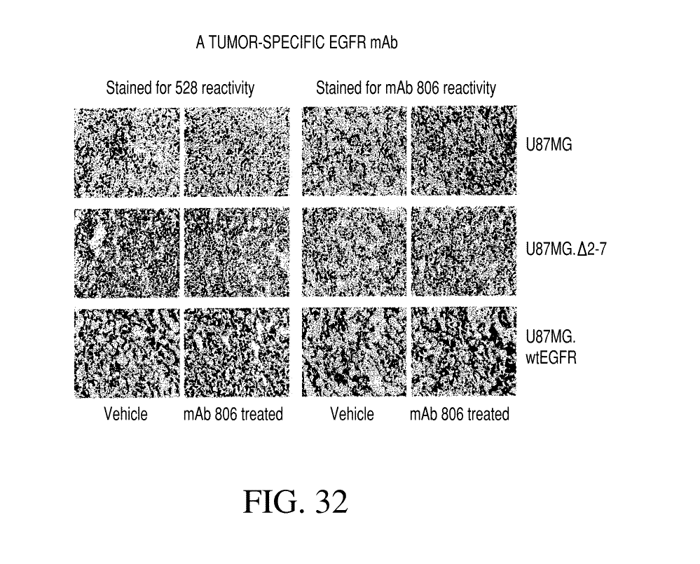

An immunohistochemical analysis of U87MG, U87MG.Δ2-7, and U87MG.wtEGFR xenograft sections was performed to determine the levels of de2-7 and wtEGFR expression after mAb806 treatment (FIG. 32). As expected, the 528 antibody stained all xenografts sections with no obvious decrease in intensity between treated and control tumors (FIG. 32). Staining of U87MG sections was undetectable with the mAb806; however, positive staining of U87MG.Δ2-7 and U87MG.wtEGFR xenograft sections was observed (FIG. 32). There was no difference in mAb806 staining intensity between control and treated U87MG.Δ2-7 and U87MG.wtEGFR xenografts, suggesting that antibody treatment does not lead to the selection of clonal variants lacking mAb806 reactivity.

To demonstrate that the antitumor effects of mAb806 were not restricted to U87MG cells, the antibody was administrated to mice containing A431 xenografts. These cells contain an amplified EGFR gene and express approximately 2×10⁶ receptors/cells. We have previously shown that mAb806 binds ˜10% of these EGFRs and targets A431 xenografts (Garcia et al. (1993) Expression of mutated epidermal growth factor receptor by non-small cell along carcinomas. Cancer Res. 53, 3217-3220). mAb806 significantly inhibited the growth of A431 xenografts when examined in the preventative xenograft model described previously (FIG. 11A). At day 13, when control animals were sacrificed, the mean tumor volume was 1400±150 mm³ in the vehicle-treated group and 260±60 mm³ for the 1 mg/injection treatment group (P<0.0001). In a separate experiment, a dose of 0.1 mg of mAb also inhibited significantly (P<0.05) the growth of A431 xenografts in a preventative model (data not shown) (see Example 10).

Given the efficacy of mAb806 in the preventative A431 xenograft model, its ability to inhibit the growth of established tumor xenografts was examined. Antibody treatment was as described in the preventative model, except it was not started until tumors had reached a mean tumor volume of 200±20 mm³ mAb806 significantly inhibited the growth of established A431 xenografts (FIG. 11B). At day 13, the day control animals were sacrificed, the mean tumor volume was 1100±100 mm³ for the control group and 450±70 mm³ for the 1 mg/injection group (P<0.0001).

### Example 19

### Construction, Expression and Analysis of Chimeric 806 Antibody

Chimeric antibodies are a class of molecules in which heavy and light chain variable regions of for instance, a mouse, rat or other species are joined onto human heavy and light chain regions, Chimeric antibodies are produced recombinantly. One advantage of chimeric antibodies is that they can reduce xenoantigenic effects, the inherent immunogenicity of non-human antibodies (for instance, mouse, rat or other species). In addition, recombinantly prepared chimeric antibodies can often be produced in large quantities, particularly when utilizing high level expression vectors.

For high level production, the most widely used mammalian expression system is one which utilizes the gene amplification procedure offered by dehydrofolate reductase deficient (“dhfr-”) Chinese hamster ovary cells. The system is well known to the skilled artisan. The system is based upon the dehydrofolate reductase “dhfr” gene, which encodes the DHFR enzyme, which catalyzes conversion of dehydrofolate to tetrahydrofolate. In order to achieve high production, dhfr-CHO cells are transfected with an expression vector containing a functional DHFR gene, together with a gene that encodes a desired protein. In this case, the desired protein is recombinant antibody heavy chain and/or light chain.

By increasing the amount of the competitive DHFR inhibitor methotrexate (MTX), the recombinant cells develop resistance by amplifying the dhfr gene. In standard cases, the amplification unit employed is much larger than the size of the dhfr gene, and as a result the antibody heavy chain is co-amplified.

When large scale production of the protein, such as the antibody chain, is desired, both the expression level, and the stability of the cells being employed, are critical. In long term culture, recombinant CHO cell populations lose homogeneity with respect to their specific antibody productivity during amplification, even though they derive from a single, parental clone.

Bicistronic expression vectors were prepaid for use in recombinant expression of the chimeric antibodies. These bicistronic expression vectors, employ an “internal ribosomal entry site” or “IRES.” In these constructs for production of chimeric anti-EGFR, the immunoglobulin chains and selectable markers cDNAs are linked via an IRES. IRES are cis-acting elements that recruit the small ribosomal subunits to an internal initiator codon in the mRNA with the help of cellular trans-acting factors. IRES facilitate the expression of two or more proteins from a polycistronic transcription unit in eukaryotic cells. The use of bicistronic expression vectors in which the selectable marker gene is translated in a cap dependent manner, and the gene of interest in an IRES dependent manner, has been applied to a variety of experimental methods. IRES elements have been successfully incorporated into vectors for cellular transformation, production of transgenic animals, recombinant protein production, gene therapy, gene trapping, and gene targeting.

Synopsis of Chimeric Antibody 806 (ch806) Construction

The chimeric 806 antibody was generated by cloning the VH and VL chains of the 806 antibody from the parental murine hybridoma using standard molecular biology techniques. The VH and VL chains were then cloned into the pREN mammalian expression vectors, the construction of which are set forth in SEQ ID NO:7 and SEQ ID NO:8, and transfected into CHO (DHFR−/−ve) cells for amplification and expression. Briefly, following trypsinization 4×10⁶ CHO cells were co-transferred with 10 μg of each of the LC and HC expression vectors using electroporation under standard conditions. Following a 10 min rest period at room temperature, the cells were added to 15 ml medium (10% fetal calf serum, hypoxanthine/thymidine supplement with additives) and transferred to 15×10 cm cell culture petri dishes. The plates were then placed into the incubator under normal conditions for 2 days.

At this point, the addition of gentamycin, 5 nM methotrexate, the replacement of fetal calf serum with dialyzed fetal calf serum and the removal of hypoxanthine/thymidine, initiated the selection for clones that were successfully transfected with both the LC and HC from the medium. At day 17 following transfection, individual clones growing under selection were picked and screened for expression of the chimeric 806 antibody. An ELISA was utilized for screening and consisted of coating an ELISA plate with denatured soluble EGF receptor (denatured EGFR is known to allow 806 binding). This assay allows for the screening of production levels by individual clones and also for the functionality of the antibody being screened. All clones were shown to be producing functional ch806 and the best producer was taken and expanded for amplification. To amplify the level of ch806 being produced, the highest producing clone was subjected to reselection under a higher methotrexate concentration (100 nM vs. 5 nM). This was undertaken using the aforementioned procedures.

Clones growing at 100 nM MTX were then passed onto the Biological Production Facility, Ludwig Institute, Melbourne, Australia for measurement of production levels, weaning off serum, cell banking. The cell line has been shown to stably produce ˜10 mg/litre in roller bottles.

The nucleic acid sequence of the pREN ch806 LC neo vector is provided in SEQ ID NO:7. The nucleic acid sequence of the pREN ch806 HC DHFR vector is provided in SEQ ID NO:8.

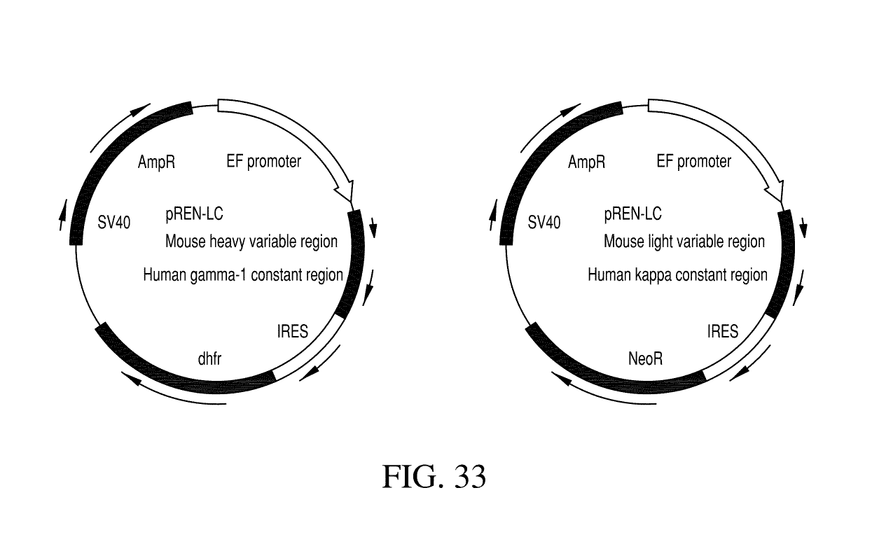

FIG. 33 depicts the vectors pREN-HC and pREN-LC, which employ an IRES. The pREN bicistronic vector system is described and disclosed in U.S. Patent Application No. 60/355,838 filed Feb. 13, 2002, which is incorporated herein by reference in its entirety.

ch806 was assessed by FACS analysis to demonstrate that the chimeric 806 displays identical binding specificity to that of the murine parental antibody. Analysis was performed using wild-type cells (U87MG parental cells), cells overexpressing the EGF receptor (A431 cells and UA87.wtEGFR cells) and UA87.42-7 cells (data not shown). Similar binding specificity of mAb806 and ch806 was obtained using cells overexpressing EGFR and cells expressing the de2-7 EGFR. No binding was observed in wild-type cells. Scatchard analysis revealed a binding affinity for radiolabeled ch806 of 6.4×10⁹ M⁻¹ using U87MGde2-7 cells (data not shown).

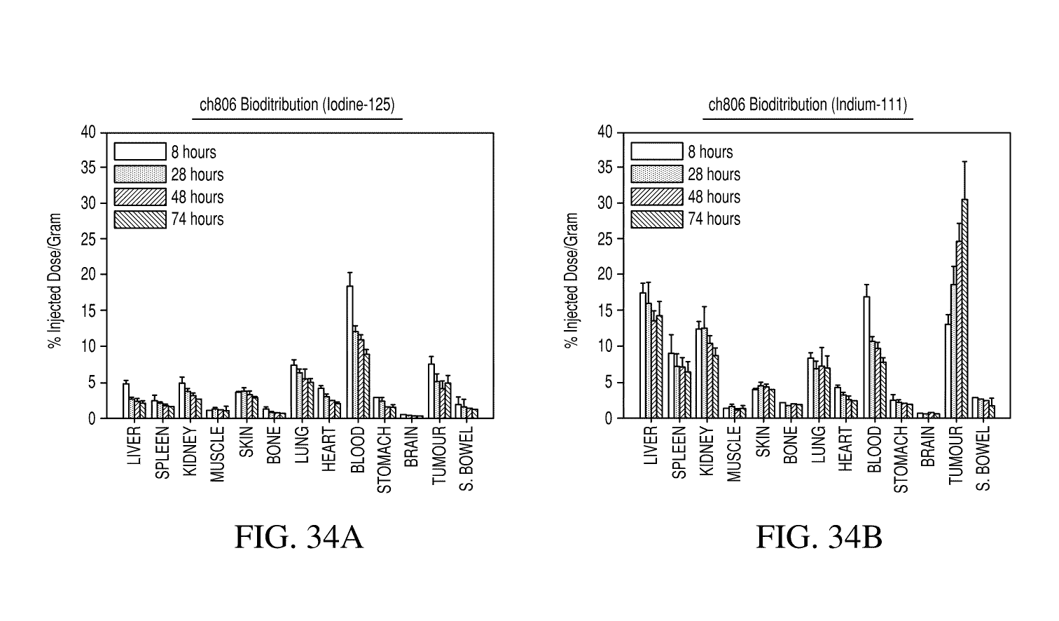

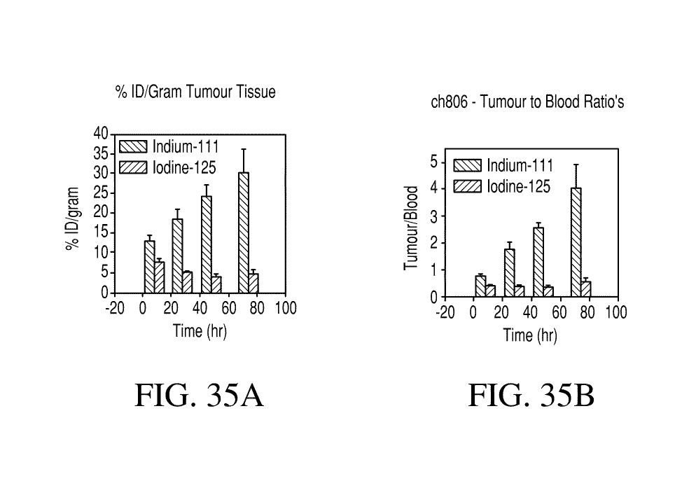

Biodistribution analysis of the ch806 antibody was performed in BALB/c nude mice bearing U87MG-de2-7 xenograft tumors, and the results are shown in FIG. 34. Mice were injected with 5 μg of radiolabelled antibody and were sacrificed in groups of four per time point at 8, 24, 48 and 74 hours. Organs were collected, weighed and radioactivity measured in a gamma counter. ¹²⁵I-labelled ch806 displays reduced targeting to the tumor compared to ¹¹¹In-labelled ch806, which has high tumor uptake and cumulative tumor retention over the 74 hour time period. At 74 hours, the ¹¹¹In-labelled antibody displays approximately 30% ID/gram tissue and a tumor to blood ratio of 4.0 (FIG. 35). The ¹¹¹In-labelled ch806 shows some nonspecific retention in the liver, spleen and kidneys. This is common for the use of this isotope and decreases with time, which supports that this binding is non-specific to ch806 and due to ¹¹¹In binding.

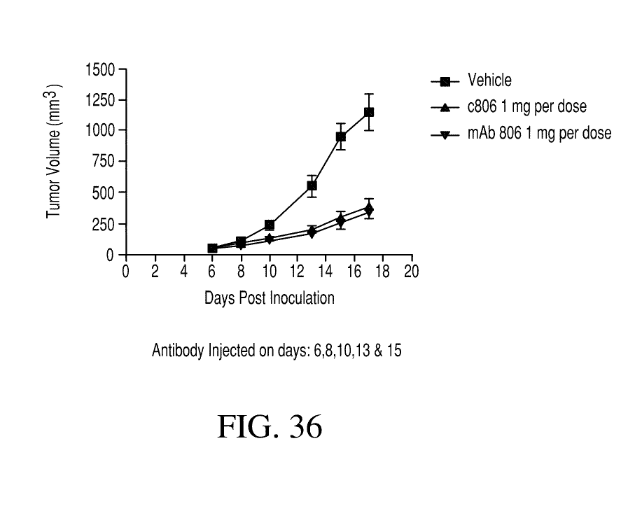

Chimeric antibody ch806 was assessed for therapeutic efficacy in an established tumor model. 3×10⁶ U87MG.Δ2-7 cells in 100 μl of PBS were inoculated s.c. into both flanks of 4-6 week old female nude mice (Animal Research Center, Western Australia, Australia). The mAb806 was included as a positive control. The results are depicted in FIG. 36. Treatment was started when tumors had reached a mean volume of 50 mm³ and consisted of 1 mg of ch806 or mAb806 given i.p. for a total of 5 injections on the days indicated. Tumor volume in mm³ was determined using the formula (Iength×width²)/2, where length was the longest axis and width the measurement at right angles to the length. Data was expressed as mean tumor volume+/−S.E. for each treatment group. The ch806 and mAb806 displayed nearly identical anti-tumor activity against U87MG.Δ2-7 xenografts.

### Analysis of Ch806 Immune Effector Function

### Materials and Methods

### Antibodies and Cell Lines

Murine anti-de2-7 EGFR monoclonal mAb806, chimeric antibody ch806 (IgG₁) and control isotype matched chimeric anti-G250 monoclonal antibody cG250 were prepared by the Biological Production Facility, Ludwig Institute for Cancer Research, Melbourne, Australia. Both complement-dependant cytotoxicity (CDC) and antibody-dependent cellular-cytotoxicity (ADCC) assays utilized U87MG.de2-7 and A431 cells as target cells. The previously described U87MG.de2-7 cell line is a human astrocytoma cell line infected with a retrovirus containing the de2-7EGFR (Nishikawa et al. (1994) Proc. Natl. Acad. Sci. U.S.A. 91, 7727-31). Human squamous carcinoma A431 cells were purchased from the American Type Culture Collection (Manassas, Va.). All cell lines were cultured in DMEM/F-12 with Glutamax (Life Technologies, Melbourne, Australia) supplemented with 10% heat-inactivated FCS(CSL, Melbourne, Australia), 100 units/ml penicillin and 100 μg/ml streptomycin. To maintain selection for retrovirally transfected U87MG.de2-7 cells, 400 μg/ml G418 was included in the media.

### Preparation of Human Peripheral Blood Mononuclear Cells (Pbmc) Effector Cells

PBMCs were isolated from healthy volunteer donor blood. Heparinized whole blood was fractionated by density centrifugation on Ficoll-Hypaque (ICN Biomedical Inc., Ohio, USA). PBMC fractions was collected and washed three times with RPMI⁺1640 supplemented with 100 U/ml penicillin and 100 μg/ml streptomycin, 2 mM L-glutamine, containing 5% heat-inactivated FCS.

### Preparation of Target Cells

CDC and ADCC assays were performed by a modification of a previously published method (Nelson, D. L. et al. (1991) In: J. E. Colignan, A. M. Kruisbeek, D. D. Margulies, E. M. Shevach, and W. Strober (eds.), Current Protocols in Immunology, pp. 7.27.1. New York: Greene Publishing Wiley Interscience). Briefly, 5×10⁶ target U87MG.de2-7 and A431 cells were labeled with 50 μCi⁵¹Cr (Geneworks, Adelaide, Australia) per 1×10⁶ cells and incubated for 2 hr at 37° C. The cells were then washed three time with PBS (0.05M, pH 7.4) and a fourth wash with culture medium. Aliquots (1×10⁴ cells/50 μl) of the labeled cells were added to each well of 96-well microtitre plates (NUNC, Roskilde, Denmark).

### CDC Assay

To 50 μl labeled target cells, 50 μl ch806 or isotype control antibody cG250 were added in triplicate over the concentration range 0.00315-10 μg/ml, and incubated on ice 5 min. Fifty μl of freshly prepared healthy donor complement (serum) was then added to yield a 1:3 final dilution of the serum. The microtitre plates were incubated for 4 hr at 37° C. Following centrifugation, the released ⁵¹Cr in the supernatant was counted (Cobra II automated Gamma Counter, Can berra Packard, Melbourne, Australia). Percentage specific lysis was calculated from the experimental ⁵¹Cr release, the total (50 μl target cells+100 μl 10% Tween 20) and spontaneous (50 μl target cells+100 μl medium) release.

### ADCC Assay

ch806-mediated ADCC effected by healthy donor PBMCs was measured by two 4-hr ⁵¹1Cr release assays. In the first assay, labelled target cells were plated with the effector cells in 96-well “U” bottom microplates (NUNC, Roskilde, Denmark) at effector/target (E:T) cell ratios of 50:1. For ADCC activity measurements, 0.00315-10 μg/ml (final concentration) test and control antibodies were added in triplicate to each well. In the second ADCC assay, the ADCC activity of ch806 was compared with the parental murine mAb806 over a range of Effector: Target cell ratios with the test antibody concentration constant at 1 μg/ml. In both assays, micotitre plates were incubated at 37° C. for 4 hours, then 50 μl supernatant was harvested from each well and released ⁵¹Cr was determined by gamma counting (Cobra II automated Gamma Counter, Can berra Packard, Melbourne, Australia). Controls included in the assays corrected for spontaneous release (medium alone) and total release (10% Tween20/PBS). Appropriate controls with the same subclass antibody were run in parallel.

The percentage cell lysis (cytotoxicity) was calculated according to the formula:

$$Percentage Cytotoxicity = \frac{Sample Counts - Spontaneous Release}{Total Release - Spontaneous Release} \times 100$$

The percent (%) cytotoxicity was plotted versus concentration of antibody (μg/ml).

### Results

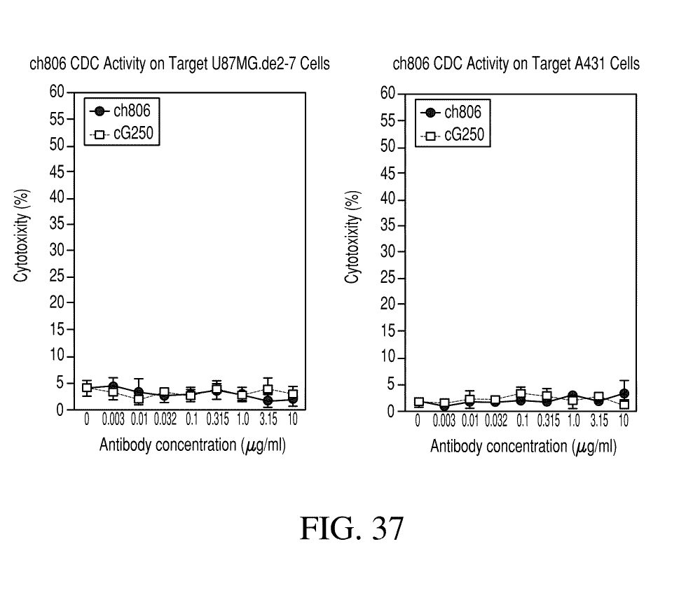

The results of the CDC analyses are presented in FIG. 37. Minimal CDC activity was observed in the presence of up to 10 μg/ml ch806 with CDC comparable to that observed with isotype control cG250.

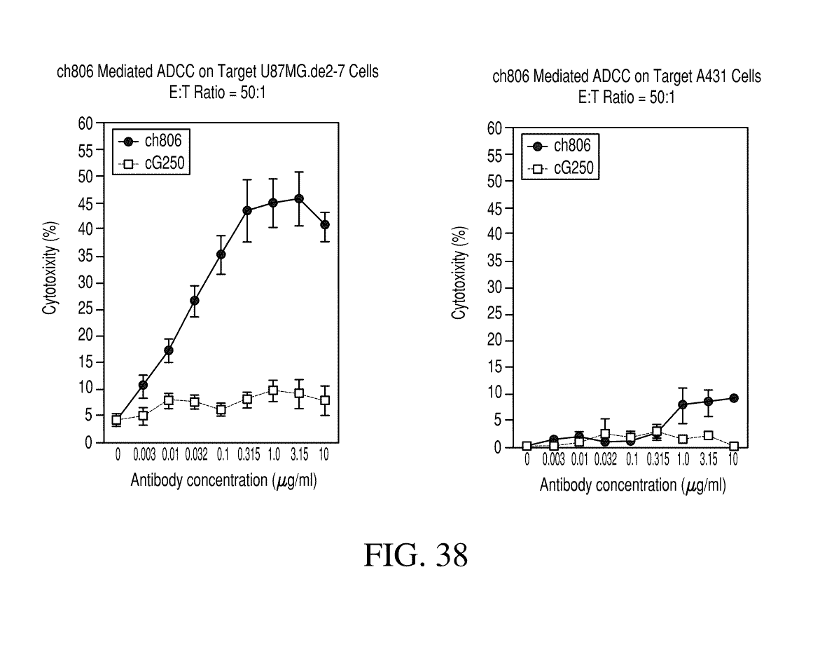

ch806 mediated ADCC on target U87MG.de2-7 and A431 cells at E:T ratio of 50:1 is presented in FIG. 38. Effective ch806 specific cytotoxicity was displayed against target U87MG.de2-7 cells, but minimal ADCC was mediated by ch806 on A431 cells. The levels of cytotoxicity achieved reflect the number of ch806 binding sites on the two cell populations. Target U87MG.de2-7 cells express ˜1×10⁶ de2-7EGFR which are specifically recognized by ch806, while only a subset of the 1×10⁶ wild-type EGFR molecules expressed on A431 cells are recognized by ch806 (see above Examples).

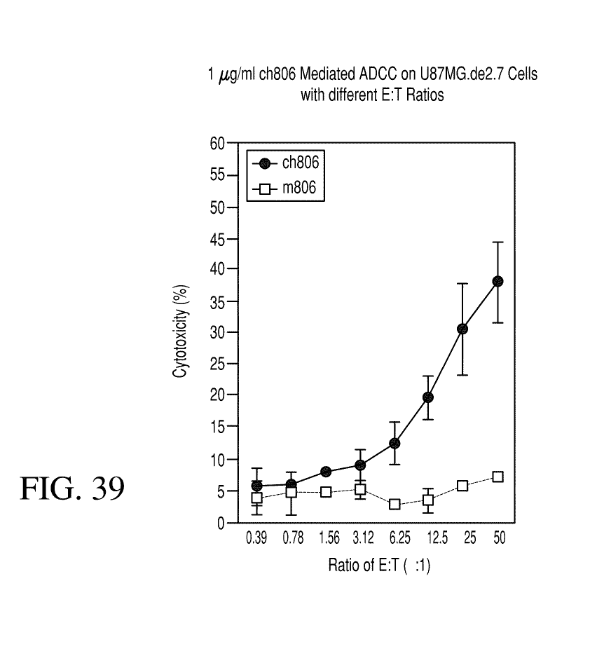

Further ADCC analyses were performed to compare the ADCC mediated by 1 μg/ml ch806 on target U87MG.de2-7 cells with that effected by 1 μg/ml parental murine mAb806. Results are presented in FIG. 39. Chimerization of mAb806 has effected marked improvement of the ADCC achieved by the parental murine mAb with greater than 30% cytotoxicity effected at E:T ratios 25:1 and 50:1.

The lack of parental murine mAb806 immune effector function has been markedly improved upon chimerization. ch806 mediates good ADCC, but minimal CDC activity.

### Example 20

### Generation of Anti-Idiotype Antibodies To Chimeric Antibody ch806

To assist the clinical evaluation of mAb806 or ch806, laboratory assays are required to monitor the serum pharmacokinetics of the antibodies and quantitate any immune responses to the mouse-human chimeric antibody. Mouse monoclonal anti-idiotypic antibodies (anti-ids) were generated and characterized for suitability as ELISA reagents for measuring ch806 in patient sera samples and use as positive controls in human anti-chimeric antibody immune response analyses. These anti-idiotype antibodies may also be useful as therapeutic or prophylactic vaccines, generating a natural anti-EGFR antibody response in patients.

Methods for generating anti-idiotype antibodies are well known in the art (Chatterjee et al., 2001; Uemura et al., 1994; Steffens et al., 1997; Safa and Foon, 2001; Brown and Ling, 1988).

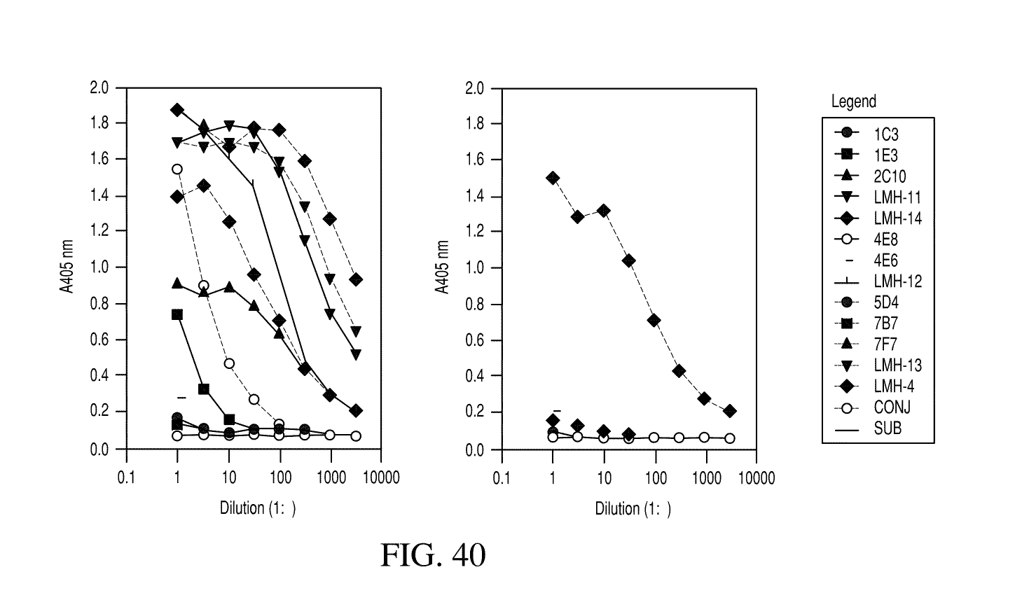

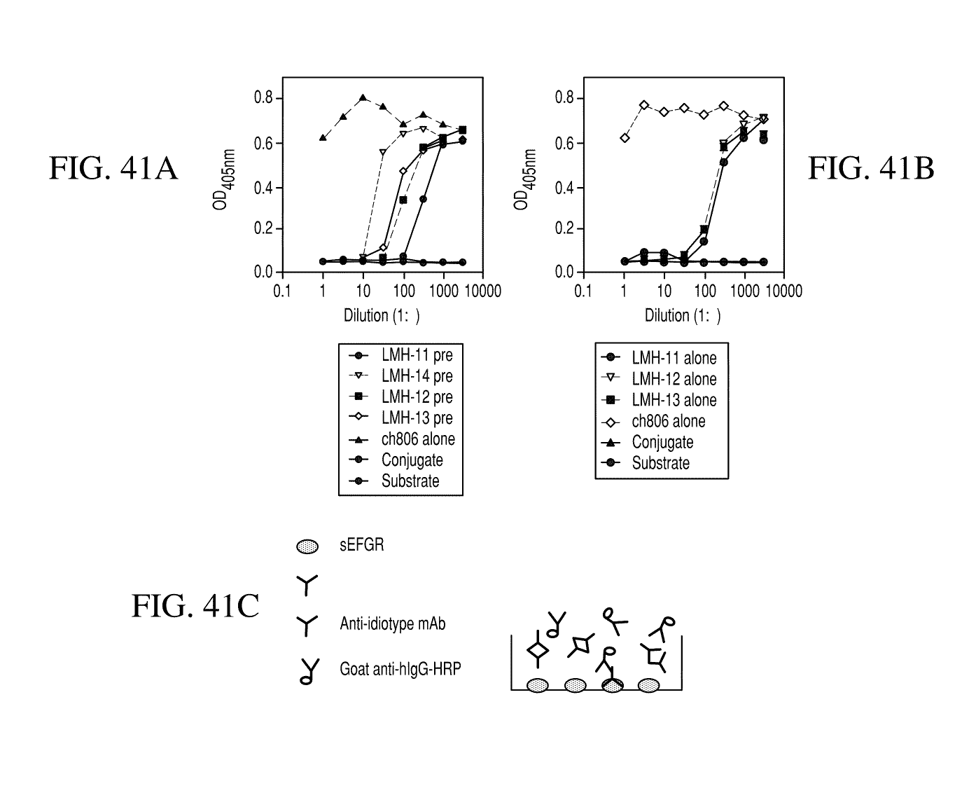

Briefly, mouse monoclonal anti-idiotypic antibodies (anti-ids) were generated as follows. Splenocytes from mice immunized with ch806 were fused with SP2/0-AG14 plasmacytoma cells and antibody producing hybridomas were selected through ELISA for specific binding to ch806 and competitive binding for antigen (FIG. 40). Twenty-five hybridomas were initially selected and four, designated LMH-11, -12, -13, and -14, secreted antibodies that demonstrated specific binding to ch806, mAb806 and were able to neutralize ch806 or mAb806 antigen binding activity (FIG. 41). The recognition of the ch806/mAb806 idiotope or CDR region was demonstrated by lack of cross-reactivity with purified polyclonal human IgG.

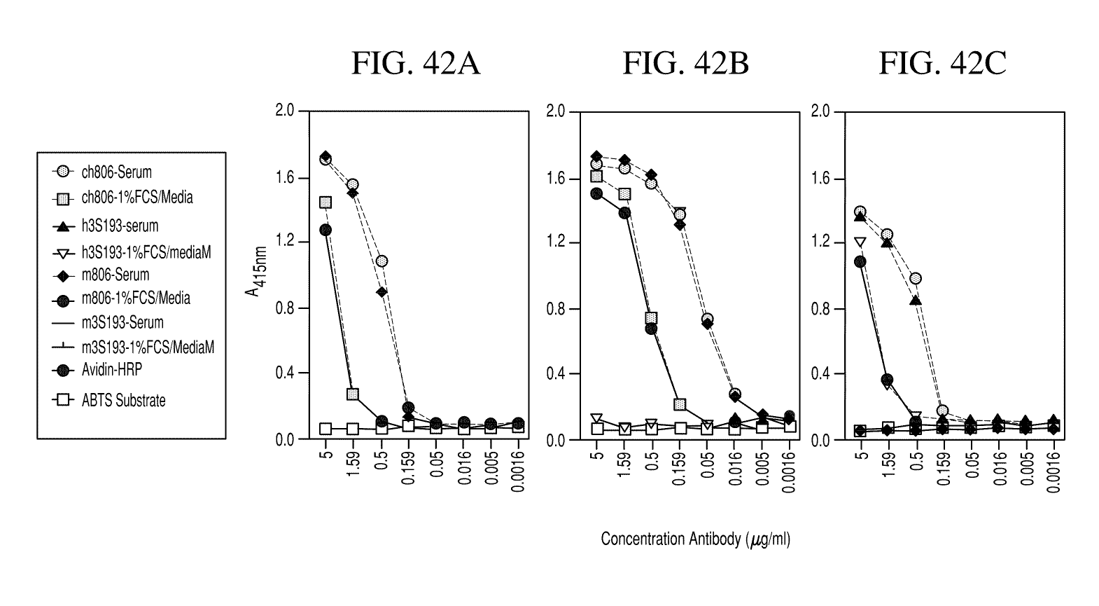

In the absence of readily available recombinant antigen de2-7 EGFR to assist with the determination of ch806 in serum samples, the ability of the novel anti-idiotype ch806 antibodies to concurrently bind 806 variable regions was exploited in the development of a sensitive, specific ELISA for measuring ch806 in clinical samples (FIG. 42). Using LMH-12 for capture and Biotinylated-LMH-12 for detection, the validated ELISA demonstrated highly reproducible binding curves for measuring ch806 (2 μg/ml-1.6 ng/ml) in sera with a 3 ng/ml limit of detection. (n=12; 1-100 ng/ml, Coefficient of Variation <25%; 100 ng/ml-5 μg/ml, Coefficient of Variation <15%). No background binding was evident with the three healthy donor sera tested and negligible binding was observed with isotype control hu3S193. The hybridoma produces high levels of antibody LMH-12, and larger scale production is planned to enable the measurement of ch806 and quantitation of any immune responses in clinical samples (Brown and Ling, 1988).

### Results

Mice Immunization and hybridoma clone selection Immunoreactivity of pre- and post-immunization sera samples indicated the development of high titer mouse anti-ch806 and anti-huIgG mAbs. Twenty-five hybridomas producing antibodies that bound ch806, but not huIgG, were initially selected. The binding characteristics of some of these hybridomas are shown in FIGS. 42A and 42B. Four of these anti-ch806 hybridomas with high affinity binding (clones 3E3, SB8, 9D6, and 4D8) were subsequently pursued for clonal expansion from single cells by limiting dilution and designated Ludwig Institute for Cancer Research Melbourne Hybridoma (LMH)-11, -12, -13, and -14, respectively (FIG. 42).

### Binding and Blocking Activities of Selected Anti-Idiotype Antibodies

The ability of anti-ch806 antibodies to concurrently bind two ch806 antibodies is a desirable feature for their use as reagents in an ELISA for determining serum ch806 levels. Clonal hybridomas, LMH-11, -12, -13, and -14 demonstrated concurrent binding (data not shown).

After clonal expansion, the hybridoma culture supernatants were examined by ELISA for the ability to neutralize ch806 or mAb806 antigen binding activity with sEGFR621. Results demonstrated the antagonist activity of anti-idiotype mAbs LMH-11, -12, -13, and -14 with the blocking in solution of both ch806 and murine mAb806 binding to plates coated with sEGFR (FIG. 41 for LMH-11, -12, -13).

Following larger scale culture in roller bottles the binding specificity's of the established clonal hybridomas, LMH-11, -12, -13, and -14 were verified by ELISA. LMH-11 through-14 antibodies were identified as isotype IgG1k by mouse monoclonal antibody isotyping kit.

ch806 in Clinical Serum Samples Pharmacokinetic ELISA Assay Development

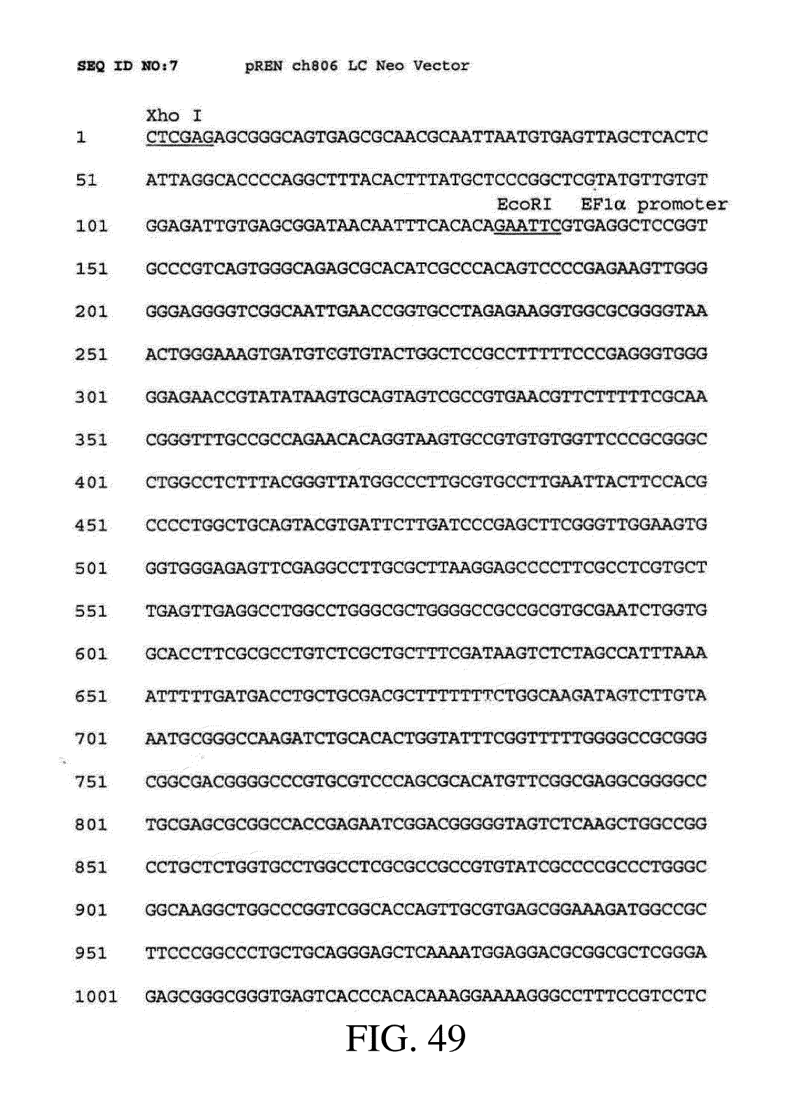

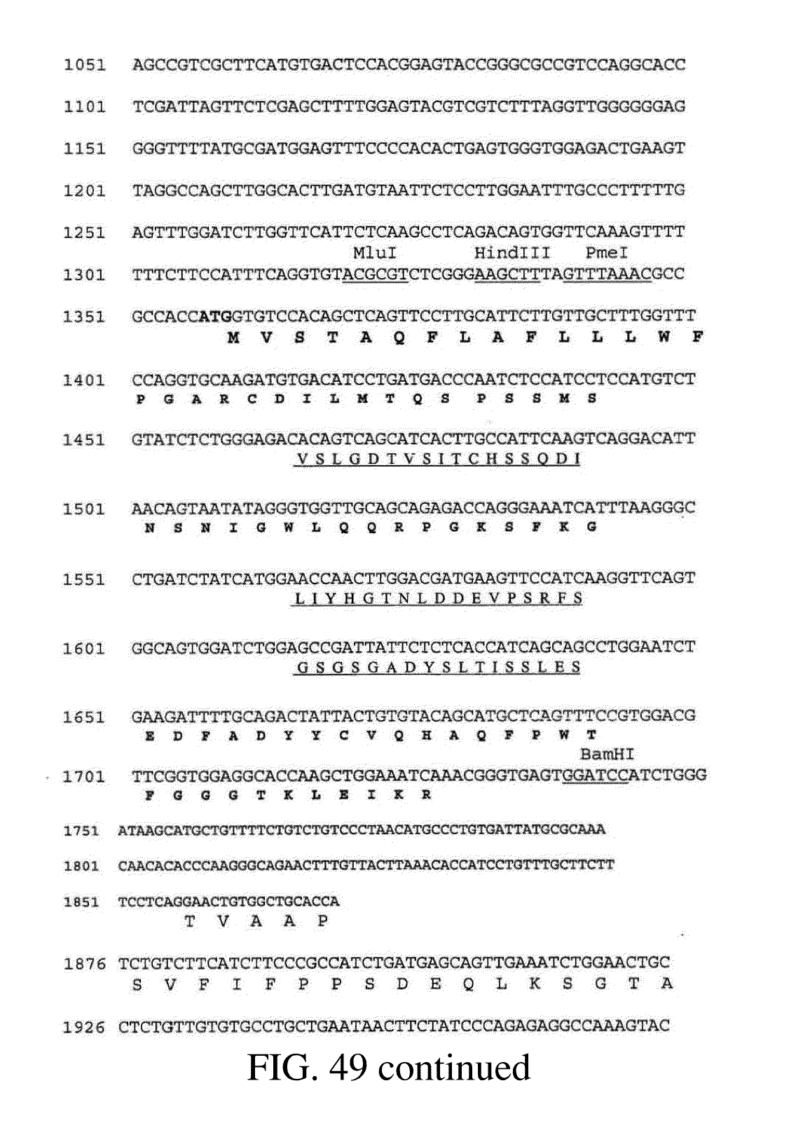

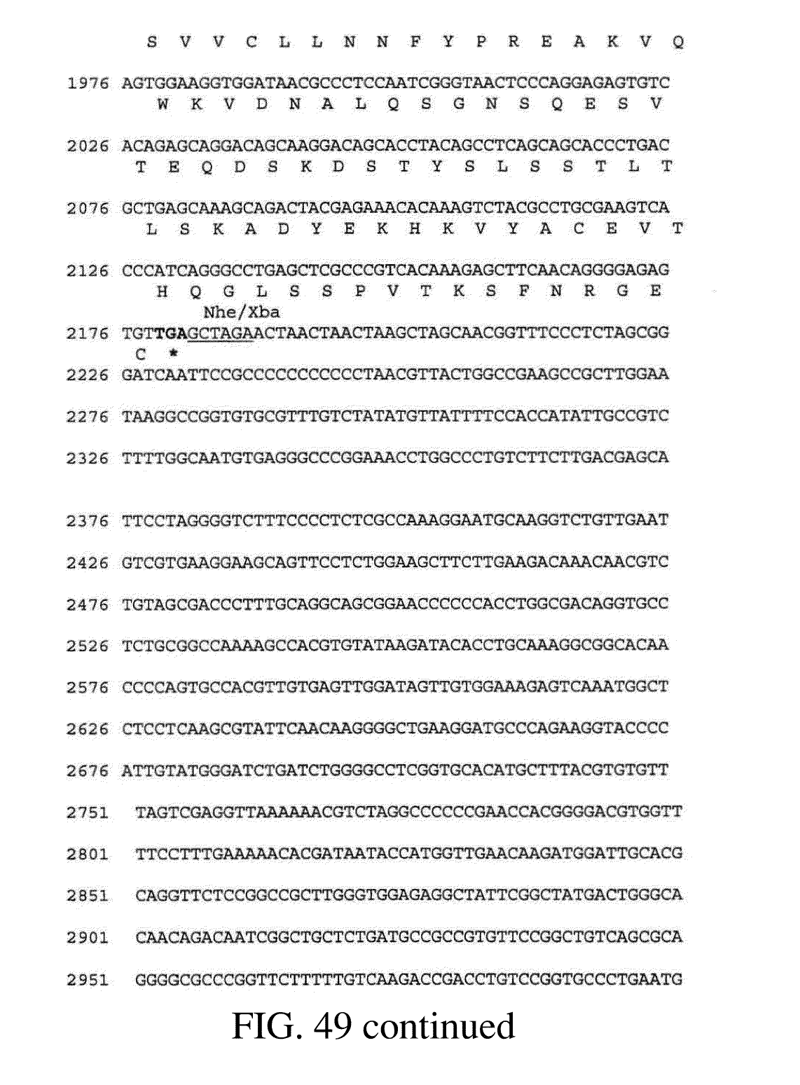

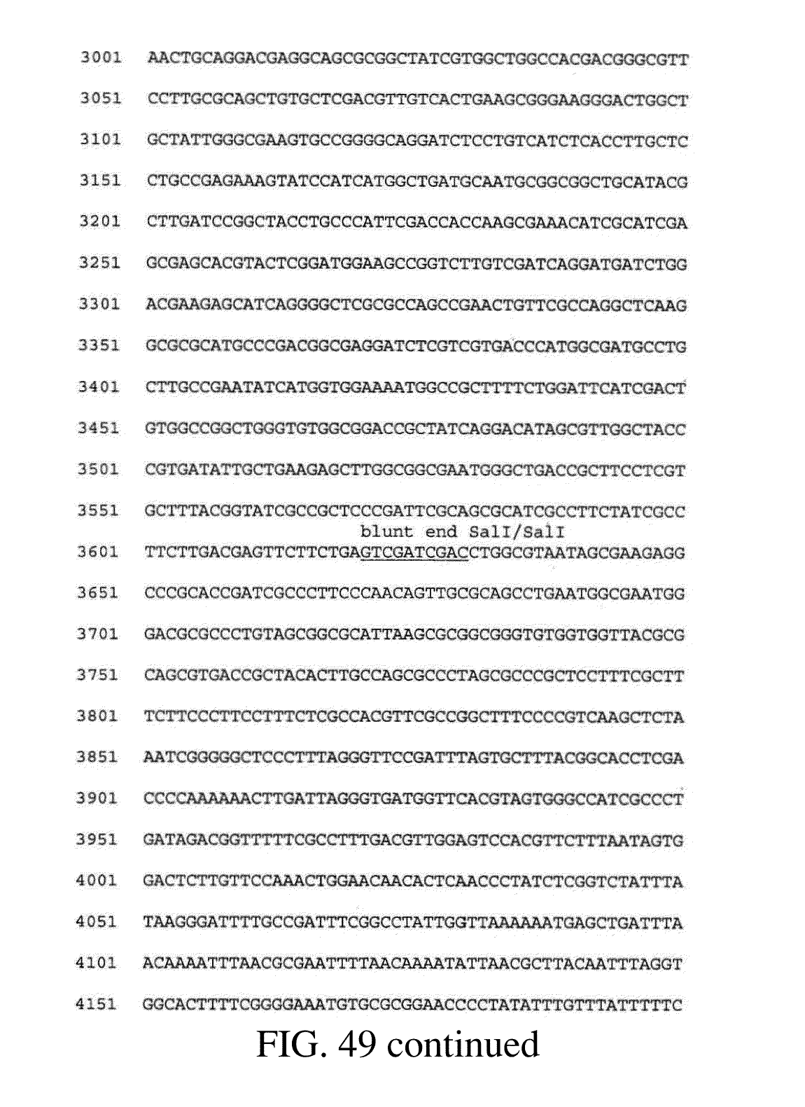

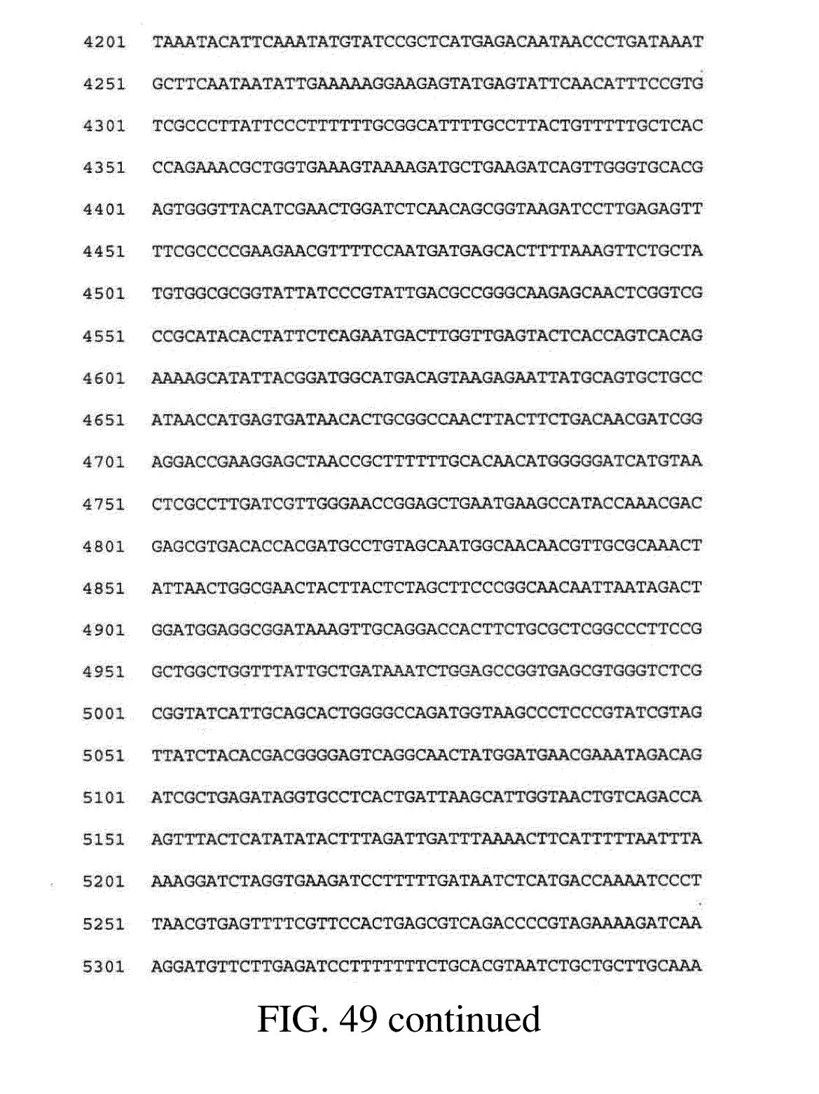

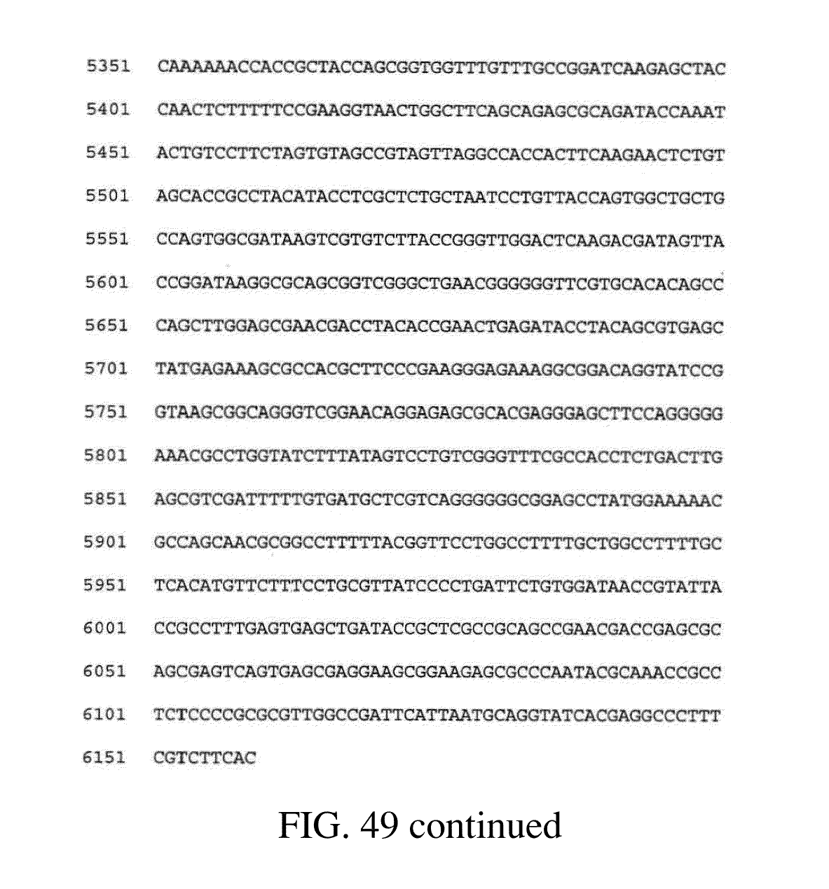

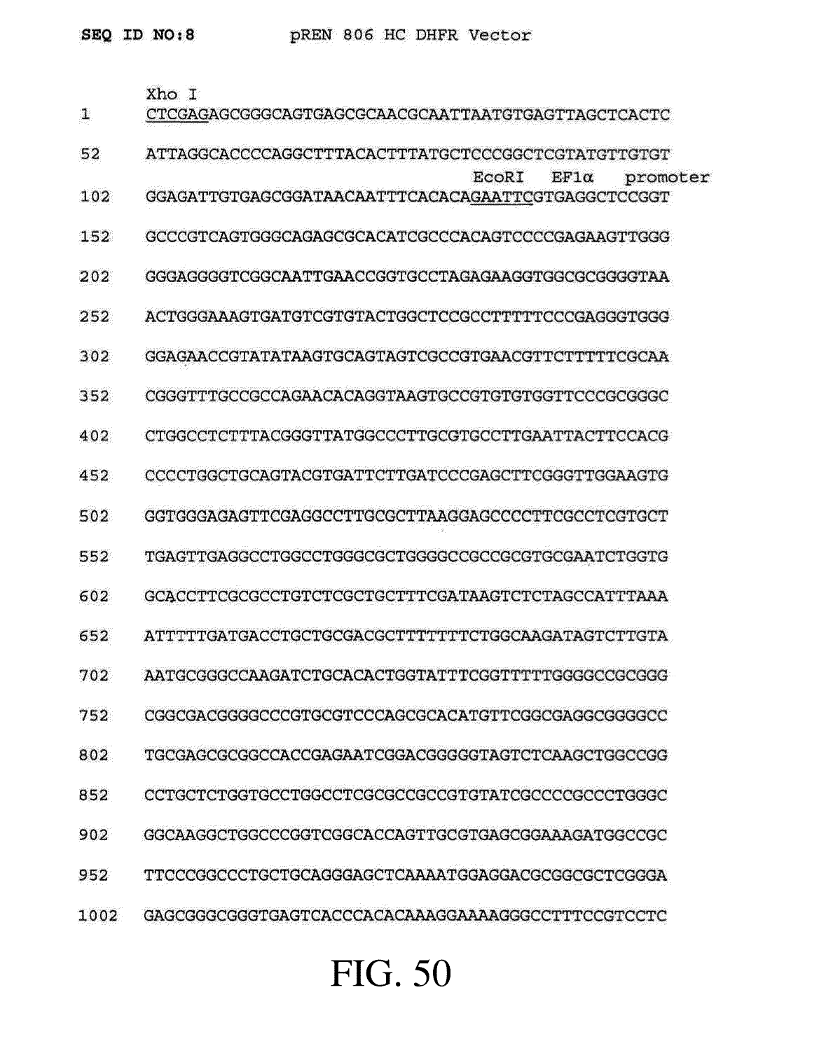

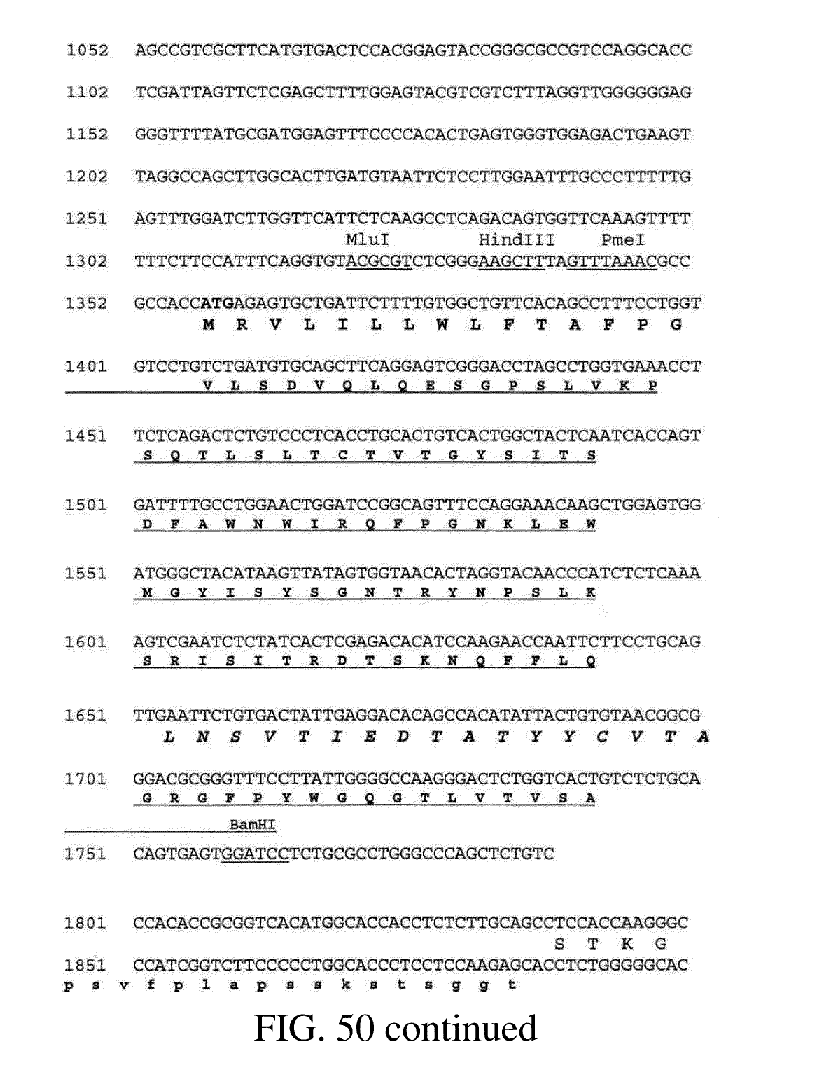

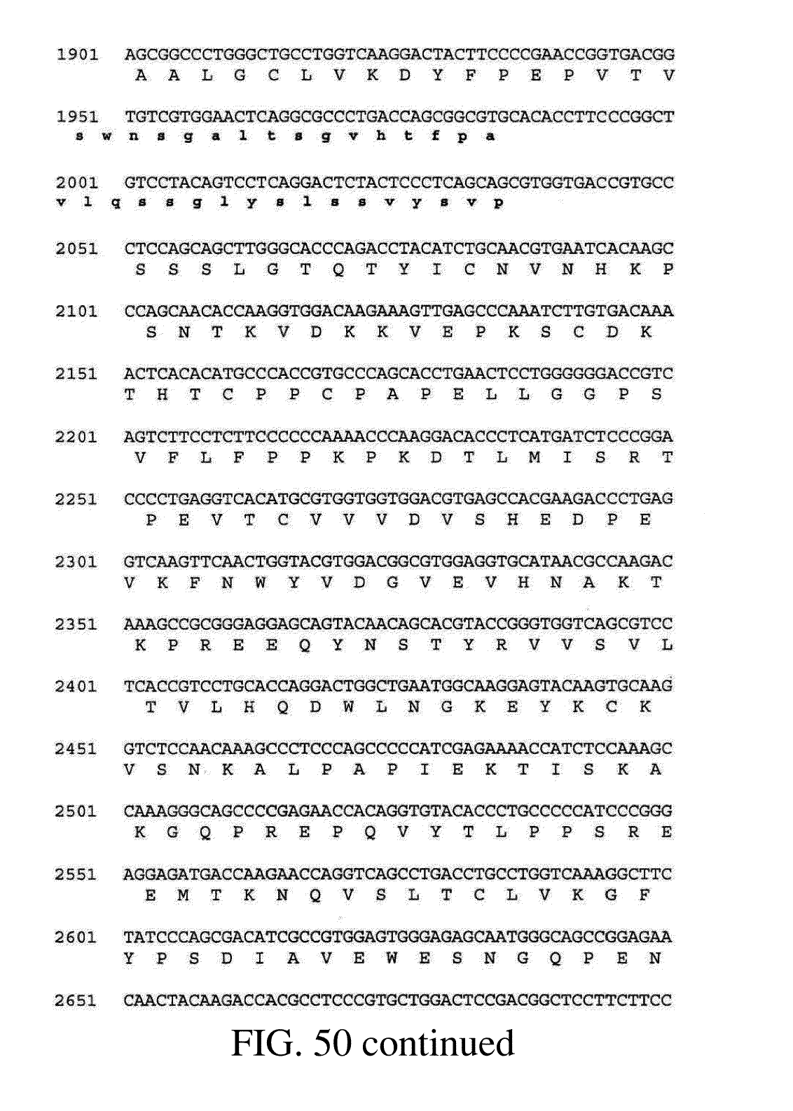

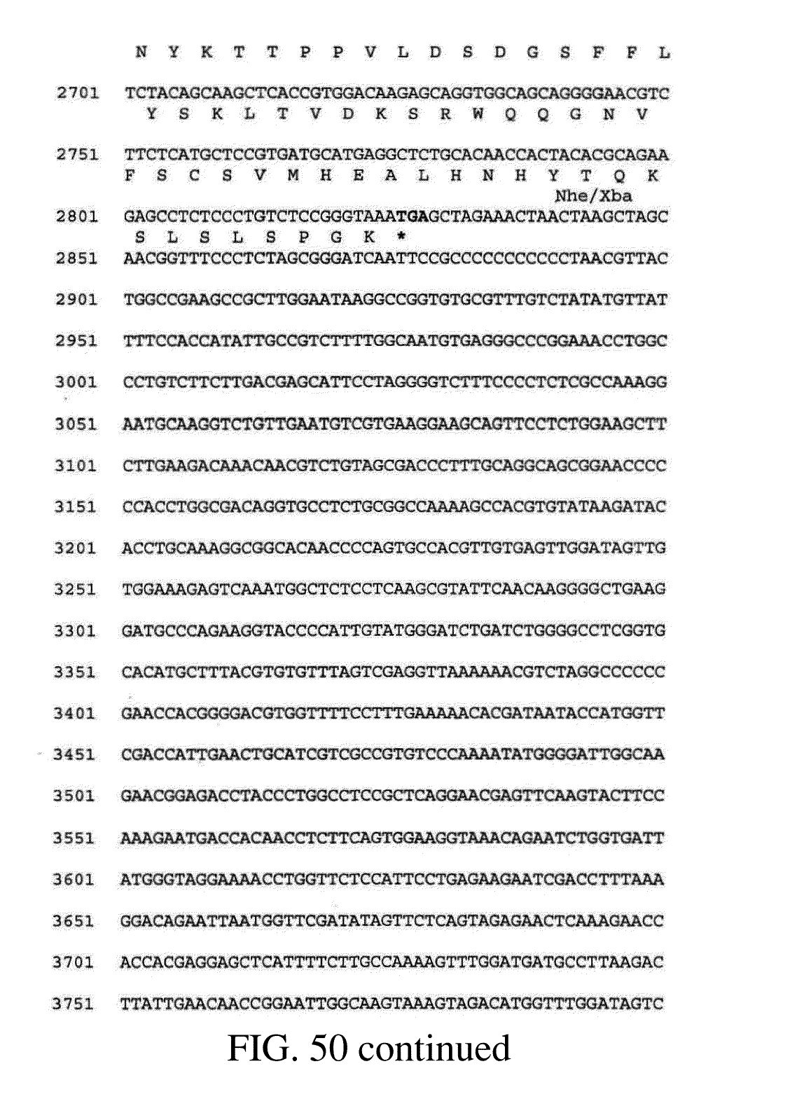

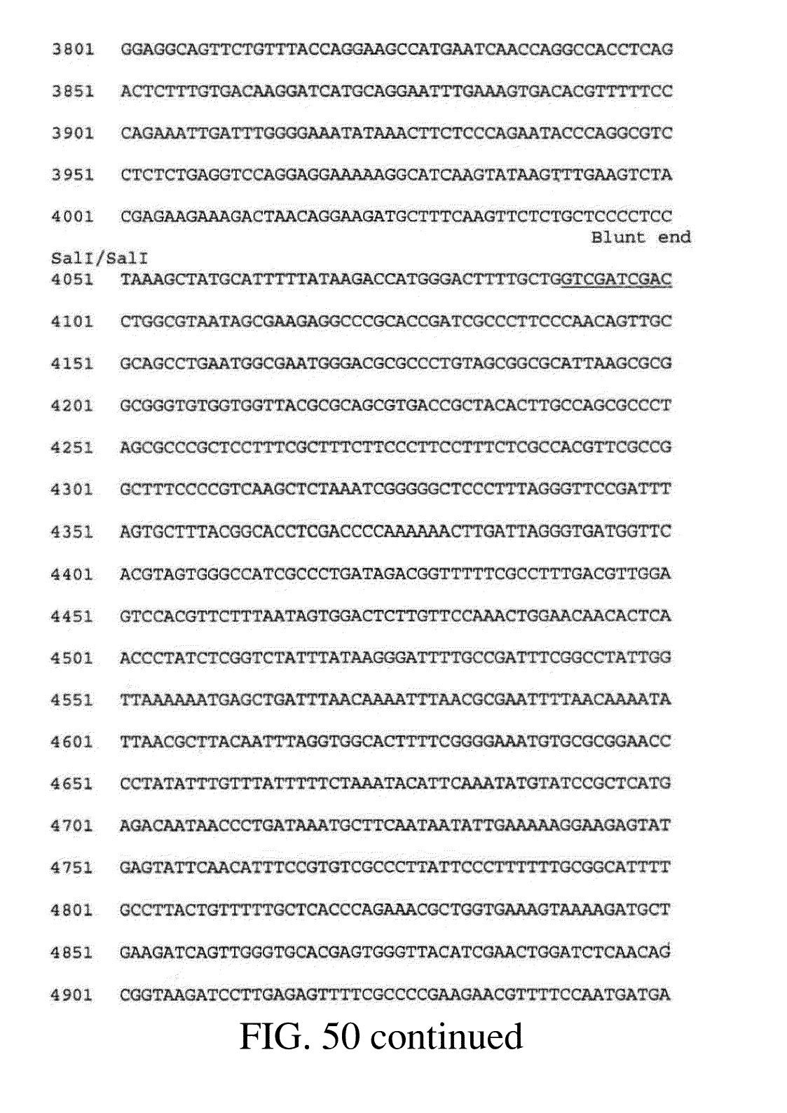

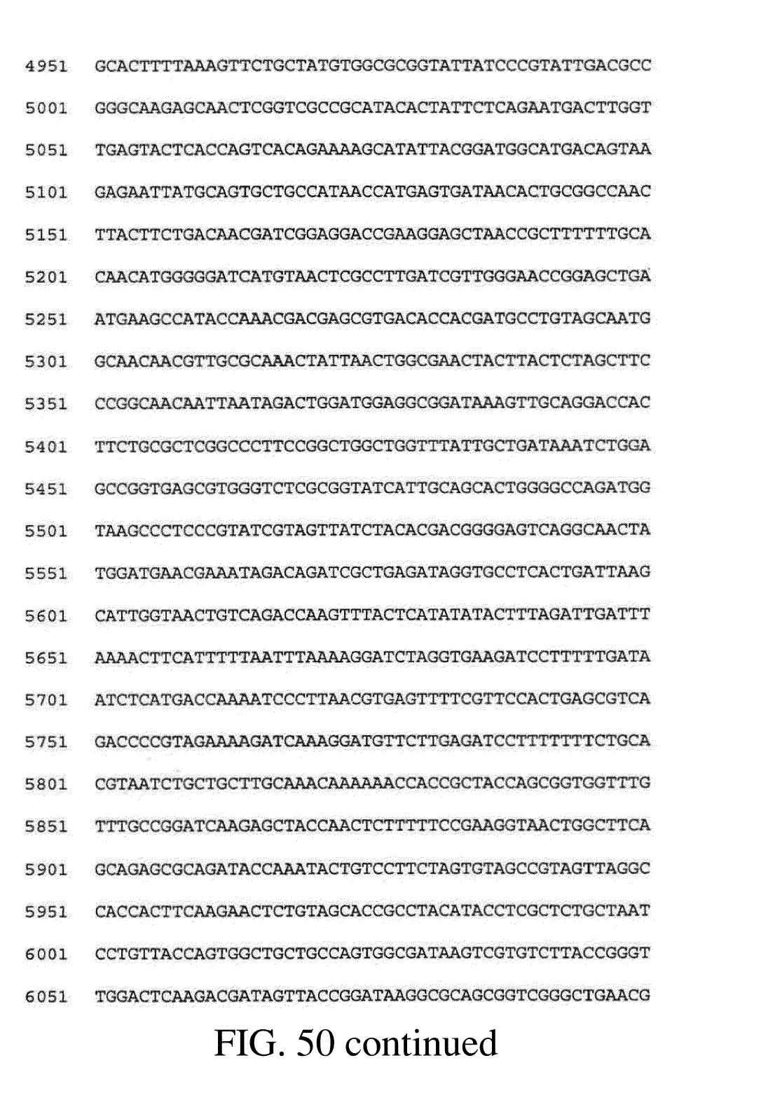

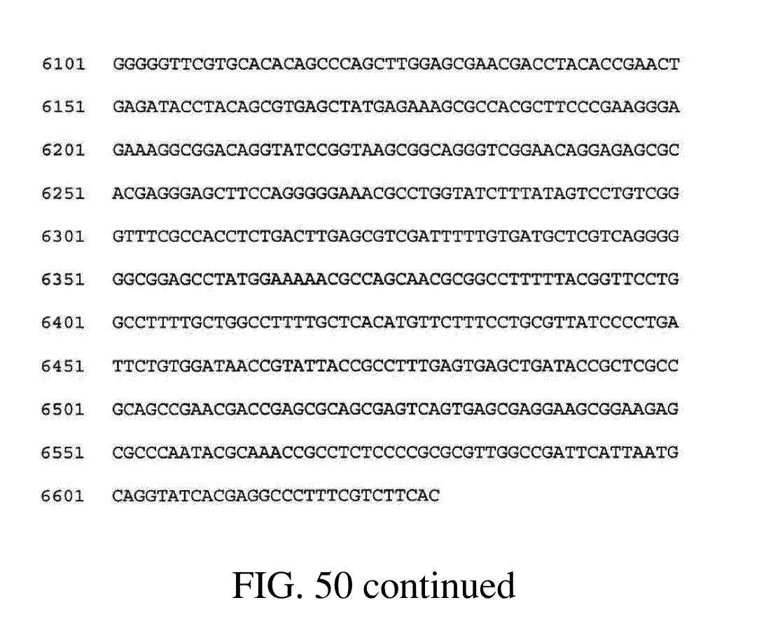

To assist with the determination of ch806 in serum samples, the ability of the anti-idiotype ch806 antibodies to concurrently bind the 806 variable region was exploited in the development of a sensitive and specific ELISA assay for ch806 in clinical samples. The three purified clones LMH-11, -12, and -13 (FIGS. 49B and 49C, respectively were compared for their ability to capture and then detect bound ch806 in sera. Results indicated using LMH-12 (10 μg/ml) for capture and biotinylated LMH-12 for detection yielded the highest sensitivity for ch806 in serum (3 ng/ml) with negligible background binding.

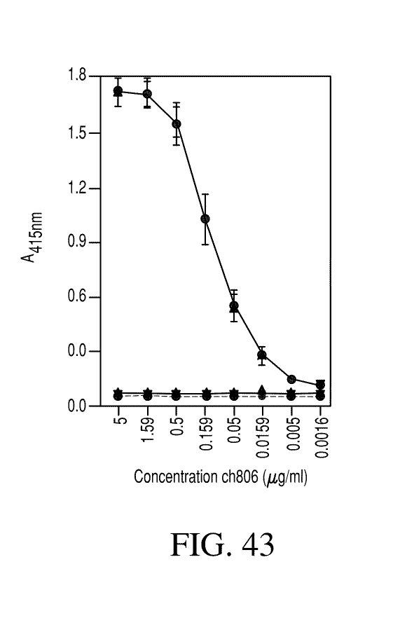

Having established the optimal pharmacokinetic ELISA conditions using 1 μg/ml anti-idiotype LMH-12 and 1 μg/mlbiotinylated LMH-12 for capture and detection, respectively, validation of the method was performed. Three separate ELISAs were performed in quadruplicate to measure ch806 in donor serum from three healthy donors or 1% BSA/media with isotype control hu3S193. Results of the validation are presented in FIG. 43 and demonstrate highly reproducible binding curves for measuring ch806 (2 μg/ml-1.6 ng/ml) in sera with a 3 ng/ml limit of detection. (n=12; 1-100 ng/ml, Coefficient of Variation <25%; 100 ng/ml-5 μg/ml, Coefficient of Variation <15%). No background binding was evident with any of the three sera tested and negligible binding was observed with isotype control hu3S193.

### Example 21

### Assessment of Carbohydrate Structures and Antibody Recognition

Experiments were undertaken to further assess the role of carbohydrate structures in the binding and recognition of the EGFR, both amplified and de2-7 EGFR, by the mAb806 antibody.

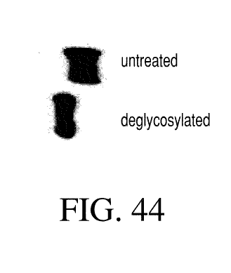

To determine if carbohydrate structures are directly involved in the mAb806 epitope, the recombinant sEGFR expressed in CHO cells was treated with PNGase F to remove N-linked glycosylation. Following treatment, the protein was run on SDS-PAGE, transferred to membrane and immunoblotted with mAb806 (FIG. 44). As expected, the deglycosylated sEGFR ran faster on SDS-PAGE, indicating that the carbohydrates had been successfully removed. The mAb806 antibody clearly bound the deglycosylated material demonstrating the antibody epitope is peptide in nature and not solely a glycosylation epitope.

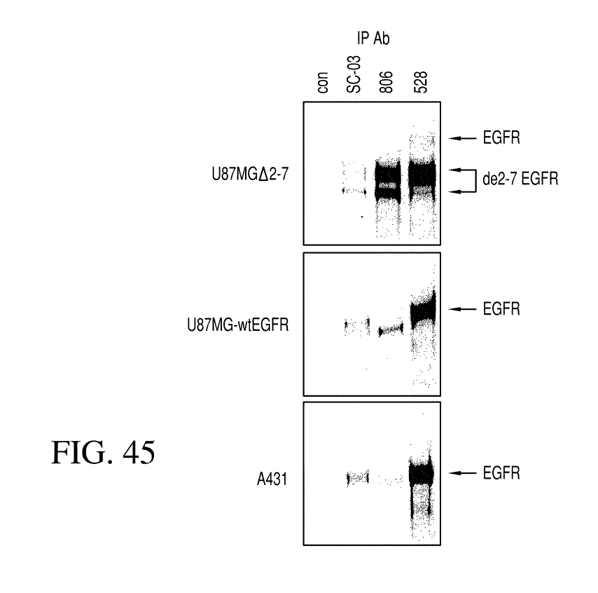

Lysates, prepared from cell lines metabolically labelled with ³⁵S, were immunoprecipitated with different antibodies directed to the EGFR (FIG. 45). As expected, the 528 antibody immunoprecipitated three bands from U87MG.Δ2-7 cells, an upper band corresponding to the wild-type (wt) EGFR and two lower bands corresponding to the de2-7 EGFR. These two de2-7 EGFR bands have been reported previously and are assumed to represent differential glycosylation (Chu et al. (1997) Biochem. J. Jun 15; 324 (Pt 3): 885-861). In contrast, mAb806 only immunoprecipitated the two de2-7 EGFR bands, with the wild-type receptor being completely absent even after over-exposure (data not shown). Interestingly, mAb806 showed increased relative reactivity with the lower de2-7 EGFR band but decreased reactivity with the upper band when compared to the 528 antibody. The SC-03 antibody, a commercial rabbit polyclonal antibody directed to C-terminal domain of the EGFR, immunoprecipitated the three EGFR bands as seen with the 528 antibody, although the total amount of receptor immunoprecipitated by this antibody was considerably less. No bands were observed when using an irrelevant IgG2b antibody as a control for mAb806 (see Example 18).

The 528 antibody immunoprecipitated a single band from U87MG.wtEGFR cells corresponding to the wild-type receptor (FIG. 45). mAb806 also immunoprecipitated a single band from these cells, however, this EGFR band clearly migrated faster than the 528 reactive receptor. The SC-03 antibody immunoprecipitated both EGFR reactive bands from U87MG.wtEGFR cells, further confirming that the mAb806 and 528 recognize different forms of the EGFR in whole cell lysates from these cells.

As observed with U87MG.wtEGFR cells, the 528 antibody immunoprecipitated a single EGFR band from A431 cells (FIG. 45). The 528 reactive EGFR band is very broad on these low percentage gels (6%) and probably reflects the diversity of receptor glycosylation. A single EGFR band was also seen following immunoprecipitation with mAb806. While this EGFR band did not migrate considerably faster than the 528 overall broad reactive band, it was located at the leading edge of the broad 528 band in a reproducible fashion. Unlike U87MG.Δ2-7 cell lysates, the total amount of EGFR immunoprecipitated by mAb806 from A431 lysates was considerably less than with the 528 antibody, a result consistent with our Scatchard data showing mAb806 only recognizes a portion of the EGFR on the surface of these cells (see Example 4). Immunoprecipitation with SC-03 resulted in a single broad EGFR band as for the 528 antibody. Similar results were obtained with HN5 cells (data not shown). Taken together, this data indicates that mAb806 preferentially reacts with faster migrating species of the EGFR, which may represent differentially glycosylated forms of the receptor.

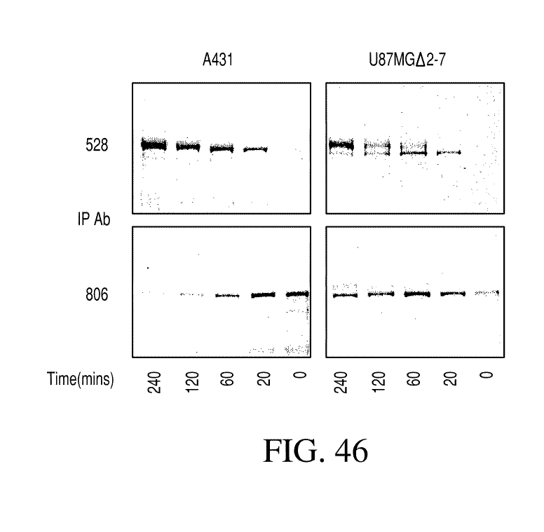

In order to determine at what stage of receptor processing mAb806 reactivity appeared a pulse/chase experiment was conducted. A431 and U87MG.Δ2-7 cells were pulsed for 5 min with ³⁵S methionine/cysteine, then incubated at 37° C. for various times before immunoprecipitation with mAb806 or 528 (FIG. 46). The immunoprecipitation pattern in A431 cells with the 528 antibody was typical for a conformational dependent antibody specific for the EGFR. A small amount of receptor was immunoprecipitated at 0 min (i.e. after 5 min pulse) with the amount of labelled EGFR increasing at each time point. There was also a concurrent increase in the molecular weight of the receptor with time. In contrast, the mAb806 reactive EGFR material was present at high levels at 0 min, peaked at 20 min and then reduced at each further time point. Thus, it appears that mAb806 preferentially recognizes a form of the EGFR found at an early stage of processing.

The antibody reactivity observed in pulse-labelled U87MG.Δ2-7 cells was more complicated. Immunoprecipitation with the 528 antibody at 0 min revealed that a small amount of the lower de2-7 EGFR band was labelled (FIG. 46). The amount of 528 reactive de2-7 EGFR lower band increased with time, peaking at 60 min and declining slowly at 2 and 4 h. No significant amount of the labelled upper band of de2-7 EGFR was detected until 60 min, after which the level continued to increase until the end of the time course. This clearly indicates that the upper de2-7 EGFR is a more mature form of the receptor. mAb806 reactivity also varied during the time course study, however mAb806 preferentially precipitated the lower band of the de27 EGFR. Indeed, there were no significant levels of mAb806 upper band seen until 4 h after labeling.

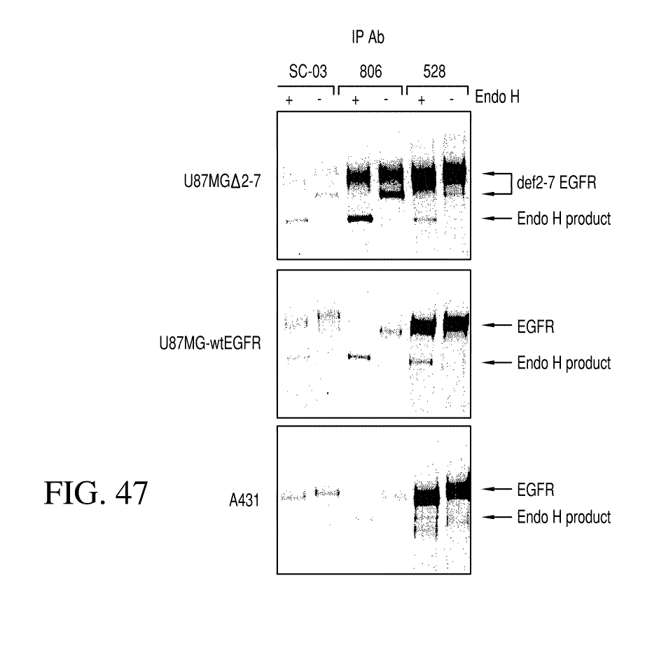

The above experiments suggest that mAb806 preferentially reacts with a more immature glycosylation form of the de2-7 and wtEGFR. This possibility was tested by immunoprecipitating the EGFR from different cells lines labelled overnight with ³⁵S methionine/cysteine and then subjecting the resultant precipitates to Endoglycosidase H (Endo H) digestion. This enzyme preferentially removes high mannose type carbohydrates (i.e. immature glycosylation) from proteins while leaving complex carbohydrates (i.e. mature glycosylation) intact. Immunoprecipitation and digestion with Endo H of labelled U87MG.Δ2-7 cell lysates with 528, mAb806 and SC-03 gave similar results (FIG. 47).

As predicted, the lower de2-7 EGFR band was fully sensitive to Endo H digestion, migrating faster on SDS-PAGE after Endo H digestion, demonstrating that this band represents the high mannose form of the de2-7 EGFR. The upper de2-7 EGFR band was essentially resistant to Endo H digestion, showing only a very slight difference in migration after Endo H digestion, indicating that the majority of the carbohydrate structures are of the complex type. The small but reproducible decrease in the molecular weight of the upper band following enzyme digestion suggests that while the carbohydrates on the upper de2-7 EGFR band are predominantly of the complex type, it does possess some high mannose structures. Interestingly, these cells also express low amounts of endogenous wtEGFR that is clearly visible following 528 immunoprecipitation. There was also a small but noticeable reduction in molecular weight of the wild-type receptor following Endo H digestion, indicating that it also contains high mannose structures.

The sensitivity of the immunoprecipitated wtEGFR to Endo H digestion was similar in both U87MG.wtEGFR and A431 cells (FIG. 47). The bulk of the material precipitated by the 528 antibody was resistant to the Endo H enzyme although a small amount of the material was of the high mannose form. Once again there was a small decrease in the molecular weight of the wtEGFR following Endo H digestion suggesting that it does contain some high mannose structures. The results using the SC-03 antibody were similar to the 528 antibody. In contrast, the majority of the EGFR precipitated by mAb806 was sensitive to Endo H in both U87MG.wtEGFR and A431 cells, confirming that mAb806 preferentially recognizes the high mannose form of the EGFR. Similar results were obtained with HN-5 cells, wherein the majority of the material precipitated by mAb806 was sensitive to Endo H digestion, while the majority of the material precipitated by mAb528 and SC-03 was resistant to Endo H digestion (data not shown).

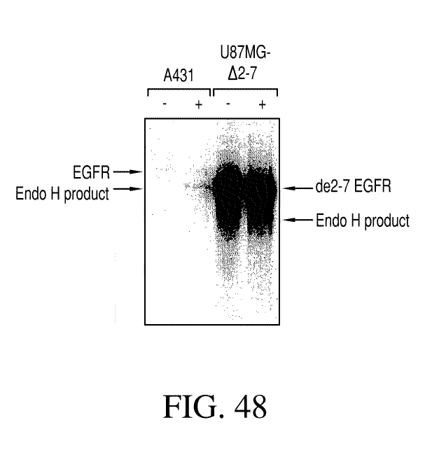

Cell surface iodination of the A431 cell line, was performed with ¹²⁵I followed by immunoprecipitation with the 806 antibody. The protocol for surface iodination was as follows: The cell lysis, immunoprecipitation, Endo H digestion, SDS PAGE and autoradiography are as described above herein. For labeling, cells were grown in media with 10% FCS, detached with EDTA, washed twice with PBS then resuspended in 400 μl of PBS (approx 2−3×10⁶ cells). To this was added 15 μl of ¹²⁵I (100 mCi/ml stock), 100 μl bovine lactoperoxidase (1 mg/ml) stock, 10 μl H₂O₂ (0.1% stock) and this was incubated for 5 min A further 10 μl H₂O₂ was then added and the incubation continued for a further 3 min. Cells were then washed again 3 times with PBS and lysed in 1% Triton. Cell surface iodination of the A431 cell line with lactoperoxidase, followed by immunoprecipitation with the 806 antibody, showed that, similar to the whole cell lysates described above, the predominant form of the EGFR recognized by 806 bound on the cell surface of A431 cells was sensitive to EndoH digestion (FIG. 48). This confirms that the form of EGFR bound by 806 on the cell surface of A431 cells is an EndoH sensitive form and thus is the high mannose type.

### Example 22

### Humanized (Veneered) Antibody 806

A. hu806 Construction

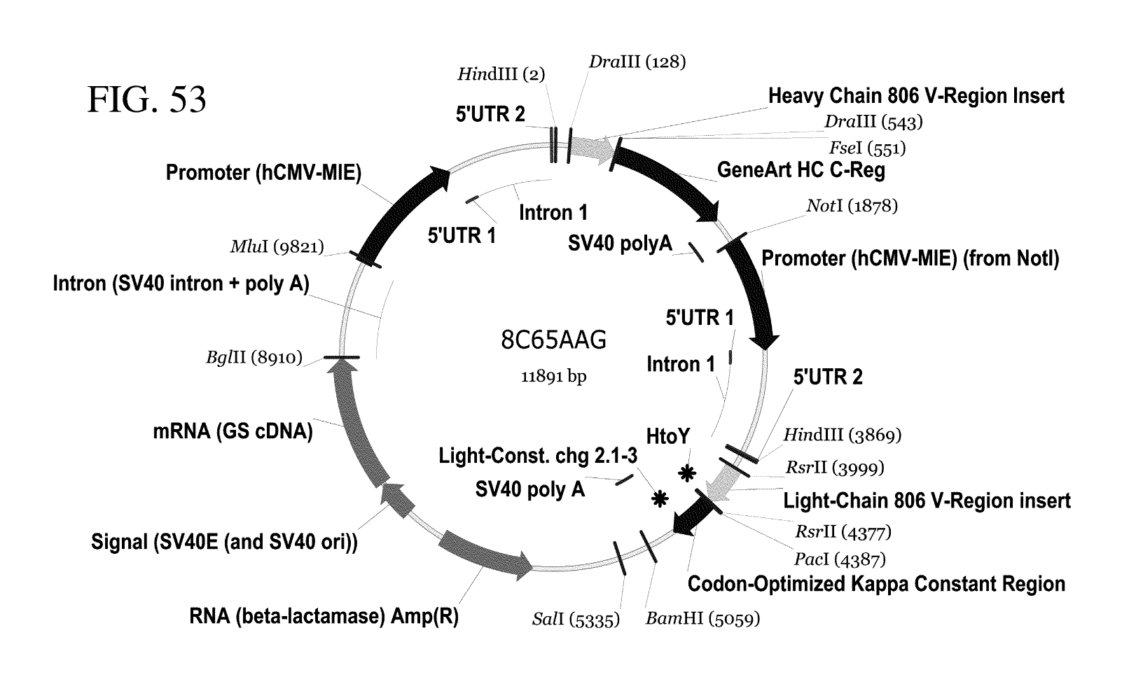

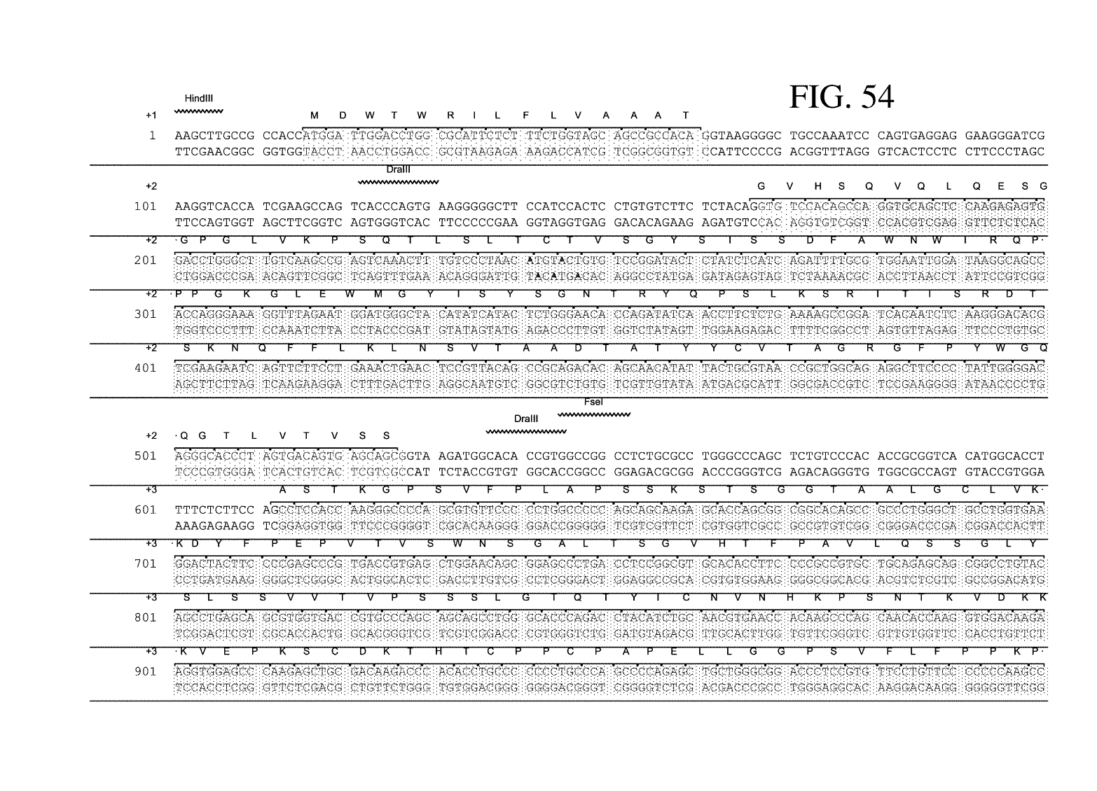

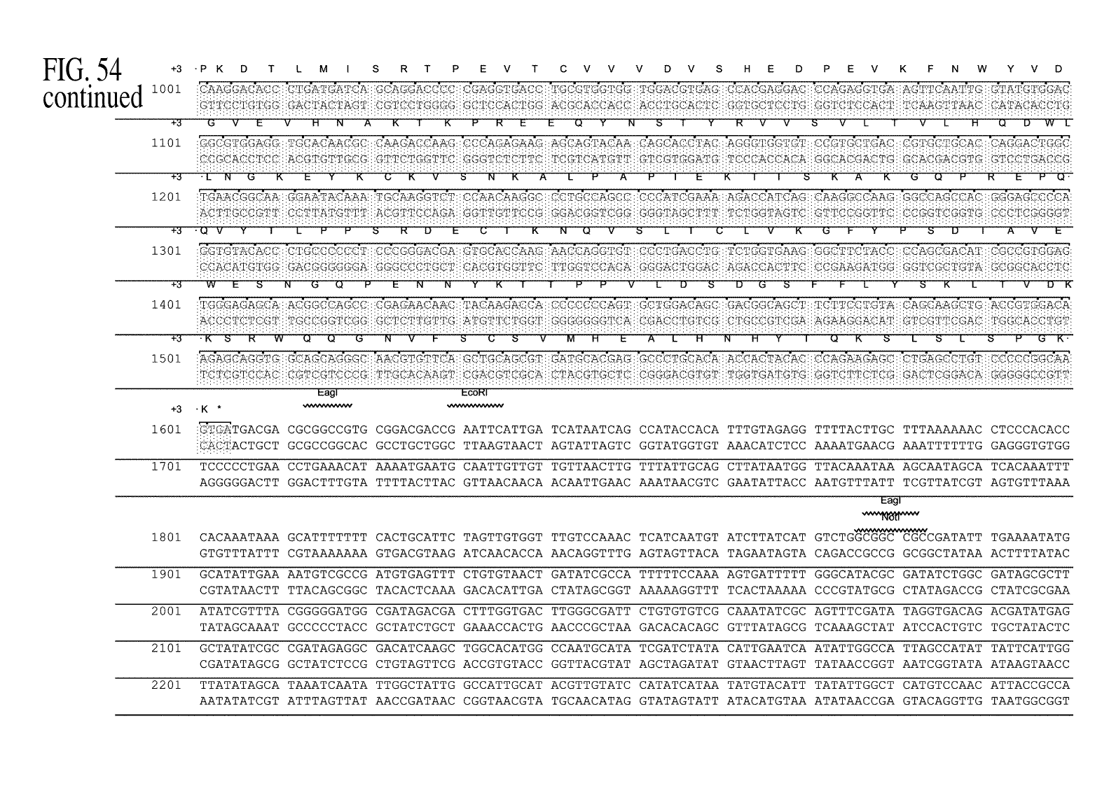

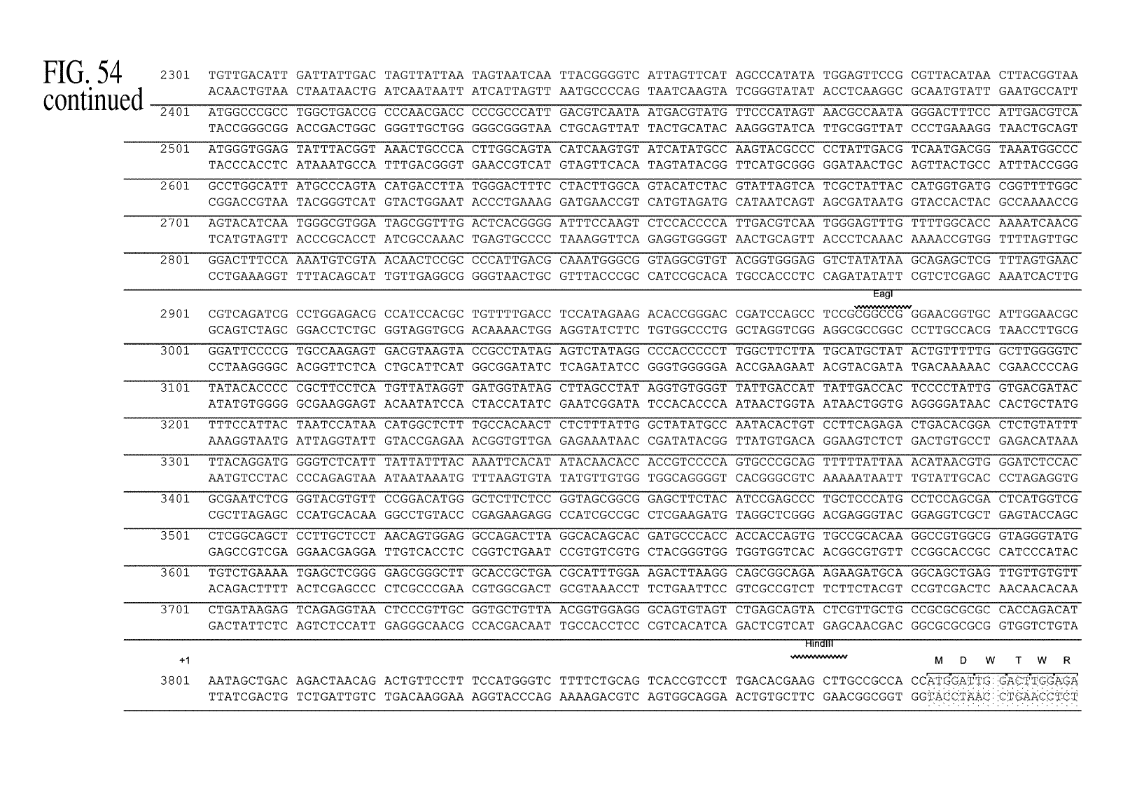

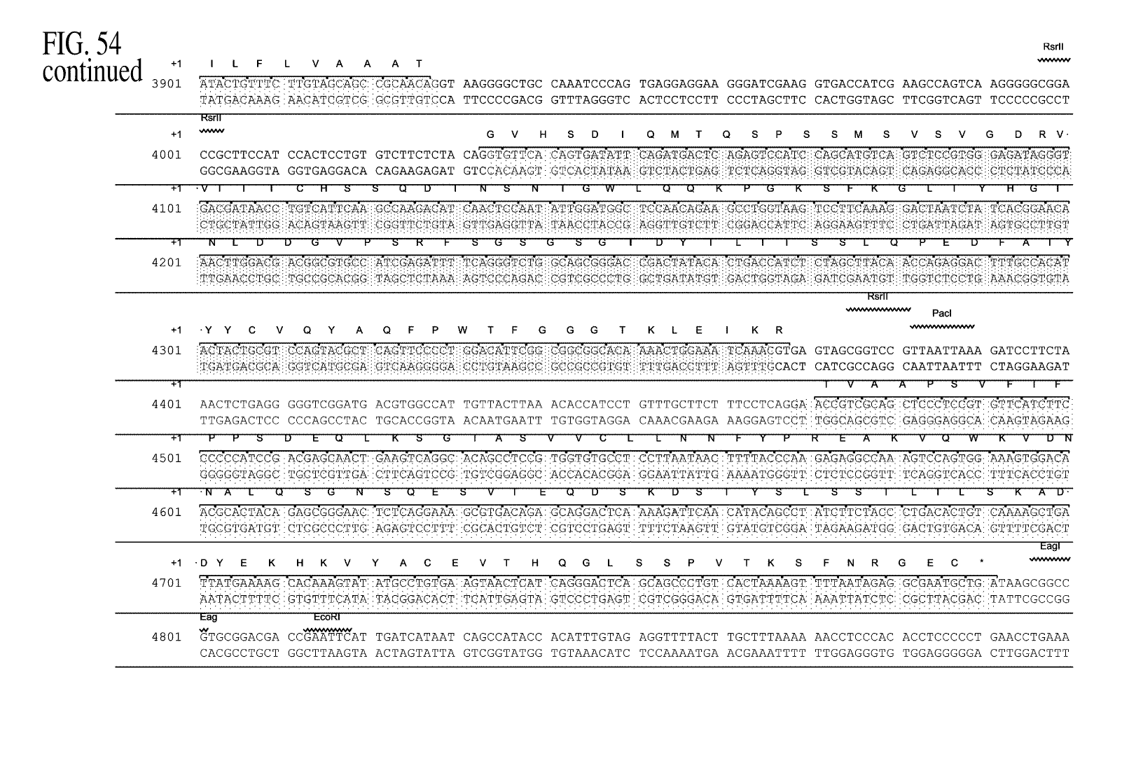

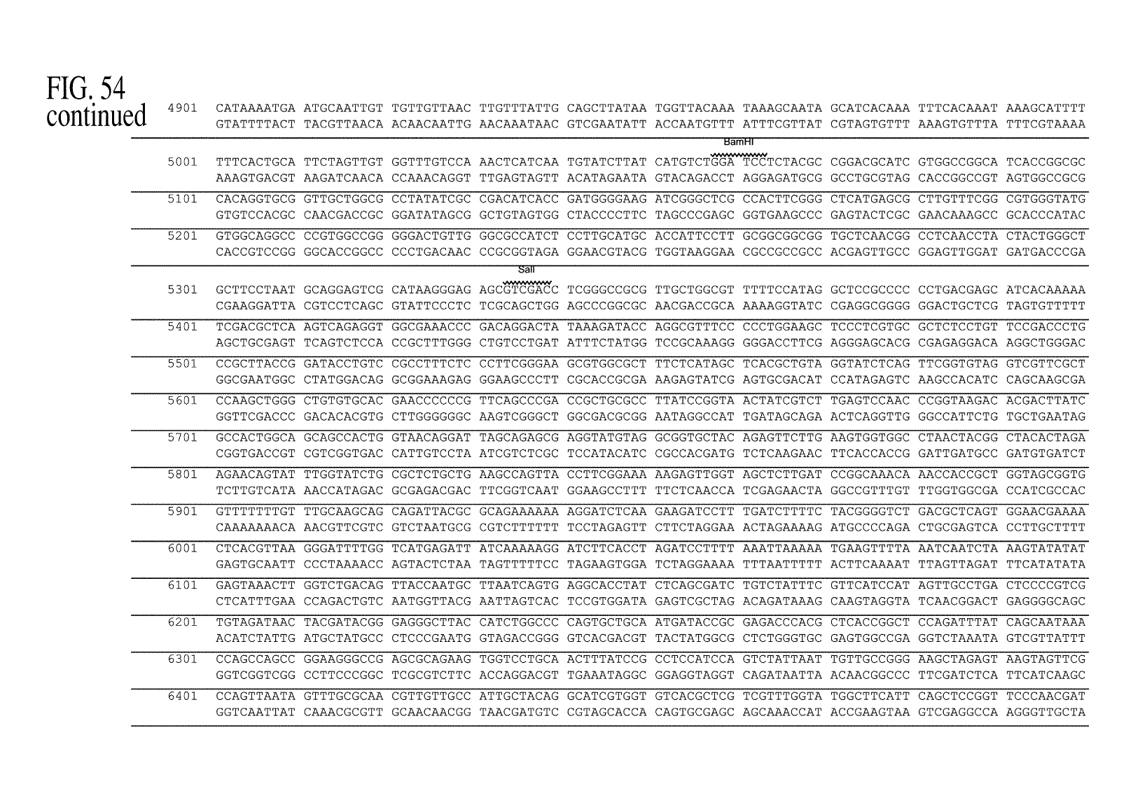

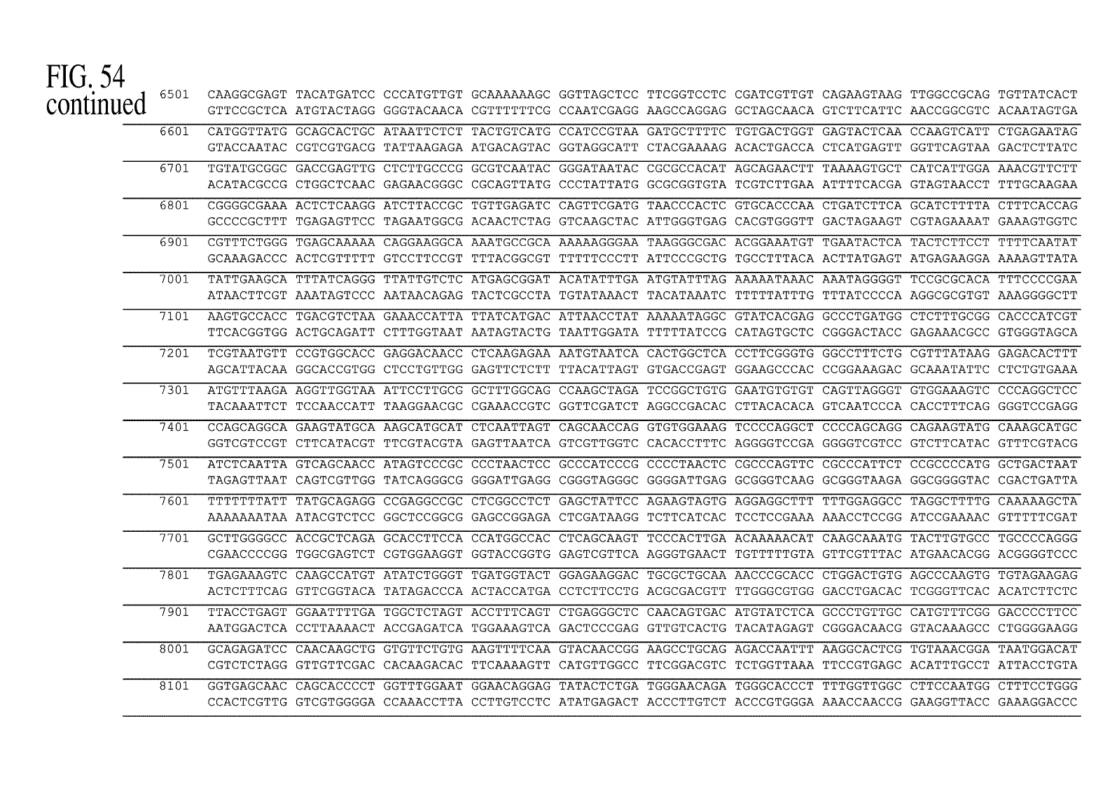

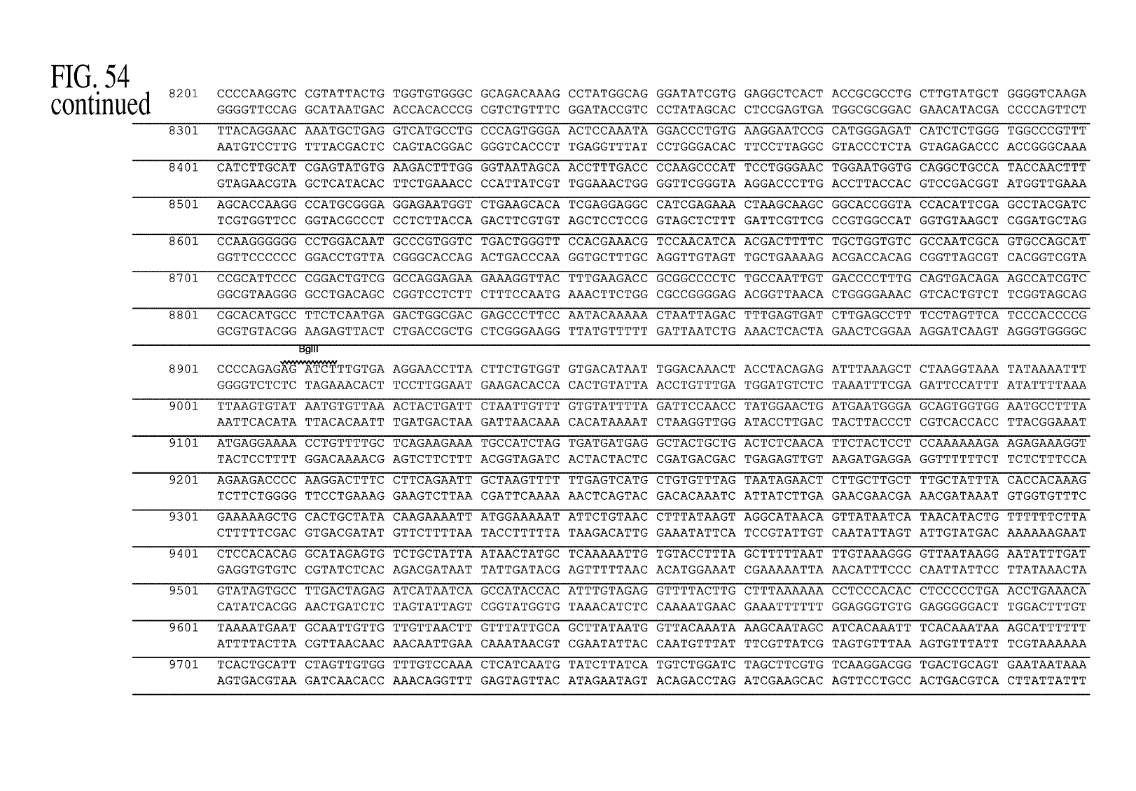

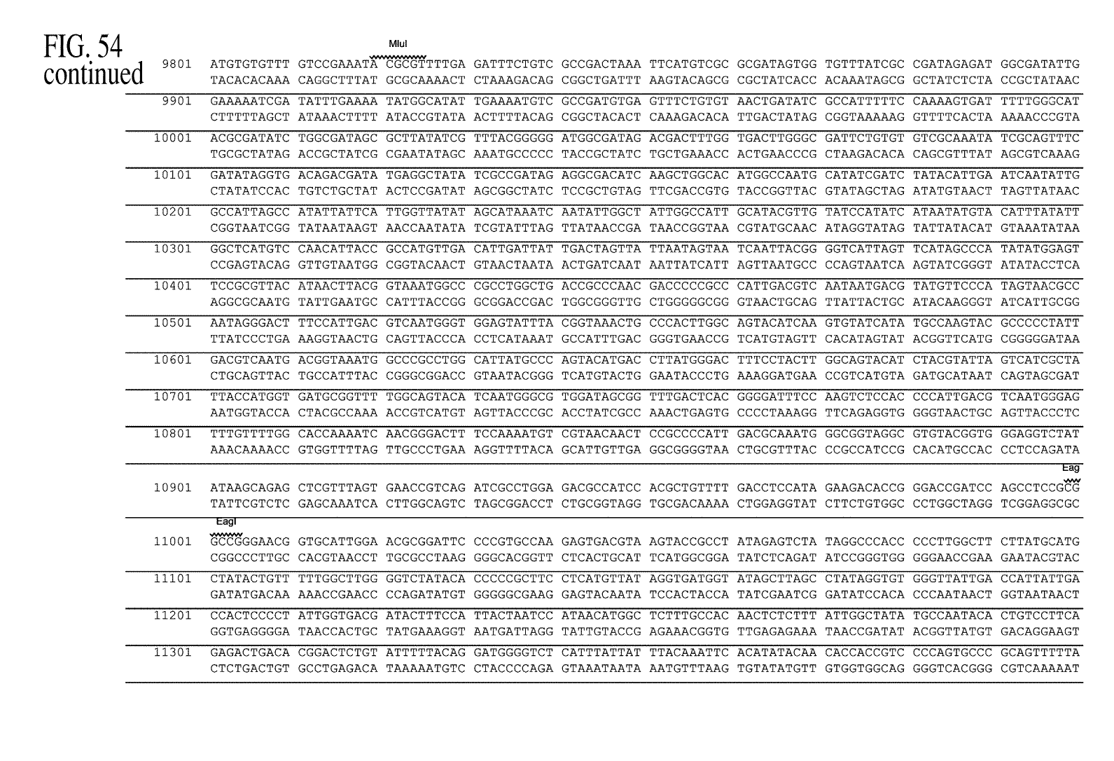

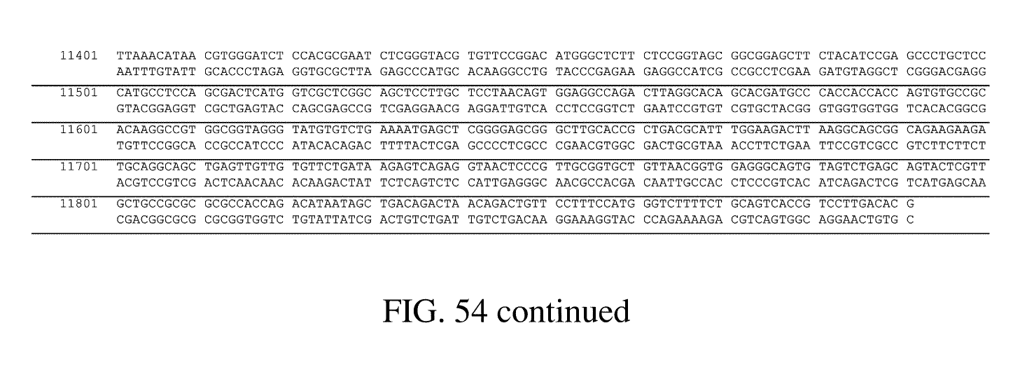

An expression vector for a humanized 806 antibody (hu806) was constructed. The vector, termed 8C65AAG (11891 bp; SEQ ID NO:41), was designed to contain both genes for a full length hu806 in a single GS promoter-driven gene expression cassette (FIGS. 53 and 54).

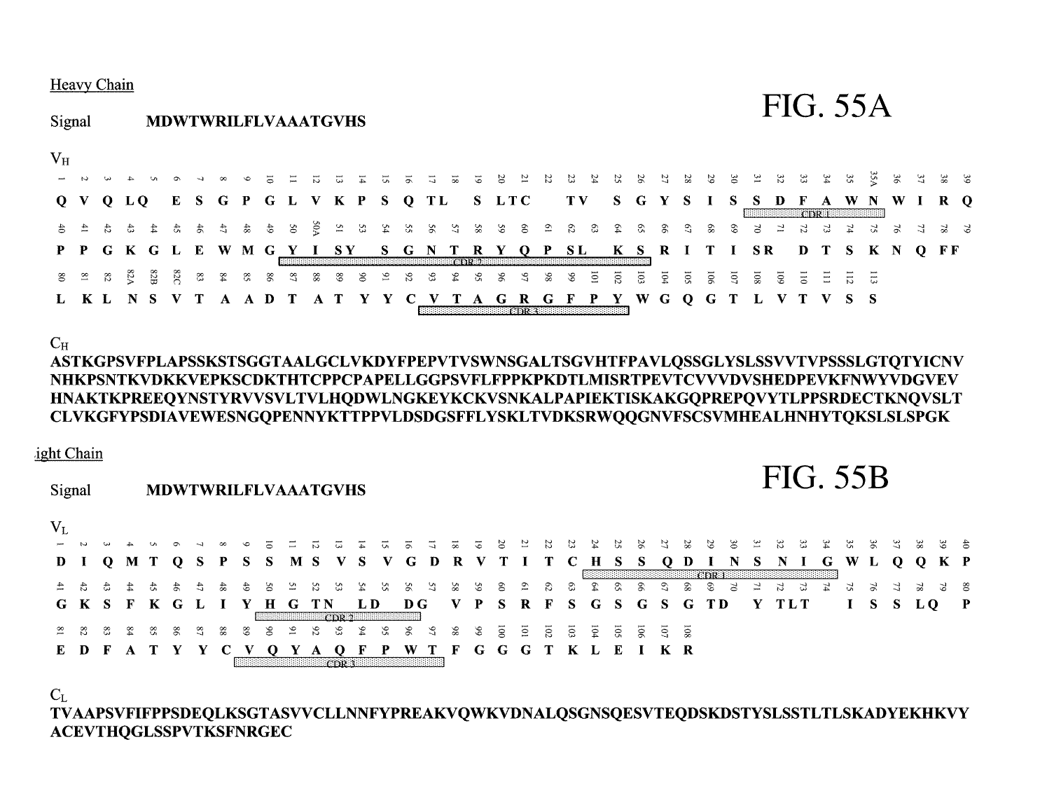

The heavy chain variable (VH) and constant (CH) regions (SEQ ID NOS:42 and 43, respectively) are shown in FIG. 55A, with the VH region CDR1, CDR2, and CDR3 (SEQ ID NOS:44, 45, and 46, respectively) indicated by underlining.

The light chain variable (VL) and constant (CL) regions (SEQ ID NOS:47 and 48, respectively) are shown in FIG. 55B, with the VL region CDR1, CDR2, and CDR3 (SEQ ID NOS:49, 50, and 51, respectively) indicated by underlining.

To obtain a humanized 806 antibody construct, the veneering (v) technology (Daugherty et al. (1991) Polymerase chain reaction facilitates the cloning, CDR-grafting, and rapid expression of a murine monoclonal antibody directed against the CD18 component of leukocyte integrins. Nucleic Acids Res. 19(9), 2471-6; U.S. Pat. No. 6,797,492 to Daugherty; Padlan, E.A. (1991) A possible procedure for reducing the immunogenicity of antibody variable domains while preserving their ligand-binding properties. Mol. Immunol. 28(4-5), 489-98; European Patent No. 519596 to Padlan et al.) was employed. In order to minimize the immunogenicity of 806 antibody variable domains, while preserving ligand-binding properties, replacement of the surface-exposed residues in the framework regions which differ from those usually found in human antibodies was undertaken. To achieve this, VL and VH chain of the mouse monoclonal antibody (mAb) 806 have been re-engineered by gene-synthesis and overlapping PCR primer technology. The CL (kappa) chain was assembled in the same manner. To demonstrate the preservation of intact binding sites, vVL and vVH were also expressed in a scFv format that demonstrated good binding to the synthetic peptide that comprises the 806 antigenic epitope by ELISA and to recombinant EGF Receptor (EGFR) extracellular domain (ECD) as measured by surface plasmon resonance (SPR) analysis.

The v806VL and v806VH have been engineered into a full length human IgG1 context using a codon-optimized kappa-LC and a newly designed codon- and splice-site optimized human IgG1 heavy chain constant region to achieve stable gene expression in NS0 and CHO cell systems. The expression system is based on the LONZA GS expression system using the pEE12.4 and pEE6.4 heavy and light chain expression vectors as provided by LONZA Biologics.

The hu806 antibody product (FIG. 55) obtained by transient expression of the 8C65AAG vector was reactive with recombinant EGFR-ECD by SPR, and with the synthetic EGFR 806 peptide epitope by ELISA. The 8C65AAG vector was transferred to LICR Affiliate Christoph Renner (University of Zurich) for generation of stable GS-NS0 hu806 cell lines and to LICR, Melbourne Centre, for the generation of GS-CHO hu806 cell lines.

Strategy for Construction, Amplification and Cloning of hu806 Antibody Genes

### Veneering and Codon Optimization

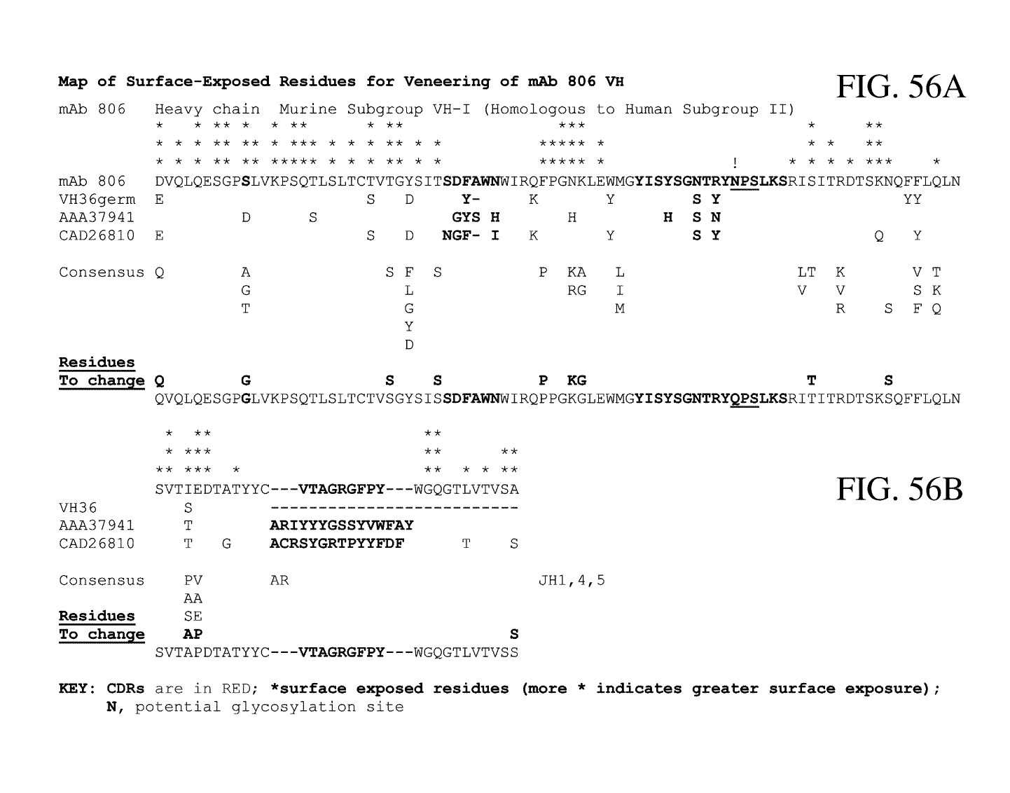

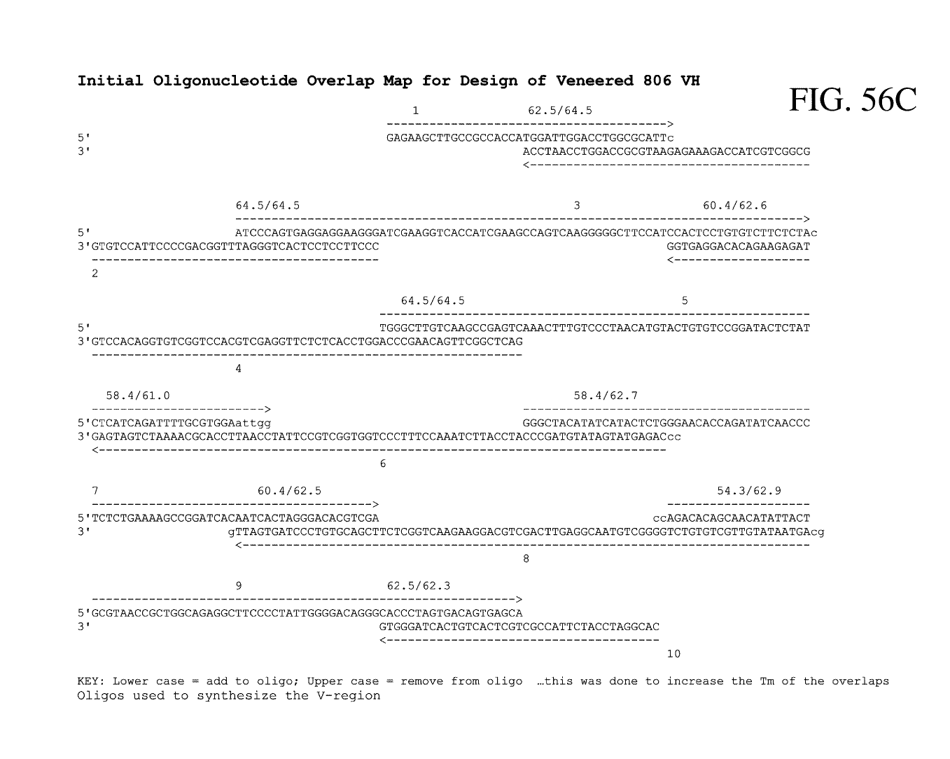

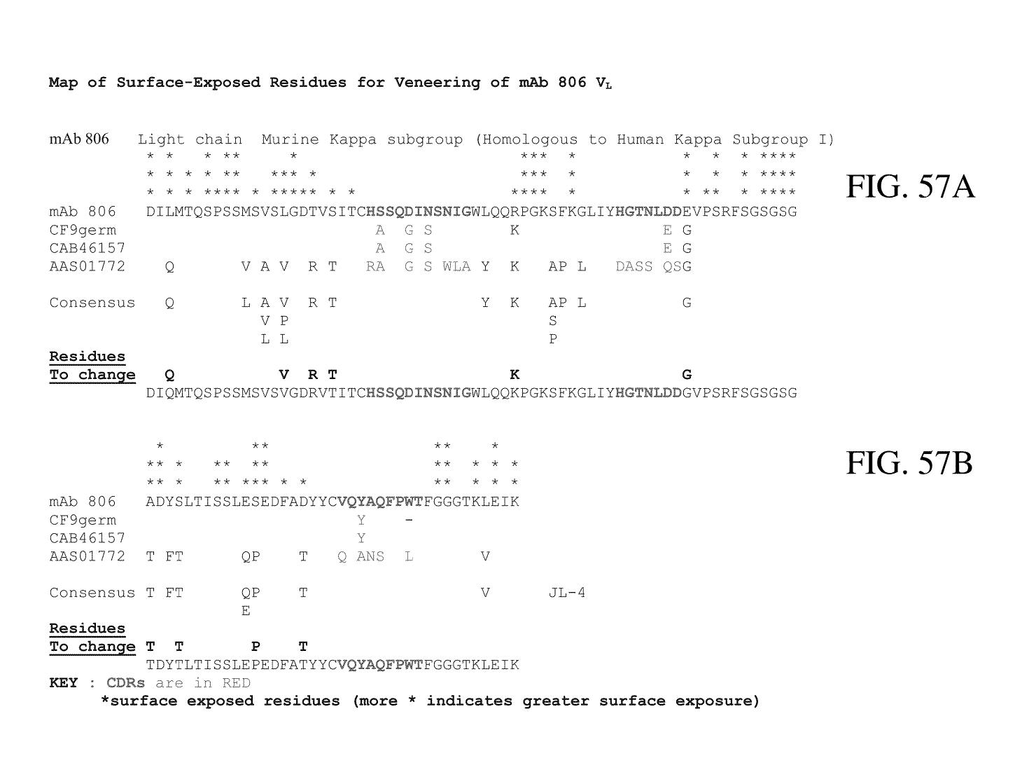

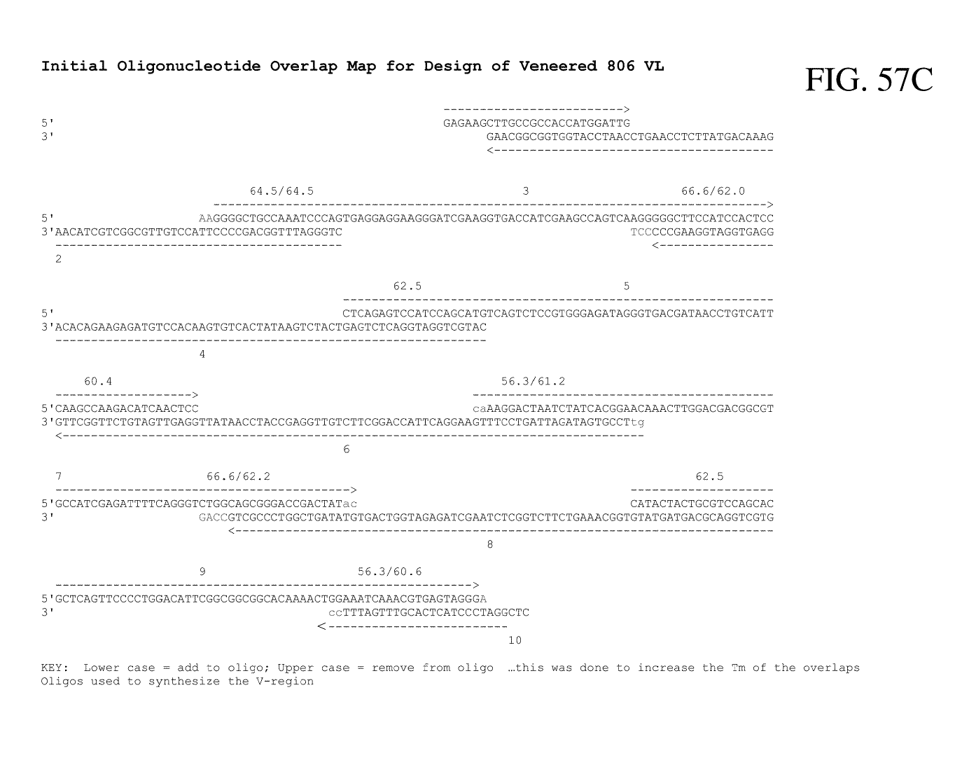

Antibody veneering is a humanization strategy aimed at countering HAMA (human anti-mouse antibody) responses. Mouse mAbs are considered “foreign” antigens by a patient's immune system and an immune response is induced, even upon a single administration, preventing further use of the reagent in those patients. In the first step of the mAb806 veneering process, the amino acid sequences of the VL and VH chains in mAb806 were analyzed, and each amino acid residue in the mAb806 protein sequence was graded for surface exposure (FIG. 56 and FIG. 57). Only those amino acids that resided on the outside of the antibody molecule were considered for possible modification, as these were the only ones that would be exposed to antibody recognition. Using BLAST, the mAb806 protein sequence was compared to three human antibody sequences (VH36 germ, CAD26810, and AAA37941). Wherever a mAb806 surface residue did not match the consensus of the human antibody sequences, that residue was identified to be changed to the consensus sequence. Initially 12 amino acids in the VL were subjected to veneering; and 14 in the VH chain of ch806 (FIG. 56 and FIG. 57).

Codon optimization is a means of improving the heterologous expression of antibodies or other proteins based on the codon bias of the system used to express these antibodies. One of the goals in the creation of hu806 was to utilize codon optimization to improve expression levels for this antibody. The expression system is based upon the LONZA GS expression system using the pEE12.4 and pEE6.4 HC and LC expression vectors as provided by LONZA Biologics and NS0 and/or CHO cells as production cells. Thus, decisions about which codon to use for a given amino acid were made with consideration for whether or not that codon would be favored in the NS0/CHO expression systems.

### Construction and Amplification of 806 DNA Sequences by PCR

The sequences for veneered, codon optimized versions of the variable heavy (VH) and variable light (VL) regions of the hu806 antibody were synthesized in the following manner: For each region (VH or VL), 8-10 oligonucleotides were designed as overlapping sense and antisense primers. These oligos would overlap each other in such a way as to cover the entire hu806 VH or VL sequence, including the signal sequence, coding sequences, introns, and include a HindIII site at the 5′ terminus and a 3′ BamHI site at the 3′ terminus The oligonucleotide maps are presented in FIGS. 56B and 57B, and the primer details are provided below.

Briefly, the hu806 VH or VL was assembled by PCR as follows: Initially v806hc- or v8061c-oligos 1, 2, 3, 4, oligos 5, 6, and oligos 7, 8, 9, 10 were combined in three separate reactions. Aliquots (50 μmol) of each flanking oligo, and 5 μmol of each internal oligo were added to a 50 μl PCR reaction containing 25 μl of 2× HotStar Taq Master Mix (Qiagen) and 48 μl of nuclease free water. The thermo cycle program was as follows: 95° C.; 15″, [94° C.; 30″, 58° C.; 30″, 72° C.; 30″]×20 cycles, 72° C.; 10″, 4° C. The products of these three reactions were excised after separation by gel electrophoresis. They were then purified using a salt column (Qiagen-Qiaspin Minipreps), and combined. These products were further amplified by PCR using primers 1 and 10. The product of this second reaction included restriction enzyme sites for HindIII and BamHI, enabling insertion into expression plasmids.

Oligonucleotides Used to PCR Synthesize the hu806 V-Regions:

| | | SEQ ID NO: |
|---|---|---|
| v806 VH: | | |
| v806hc-1: | GAGAAGCTTGCCGCCACCATGGATTGGACCTGGCGCATTC | 52 |
| v806hc-2: | CCCTTCCTCCTCACTGGGATTTGGCAGCCCCTTACCTGTGGCGGCTGCTA | 53 |
| | CCAGAAAGAGAATGCGCCAGGTCCAATCC | |
| v806hc-3: | CCCAGTGAGGAGGAAGGGATCGAAGGTCACCATCGAAGCCAGTCAAGG | 54 |
| | GGGCTTCCATCCACTCCTGTGTCTTCTCTAC | |
| v806hc-4: | GACTCGGCTTGACAAGCCCAGGTCCACTCTCTTGGAGCTGCACCTGGCT | 55 |
| | GTGGACACCTGTAGAGAAGACACAGGAGTGG | |
| v806hc-5: | GGGCTTGTCAAGCCGAGTCAAACTTTGTCCCTAACATGTACTGTGTCCG | 56 |
| | GATACTCTATCTCATCAGATTTTGCGTGGAATTGG | |
| v806hc-6: | CCCAGAGTATGATATGTAGCCCATCCATTCTAAACCTTTCCCTGGTGGCT | 57 |
| | GCCTTATCCAATTCCACGCAAAATCTGATG | |
| v806hc-7: | GGGCTACATATCATACTCTGGGAACACCAGATATCAACCCTCTCTGAAA | 58 |
| | AGCCGGATCACAATCACTAGGGACACGTCG | |
| v806hc-8: | GCAGTAATATGTTGCTGTGTCTGGGGCTGTAACGGAGTTCAGCTGCAGG | 59 |
| | AAGAACTGGCTCTTCGACGTGTCCCTAGTGATTG | |
| v806hc-9: | CCAGACACAGCAACATATTACTGCGTAACCGCTGGCAGAGGCTTCCCCT | 60 |
| | ATTGGGGACAGGGCACCCTAGTGACAGTGAGC | |
| v806hc-10: | CACGGATCCATCTTACCGCTGCTCACTGTCACTAGGGTG | 61 |
| v806 VL: | | |
| v806lc-1: | GAGAAGCTTGCCGCCACCATGGATTG | 62 |
| v806lc 2: | CTGGGATTTGGCAGCCCCTTACCTGTTGCGGCTGCTACAAGAAACAGTA | 63 |
| | TTCTCCAAGTCCAATCCATGGTGGCGGCAAG | |
| v806lc 3: | GGGGCTGCCAAATCCCAGTGAGGAGGAAGGGATCGAAGGTGACCATCG | 64 |
| | AAGCCAGTCAAGGGGGCTTCCATCCACTCC | |
| v806lc 4: | CATGCTGGATGGACTCTGAGTCATCTGAATATCACTGTGAACACCTGTA | 65 |
| | GAGAAGACACAGGAGTGGATGGAAGCCC | |
| v806lc 5: | CTCAGAGTCCATCCAGCATGTCAGTCTCCGTGGGAGATAGGGTGACGAT | 66 |
| | AACCTGTCATTCAAGCCAAGACATCAACTCC | |
| v806lc 6: | GTTCCGTGATAGATTAGTCCTTTGAAGGACTTACCAGGCTTCTGTTGGA | 67 |
| | GCCATCCAATATTGGAGTTGATGTCTTGGCTTG | |
| v806lc 7: | CAAAGGACTAATCTATCACGGAACAAACTTGGACGACGGCGTGCCATC | 68 |
| | GAGATTTTCAGGGTCTGGCAGCGGGACCGACTATAC | |
| v806lc 8: | GTGCTGGACGCAGTAGTATGTGGCAAAGTCTTCTGGCTCTAAGCTAGAG | 69 |
| | ATGGTCAGTGTATAGTCGGTCCCGCTG | |
| v806lc-9: | CATACTACTGCGTCCAGCACGCTCAGTTCCCCTGGACATTCGGCGGCGG | 70 |
| | CACAAAACTGGAAATCAAACGTGAGTAGGG | |
| v806lc 10: | CTCGGATCCCTACTCACGTTTGATTTCC | 71 |

hu806 CL:

A codon-optimized version of the constant kappa light chain (CL) was prepared in a manner similar to that used for the variable regions However, the initial PCR step involved the creation of only two preliminary products using oligos VKlcons-1, 2, 3, 4; and 5, 6, 7, 8. In addition, the flanking restriction sites for this product were BamHI and NotI prior to plasmid insertion.

Oligonucleotides used to PCR Synthesize the hu806 CL-Regions:

| | | SEQ ID NO: |
|---|---|---|
| VK1cons-1: | GACGGATCCTTCTAAACTCTGAGGGGGTCGGATGACG | 72 |
| VK1cons-2: | GGAGCTGCGACGGTTCCTGAGGAAAGAAGCAAACAGGATGGTGTTTAA | 73 |
| | GTAACAATGGCCACGTCATCCGACCCCCTC | |
| VK1cons-3: | GGAACCGTCGCAGCTCCCTCCGTGTTCATCTTCCCCCCATCCGACGAGC | 74 |
| | AACTGAAGTCAGGCACAGCCTCCGTGGTG | |
| VK1cons-4: | GTGCGTTGTCCACTTTCCACTGGACTTTGGCCTCTCTTGGGTAAAAGTTA | 75 |
| | TTAAGGAGGCACACCACGGAGGCTGTGC | |
| VK1cons-5: | GTGGAAAGTGGACAACGCACTACAGAGCGGGAACTCTCAGGAAAGCGT | 76 |
| | GACAGAGCAGGACTCAAAAGATTCAACATACAGCC | |
| VK1cons-6: | CTTCACAGGCATATACCTTGTGCTTTTCATAATCAGCTTTTGACAGTGTC | 77 |
| | AGGGTAGAAGATAGGCTGTATGTTGAATCTTTTGAGTC | |
| VK1cons-7: | GCACAAGGTATATGCCTGTGAAGTAACTCATCAGGGACTCAGCAGCCCT | 78 |
| | GTCACTAAAAGTTTTAATAGAG | |
| VK1cons-8: | CCTGCGGCCGCTTATCAGCATTCGCCTCTATTAAAACTTTTGGTGAGAG | 79 |
| | GG | |

hu806 CH:

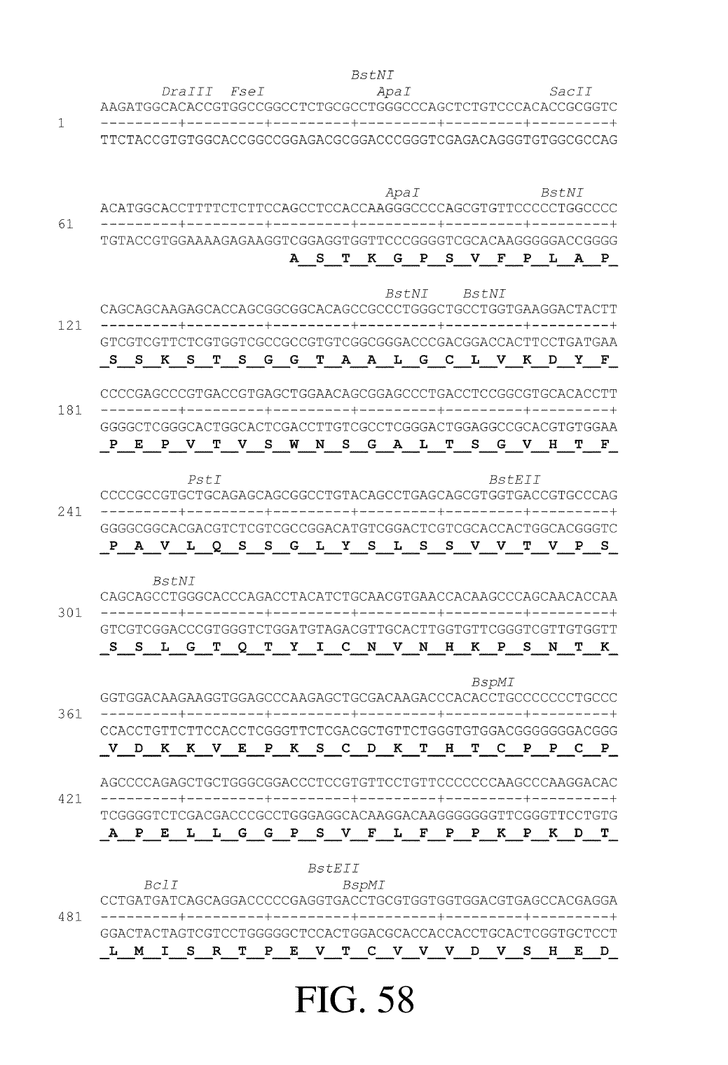

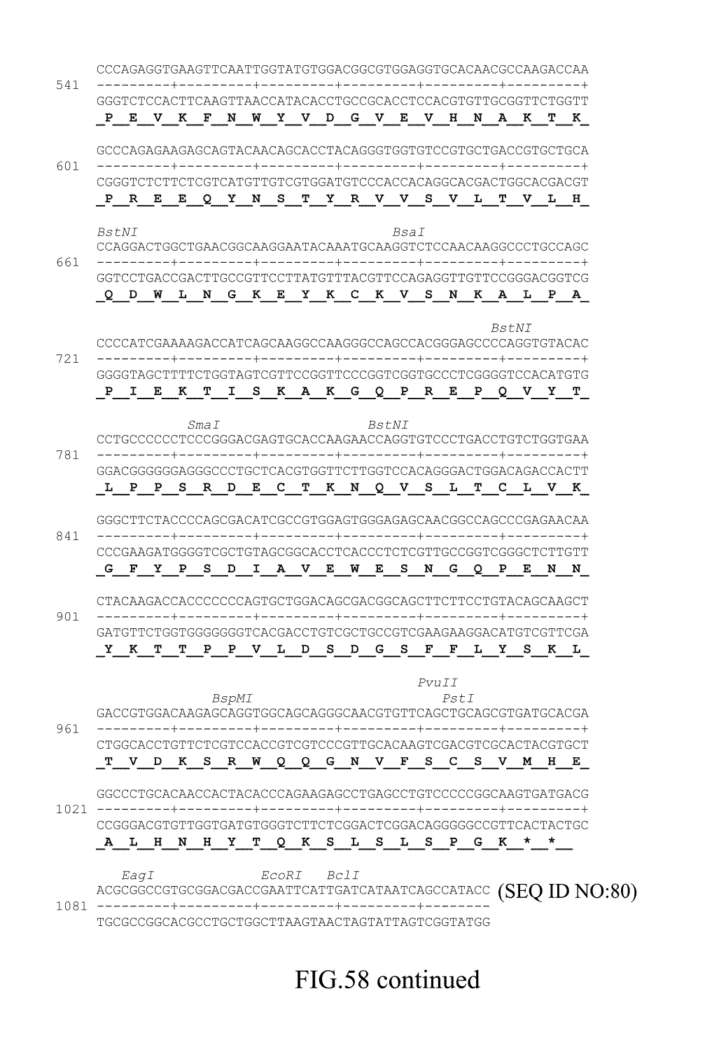

A synthetic, humanized version of the IgG1 constant heavy chain (CH) gene (SEQ ID NO:80) was purchased from GeneArt, Regensburg, Germany. The gene was codon optimized for expression in CHO/NS0 cells. Details of the gene sequence, restriction sites, etc, are shown in FIG. 58.

### Construction of Expression Plasmids

For transient transfection and preliminary testing, hu806 VH and VL sequences prepared in the manner described above were ligated into expression vectors containing generic constant regions. These vectors, provided by LICR Affiliate Christoph Renner (University of Zurich, Switzerland), were known as pEAK8 HC (which contained a generic CH), and a 33-xm-lc (which contained a generic CL). Vectors were digested using BamHI and HindIII in the presence of CIP then hu806 VH and VL were ligated into the corresponding vectors. The resulting plasmids were used to transform Top10 chemically competent E. coli (Invitrogen) according to the manufacturer's directions. Transformed E. Coli were plated on LB+Ampicillin plates, and resistant clones were screened by restriction digestion and PCR. In general, eight positive clones detected in this manner would be isolated and further amplified. DNA purified from these colonies were analyzed by automated DNA sequencing.

Codon-optimized versions of the constant regions were added to these constructs by restriction enzyme-digestion and ligation using BamHI and NotI. These transformants were selected, sequenced, and analyzed as stated above. Prior to the full-length antibody chains being ligated into the Lonza GS system the BamHI site between the variable and constant region sequences was destroyed, in one case, by digestion using BamHI, fill-in using DNA Polymerase, and blunt-end ligation.

Restriction fragments containing hu806 (VH+CH) or hu806 (VL+CL) were then digested with NotI followed by HindIII. These digestions were designed to create a blunt end at the NotI site, and thus were done in series in the following manner: The plasmid was first digested with NotI. Fully digested (single-cut) plasmid was separated by electrophoresis using a 1% agarose gel. This product was then excised and purified on a salt column and filled-in using DNA Polymerase. The product of this reaction was salt-column purified and then digested with HindIII. This product (˜1.3 Kb for hu806 (VH+CH), and ˜0.8 Kb for hu806 (VL+CL) was then separated by gel electrophoresis, excised, and purified.

Vectors pEE12.4 and pEE6.4 (Lonza Biologics plc, Slough, UK) were each digested on HindIII and PmlI. hu806 (VH+CH) was ligated to pEE12.4 to create pEE12.4-hu806H, and hu806 (VL+CL) was ligated to pEE6.4 to create pEE6.4-hu806L.

After screening, a combined, double gene Lonza plasmid was created to contain both the hu806 heavy and light chain sequences. Briefly, the pEE12.4-hu806H and pEE6.4-hu806L vectors were digested with NotI and SalI restriction enzymes. The resultant fragments, which contained the GS transcription unit and hCMV-MIE promoter, followed by the hu806 Heavy or Light chain expression cassette, were isolated and ligated together. The resulting “combined” Lonza plasmid (Designated 8C65AAG) was used for single-plasmid transient transfections in a HEK 293 system and stable transfections in NS0 and CHO systems. A plasmid map is shown in FIG. 53.

### Modifications to Constructs

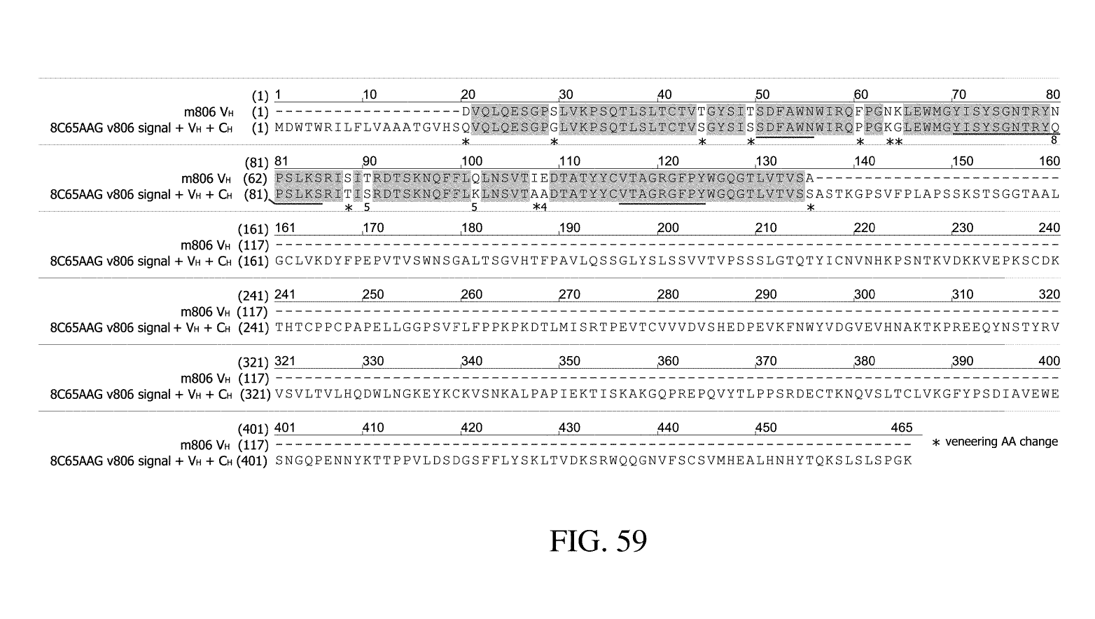

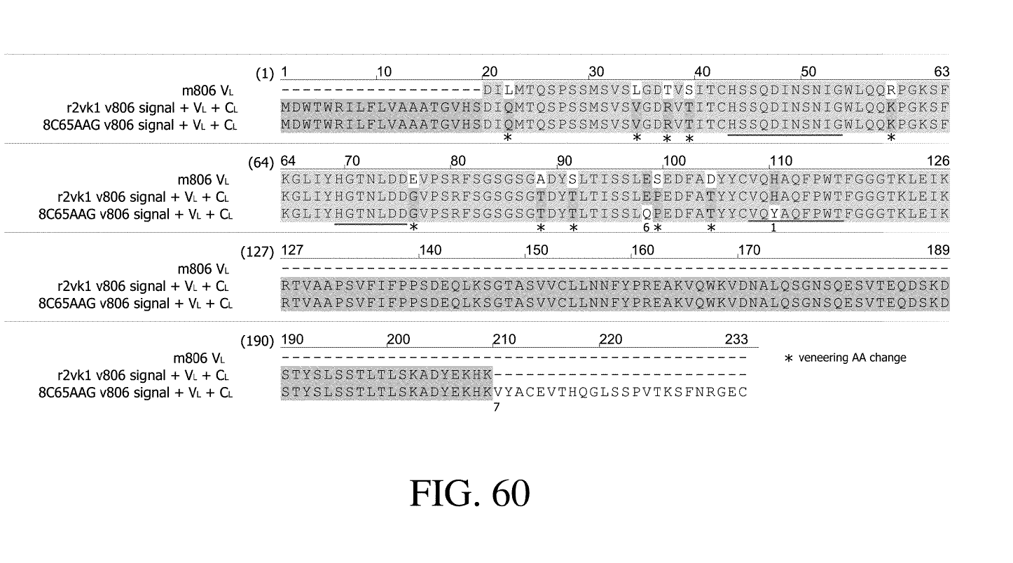

The complete sequence verified amino acid sequences of the veneered hu806 Hc and hu806 Lc are shown in comparison to mAb806 in FIG. 59 and FIG. 60, respectively. Flanking the hu806 sequence within the appendices are asterisks (*) indicating initial veneering changes and numbers (1-8) refer to the numbered modifications No. 1 to No. 8 described herein.

With regard to FIG. 60, the reference file (mAb806 LC) incorrectly indicates Histidine (H), not the correct Tyrosine (Y) at position 91; the subject of modification #1. The original, uncorrected file sequence is included in FIG. 60, to illustrate the necessary modification made to hu806 at position 91.

A number of modifications were made to the hu806 cDNA sequences after the initial construction and sequencing phase. The reasons for making these modifications included: introduction of 4 restriction enzyme sites for sequence modification purposes, to correct 2 amino acid errors in the sequence introduced during PCR, to correct one amino acid error arising from the initial mAb806 documentation, and to engineer 4 additional amino acid changes to effect additional veneering variants. The following 8 stages of modifications were performed:

1. hu806 VL: CDR3 H91Y

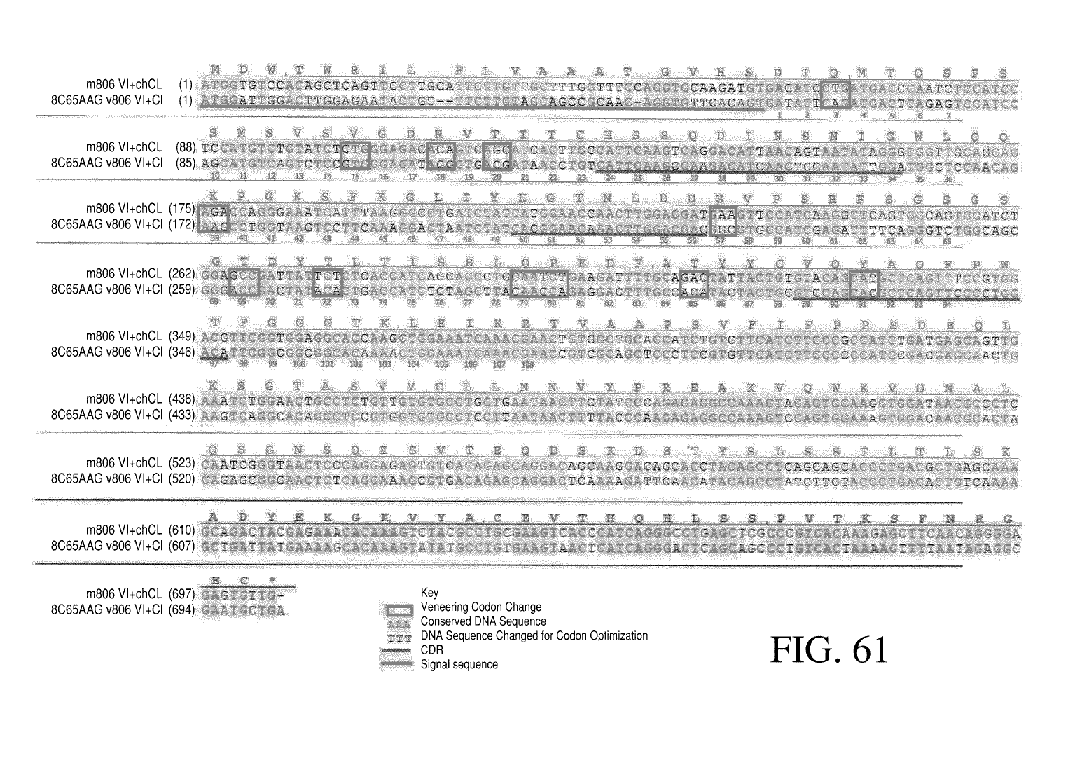

The document from which the original oligonucleotides were created incorrectly stated that there was a CAC (Histidine, H) at position 91 in the CDR3 of the mAb806 VL sequence. Site-directed mutagenesis was used to generate the correct sequence of TAC (Tyrosine, Y; Patent WO02/092771). The consequent change in the amino acid sequence at this position was from CVQHAQF (SEQ ID NO:84) to CVQYAQF (SEQ ID NO:85). The final DNA and translated protein sequence in comparison to ch806 are shown in FIG. 61.

Sense primer for the histidine to tyrosine modification of the hu806 VL region (PDV1; 40mer)

(SEQ ID NO: 86)
5′- CCACATACTACTGCGTCCAGTACGCTCAGTTCCCCTGGAC -3′

Antisense primer for the histidine to tyrosine modification of the hu806 VL region (PDV2; 20mer)

| | |
|---|---|
| 5′- CTGGACGCAGTAGTATGTGG -3′ | (SEQ ID NO: 87) |

2. hu806 Heavy Chain: Addition of Restriction Sites DraIII and FseI

Restriction enzyme sites were added to the introns surrounding the hu806 VH and VL regions. These restriction sites (unique in the pREN vector system, LICR) were designed to ease the process of making modifications to the expression cassettes. The hu806 VH sequence, not including the initial signal region, could be removed or inserted by single-digestion on DraIII. In addition, FseI could be used, in concert with NotI (pREN system) or EcoRI (Lonza System) to cut out the constant region, fulfilling the function of BamHI from the original sequence.

These modifications were achieved using a two-step PCR process. The products were then digested with HindIII and BglII. They were then ligated into pREN vectors containing codon-optimized constant regions, which had been digested on HindIII and BamHI. This re-ligation process destroyed the BamHI site.

Sense primer for variable region upstream of first DraIII site (806 heavy chain DraIII Up; 26mer)

(SEQ ID NO: 88)
5′- GAGAAGCTTGCCGCCACCATGGATTG -3′

Antisense primer incorporating DraIII site I (806 heavy chain DraIII Down; 28mer)

(SEQ ID NO: 89)
5′- CACTGGGTGACTGGCTTCGATGGTGACC -3′

Sense primer for the HC variable region between the two DraIII sites (806 heavy chain DraIII-FseI Up; 49mer)

(SEQ ID NO: 90)
5′- GGTCACCATCGAAGCCAGTCACCCAGTGAAGGGGGCTTCCATCCAC
TCC -3′

Antisense primer incorporating the DraIII site II, and the FseI site (806 heavy chain DraIII-FseI Down; 44mer)

(SEQ ID NO: 91)
5′- CCAAGATCTGGCCGGCCACGGTGTGCCATCTTACCGCTGCTCAC -
3′

3. hu806 Light Chain: Addition of Restriction Sites RsrII and PacI

For the hu806 light chain, the restriction sites added were RsrII, having the same function as DraIII in the heavy chain, and PacI, which matched the function of FseI.

Sense primer for variable region upstream of first RsrII site (806 light chain RsrII Up; 22mer)

| | |
|---|---|
| 5′- GAGAAGCTTGCCGCCACCATGG -3′ | (SEQ ID NO: 92) |

Antisense primer incorporating RsrII site I (806 light chain RsrII Down; 25mer)

| | |
|---|---|
| 5′- CGGTCCGCCCCCTTGACTGGCTTCG -3′ | (SEQ ID NO: 93) |

Sense primer for the LC variable region between the two RsrII sites (806 light chain RsrII-PacI Up; 45mer)

(SEQ ID NO: 94)
5′- CGAAGCCAGTCAAGGGGGCGGACCGCTTCCATCCACTCCTGTGT
C -3′

Antisense primer incorporating the RsrII site II, and the PacI site (806 light chain RsrII-PacI Down: 50mer)

(SEQ ID NO: 95)
5′- CCAAGATCTTTAATTAACGGACCGCTACTCACGTTTGATTTCCAGT
TTTG -3′

4. hu806 VH: Reveneering P85A

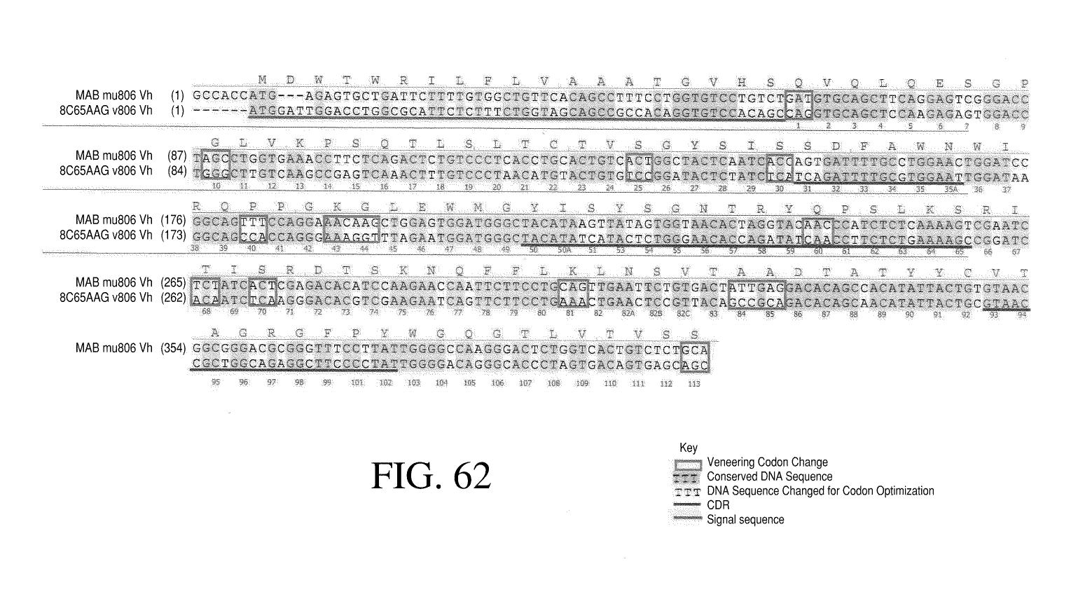

The protein sequence for the parental mAb806 at VH amino acids 81-87 is SVTIEDT (SEQ ID NO:96). As part of the veneering process, isoleucine and glutamic acid at positions 84 and 85 were changed to alanine-proline to read SVTAPDT (SEQ ID NO:97; FIG. 56). Upon further analysis, it was decided that alanine might have been a better choice than proline in this case. Site-directed mutagenesis was used to generate this secondary change (SVTAADT, SEQ ID NO:98) using the primers listed below. Final DNA and translated protein sequences are presented in FIG. 62.

Sense primer (Fx3; 49mer)

(SEQ ID NO: 99)
5′- CTGCAGCTGAACTCCGTTACAGCCGCAGACACAGCAACATATTACT
GCG -3′

Antisense primer (Fx4; 49mer)

(SEQ ID NO: 100)
5′- CGCAGTAATATGTTGCTGTGTCTGCGGCTGTAACGGAGTTCAGCTG
CAG -3′

5. hu806 VH: Additional Veneering

The hu806 heavy chain variable region sequence underwent three further mutations following the initial veneering: T70S, S76N and Q81K. The change at position 76 from serine to asparagine represented a correction back to the original sequence of mAb806 molecule. The additional changes in the framework were included because they represent residues that are not found in mouse antibodies but are found in human antibodies. Accordingly, the protein sequence TRDTSKSQFFLQ (SEQ ID NO:101) was veneered to SRDTSKNQFFLK (SEQ ID NO:102). Final DNA and translated protein sequences in comparison to mAb806 are presented in FIG. 62.

Sense Primer for HC variable region 5′ PCR fragment (hu806HCfx2-5p-U; 49mer)

(SEQ ID NO: 103)
5′- GGTCACCATCGAAGCCAGTCACCCAGTGAAGGGGGCTTCCATCCAC
TCC -3′

Antisense Primer for 5′ PCR fragment, incorporates first two changes (hu806HCfx2-5p-D; 45mer)

(SEQ ID NO: 104)
5′- GATTCTTCGACGTGTCCCTTGAGATTGTGATCCGGCTTTTCAGA
G -3′

Sense Primer for 3′ PCR fragment, incorporates all changes (hu806HCfx2-3p-U; 55mer)

(SEQ ID NO: 105)
5′- CAAGGGACACGTCGAAGAATCAGTTCTTCCTGAAACTGAACTCCGT
TACAGCCGC -3′

Antisense Primer for HC variable region 3′ PCR fragment (hu806HCfx2-3p-D; 44mer)

(SEQ ID NO: 106)
5′- CCAAGATCTGGCCGGCCACGGTGTGCCATCTTACCGCTGCTCAC -
3′

6. hu806 VL: E79Q Veneering

This was the only post-construction VL veneering modification performed. At position 79 site directed mutagenesis was employed to correct the sequence SSLEPE (SEQ ID NO:107) to SSLQPE (SEQ ID NO:108). Final DNA and translated protein sequences in comparison to ch806 are presented in FIG. 61.

Sense Primer for LC variable region 5′ PCR fragment (hu806LC-5p-U; 45mer)

(SEQ ID NO: 109)
5′- CGAAGCCAGTCAAGGGGGCGGACCGCTTCCATCCACTCCTGTGT
C -3′

Antisense Primer for 5′ PCR fragment, incorporates intended mutation (hu806LC-5p-D; 34mer)

(SEQ ID NO: 110)
5′- CTCTGGTTGTAAGCTAGAGATGGTCAGTGTATAG -3′

Sense Prime for LC variable region 3′ PCR fragment incorporates intended mutation (hu806LC-3p-U; 45mer)

(SEQ ID NO: 111)
5′- CCATCTCTAGCTTACAACCAGAGGACTTTGCCACATACTACTGC
G -3′

Antisense Primer for LC variable region 3′ PCR fragment (hu806LC-3p-D; 50mer)

(SEQ ID NO: 112)
5′-CCAAGATCTTTAATTAACGGACCGCTACTCACGTTTGATTTCCAG
TTTTG-3′

7. hu806 Light Chain: Kappa Constant Region Splice-Junction Modification

This point mutation was required to correct an error in the splicing of the codon-optimized version of the kappa constant region. Prior to this change, the portion of the amino acid chain beginning with VYACEVTH (SEQ ID NO:113) and continuing to the end of the molecule would not have been included in the final antibody (FIG. 60).

Sense primer for LC constant kappa 5′ PCR fragment (F1: 21merl

| | |
|---|---|
| 5′-GGCGGCACAAAACTGGAAATC-3′ | (SEQ ID NO: 114) |

Antisense primer for LC constant kappa 5′ PCR fragment, incorporates correction (F2; 59mer)

(SEQ ID NO: 115)
5′-GATGAGTTACTTCACAGGCATATACTTTGTGCTTTTCATAATCAG
CTTTTGACAGTGTC-3′

Sense primer for LC constant kappa 3′ PCR fragment, incorporates correction (F3; 26mer)

| | |
|---|---|
| 5′-AGTATATGCCTGTGAAGTAACTCATC-3′ | (SEQ ID NO: 116) |

Antisense primer for LC constant kappa 3′ PCR fragment. (F4; 17mer)

| | |
|---|---|
| 5′-GCCACGATGCGTCCGGC-3′ | (SEQ ID NO: 117) |

8. hu806 VH:N60Q

In addition to the veneering changes made to antibody 806 in the initial stages of construction, Asparagine at position 60 in VH CDR2 was changed to Glutamine at this time. N-Glycosylation follows the scheme: N X S/T, where X is any amino acid. The amino acid sequence from position 60 was N P S, which follows this scheme. However, it is infrequently the case that proline (as in our example) or cysteine is found at the X position for N-glycosylation. It was of concern that inconsistent glycosylation could lead to variations in the reactivity of the antibody. Thus, asparagine was removed, and replaced with its most closely related amino acid, glutamine, removing any potential for this site to be glycosylated (FIG. 59 and FIG. 62).

Binding of Veneered hu806 Antibody 8C65AAG Construct

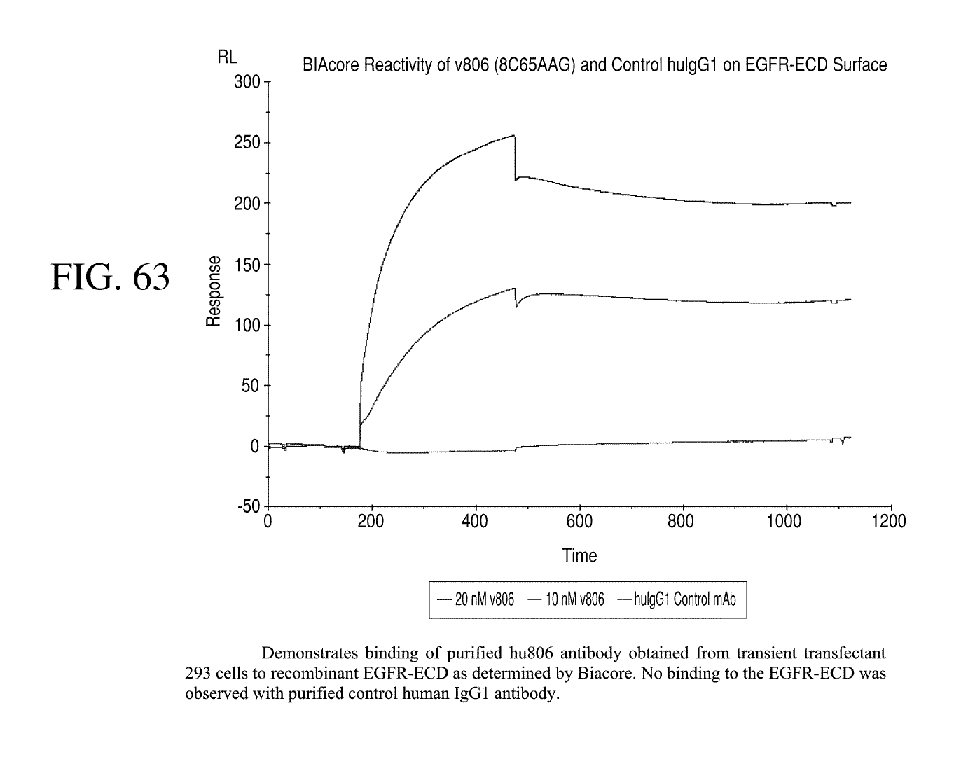

Transient transfection of 293FT cells with the final plasmid 8C65AAG was performed to enable the preparation of small quantities of hu806 for initial antigen binding verification. Culture supernatants from several small-scale replicate transient transfections were pooled, concentrated and hu806 antibody was collected using a protein-A chromatography step. Approximately 1-2 μg of hu806 antibody was obtained as measured by a quantitative huIgG1 ELISA and the antibody was analyzed by Biacore for binding to recombinant EGFR-ECD (FIG. 63). Bovine immunoglobulin from the cell culture medium co-purified with hu806 and represented the major fraction of total IgG, limiting quantitative assessment of hu806 binding.

### Sequencing Primers

RenVecUPSTREAM: Sense primer, begins sequencing upstream of variable region in peak8, and a33xm vectors.

| | |
|---|---|
| 5′-GCACTTGATGTAATTCTCCTTGG-3′ | (SEQ ID NO: 118) |

RenVecDwnstrmHC: Antisense primer begins sequencing downstream of variable region on peak8 heavy-chain plasmid. Anneals within non-codon-optimized HC constant region.

| | |
|---|---|
| 5′-GAAGTAGTCCTTGACCAGG-3′ | (SEQ ID NO: 119) |

RenVecDwnstrmLC: Antisense primer, begins sequencing downstream of variable region on a33-xm-lc light-chain plasmid. Anneals within non-codon-optimized LC constant region.

| | |
|---|---|
| 5′-GAAGATGAAGACAGATGGTGCAG-3′ | (SEQ ID NO: 120) |

Upstrm Lonza: Sense primer, begins sequencing upstream of variable region in Lonza vectors pEE 12.4 and pEE 6.4. Cannot be used with combined Lonza because this is a duplicate region in the combined plasmid.

| | |
|---|---|
| 5′-CGGTGGAGGGCAGTGTAGTC-3′ | (SEQ ID NO: 121) |

Dnstrm 6-4: Antisense primer, begins sequencing downstream of constant region in Lonza vector pEE 6.4

| | |
|---|---|
| 5′-GTGATGCTATTGCTTTATTTG-3′ | (SEQ ID NO: 122) |

Dnstrm 12-4: Antisense primer, begins sequencing downstream of constant region in Lonza vector pEE12.4

| | |
|---|---|
| 5′-CATACCTACCAGTTCTGCGCC-3′ | (SEQ ID NO: 123) |

Cod-Opt LC const E: Sense primer, internal to the codon-optimized light-chain v-kappa constant region

| | |
|---|---|
| 5′-CCATCCTGTTTGCTTCTTTCC-3′ | (SEQ ID NO: 124) |

Cod-Opt LC const F: Antisense primer, internal to the codon-optimized light-chain v-kappa constant region (vk).

| | |
|---|---|
| 5′-GACAGGGCTGCTGAGTC-3′ | (SEQ ID NO: 125) |

806HCspec: Sense primer, internal and unique to the veneered version of the 806 HC variable region.

| | |
|---|---|
| 5′-GTGCAGCTCCAAGAGAGTGGAC-3′ | (SEQ ID NO: 126) |

806LCspec: Sense primer, internal and unique to the veneered version of the 806 LC variable region.

| | |
|---|---|
| 5′-CAGAGTCCATCCAGCATGTC-3′ | (SEQ ID NO: 127) |

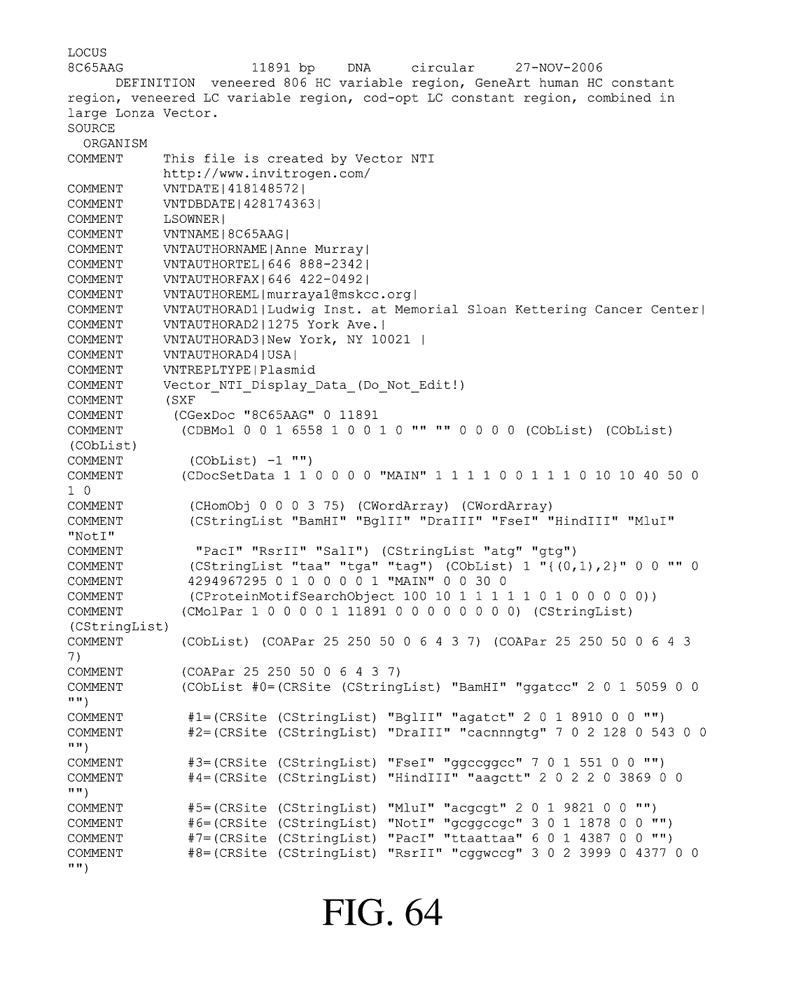

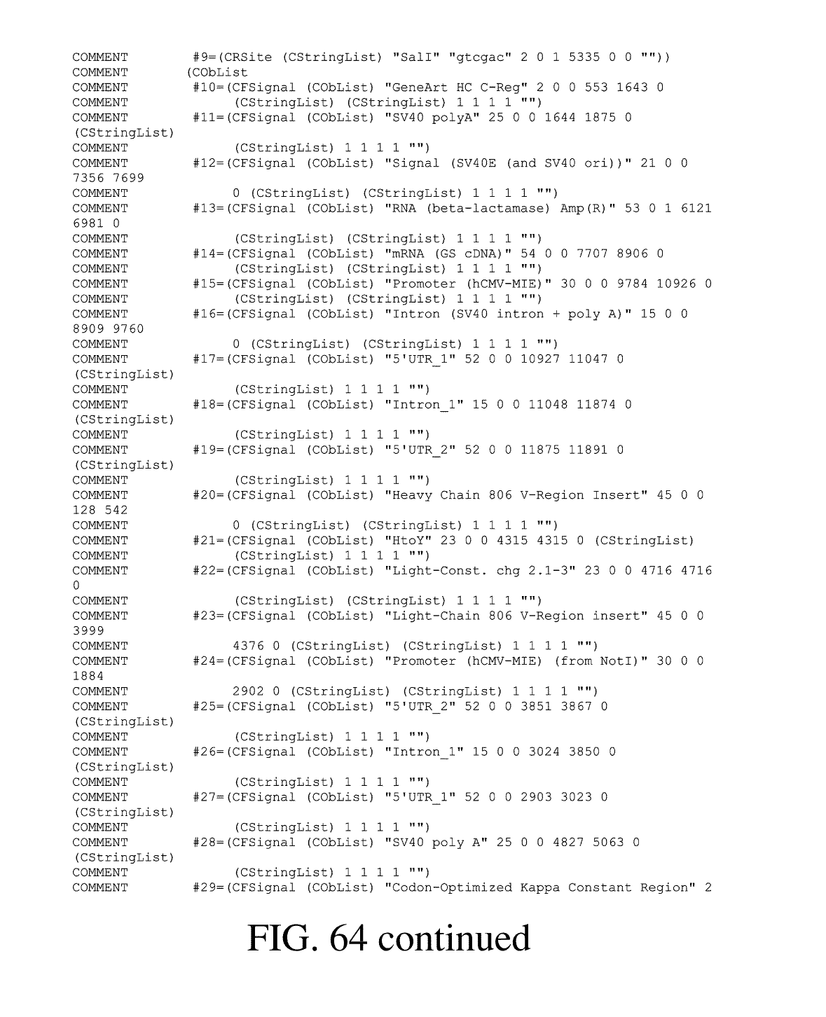

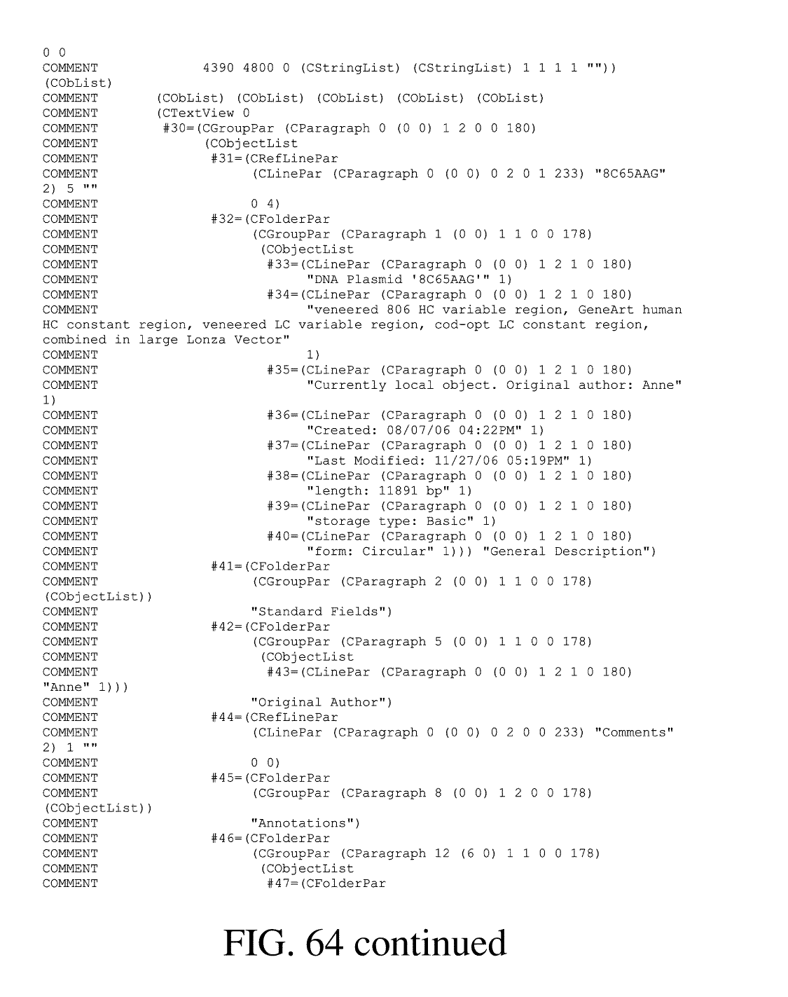

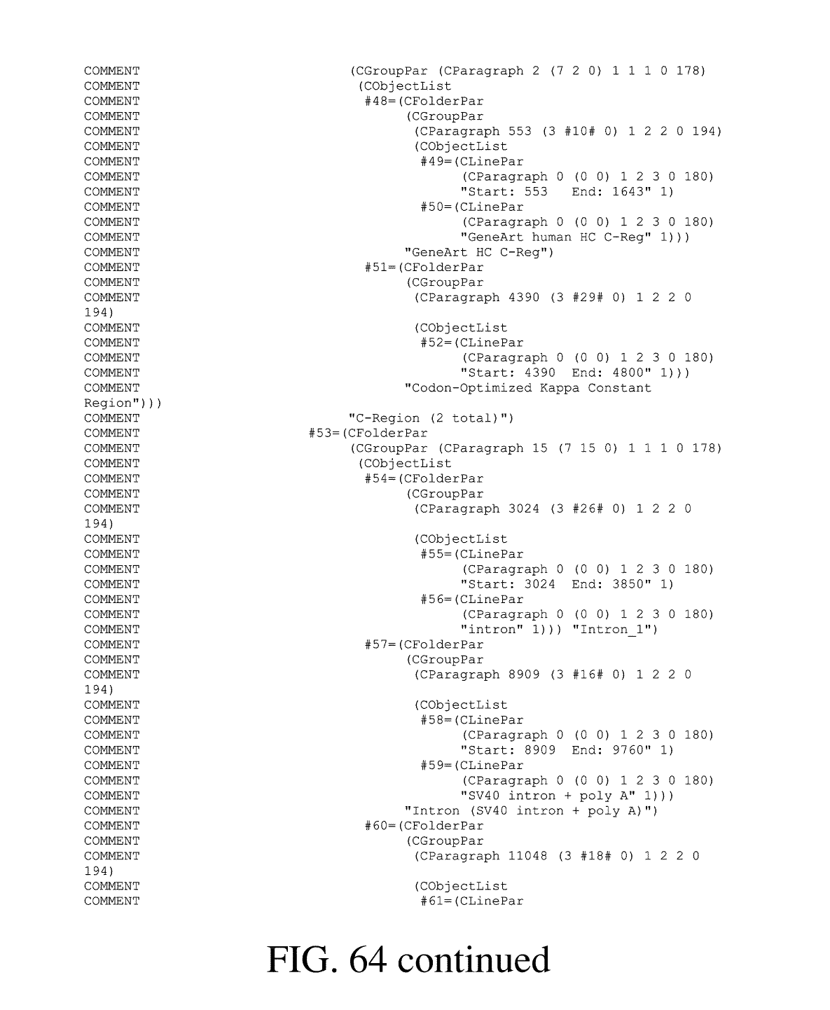

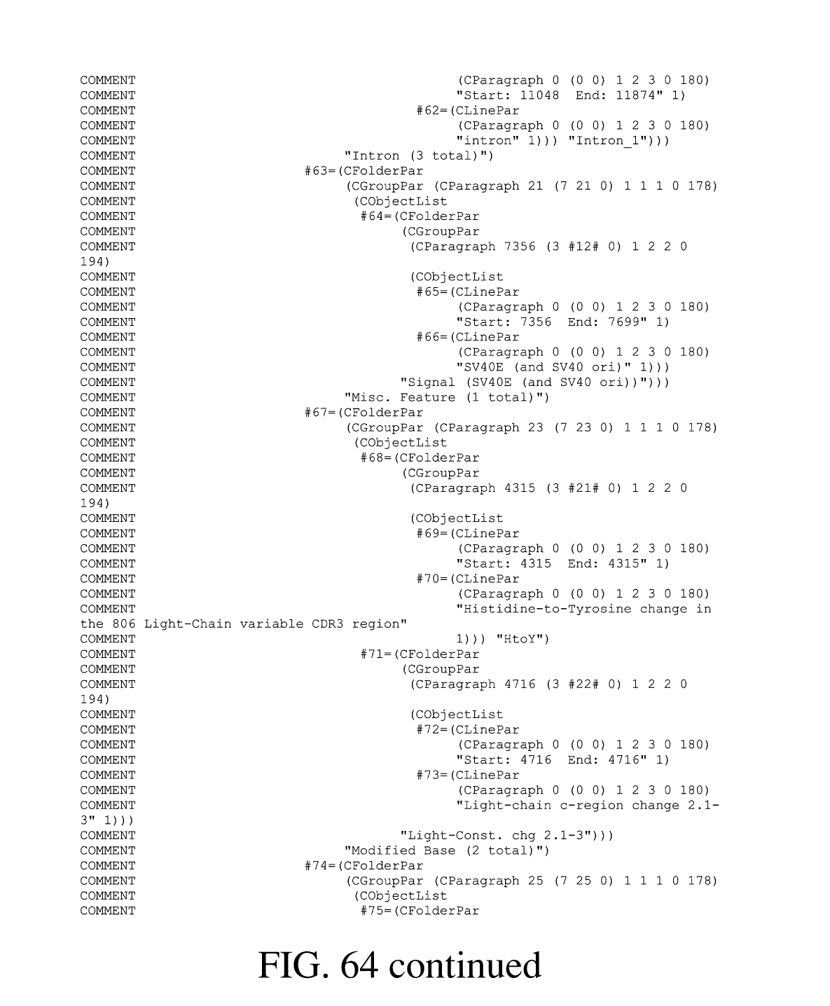

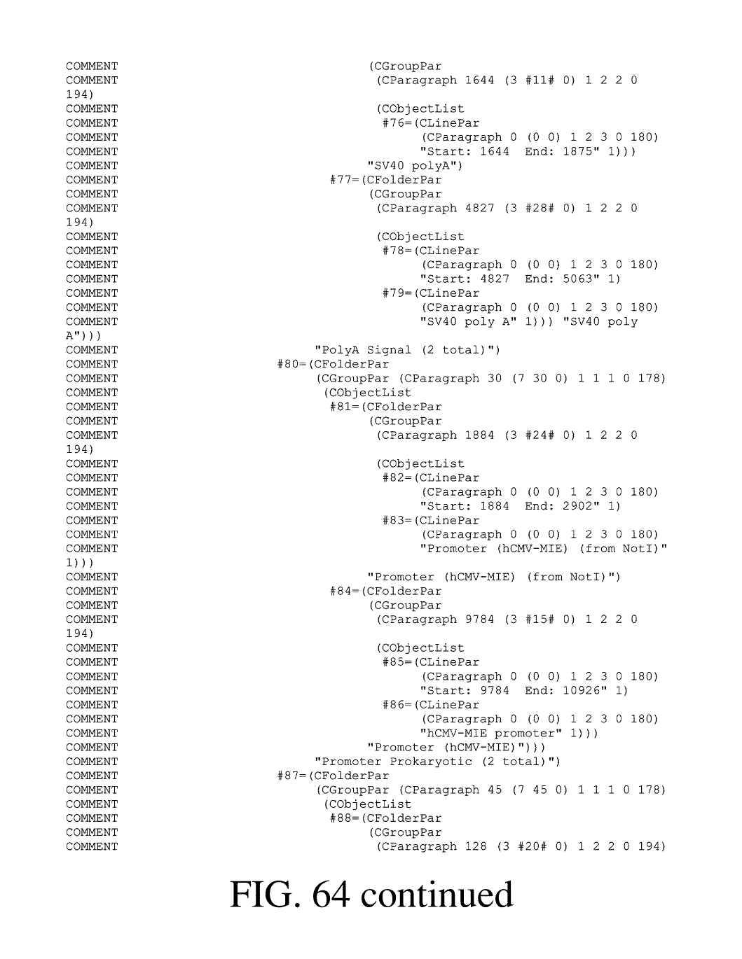

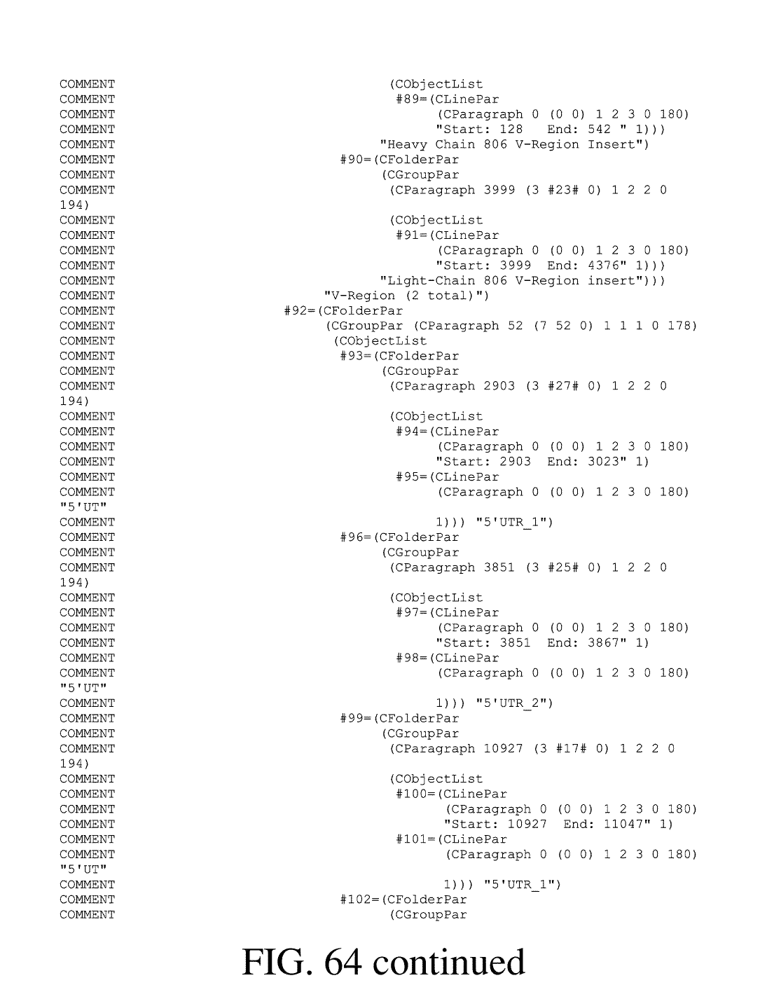

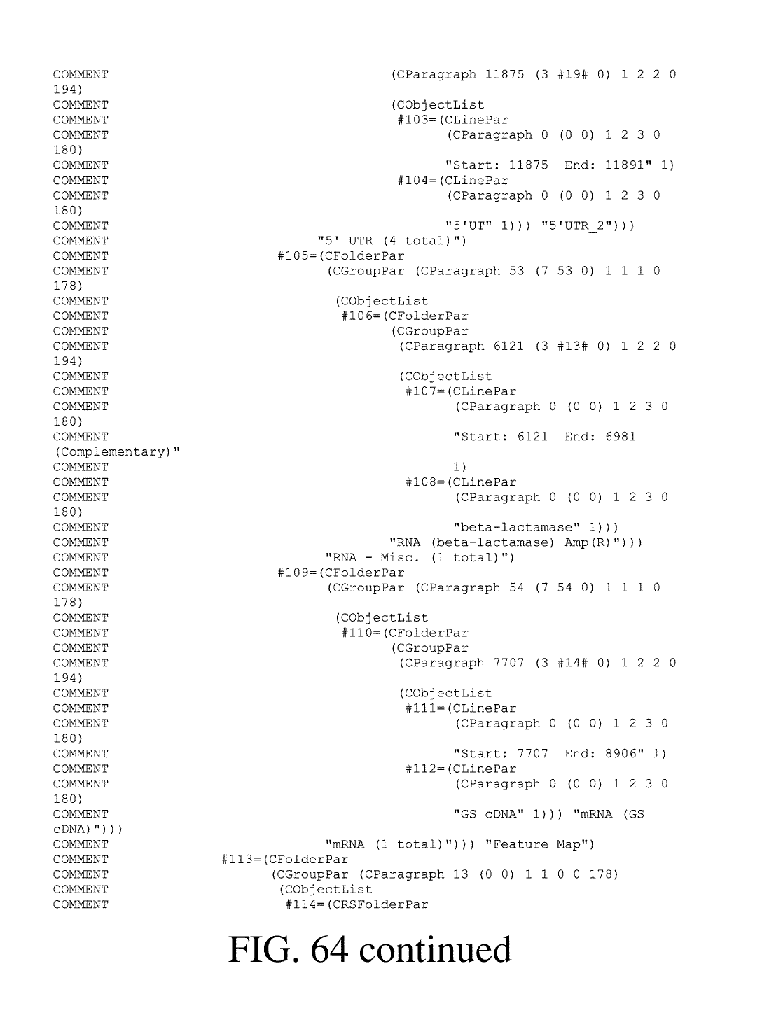

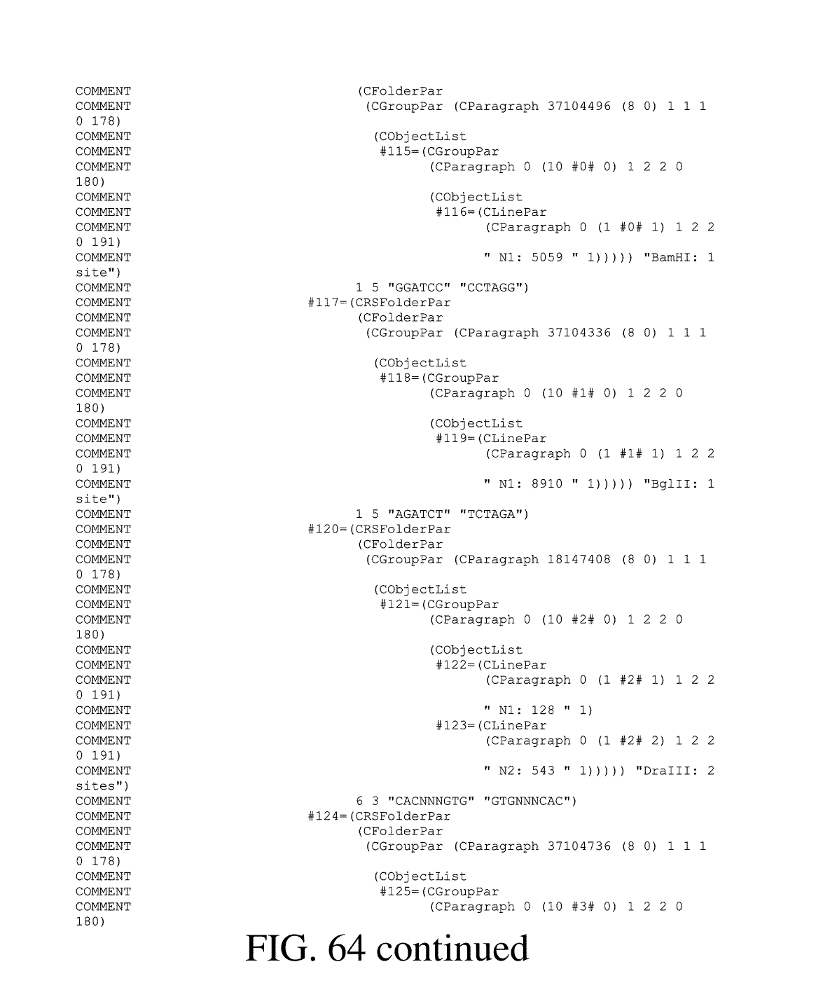

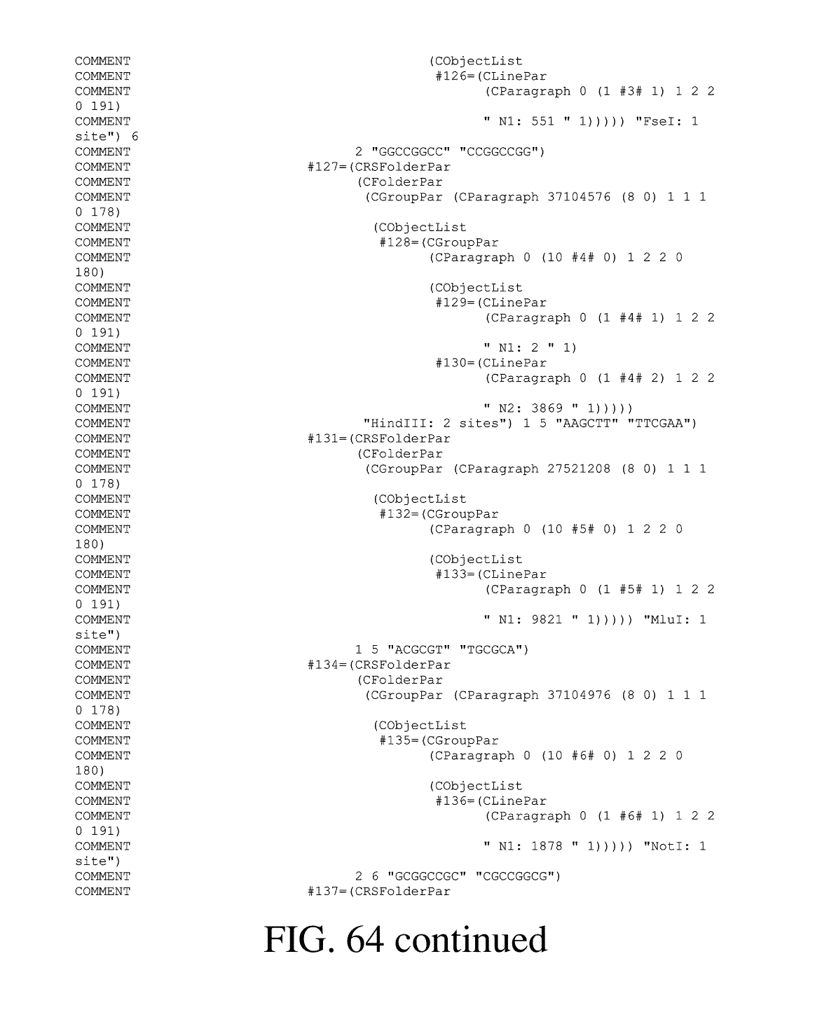

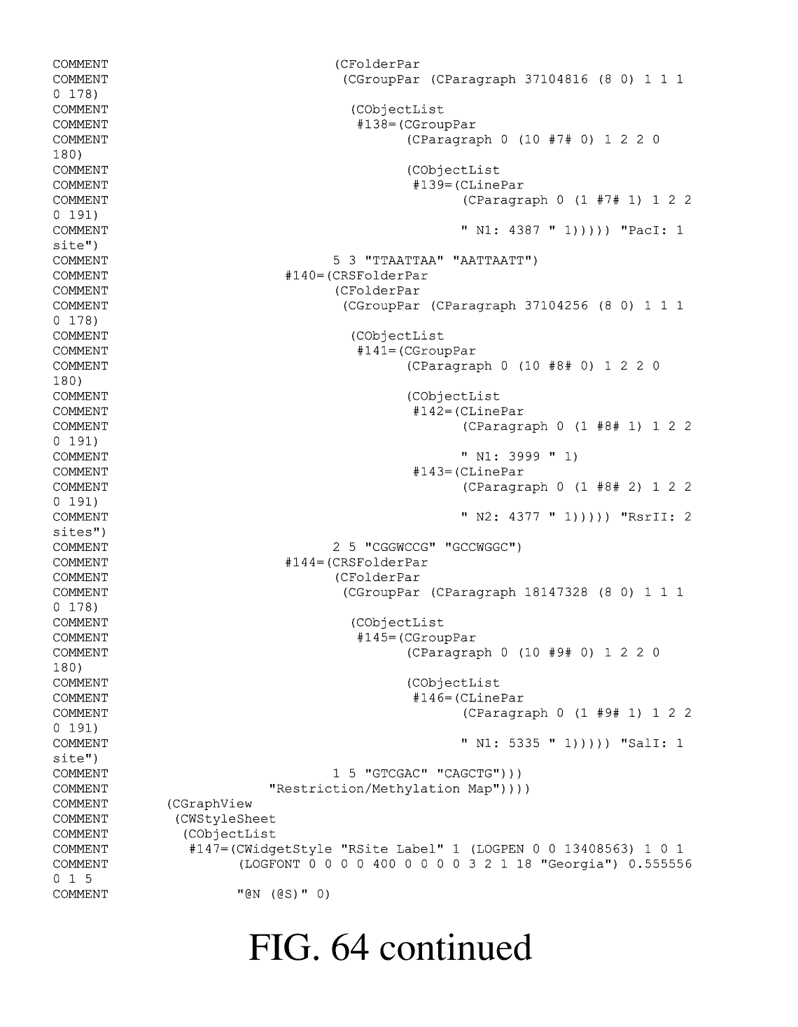

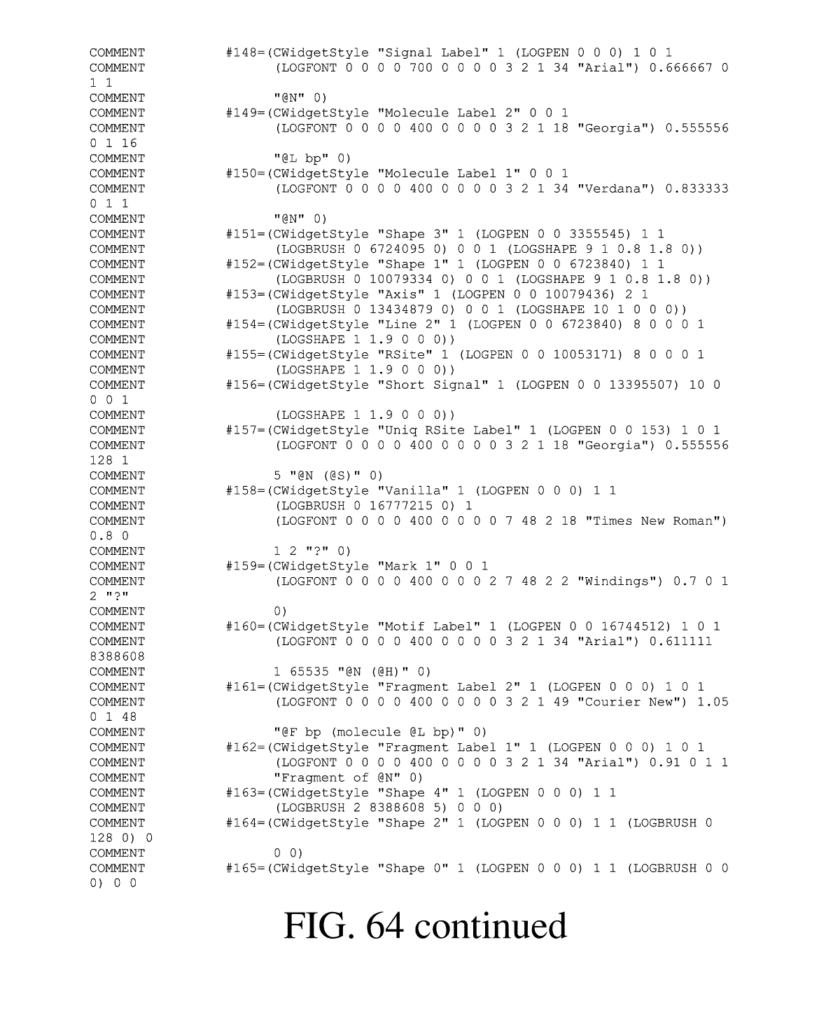

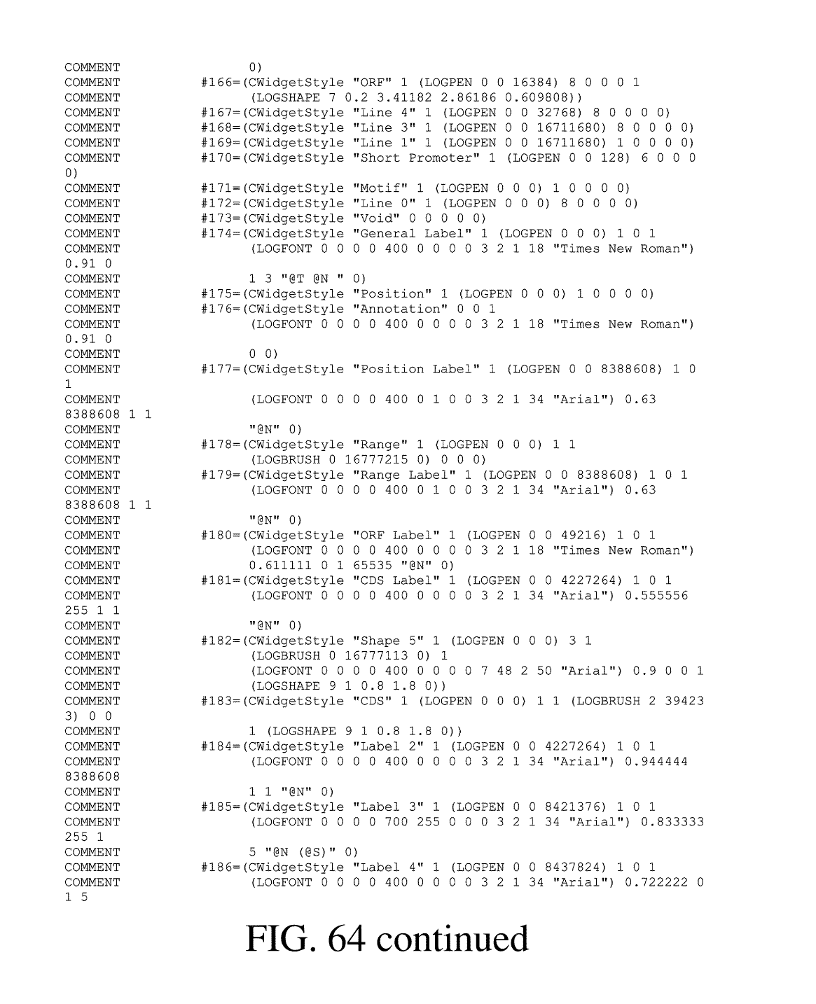

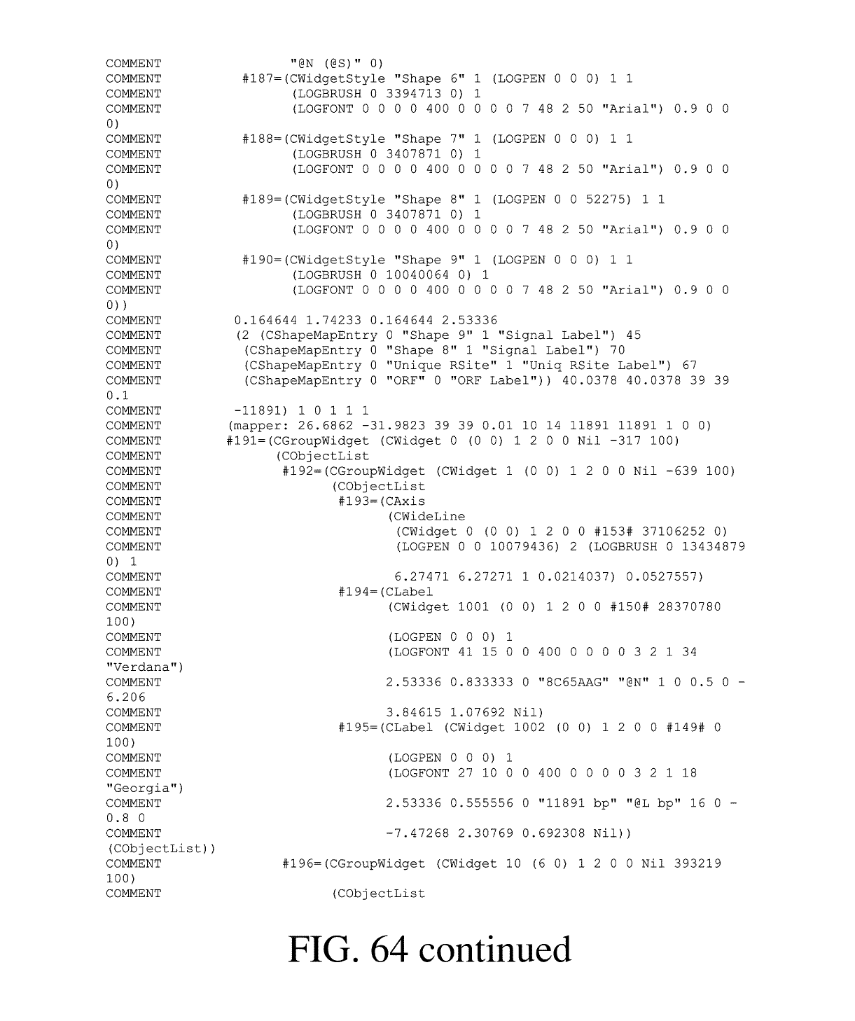

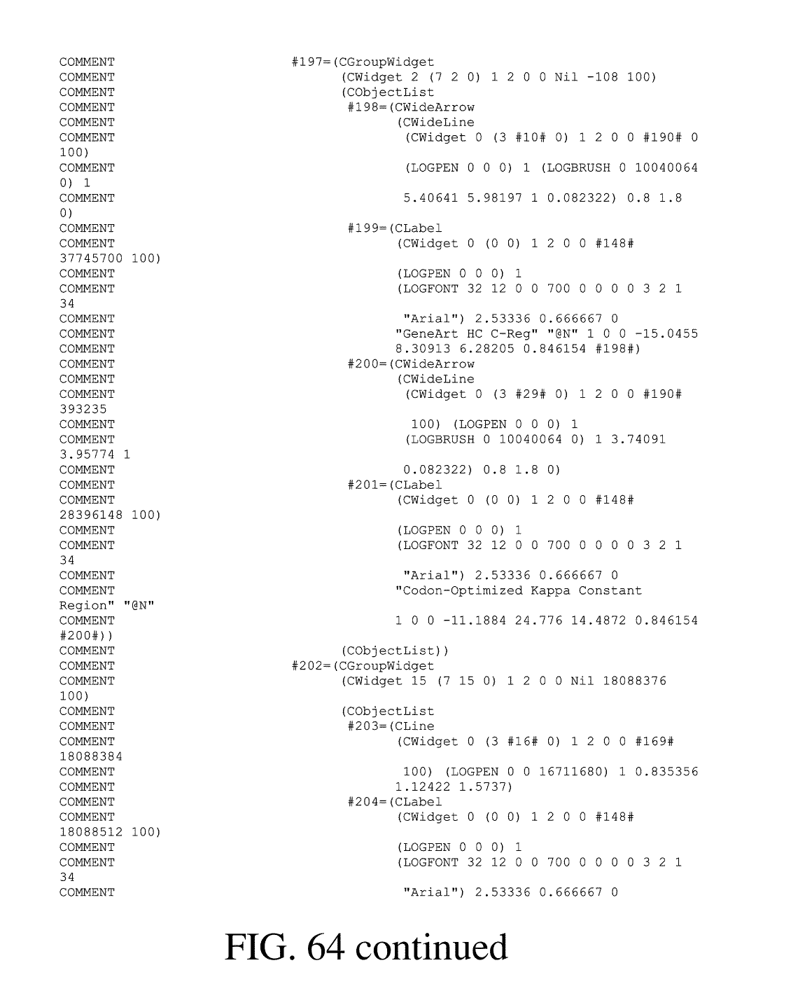

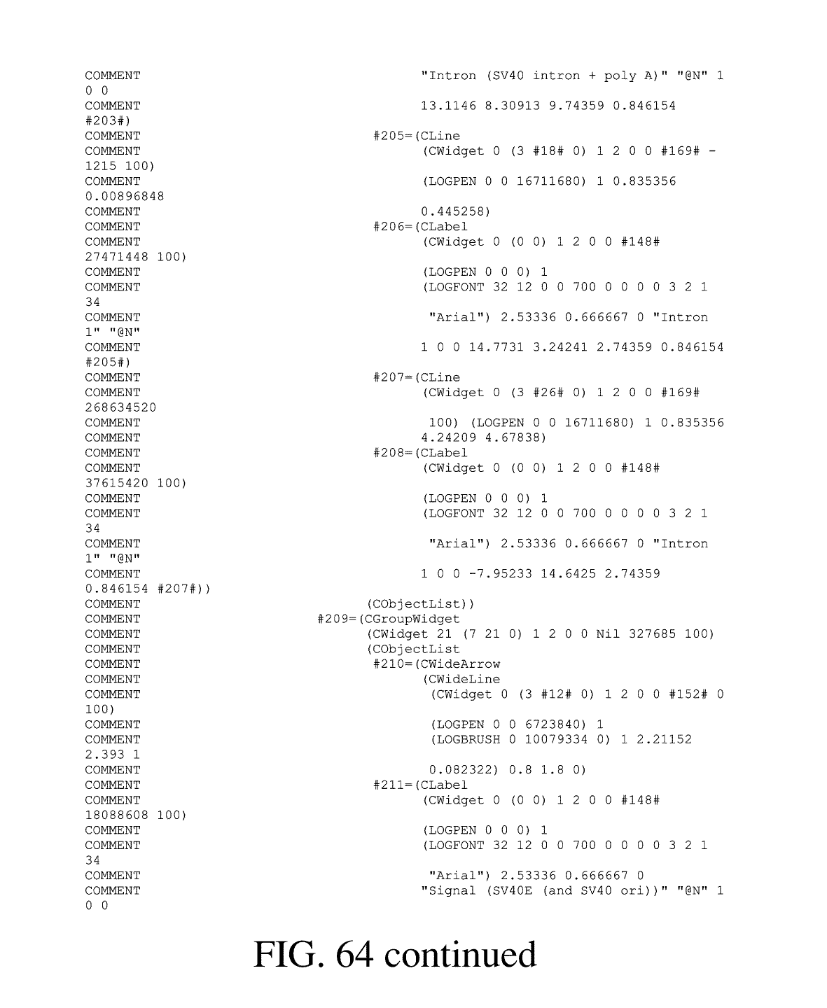

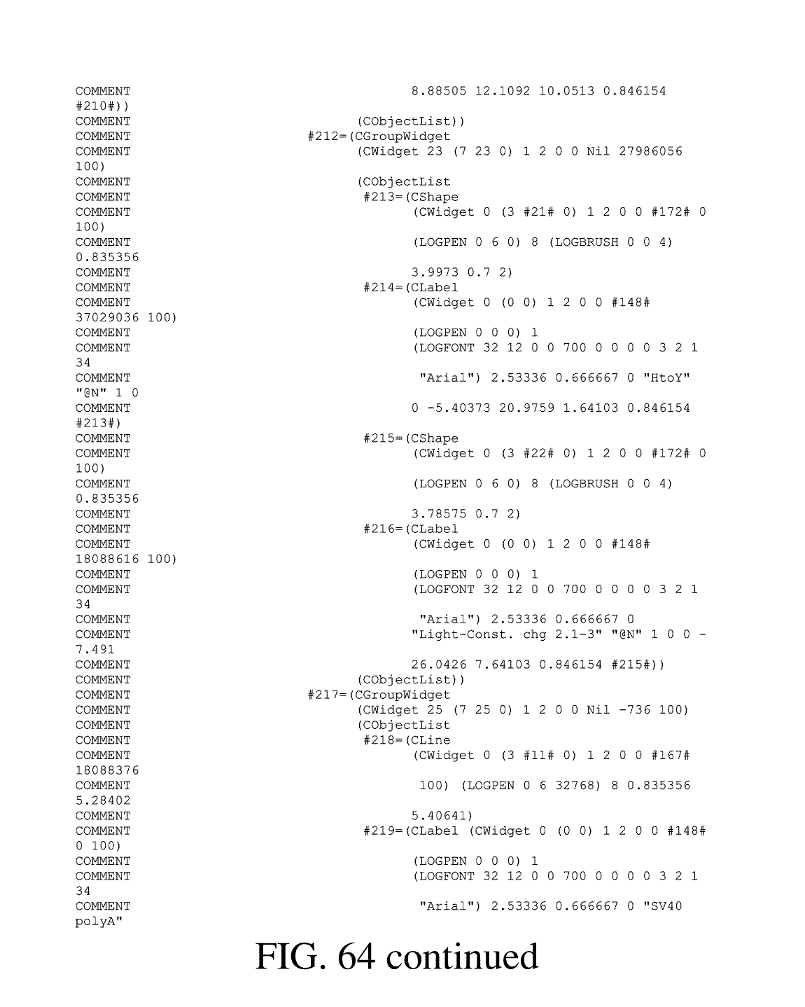

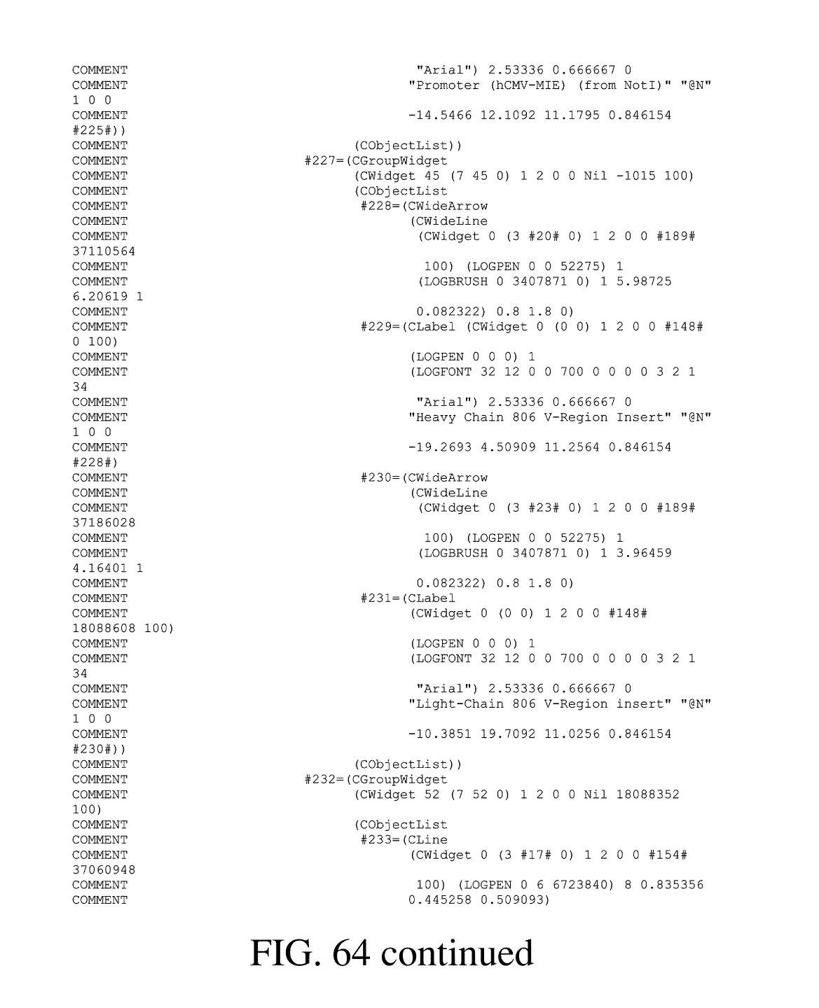

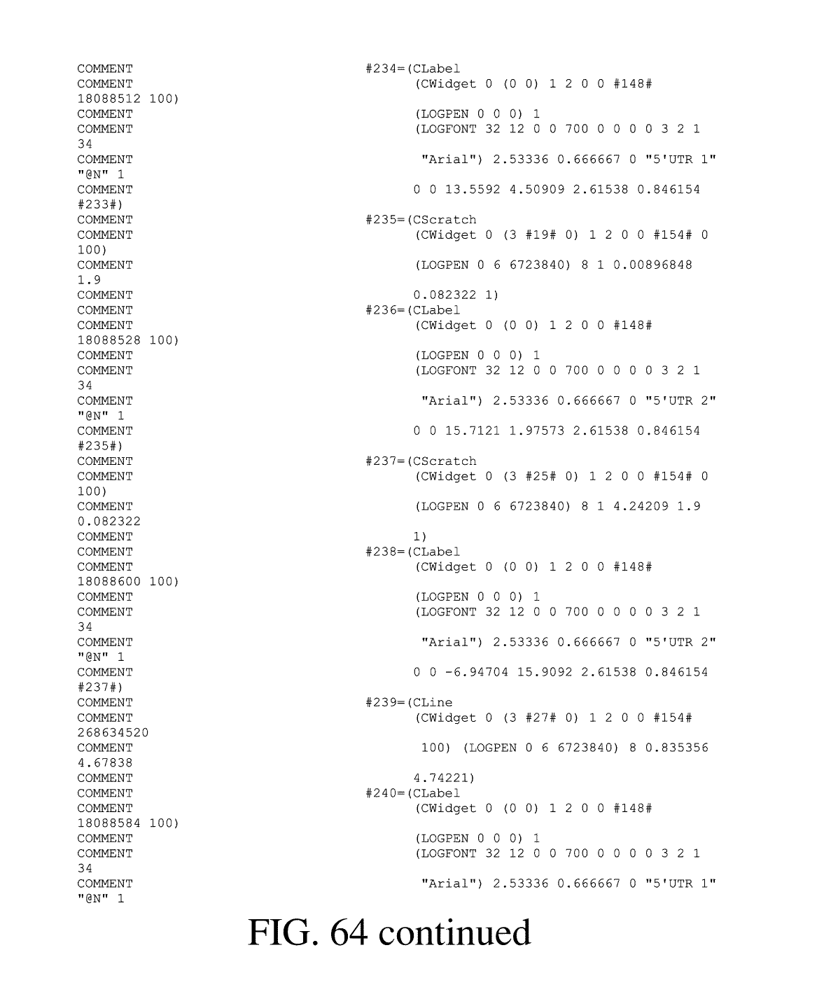

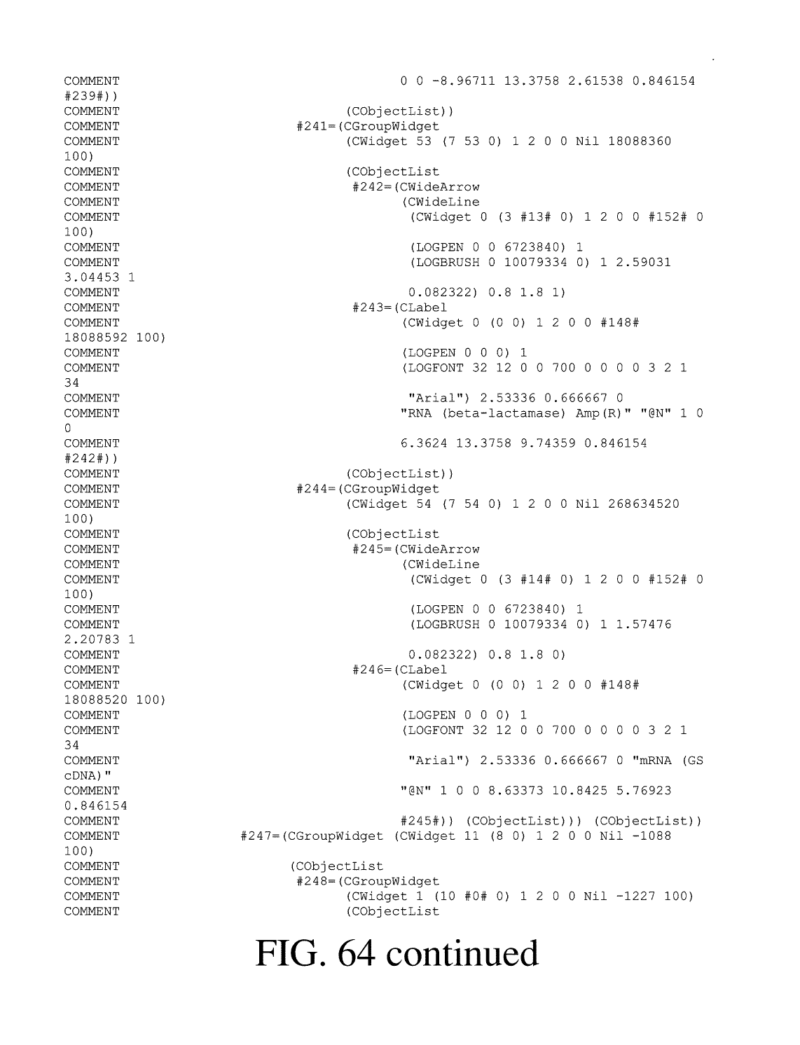

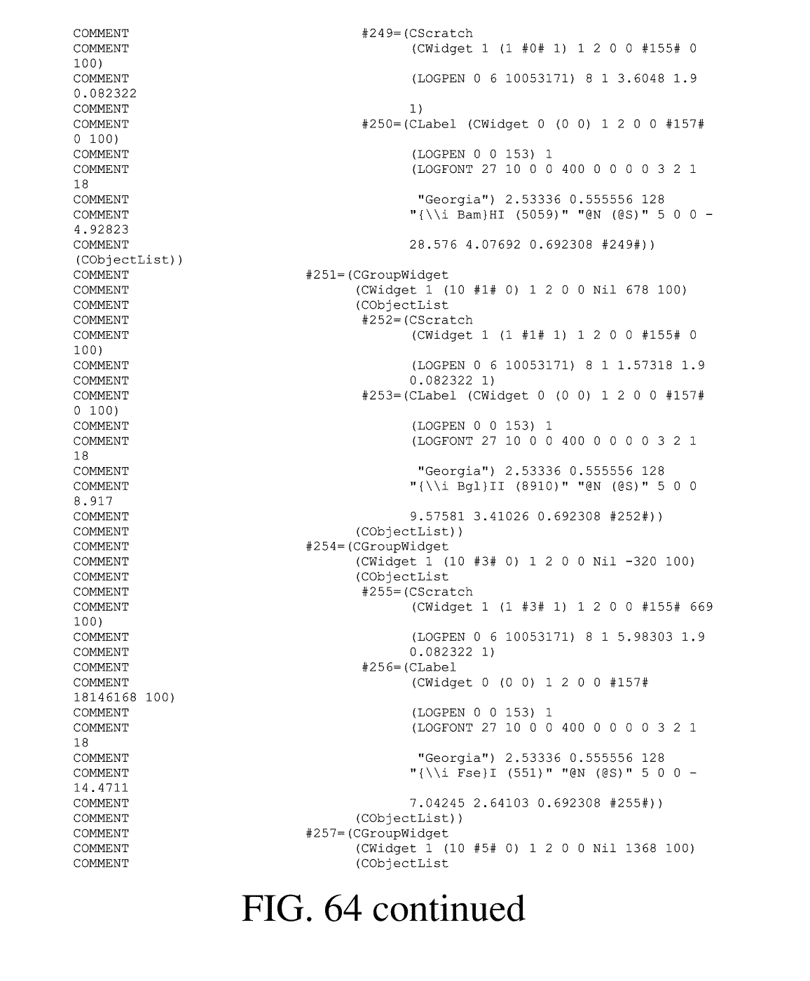

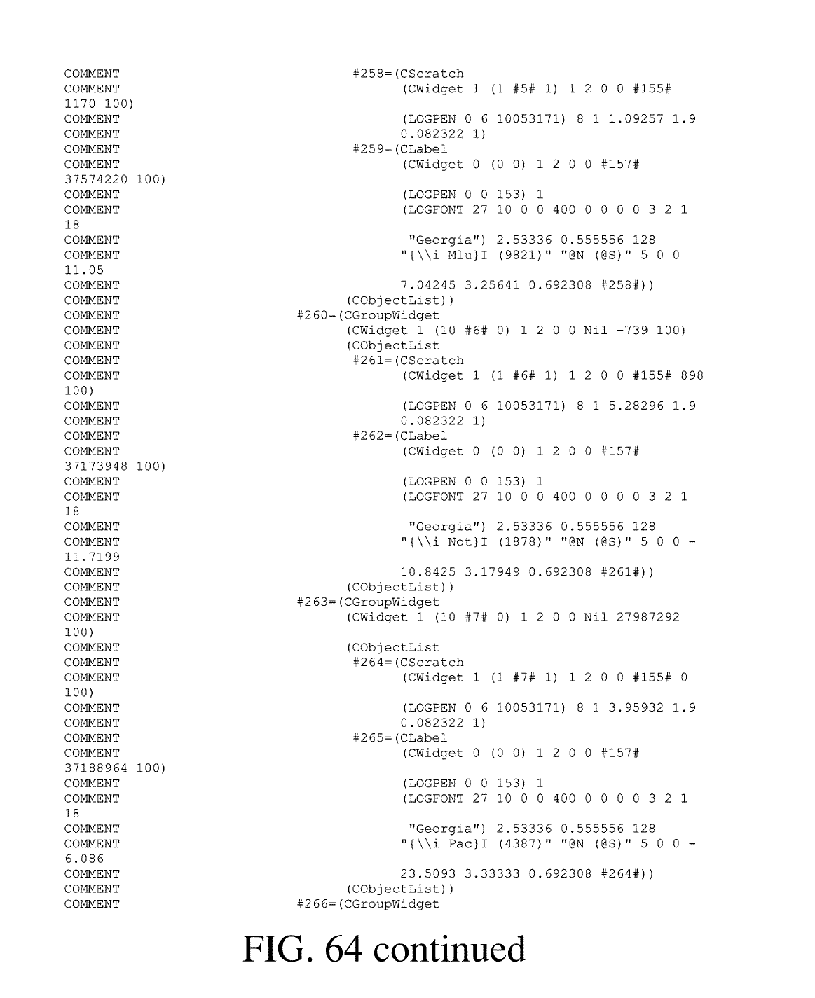

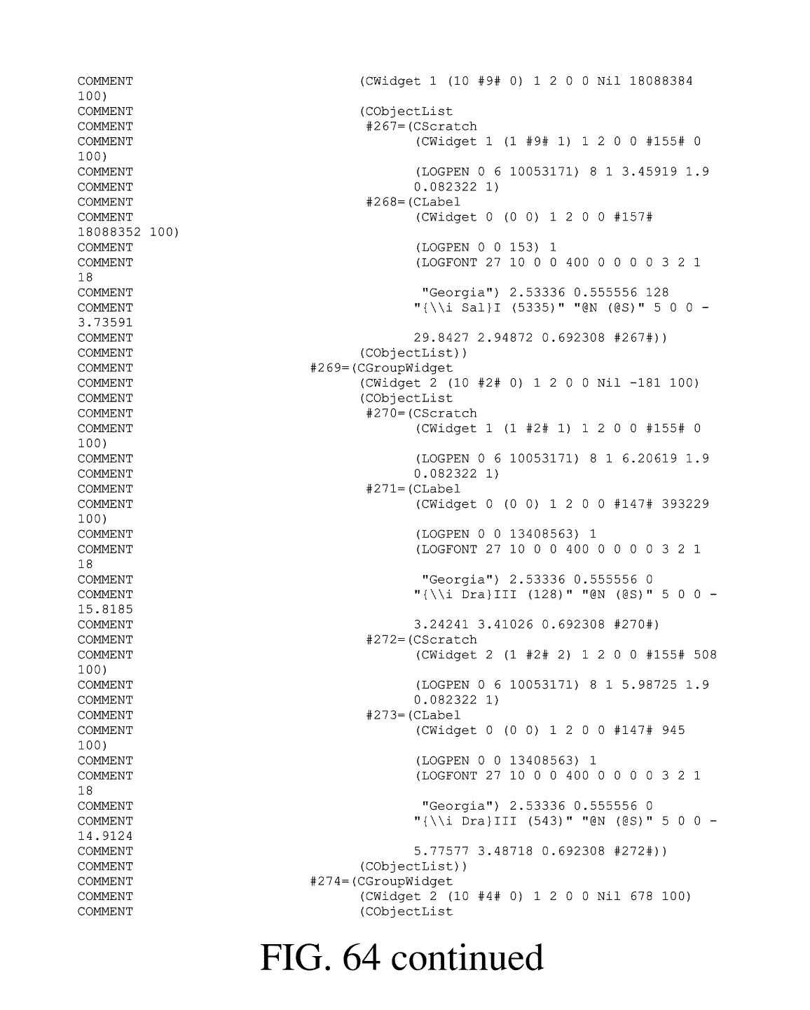

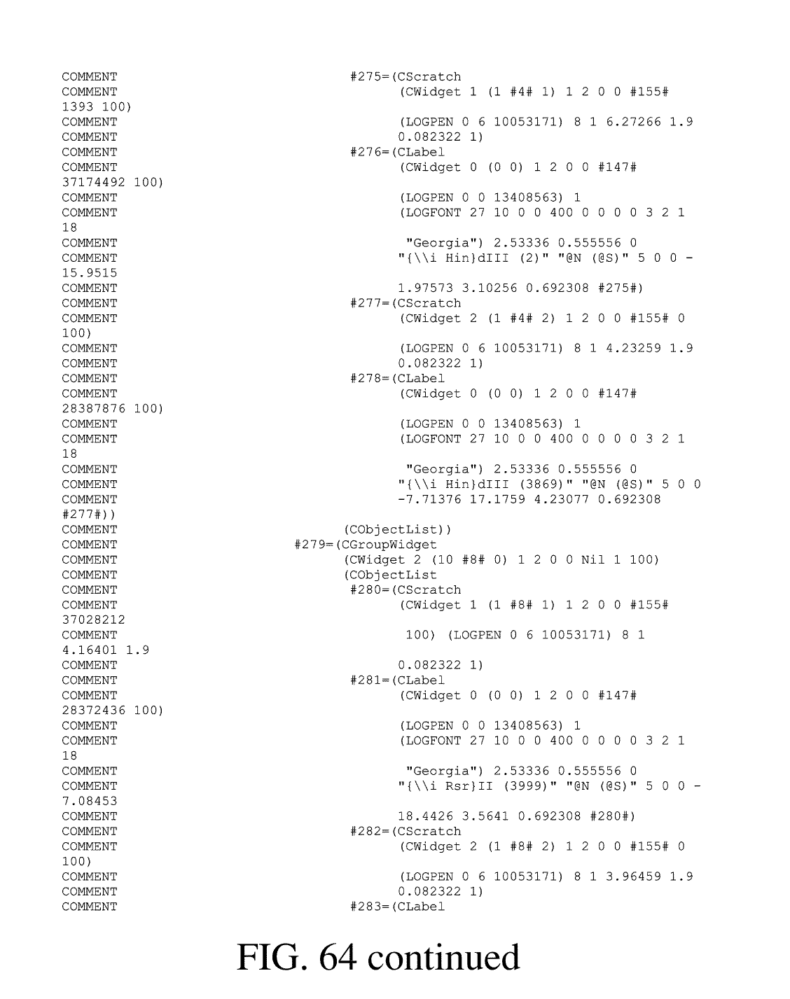

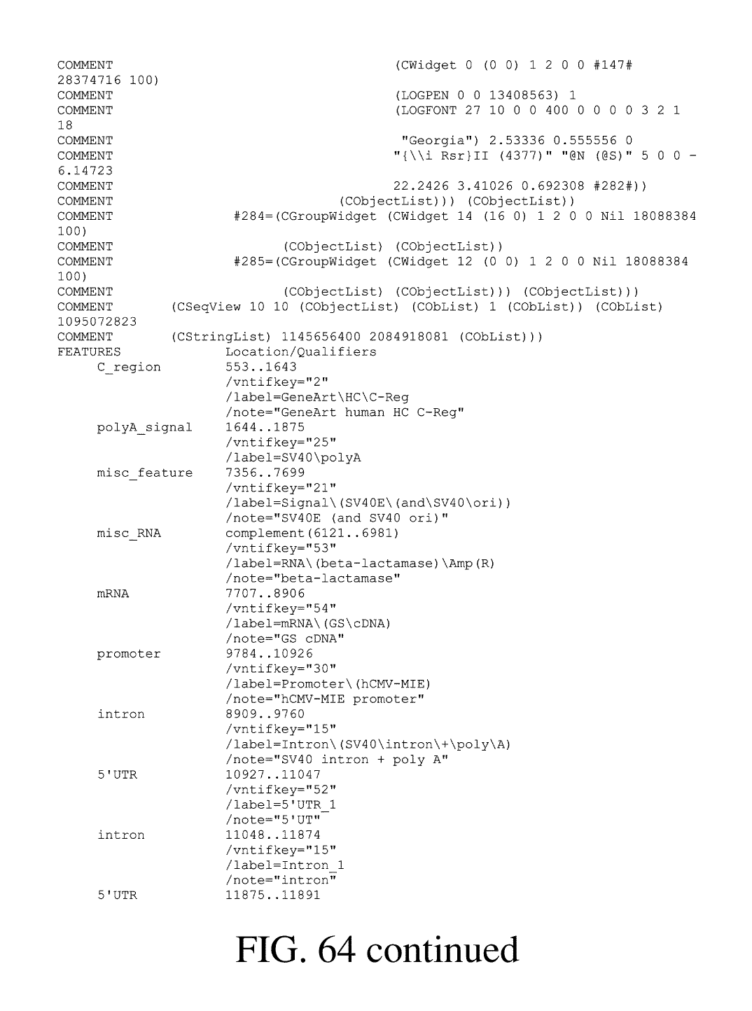

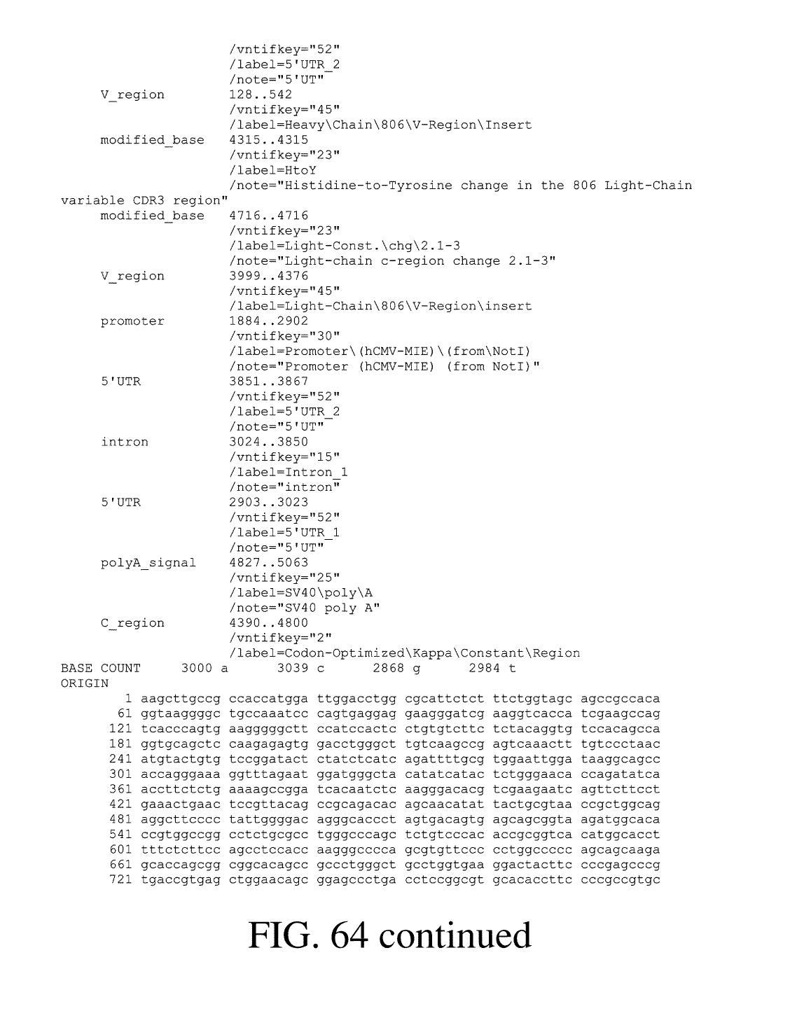

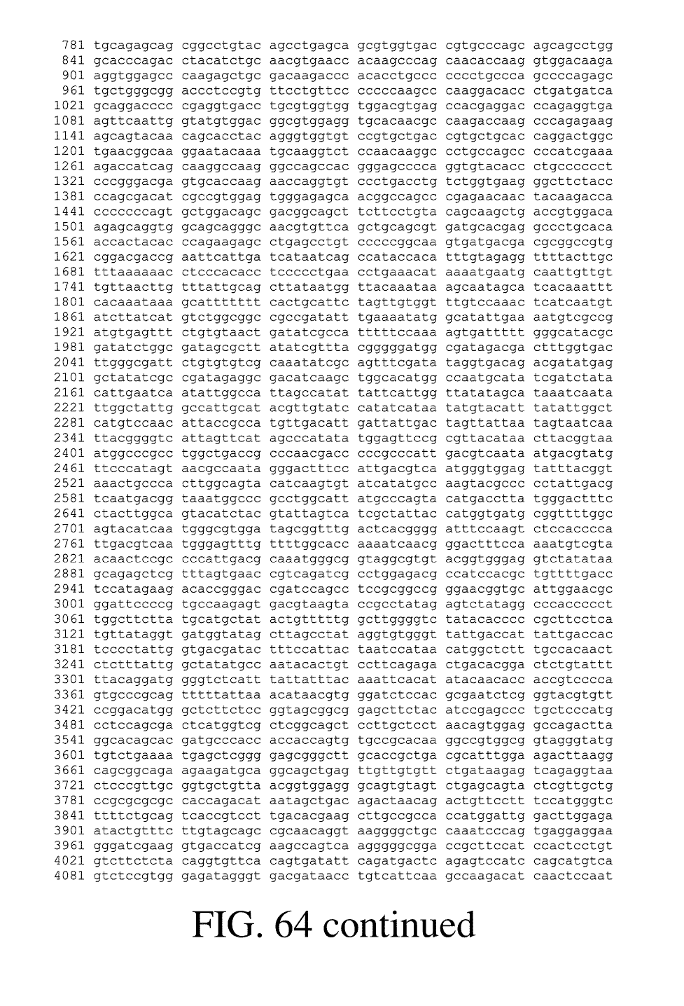

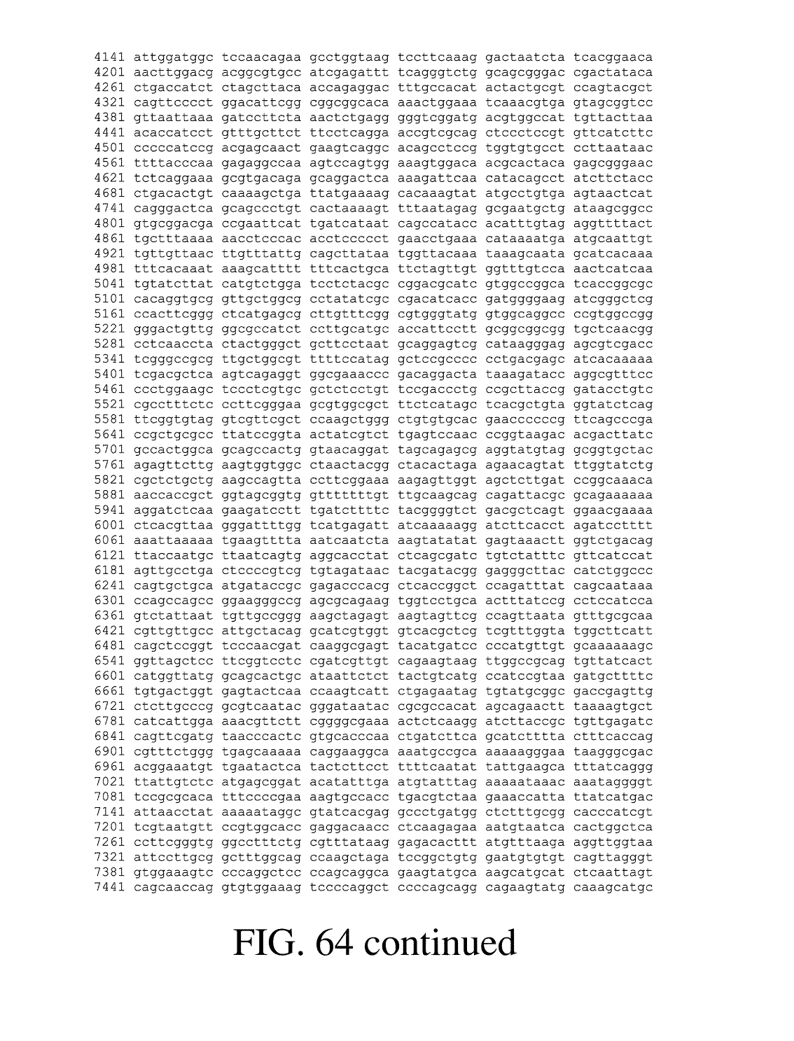

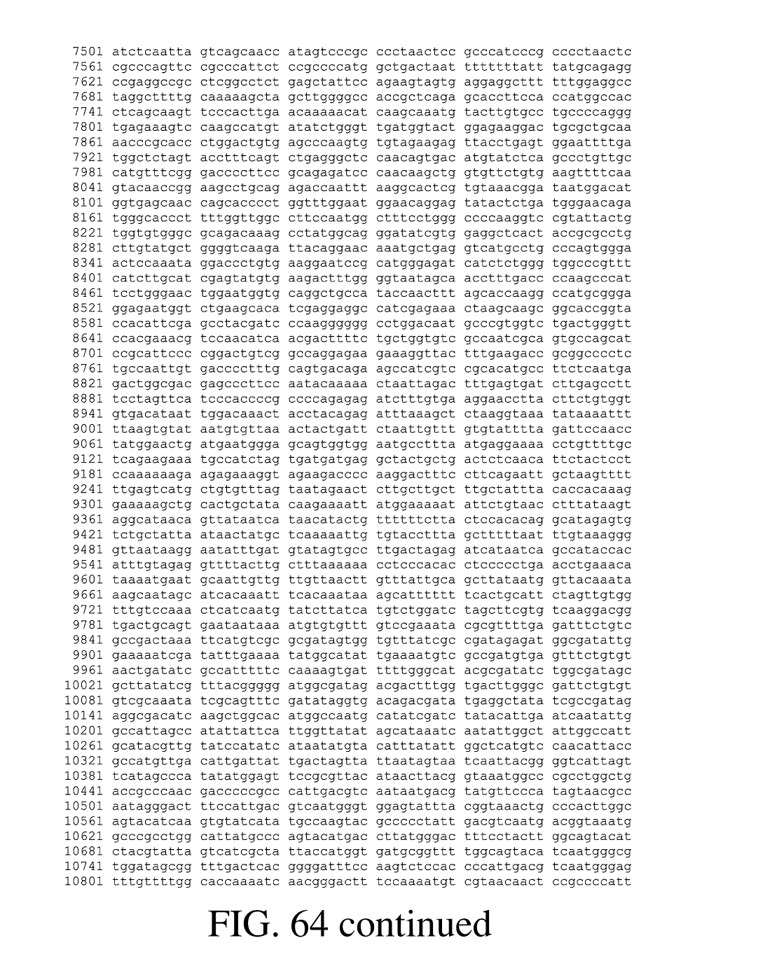

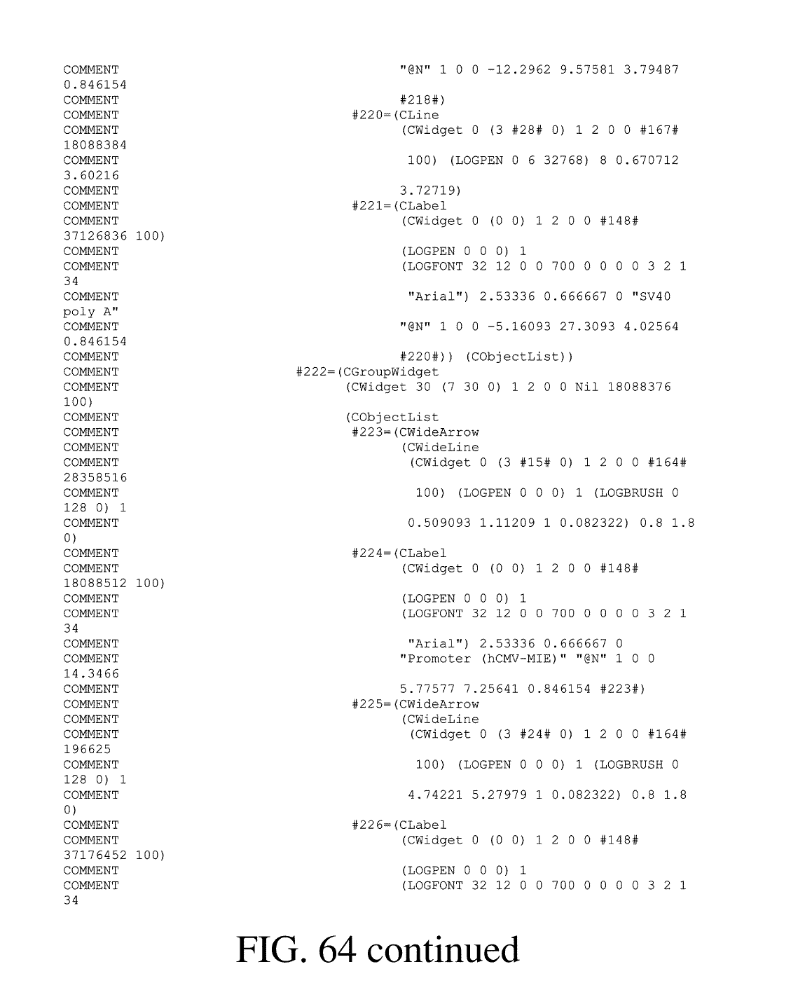

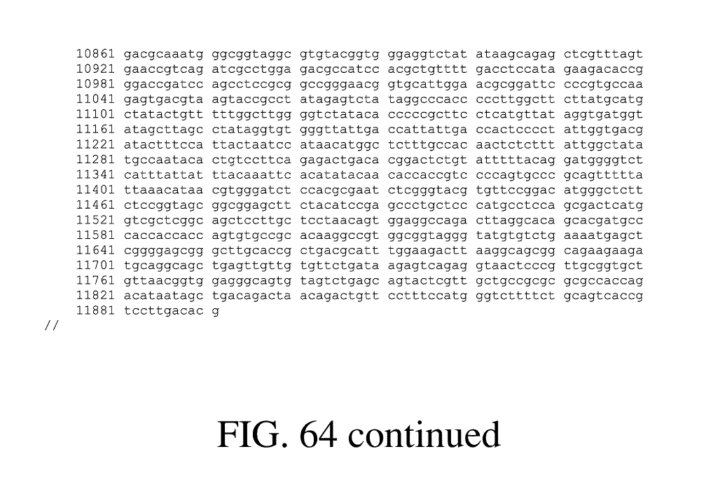

A GenBank formatted text document of the sequence and annotations of plasmid 8C65AAG encoding the IgG1 hu806 is set forth in FIG. 64.

FIG. 53 was created using Vector NTI (Invitrogen).

FIGS. 59-62 were created using Vector NTI AlignX.

### Discussion

The veneering of the 806 anti-EGF receptor antibody involved mutation of 14 amino acids in the VH (FIG. 59 and FIG. 62), and 12 changes to the VL chain (FIG. 60 and FIG. 61) with codon optimization as indicated for expression in mammalian CHO or NS0 cells. The final double gene vector, designated 8C65AAG, has been sequence-verified, and the coding sequence and translation checked. Binding to recombinant EGFR extracellular domain was confirmed by Biacore analyses using transiently expressed hu806 product.

Stable single clones producing high levels of intact hu806 antibody have been selected in glutamine-free medium as recommended by LONZA. Stable clones have been gradually weaned off serum to obtain serum-free cultures.

### B. In Vitro and In Vivo Characterization of Hu806

The higher producing stable GS-CHO hu806 transfectants 14D8, 15B2 and 40A10 and GS-NS0 hu806 transfectant 36 were progressed and small scale cultures instigated to enable preliminary hu806 product purification and characterization. Results indicated similar physicochemical properties. Accordingly a larger scale (15 L) stirred tank culture was undertaken for the highest producing transfectant (GS-CHO hu806 40A10) and purified product underwent additional in vitro characterization and in vivo therapy studies in U87MG.de2-7 and A431 xenograft models.

### Methodology and Results

### Production and Down Stream Processing:

### Small Scale

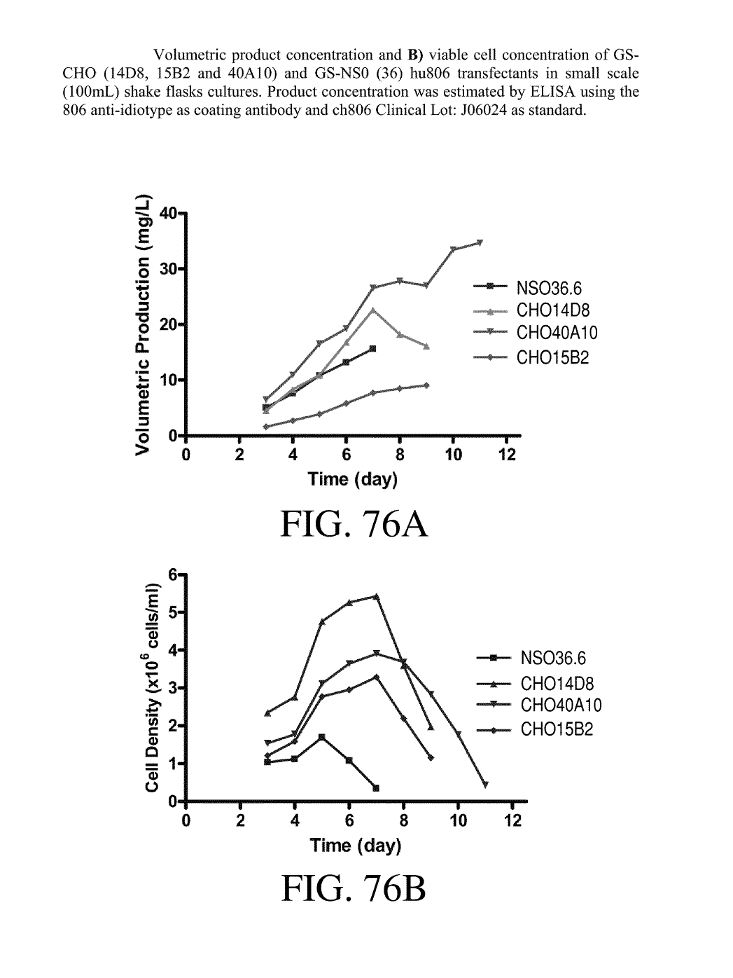

The shake flasks experiments were performed with E500 shake flasks with a 100 mL cell culture volume. FIG. 76 presents the cell viability and antibody productivity charts for the four transfectants during the culture. Product concentration was estimated by ELISA using the 806-anti-idiotype antibody LMH-12 (Liu et al. (2003) Generation of anti-idiotype antibodies for application in clinical immunotherapy laboratory analyses. Hybrid Hybridomics. 22(4), 219-28) as coating antibody, and ch806 Clinical Lot: J06024 as standard. Material at harvest was centrifuged and supernatant was 0.2 μm filtered then the antibodies were affinity purified by Protein-A chromatography.

### Large Scale

The CHO-K1SV transfectant cell line expressing hu806 candidate clone 40A10 was cultured in a 15 L stirred tank bioreactor with glucose shot feeding for 16 days using CD-CHO (Invitrogen)/25 μlVI L-Methionine sulfoximine (MSX; Sigma)/GS supplements (Sigma) as the base media. FIG. 76C presents the cell growth and volumetric production in the 15 L stirred tank bioreactor. Final yield was 14.7 L at 58 mg/L by ELISA.

Material at harvest was centrifuged and supernatant was 0.2 μm filtered then concentrated to 2 L using 2×30K membranes in Pall Centrimate concentrator. Aliquots (4×500 ml) were subsequently applied to a 250 mL Protein A column and eluted with 50 mM Citrate pH 4.5 containing 200 mM NaCl. Eluted antibody from the 4 runs was then pooled, concentrated and dialyzed into PBS, pH 7.4.

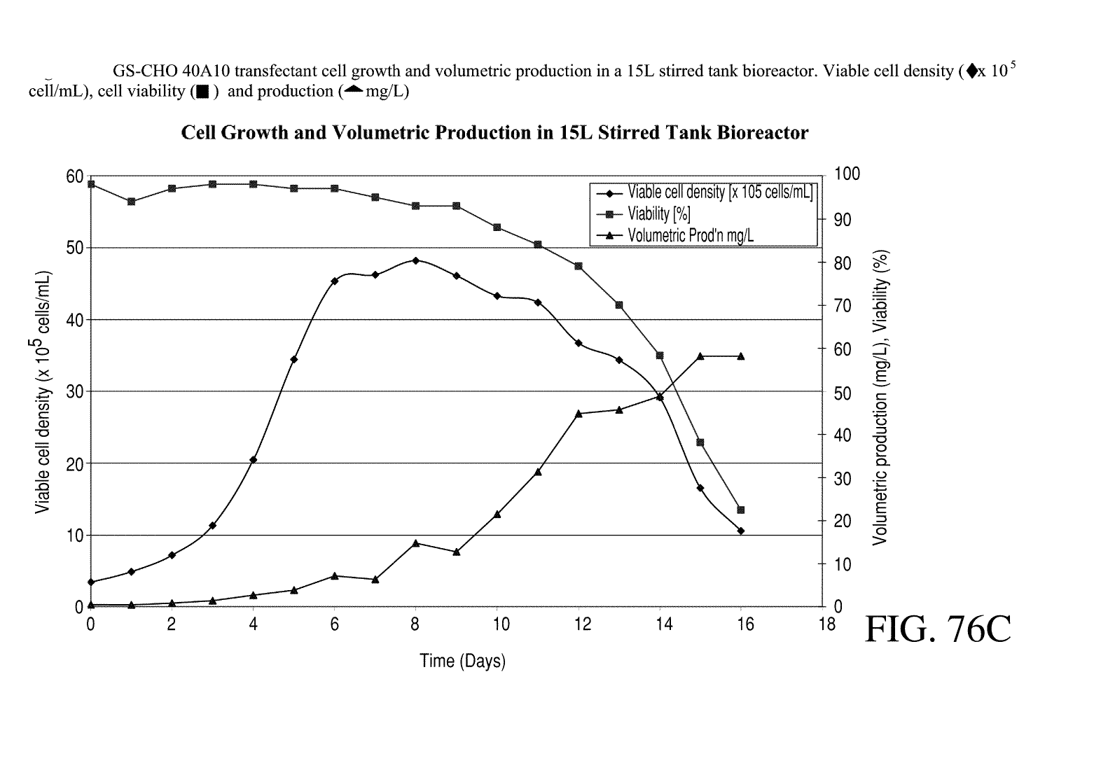

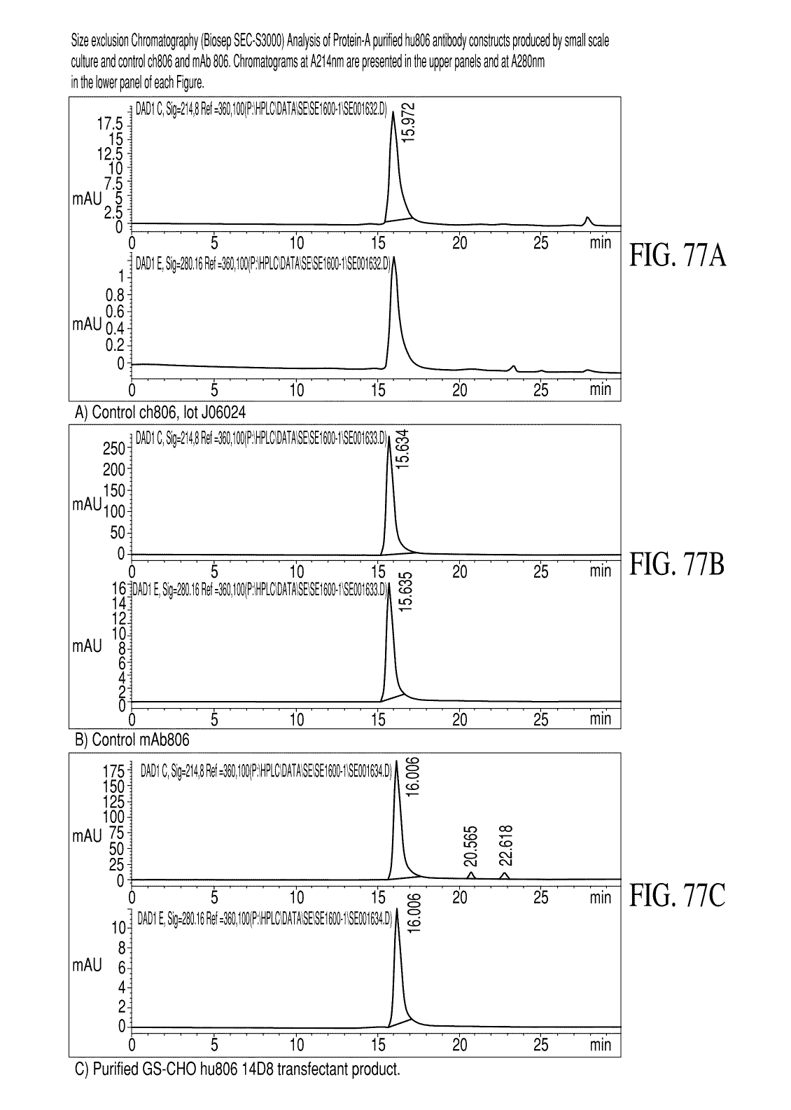

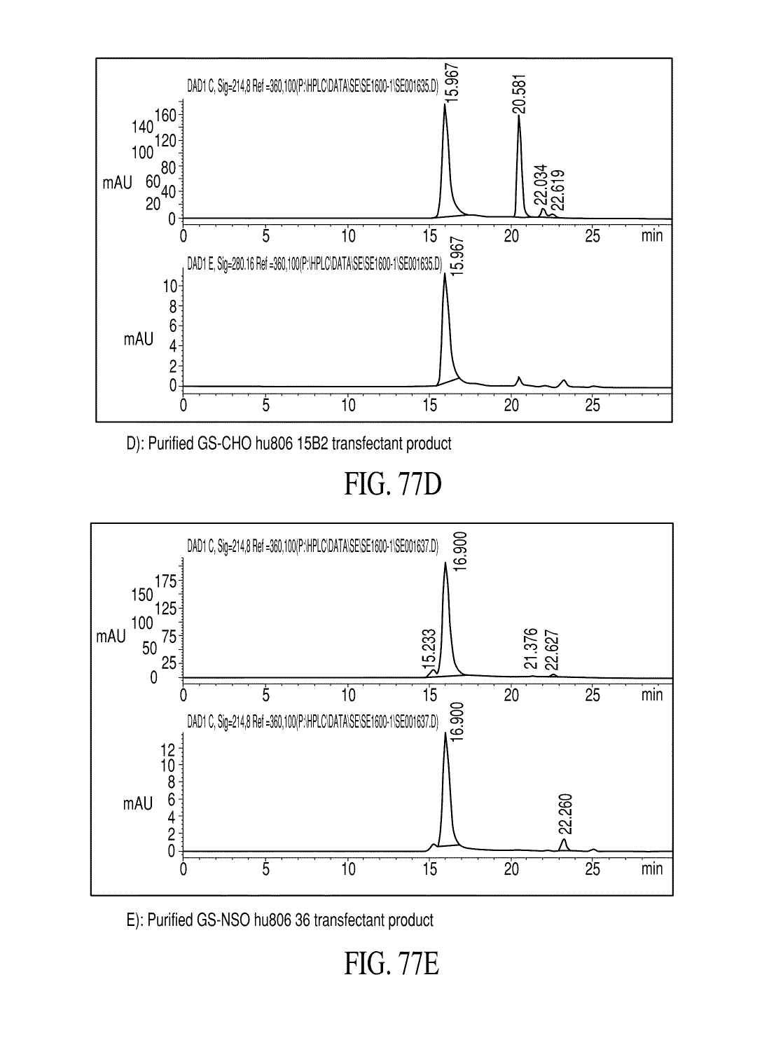

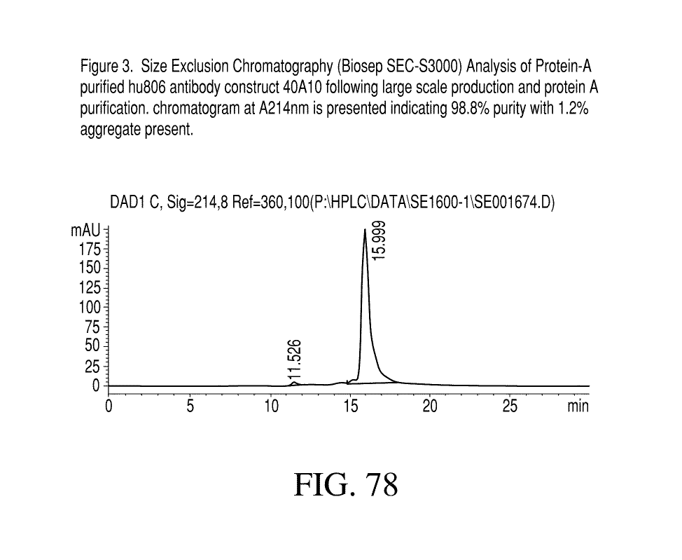

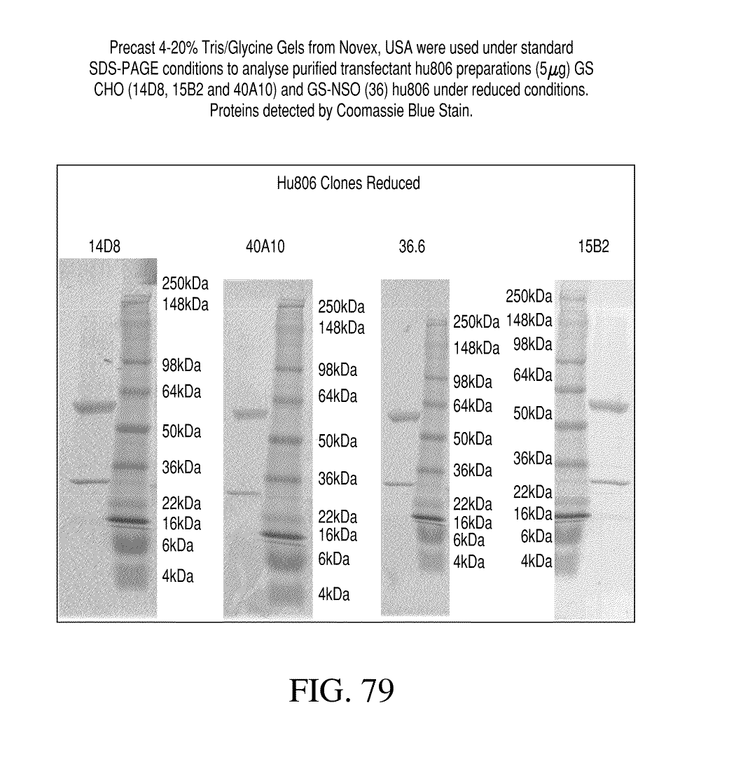

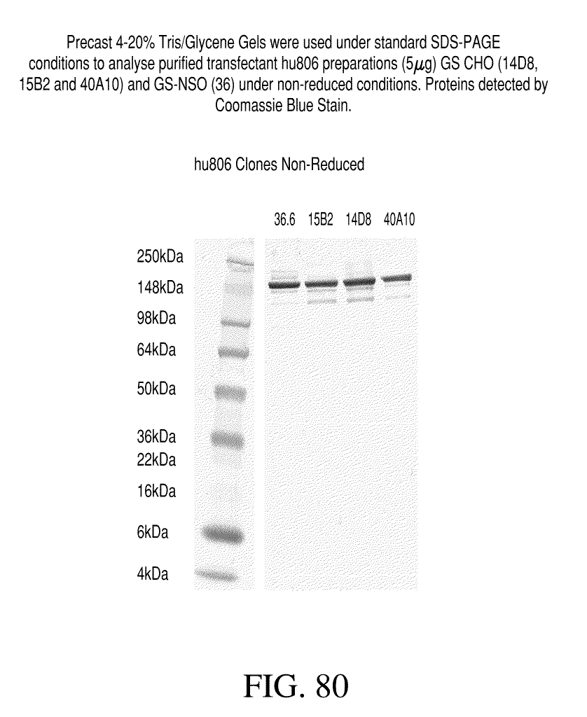

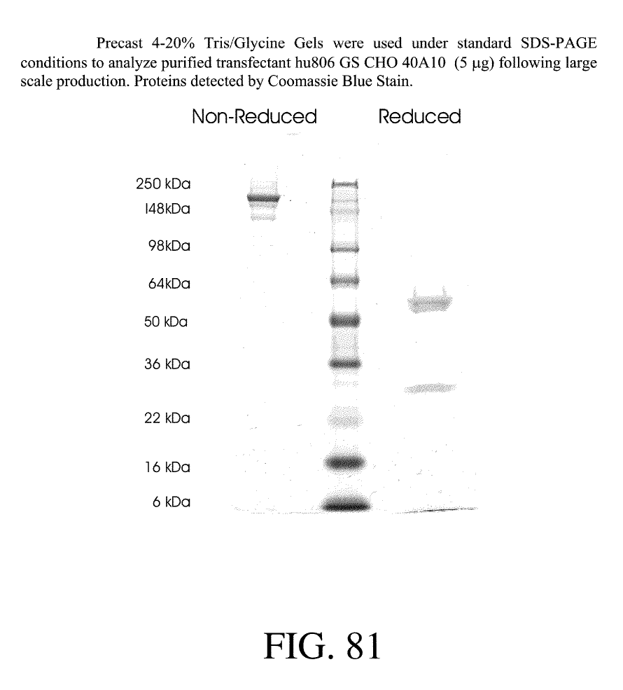

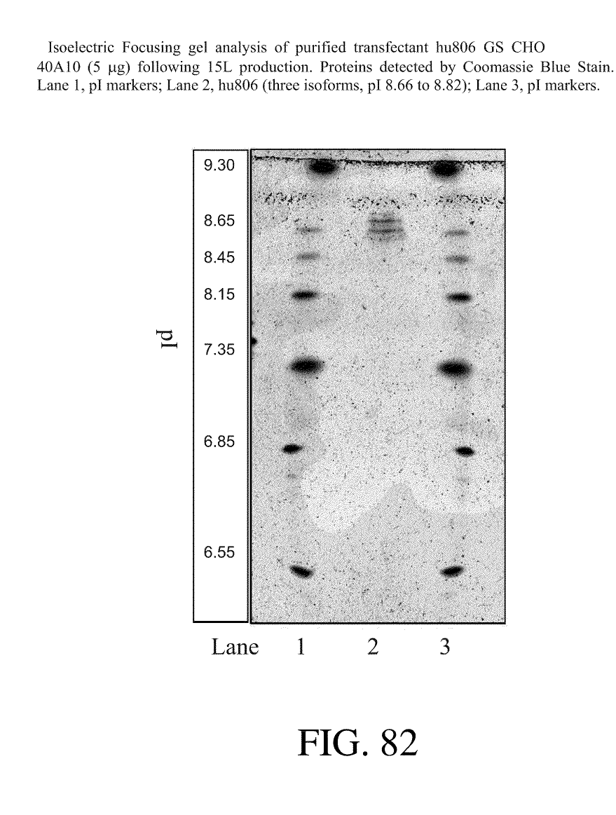

The hu806 products from the small and large scale cultures were quantified by OD A280 nm The antibody samples recovered from rProtein-A were assessed by Size Exclusion Chromatography (SEC) (small scale, FIG. 77; large scale, FIG. 78), 4-20% Tris-Glycine SDS-PAGE under reduced and non-reduced conditions (FIGS. 79-81), and Isoelectric Focusing was performed with an Amersham Multiphor II Electrophoresis system on an Ampholine PAG plate (pH 3.5-9.5) according to the manufacturer's instructions (FIG. 82).

The Protein-A affinity purified hu806 antibodies displayed symmetrical protein peaks and identical SEC elution profiles to the ch806 clinical reference material. The SDS-PAGE gel profiles were consistent with an immunoglobulin. The IEF pattern indicated three isoforms with pI ranging from 8.66 to 8.82 which was consistent with the calculated pI of 8.4 for the protein sequence.

### Binding Analyses

### FACS Analysis

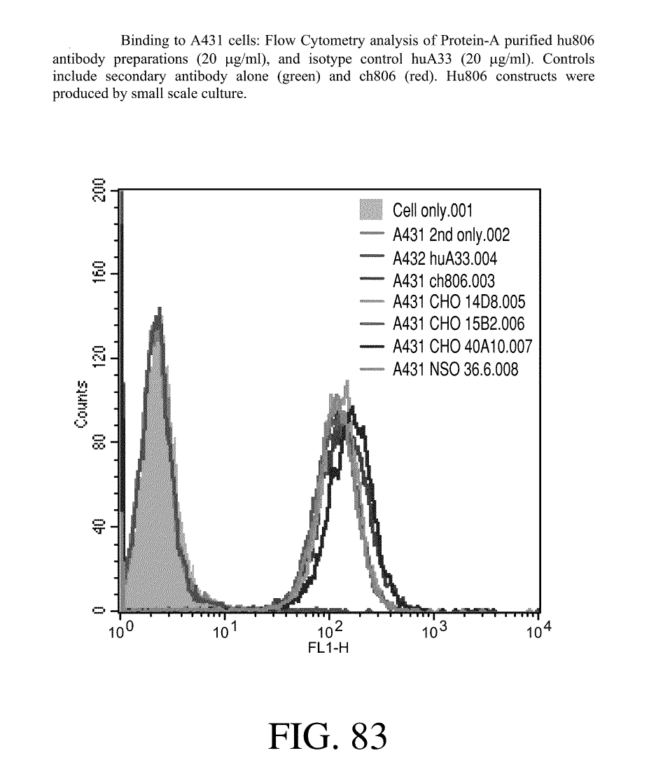

The estimates of antibody concentration determined for each sample by the OD A280 nm were utilised for FACS analyses with the adenocarcinoma cell line A431 cells (containing EGFR gene amplification). We have previously observed that mAb806 bound approximately 10% of the ˜2×10⁶ wtEGFR expressed on A431 tumor cells compared with the wtEGFR-specific mAb528 (Johns et al. (2002) Novel monoclonal antibody specific for the de2-7 epidermal growth factor receptor (EGFR) that also recognizes the EGFR expressed in cells containing amplification of the EGFR gene. Int. J. Cancer. 98(3), 398-408). Cells were stained with either one of the four hu806 samples, an irrelevant IgG2b antibody, or positive control ch806; each were assessed at a concentration of 20 μg/ml. Control for secondary antibody alone was also included [Goat anti hu-IgG (Fc specific) FITC conjugated]. Composite FACS binding curves are presented in FIG. 83 and demonstrate equivalent staining for all constructs.

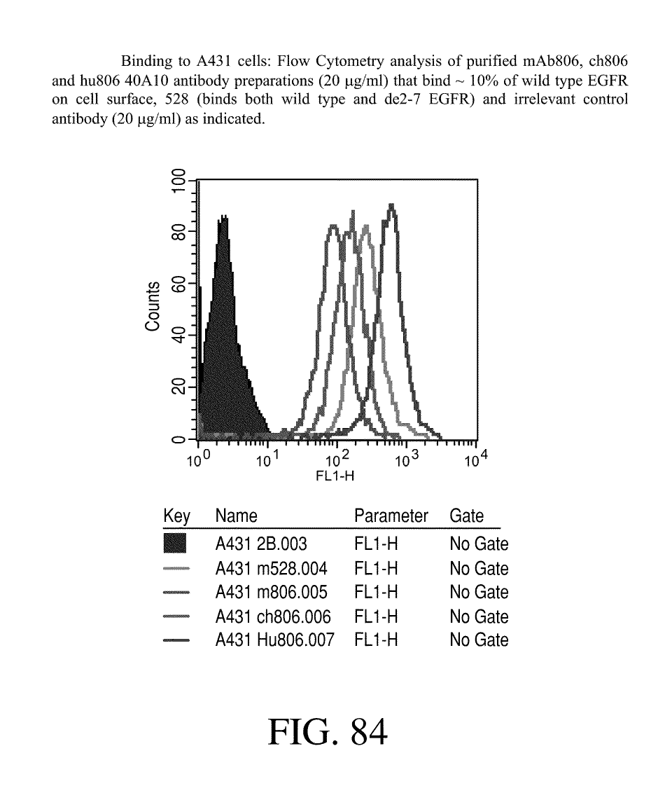

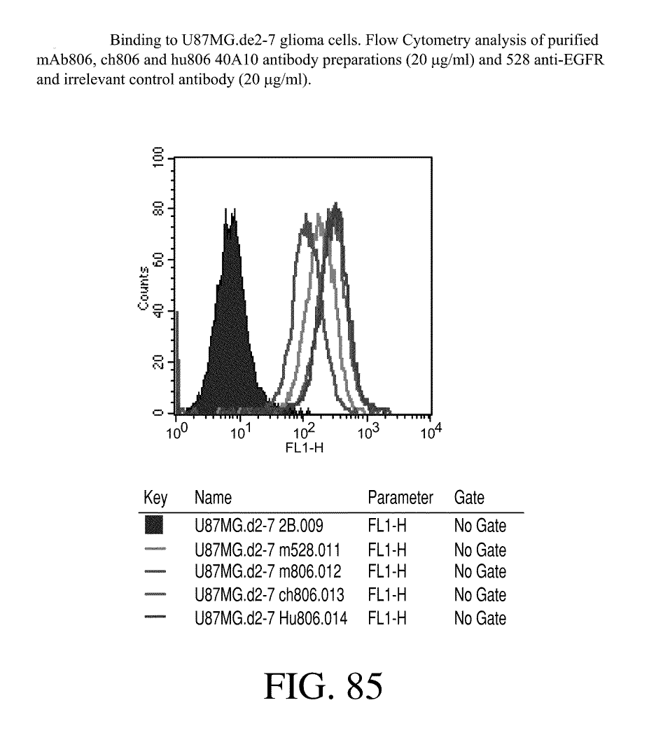

The cell binding characteristics of hu806 40A10 sample produced by large scale culture was also assessed by FACS for binding A431 as well as U87MG.de2-7 glioma cells expressing the variant EGFRvIII receptor (Johns et al., 2002). Representative results of duplicate analyses are presented in FIG. 84 and FIG. 85, respectively. Controls included an irrelevant IgG2b antibody (shaded histograms), ch806 or 528 (binds both wild-type and de2-7 EGFR) as indicated.

The ch806 and the hu806 antibody demonstrated similar staining of the A431 and U87MG.de2-7 cell lines supporting our previous observations that mAb806 specifically recognized the de2-7 EGFR and a subset of the over-expressed EGFR (Luwor et al. (2001) Monoclonal antibody 806 inhibits the growth of tumor xenografts expressing either the de2-7 or amplified epidermal growth factor receptor (EGFR) but not wild-type EGFR. Cancer Res. 61(14), 5355-61). As expected, the 528 antibody stained both the U87MG.de2-7 and A431 cell lines (FIGS. 84 and 85).

### Cell Binding Analyses

The antigen binding capabilities of the radioimmunoconjugates were assessed by cell adsorption assays (Lindmo et al. (1984) Determination of the immunoreactive fraction of radiolabeled monoclonal antibodies by linear extrapolation to binding at infinite antigen excess. J. Immunol. Methods. 72(1), 77-89) using the U87MG.de2-7 glioma cell line and A431 epidermoid carcinoma cells expressing the amplified EGFR gene.

Immunoreactive fractions of hu806 and ch806 radioconjugates were determined by binding to antigen expressing cells in the presence of excess antigen. Results for U87MG.de2-7 cell binding of ¹²⁵I-hu806 and ¹²⁵I-ch806 are presented in FIG. 86A over the cell concentration range 20×10⁶ to 0.03×10⁶ cells/sample. Results for A431 cell binding of ¹²⁵I-hu806 and ¹²⁵I-ch806 are presented in FIG. 86B over the cell concentration range 200×10⁶ to 0.39×10⁶ cells/sample.

Scatchard analyses were used to calculate the association constant (Ka) (Lindmo et al., 1984). The binding of low levels (20 ng) of labeled antibody alone was compared with binding in the presence of excess unlabeled antibody. The immunoreactive fraction was taken into account in calculating the amount of free, reactive antibody as previously described (Clarke et al. (2000) In vivo biodistribution of a humanized anti-Lewis Y monoclonal antibody (hu3S193) in MCF-7 xenografted BALB/c nude mice. Cancer Res. 60(17), 4804-11) and specific binding (nM; total antibody×% bound) was graphed against specific binding/reactive free (FIGS. 87 and 88). The association constant was determined from the negative slope of the line.

The binding affinity for ¹²⁵I-hu806 binding EGFRvIII on U87MG.de2-7 cells was determined to be 1.18×10⁹ M⁻¹. The Ka for ¹²⁵I-ch806 was 1.06×10⁹ M⁻¹. These observations are in agreement with the reported results of Ka values for ¹¹¹In- and ¹²⁵I-ch806 of 1.36×10⁹ M−1 and 1.90×10⁹ M⁻¹, respectively, which is highly comparable to that of the parental murine mAb806 of 1.1×10⁹ M⁻¹ (Panousis et al. (2005) Engineering and characterization of chimeric monoclonal antibody 806 (ch806) for targeted immunotherapy of tumours expressing de2-7 EGFR or amplified EGFR. Br. J. Cancer. 92(6), 1069-77).

The scatchard analysis on A431 cells demonstrated high affinity binding by both 806 constructs to a minor population of EGFR on these cells. The Ka for ¹²⁵I-ch806 was 0.61×10⁹ M⁻¹; and for ¹²⁵I-hu806 the Ka=0.28×10⁹ M⁻¹.

### Biosensor Analysis

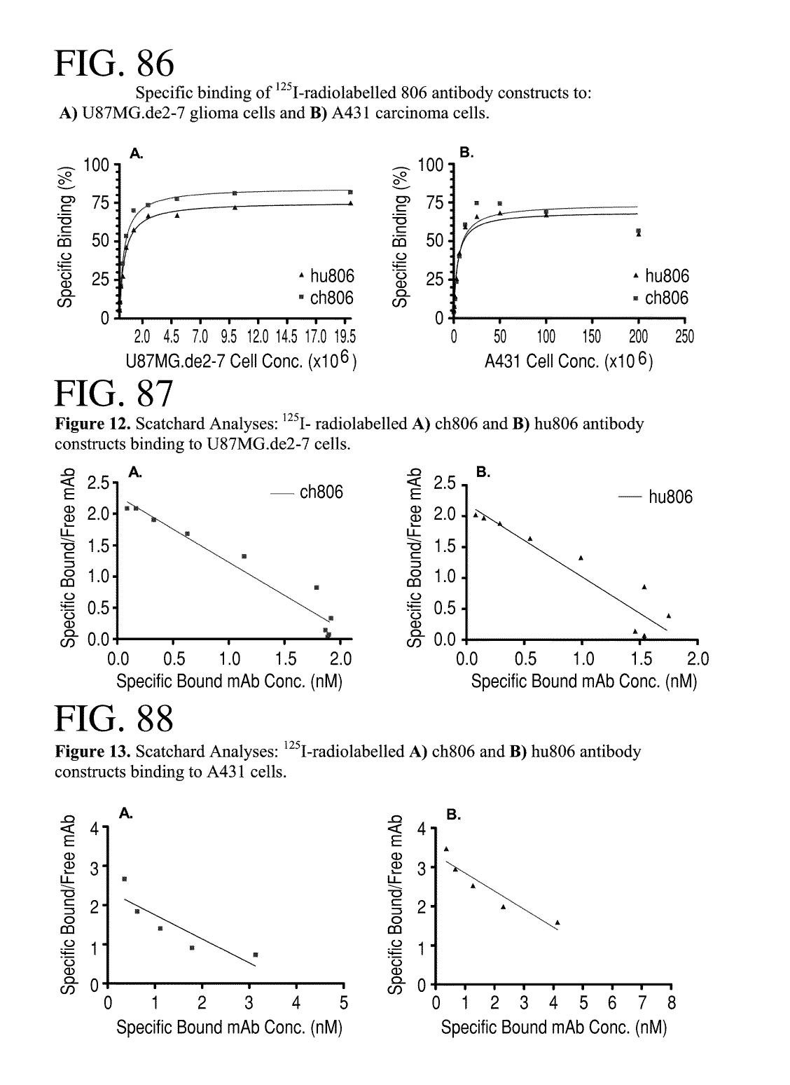

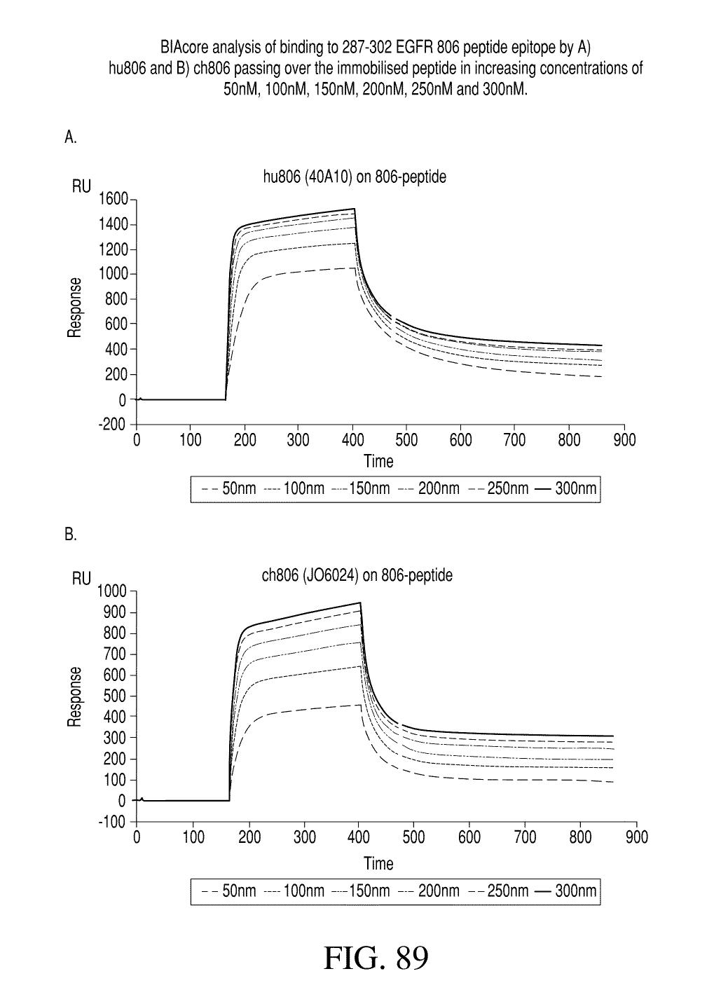

Biosensor analyses were performed on a BIAcore 2000 biosensor using a carboxymethyldextran-coated sensor chip (CM5). The chip was derivatized on channel 3 with the 806 epitope peptide (EGFR amino acids 287-302; SEQ ID NO:14; see U.S. patent application Ser. No. 11/060,646, filed Feb. 17, 2005; U.S. Provisional Patent Application No. 60/546,602, filed Feb. 20, 2004; and U.S. Provisional Patent Application No. 60/584,623, filed Jul. 1, 2004, the disclosure of each is which is hereby incorporated in its entirety), using standard amine coupling chemistry. Channel 2 was derivatized with a control antigen used for system suitability determination. Channel 1 was derivatized with ethanolamine and used as a blank control channel for correction of refractive index effects. Samples of hu806 were diluted in HBS buffer (10 mM HEPES, pH 7.4; 150 mM NaCl; 3.4 mM di-Na-EDTA; 0.005% Tween-20), and aliquots (120 μl) containing 50 nM, 100 nM, 150 nM, 200 nM, 250 nM and 300 nM were injected over the sensor chip surface at a flow rate of 30 μl/min After the injection phase, dissociation was monitored by flowing HBS buffer over the chip surface for 600s. Bound antibody was eluted and the chip surface regenerated between samples by injection of 20 μl of 10 mM sodium hydroxide solution. Positive control, ch806, was included. The binding parameters were determined using the equilibrium binding model of the BlAevaluation software. FIG. 89 present the sensorgrams generated.

Dose dependant binding was observed with both hu806 and the positive control, ch806, on channel 3. System suitability was confirmed by dose dependant binding of the appropriate monoclonal antibody to control channel 2. No cross reactivity was observed between hu806 (or ch806) and the control antibody. Our analyses determined that the apparent K_{D} (1/Ka) was 37 nM for hu806 and 94 nM for ch806.

### Antibody Dependent Cellular Cytotoxicity Analyses

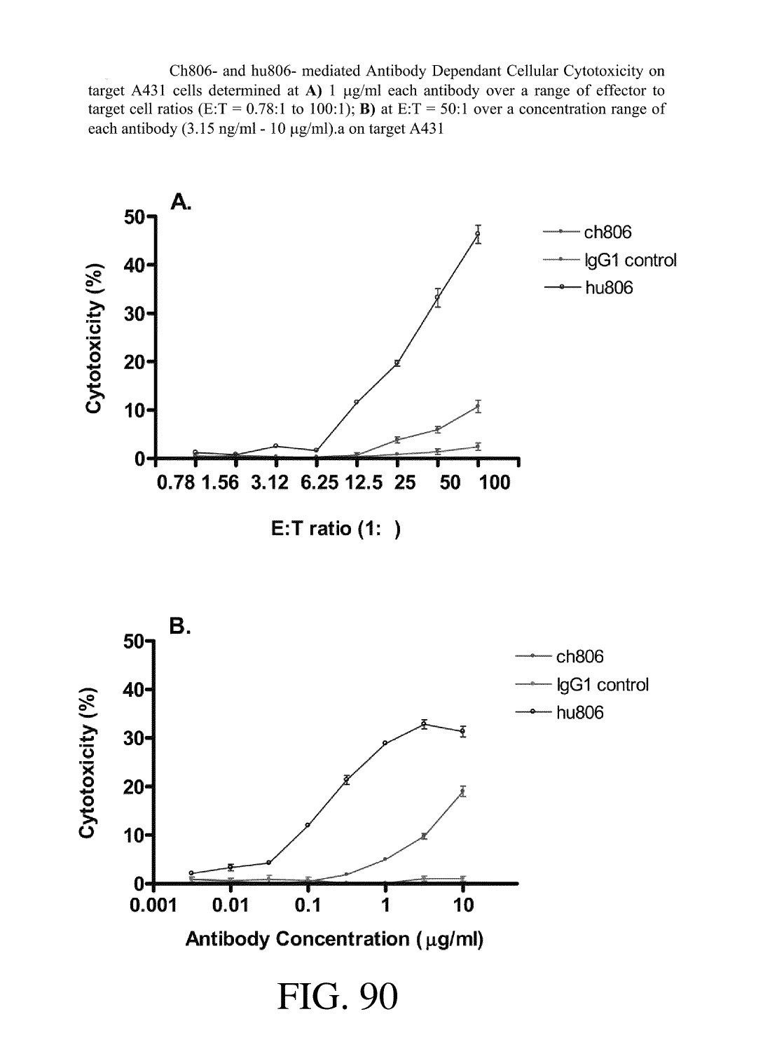

ADCC analyses were performed using purified hu806 antibody 40A10 preparation with target A431 adenocarcinoma cells and freshly isolated healthy donor peripheral blood mononuclear effector cells. Briefly, all analyses were performed in triplicate with 1) 1 μg/ml each antibody over a range of effector to target cell ratios (E:T=0.78:1 to 100:1) and also 2) at E:T=50:1 over a concentration range of each antibody (3.15 ng/ml-10 μg/ml). Controls for antibody isotype, spontaneous and total cytotoxicity were included in triplicate and calculations for specific cytotoxicity were as previously described (Panousis et al., 2005). Results are presented in FIG. 90.

The hu806 consistently demonstrated superior ADCC activity to the chimeric ch806 IgG1. In the representative experiment shown, hu806 at 1 μg/mL effected an ADCC of 30% cytotoxicity in contrast to ch806 5% cytoxicity.

### In Vivo 806 Therapy Study

The therapeutic efficacy of hu806 was investigated using established A431 adenocarcinoma or U87MG-de2-7 glioma xenografts in BALB/c nude mice. To establish xenografts, mice were injected subcutaneously into the right and left inguinal mammary line with 1×10⁶ A431 adenocarcinoma cells or 1×10⁶ U87MG.de2-7 glioma cells in 100 μl of PBS. Tumor volume (TV) was calculated by the formula [(length×width²)/2] where length was the longest axis and width the measurement at right angles to length. In an initial experiment, groups of five BALB/c nude mice (n=10 tumours/group) with established A431 or U87MG.de2-7 xenografts received treatment of 1 mg hu806, or 1 mg ch806 antibody or PBS vehicle control by IP injection. Therapy was administered on days 6, 8, 11, 13, 15 and 18 for A431, and days 4, 6, 8, 11, 13 and 15 for the U87MG.de2-7 cell lines respectively. Mean±SEM tumor volumes until termination of the experiments due to ethical considerations of tumor burden are presented in FIG. 91 for the A431 xenograft until day 25, and in FIG. 92 for U87MG.de2-7 xenografts until day 31.

The in vivo therapy assessments with hu806 showed a marked reduction in A431 xenograft growth compared with PBS vehicle control. The A431 xenograft growth curve observed for hu806 was highly comparable to the ch806 treatment group. In the established U87MG.de2-7 xenografts, the PBS control group was euthanized at day 20. The hu806 therapy demonstrated significant reduction in tumor growth by day 20 compared to the PBS controls (P<0.001), and continued tumor growth retardation after day 20 similar to the ch806 group.

### Discussion

The Protein-A affinity purified hu806 antibodies displayed identical SEC elution profiles to the ch806 clinical reference material, and SDS-PAGE gel profiles consistent with an immunoglobulin. The IEF pattern was consistent with the anticipated pI of 8.4.

Through Scatchard cell binding and Biosensor epitope binding analyses the hu806 antibody demonstrated highly comparable binding curves and affinity parameters to the ch806 antibody. The binding affinity of hu806 and ch806 to EGFRvIII and over expressed wild-type EGFR are similar and in the low nanomolar range. Cell binding through FACS analyses supported these observations.

Furthermore, the hu806 demonstrates markedly improved ADCC over the ch806 construct on target antigen positive A431 cells.

The in vivo therapeutic assessments with hu806 showed a marked reduction in A431 xenograft growth, which was highly comparable to the ch806 treatment group. In the established U87MG.de2-7 xenografts, hu806 therapy demonstrated significant reduction in tumor growth by day 20 compared to the PBS controls and continued tumor growth retardation after day 20 similar to the ch806 group.

### Example 23

### Monoclonal Antibody 175

As discussed in Example 1, clone 175 (IgG2a) was selected for further characterization.

a. Materials and Methods

### Cell Lines

The Δ2-7EGFR transfected U87MG.Δ2-7 (Huang et al. (1997) J. Biol. Chem. 272, 2927-2935) and the A431 cell lines (Ullrich et al. (1984) Nature. 309, 418-425) have been described previously. The hormone-independent prostate cell line DU145 (Mickey et al. (1977) Cancer Res. 37, 4049-4058) was obtained from the ATCC (atcc.org).

All cell lines were maintained in DMEM (Life Technologies, Grand Island, N.Y.) containing 10% FCS(CSL, Melbourne), 2 mM glutamine (Sigma Chemical Co, St. Louis), and penicillin/streptomycin (Life Technologies, Grand Island). In addition, the U87MG.Δ2-7 cell line was maintained in 400 mg/ml of Geneticin (Life Technologies, Inc, Grand Island). BaF/3 (Palacios et al. (1984) Nature. 309, 126-131) and BaF/3 cell lines expressing different EGF receptors (Walker et al. (2004) J. Biol. Chem. 2(79), 22387-22398) were maintained routinely in RPMI 1640 (GIBCO BRL.) supplemented with 10% fetal calf serum (GIBCO BRL) and 10% WEHI-3B conditioned medium (Ymer et al. (1985) Nature. 19-25; 317, 255-258) as a source of IL-3. All cell lines were grown at 37° C. in an air/CO₂ (95%-5%) atmosphere.

### Antibodies and Peptides

mAb806 and mAb175 were generated at the Ludwig Institute for Cancer Research (LICR) New York Branch and were produced and purified in the Biological Production Facility (Ludwig Institute for Cancer Research, Melbourne). The murine fibroblast line NR6_{ΔEGFR} was used as immunogen. Mouse hybridomas were generated by immunizing BALB/c mice five times subcutaneously at 2- to 3-week intervals, with 5×10⁵-2×10⁶ cells in adjuvant. Complete Freund's adjuvant was used for the first injection. Thereafter, incomplete Freund's adjuvant (Difco) was used. Spleen cells from immunized mice were fused with mouse myeloma cell line SP2/0. Supernatants of newly generated clones were screened in hemadsorption assays for reactivity with cell line NR6, NR6_{wtEGFR}, and NR6_{ΔEGFR} and then analyzed by hemadsorption assays with human glioblastoma cell lines 1:187 MG, U87MG_{wtEGFR}, and U87MG_{ΔEGFR}.

Intact mAbs (50 mg) were digested in PBS with activated papain for 2-3 hours at 37° C. at a ratio of 1:20 and the papain was inactivated with iodoacetanaide. The digestion was then passed over a column of Protein-A sepharose (Amersham) in 20 mM sodium phosphate buffer pH 8.0, with the flow-through further purified by cation exchange using on a Mono-S column (Amersham). Protein was then concentrated using a 10,000 MWCO centrifugal concentrator (Millipore). For Fab-peptide complexes a molar excess of lyophilized peptide was added directly to the Fab and incubated for 2 hours at 4° C. before setting up crystallization trials.

Mapping of mAb175 Using Egfr Fragments Expressed in Mammalian Cells

The day prior to transfection with these fragments, human 293T embryonic-kidney fibroblasts were seeded at 8×10⁵ per well in 6-well tissue culture plates containing 2 ml of media. Cells were transfected with 3-4 μg of plasmid DNA complexed with Lipofectamine 2000 (Invitrogen) according to the manufacturer's instructions. 24 to 48 h after transfection, cell cultures were aspirated and cell mono layers lysed in 250μ; of lysis buffer (1% Triton X-100, 10% glycerol, 150 mM NaCl, 50 mM HEPES pH 7.4, 1 mM EGTA and Complete Protease Inhibitor mix (Roche). Aliquots of cell lysate (10-15 ni) were mixed with SDS sample buffer containing 1.5% β-mercaptoethanol, denatured by heating for 5 min at 100° C. and electrophoresed on 10% NuPAGE Bis-Tris polyacrylamide gels (Invitrogen). Samples were then electro-transferred to nitrocellulose membranes that were rinsed in TBST buffer (10 mM Tris-HCl, pH 8.0, 100 mM NaCl and 0.1% Tween-20) and blocked in TBST containing 2.5% skim milk for 30 min at room temperature. Membranes were incubated overnight at 4° C. with 0.5 μg/ml of mAb175 in blocking buffer. Parallel membranes were probed overnight with mAb 9B11 (1:5000, Cell Signaling Technology, Danvers, Mass.) to detect the c-myc epitope. Membranes were washed in TBST, and incubated in blocking buffer containing horseradish peroxidase-conjugated rabbit anti-mouse IgG (Biorad) at a 1:5000 dilution for 2 h at room temperature. Blots were then washed in TBST, and developed using autoradiographic film following incubation with Western Pico Chemiluminescent Substrate (Pierce, Rockford, Ill.).

Mapping of mAb175 using EGFR Fragments Expressed in Mammalian Cells and Yeast

A series of overlapping c-myc-tagged EGFR ectodomain fragments, starting at residues 274, 282, 290 and 298 and all terminating at amino acid 501 and fused to growth hormone have been described previously (Johns et al. (2004) J. Biol. Chem. 279, 30375-30384). Expression of EGFR proteins on the yeast cell surface was performed as previously described (Johns et al., 2004).

Briefly, transformed colonies were grown at 30° C. in minimal media containing yeast nitrogen base, casein hydrolysate, dextrose, and phosphate buffer pH 7.4, on a shaking platform for approximately one day until an OD₆₀₀ of 5-6 was reached. Yeast cells were then induced for protein display by transferring to minimal media containing galactose, and incubated with shaking at 30° C. for 24 h. CuItures were then stored at 4° C. until analysis. Raw ascites fluid containing the c-myc monoclonal antibody 9E10 was obtained from Covance (Richmond, Calif.). 1×10⁶ yeast cells were washed with ice-cold FACS buffer (PBS containing 1 mg/ml BSA) and incubated with either anti-c-myc ascites (1:50 dilution), or human EGFR monoclonal antibody (10 ng/ml) in a final volume of 50).t.1, for 1 hr at 4′C. The cells were then washed with ice cold FACS buffer and incubated with phycoerythrin-labelled anti-mouse IgG (1:25 dilution), in a final volume of 50 μl for 1 h at 4″C, protected from light. After washing the yeast cells with ice-cold FACS buffer, fluorescence data was obtained with a Coulter Epics XL flow cytometer (Beckman-Coulter), and analyzed with WinMDI cytometry software (J. Trotter, Scripps University). For determination of linear versus conformational epitopes, yeast cells were heated at 80° C. for 30 min, then chilled on ice 20 min prior to labeling with antibodies. The series of EGFR mutants listed in Table 7 have been described previously (Johns et al., 2004).

### Surface Plasmon Resonance (BIAcore)

A BIAcore 3000 was used for all experiments. The peptides containing the putative mAb806 epitope were immobilized on a CM5 sensor chip using amine, thiol or Pms coupling at a flow rate of 5 μl/min (Wade et al. (2006) Anal. Biochem. 348, 315-317). The mAb806 and mAb175 were passed over the sensor surface at a flow rate of 5 μl/min at 25° C. The surfaces were regenerated between runs by injecting 10 mM HCI at a flow rate of 10 μl/min.

### Immunoprecipitation and Western Blotting

Cells were lysed with lysis buffer (1% Triton X-100, 30 mM HEPES, 150 mM NaCl, 500 mM 4-(2-aminoethyl) benzenesulfonylfluoride, 150 nM aprotinin, 1 mM E-64 protease inhibitor, 0.5 mM EDTA, and 1 mM leupeptin, pH 7.4) for 20 minutes, clarified by centrifugation at 14,000×g for 30 minutes, immunoprecipitated with the relevant antibodies at a final concentration of 5 μg/ml for 60 minutes and captured by Sepharose-A beads overnight. Samples were then eluted with 2× NuPAGE SDS Sample Buffer (invitrogen), resolved on NuPAGE gels (either 3-8% or 4-12%), electro-transferred onto Immobilon-P transfer membrane (Millipore) then probed with the relevant antibodies before detection by chemoluminescence radiography.

### Immunohistochemistry

Frozen sections were stained with 5 μg/ml mAb175 or irrelevant isotype control for 60 min at room temperature. Bound antibody was detected using the Dako Envision+HRP detection system as per manufacturer's instructions. Sections were finally rinsed with water, counterstained with hematoxylin and mounted.

### Xenograft Models

U87MG.Δ2-7 cells (3×10⁶) in 100 μL of PBS were inoculated s.c. into both flanks of 4- to 6-week-old, female Balb/c nude mice (Animal Research Centre, Perth, Australia). All studies were conducted using established tumor models as reported previously (Perera et al. (2005) Clin. Cancer Res. 11, 6390-6399). Treatment commenced once tumors had reached the mean volume indicated in the appropriate figure legend. Tumor volume in mm³ was determined using the formula (length×width²)/2, where length was the longest axis and width was the perpendicular measurement. Data are expressed as mean tumor volume±SE for each treatment group. All data was analyzed for significance by one-sided Student's t test where p<0.05 was considered statistically significant. This research project was approved by the Animal Ethics Committee of the Austin Hospital.

### Generation and Characterization of Stable Cell Lines Expressing EGFR Mutant Constructs

Mutations of the wtEGFR were generated using a site-directed mutagenesis kit (Stratagene, La Jolla, Calif.). The template for each mutagenesis was the human EGFR cDNA (accession number x00588) (Ullrich et al. (1984) Nature. 309, 418-425). Automated nucleotide sequencing of each construct was performed to confirm the integrity of the EGFR mutations. Wild-type and mutant (C173A/C281A) EGFR were transfected into BaF/3 cells by electroporation.

Stable cell lines expressing the mutant EGFR were obtained by selection in neomycin-containing medium. After final selection, mRNA was isolated from each cell line, reverse transcribed and the EGFR sequence amplified by PCR. All mutations in the expressed EGFR were confirmed by sequencing the PCR products. The level of EGFR expression was determined by FACS analysis on a FACStar (Becton and Dickinson, Franklin Lakes, N.J.) using the anti-EGFR antibody mAb528 (Masui et al. (1984) Cancer Res. 44, 1002-1007; Gill et al. (1984) J. Biol. Chem. 259, 7755-7760) at 10 μg/ml in PBS, 5% FCS, 5 mM EDTA followed by Alexa 488-labeled anti-mouse Ig (1:400 final dilution). Background fluorescence was determined by incubating the cells with an irrelevant, class-matched primary antibody. All cells were routinely passaged in RPMI, 10% FCS, 10% WEHI3B conditioned medium and 1.5 mg/ml G418.

### EGF Activation of Mutant EGFR

Cells expressing the wtEGFR or C271A/C283A-EGFR were washed and incubated for 3 hr in medium without serum or IL-3. Cells were collected by centrifugation and resuspended in medium containing EGF (100 ng/ml) or an equivalent volume of PBS. Cells were harvested after 15 min, pelleted and lysed directly in SDS/PAGE sample buffer containing p-mercaptoethanol. Samples were separated on NuPAGE 4-12% gradient gels, transferred to Immobilon PVDF membrane and probed with anti-phosphotyrosine (4G10, Upstate Biotechnologies) or anti-EGFR antibodies (mAb806, produced at the LICR). Reactive bands were detected using chemiluminescence.

### Effect of EGF and Antibodies on Cell Proliferation

Cells growing in log phase were harvested and washed twice with PBS to remove residual IL-1 Cells were resuspended in RPMI 1640 plus 10% FCS and seeded into 96-well plates at 10⁵ cells/well with carrier only or with increasing concentrations of EGF. Where appropriate, a fixed concentration of mAb528 or mAb806 (2 μg/well) was also added to the cultures. Proliferation was determined using the MTT assay (van de Loosdrecht et al. (1994) J. Immunol. Methods. 174, 311-320).

Reactivity with Conformation-Specific Antibodies

Cells were collected by centrifugation and stained with the control or test antibodies (all at 10 μg/ml in FACS buffer for 40 min on ice, washed in FACS buffer) followed by Alexa 488-labeled anti-mouse Ig (1:400 final dilution, 20 min on ice). The cells were washed with ice-cold F ACS buffer, collected by centrifugation, and analyzed on a FACScan; peak fluorescence channel and median fluorescence were determined for each sample using the statistical tool in Cell Quest (Becton and Dickinson). Background (negative control) fluorescence was deducted from all measurements. The median fluorescence values were chosen as most representative of peak shape and fluorescence intensity and were used to derive the ratio of mAb806 to mAb528 binding.

### Crystal Structure Determinations of Fab 175, and Fab 806, Fab-Peptide Complexes and the NMR Structure of the 806 Peptide Epitope in Solution

Structures were determined by molecular replacement and refinement converged with R=0.225/Rfree-=0.289 for Fab806 and R=0.226/Rfree=0.279 for Fab806:peptide; R=0.210/Rfree=0.305 for Fab806 and R=0.203/Rfree=0.257 for Fab806:peptide.

Crystals of native 806 Fab were grown by hanging drop vapor diffusion using 10 mg/ml Fab and a reservoir containing 0.1M Sodium acetate buffer pH 4,6, 6-8% PEG6000 and 15-20% Isopropanol. For data collection crystals were transferred to a cryoprotectant solution containing 0.1M Sodium acetate buffer pH 4.6, 10% PEG6000, 15-20% Isopropanol and 10% glycerol. Crystals were then mounted in a nylon loop and flash frozen directly into liquid nitrogen.

Crystals of 806 Fab-peptide complex were grown by hanging drop vapor diffusion using 10 mg/ml Fab-peptide complex and a reservoir containing 0.2M ammonium acetate 16-18% PEG 5,000 monomethylether, crystals quality was then improved through seeding techniques. For data collection crystals were transferred to a cryoprotectant solution consisting of reservoir supplemented with 25% glycerol. Crystals were then mounted in a nylon loop and flash frozen directly into liquid nitrogen.

Crystals of 175 Fab-peptide complex were initially grown by free interface diffusion using a Topaz crystallization system (Fluidigm, San Francisco). Microcrystals were grown by hanging drop vapor diffusion using 7 mg/ml Fab with similar conditions 0.1M Bis-tris propane buffer, 0.2M ammonium acetate and 18% PEG 10,000. Microcrystals were then improved by streak seeding into 0.15 m Sodium formate and 15% PEG 1500 to yield small plate shaped crystals. For data collection crystals were transferred to a cryoprotectant solution consisting of reservoir supplemented with 25% glycerol. Crystals were then mounted in a nylon loop and flash frozen directly into liquid nitrogen.

Diffraction data on 806 Fab and 175 Fab complex crystals were collected in-house using a R-AXIS IV detector on a Rigaku micromax-007 generator fitted with AXCO optics, these data were then processed using CrystalClear. 806 Fab-peptide complex data were collected on an ADSC quantum315 CCD detector at beamline X29, Brookhaven National Laboratory, these data were processed with HKL2000 (Otwinowski, Z. and Minor, W. (1997) Processing of X-ray diffraction data collected in oscillation mode. Academic Press (New York)) (data collection statistics are shown in Table 9). Native 806 Fab was solved by molecular replacement using the program MOLREP (Vagin, A. and Teplyakov, A. (1997) J. Appl. Cryst. 30, 1022-1025) using the coordinates of the Fab structure 2E8 refinement of the structure was performed in REFMAC5 (Murshudov et al. (1997) Acta crystallographica 53, 240-255) and model building in Coot (Emsley, P. and Cowtan, K. (2004) Acta crystallographica 60, 2126-2132).

Both 806-peptide and 175 Fab-peptide structures were solved by molecular replacement using the program MOLREP using the coordinates of the 806 Fab structure, refinement and rebuilding were again performed in REFMAC5, and COOT and O. Validation of the final structures were performed with PROCHECK (Laskowski et al. (1993) J. Appl. Cryst. 26, 283-291) and WHATCHECK (Hooft et al. (1996) Nature 381, 272).

### NMR Studies

For NMR studies, ¹⁵N-Iabelled peptide was produced recombinantly as a fusion to the SH2 domain of SHP2 using the method previously described by Fairlie et al. (Fairlie et al. (2002) Protein expression and purification 26, 171-178) except that the E. coli were grown in Neidhardt's minimal medium m suppleented with ¹⁵NH₄Cl (Neidhardt et al. (1974) Journal of bacteriology 119, 736-747). The peptide was cleaved from the fusion partner using CNBr, purified by reversed-phase HPLC and its identity confirmed by MALDI-TOF mass spectrometry and N-terminal sequencing. The methionine residue within the 806 antibody-binding sequence was mutated to leucine to enable cleavage from the fusion partner, but not within the peptide itself.

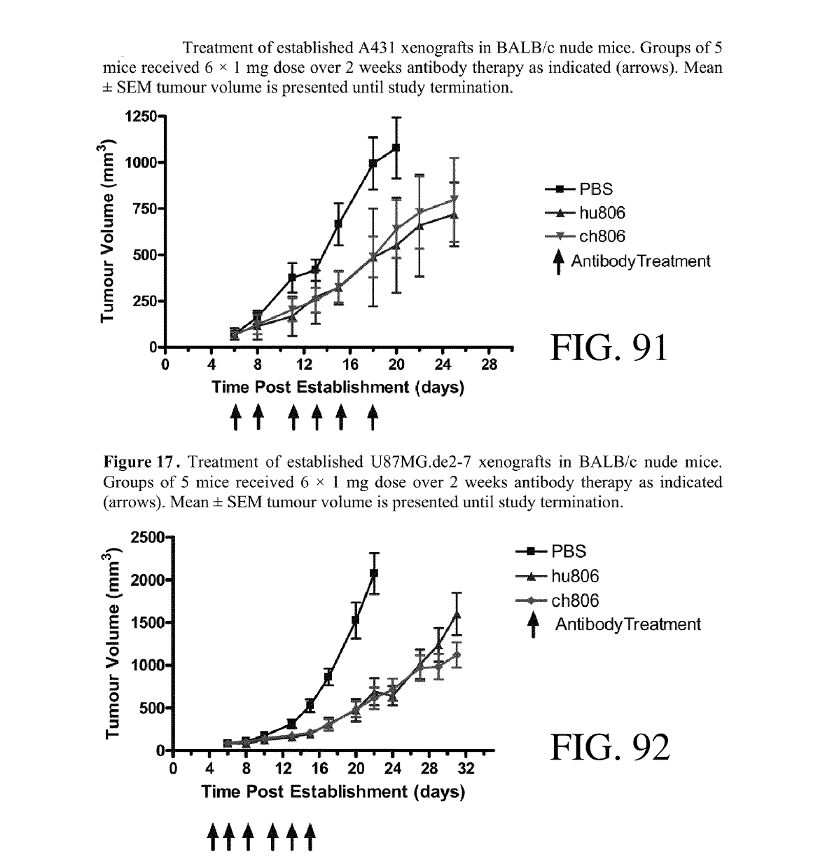

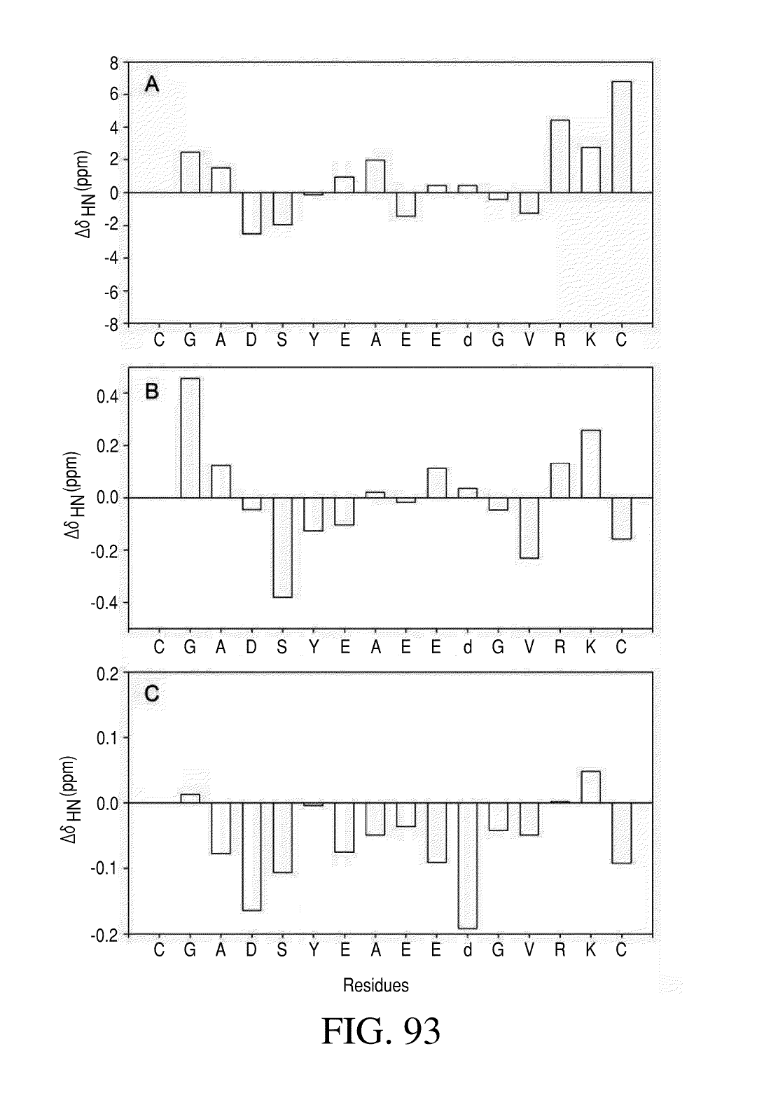

Samples used for NMR studies were prepared in H_{2O} solution containing 5% ²H₂O, 70 mM NaCl and 50 mM NaPO₄ at pH 6.8. All spectra were acquired at 298K on a Bruker Avance500 spectrometer using a cryoprobe. Sequential assignments of the peptide in the absence of m806Fab were established using standard 2D TOCSY and NOESY as well as ¹⁵N-edited TOCSY and NOESY spectra. Interaction between the peptide and fAb806 was examined by monitoring ¹⁵N HSQC spectra of the peptide in the absence and presence of fAb806. Spectral perturbation of ¹⁵N HSQC spectra of the peptide in the presence of fAb806 clearly indicates the peptide was able to bind to the fAb806 under the presence solution conditions. Detailed conformation of the peptide in the complex form was not determined. Deviations from random coil chemical shift values for the mAb806 peptide are shown in FIG. 93.

Biodistribution of chAb806 Tumor in Patients

To demonstrate the tumor specificity of mAb806 in vivo, a chimeric version (ch806) was engineered and produced under cGMP conditions (Panousis et al. (2005) Br. J. Cancer. 92, 1069-1077). A Phase I first-in-man trial was conducted to evaluate the safety, biodistribution and immune response of ch806 in patients with 806 positive tumors, and the results of safety, biodistribution and pharmacokinetics have been reported previously (Scott et al. (2007) Proc. Natl. Acad. Sci. U.S.A. 104, 4071-4076). To define the specificity of ch806 in tumor compared to normal tissue (i.e., liver) in patients, the quantitative uptake of ch806 in tumor and liver was performed by calculation of % injected dose (ID) of ¹¹¹In-ch806 from whole body gamma camera images obtained over one week following injection of 5-7mCi (200-280 MBq) ¹¹¹In-ch806. Liver and tumor dosimetry calculations were performed based on regions of interest in each individual patient. ¹¹¹In-ch806 infusion image dataset, corrected for background and attenuation, allowing calculation of cumulated activity. Dosimetry calculation was performed to derive the concentration of ¹¹¹In-ch806 in tumor and liver over a one week period post injection.

b. Sequencing

The variable heavy (VH) and variable light (VL) chains of mAb175 were sequenced, and their complementarity determining regions (CDRs) identified, as follows:

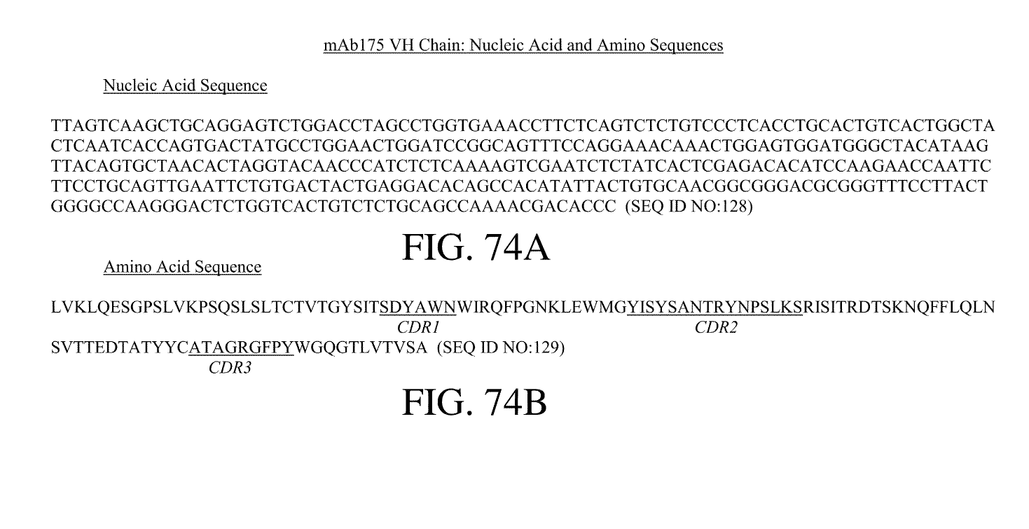

mAb175 VH chain: nucleic acid (SEQ ID NO:128) and amino acid (SEQ ID NO:129) sequences are shown in FIGS. 74A and 74B, respectively. Complementarity determining regions CDR1, CDR2, and CDR3 (SEQ ID NOS:130, 131, and 132, respectively) are indicated by underlining in FIG. 74B.

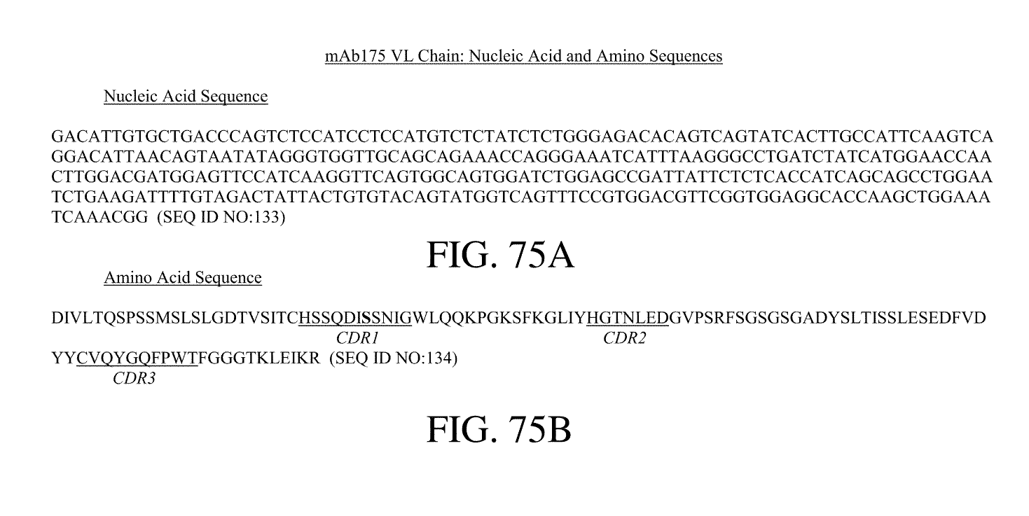

mAb175 VL chain: nucleic acid (SEQ ID NO:133) and amino acid (SEQ ID NO:134) sequences are shown in FIGS. 75A and 75B, respectively. Complementarity determining regions CDR1, CDR2, and CDR3 (SEQ ID NOS: 135, 136, and 137, respectively) are indicated by underlining in FIG. 75B.

The sequence data for mAb175 is based on both sequence and crystal structure data, as the cell line is not clonal, and therefore multiple sequences have been obtained from the cell line. The sequences of mAb175 set forth above have been confirmed by crystal structure, and differ by a single amino acid in each of the VL chain CDR1 and CDR2 from previous sequences based on standard sequence data alone. A different isotype of mAb175 (an unusual IgG2a isotype) has also been obtained, based on the final sequence and crystal structure data.

mAb175 Specificity

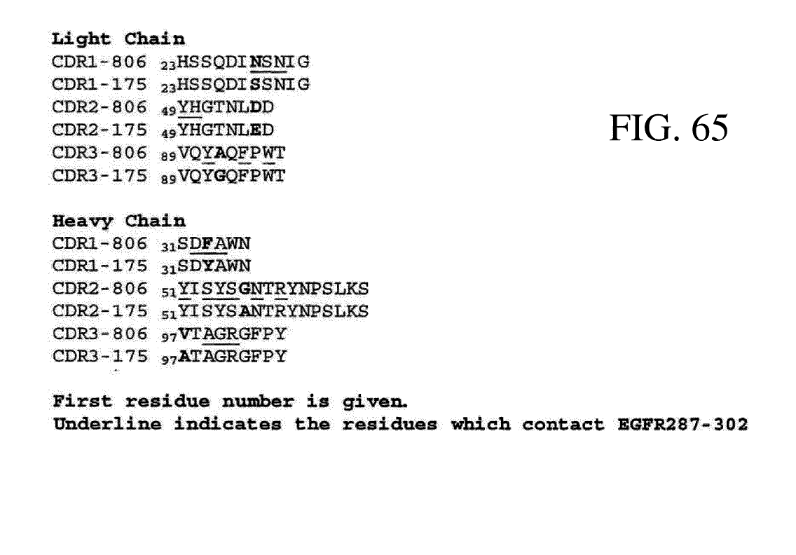

Preliminary binding studies suggested that mAb175 displayed similar specificity for EGFR as mAb806. In the CDR regions of mAb806 (IgG2b) and mAb175 (IgG2a), the amino acid sequences are almost identical, with only one amino acid difference in each (FIG. 65; See Example 26, below). All these differences preserve the charge and size of the side-chains. Clearly these antibodies have arisen independently.

c. Experiments

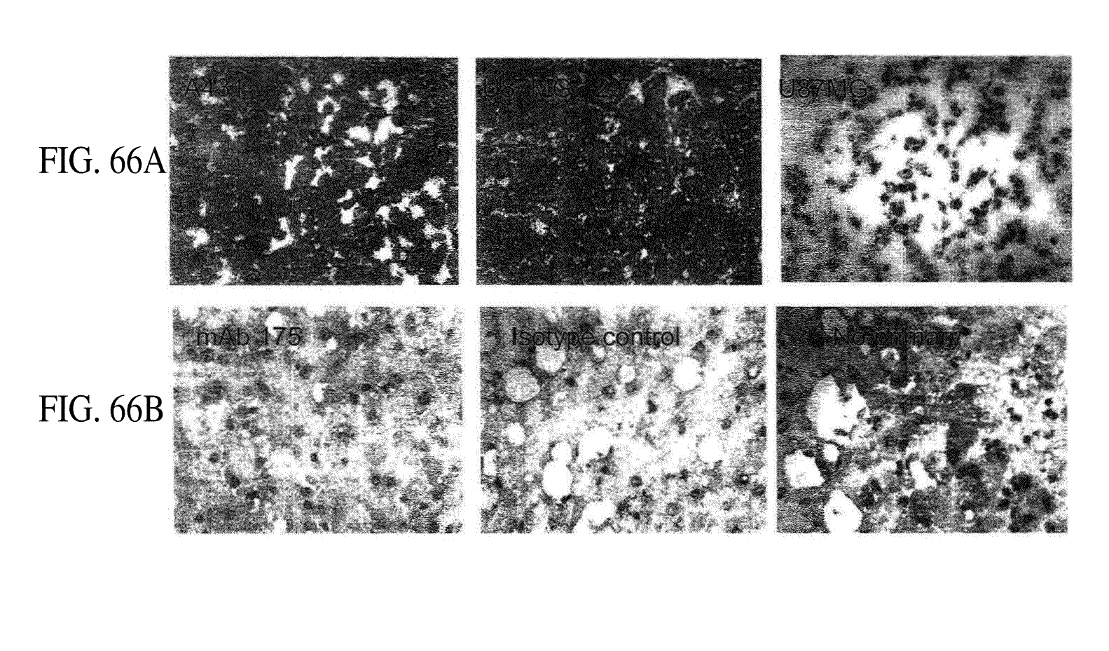

A set of immunohistochemistry experiments were conducted to analyze the specificity of mAb175 binding. mAb175 stains sections of A431 xenografts that overexpress the EGFR (FIG. 66A) and sections of U87MG.Δ2-7 glioma xenografts that express the Δ2-7EGFR (FIG. 66A). In contrast, mAb175 does not stainU87MG xenograft sections. The U87MG cell line only expresses modest levels of the wild-type EGFR (FIG. 66A) and has no detectable EGFR autocrine loop. Most importantly, .mAb175 does not bind to normal human liver sections (FIG. 66B). Thus, mAb175 appears to demonstrate the same specificity as mAb806, i.e. it detects over-expressed and truncated human EGFR, but not the wtEGFR expressed at modest levels.

Identification of the mAb175 Epitope

Since mAb175 also binds the Δ2-7EGFR, in which amino acids 6-273 are deleted, and EGFR₁₋₅₀₁, the mAb175 epitope must be contained within residues 274-501. When determining the epitope of mAb806, we expressed a series of c-myc-tagged EGFR fragments fused to the carboxy terminus of human GH, all terminating at amino acid 501 (Chao et al. (2004) J. Mol. Biol. 342, 539-550; Johns et al. (2004) J. Biol. Chem. 279, 30375-30384).

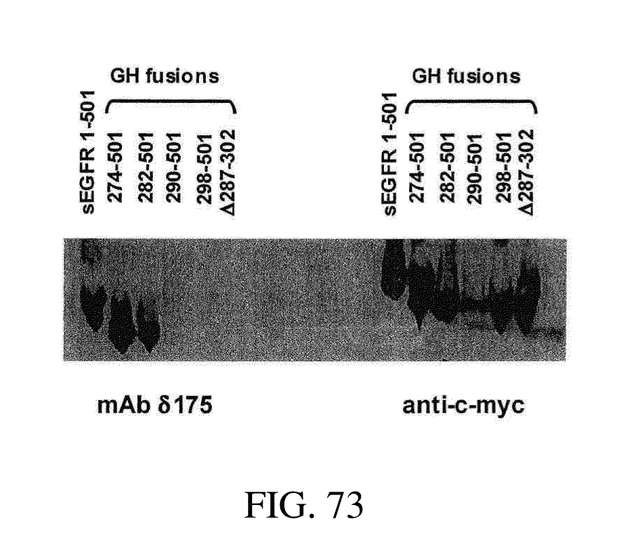

The mAb175 also reacted with both the 274-501 and 282-501 EGFR fragments in Western blots, but did not detect fragments commencing at amino acid 290 or 298 (FIG. 73). The presence of all GH-EGFR fusion proteins was confirmed using the c-myc antibody, 9E10 (FIG. 73). Therefore, a critical determinant of the mAb175 epitope is located near amino acid 290. Finally, a 274-501 EGFR fragment with the mAb806 epitope deleted (4287-302) was also negative for mAb175 binding (FIG. 73), suggesting that this region similarly determined most of the mAb175 binding.

A second approach was used to characterize the mAb175 epitope further. Fragments encompassing extracellular domains of the EGFR were expressed on the surface of yeast and tested for mAb175 binding by indirect immunofluorescence using flow cytometry. The mAb175 recognized the yeast fragment 273-621, which corresponds to the extracellular domain of the Δ2-7 EGFR, but not to fragments 1-176, 1-294, 294-543, or 475-621 (FIG. 67A and FIG. 67B). Thus, at least part of the mAb175 epitope must be contained within the region between amino acids 274-294, agreeing with immunoblotting data using EGFR fragments. Since mAb175 binds to the denatured fragment of the 273-621 (FIG. 67C), the epitope must be linear in nature (FIG. 73). It is clear that mAb806 and mAb175 recognize a similar region and conformation of the EGFR.

Using surface plasmon resonance (BIAcore) the binding of mAb175 to the EGFR peptide (₂₈₇CGADSYEMEEDGVRKC₃₀₂; SEQ ID NO:138)) was investigated. The EGFR₂₈₇₋₃₀₂ was immobilized on the biosensor surface using amine, thiol-disulfide exchange or Pms-Ser coupling chemistries. The latter method immobilizes the peptide exclusively through the N-terminal cysteine (Wade et al. (2006) Anal. Biochem. 348, 315-317).

mAb175 bound the EGFR₂₈₇₋₃₀₂ in all orientations (Table 6). The affinity of mAb175 for EGFR₂₈₇₋₃₀₂ ranged from 35 nM for Pms-serine coupling to 154 nM for amine coupling. In all cases the binding affinity of mAb175 for EGFR₂₈₇₋₃₀₂ was lower than that obtained for mAb806 (Table 6). We also determined the affinity of mAb175 to two different extracellular fragments of the EGFR. mAb175 bound the 1-501 fragment with an affinity similar to that obtained using the peptide (16 nM versus 35 nM) (Table 6). As expected, the affinity of mAb175 against the 1-621 full length extracellular domain, which can form the tethered conformation, was much lower (188 nM). Although mAb806 and mAb 175 have similar affinities for EGFR₂₈₇₋₃₀₂, mAb175 appears to display a higher affinity for the extra-cellular domain of the EGFR (Table 6). Clearly, the mAb175 epitope is contained within the EGFR₂₈₇₋₃₀₂ and, like mAb806, the binding affinity to extra-cellular domain of the EGFR is dependent on conformation.

TABLE 6
BIAcore determination of antibody affinities for
mAb806 and mAb175 binding to EGFR epitopes

| | K_{D} for | K_{D} for |
|---|---|---|
| EGFR Fragment | mAb175 (nM) | mAb806 (nM) |
| 287-302 (Pms-Ser coupling) | 35 | 16 |
| 287-302 (Thiol coupling) | 143 | 84 |
| 287-302 (Amine coupling) | 154 | 85 |
| 1-501 (Unable to form tether) | 16 | 34 |
| 1-621 (Can form tether) | 188 | 389 |

The panel of mutants of the 273-621 EGFR fragment, expressed on the surface of yeast (Chao et al. (2004) J. Mol. Biol. 342, 539-550; Johns et al. (2004) J. Biol. Chem. 279, 30375-30384) was used to characterize the fine structure of the mAb175 epitope. mAb 175 and mAb806 displayed a near identical pattern of reactivity to the mutants (Table 7). Disruption of the 287-302 disulfide bond only had a moderate effect on the epitope reactivity as the antibody bound to all mutants at C287 and to some but not all mutants at C302 (Table 7) Amino acids critical for mAb175 binding include E293, G298, V299, R300 and C302 (Table 7). mAb175 appeared moderately more sensitive to mutations V299 and D297 but mAb806 also showed reduced binding to some mutations at these sites (Table 7). Again, the mAb175 epitope appears to be essentially the same as the epitope recognized by mAb806.

TABLE 7
Display of EGFR Epitope 287-302 mutations on yeast
and the binding scores for mAb806 and mAb175

| EGFR Mutant | mAb806 Binding | mAb175 Binding |
|---|---|---|
| C287A | + | + |
| C287G | + | + |
| C287R | + | + |
| C287S | + | + |
| C287W | + | + |
| C287Y | + | + |
| G288A | ++ | ++ |
| A289K | ++ | ++ |
| D290A | ++ | ++ |
| S291A | ++ | ++ |
| Y292A | ++ | ++ |
| E293A | + | + |
| E293D | + | + |
| E293G | + | + |
| E293K | — | — |
| M294A | ++ | ++ |
| E295A | ++ | ++ |
| E296A | ++ | ++ |
| D297A | ++ | + in contact |
| D297Y | + | + |
| G298A | + | + |
| G298D | — | — |
| G298S | — | — |
| V299A | ++ | + in contact |
| V299D | — | — |
| V299K | ++ | + in contact |
| R300A | ++ | ++ |
| R300C | + | + |
| R300P | — | — |
| K301A | ++ | ++ |
| K301E | + | + |
| C302A | — | — |
| C302F | + | + |
| C302G | — | — |
| C302R | + | + |
| C302S | — | — |
| C302Y | + | + |

Efficacy of mAb175 Against Tumor Xenografts Stimulated by Δ2-7EGFR or an EGFR Autocrine Loop

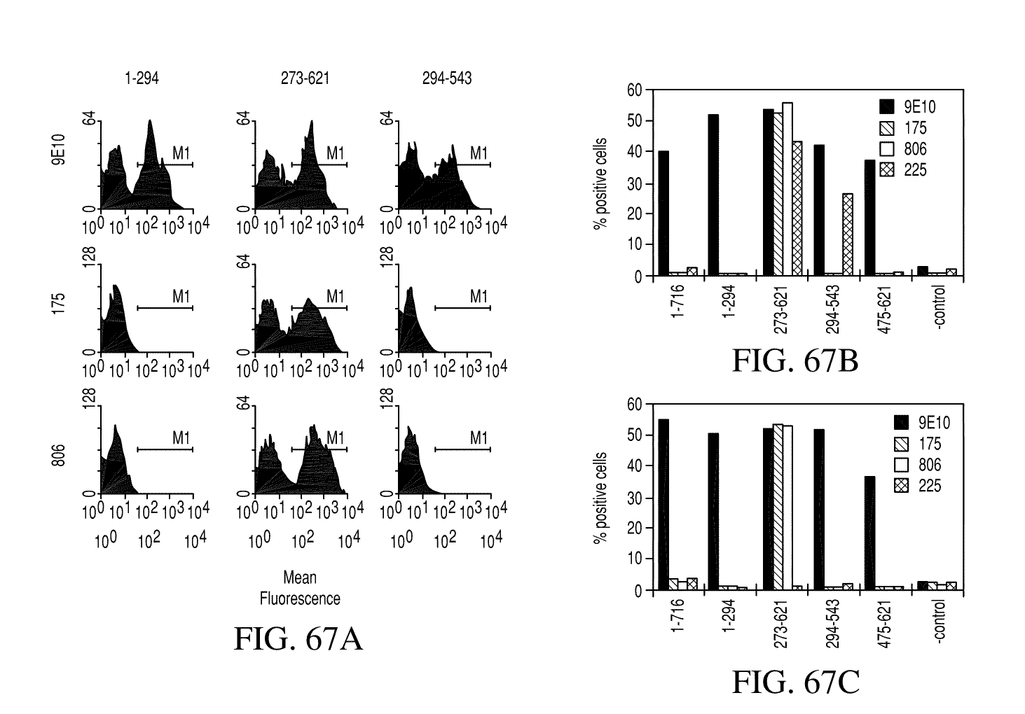

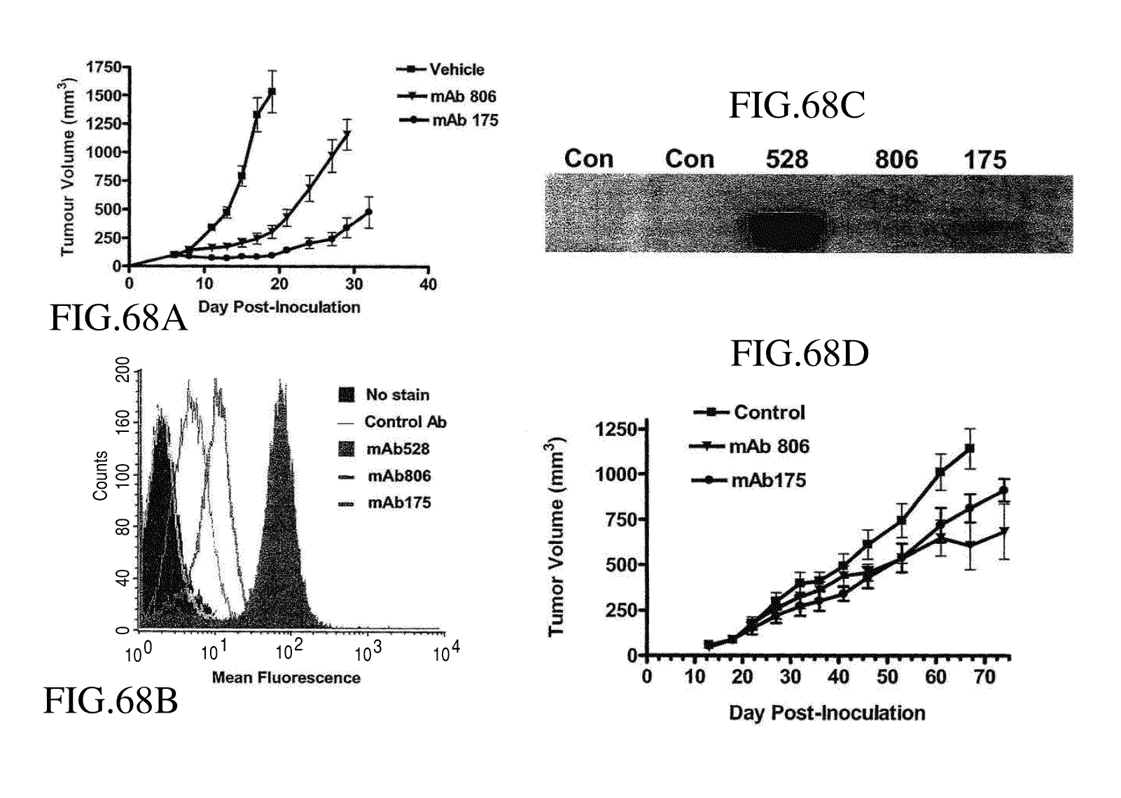

The in vivo anti-tumor activity of mAb806 and mAb175 against U87MG.Δ2-7 glioma xenografts was examined. Xenografts were allowed to establish for 6 days before antibody therapy (3 times a week for 2 weeks on days indicated) commenced. At this time, the average tumor volume was 100 mm³ (FIG. 68A). mAb175 treatment resulted in a reduction in overall tumor growth rate compared to treatment with vehicle or mAb806 and was highly significant at day 19 post-inoculation (P<0.0001 versus control and P<0.002 versus mAb806), when the control group was sacrificed for ethical reasons. The average tumor volume at this time was 1530, 300 and 100 mm³ for the vehicle, mAb806 and mAb175 treatment groups, respectively (FIG. 68A), confirming the antitumor activity of mAb175 activity against xenografts expressing the Δ2-7 EGFR.

Even though U87MG cells express approximately 1×10⁵ EGFR per cell, mAb 806 is not able to recognize any of the surface EGFR, and not surprisingly, does not inhibit U87MG in vivo growth. Furthermore these cells do not co-express any EGFR ligand. A study was conducted as to whether the EGFR epitope is transiently exposed, and hence able to be recognized by mAb806 and mAb175 in cells containing an EGFR autocrine loop. The prostate cell line DU145 expresses the wtEGFR at levels similar to that observed in U87MG cells, however unlike the U87MG cells, the DU145 cells contain an amplification of the TGF-α gene and thus exhibit an EGFR/TGF-α autocrine loop. Both mAb175 and 806 bind to DU145 cells as determined by FACS analysis (FIG. 68B) and both are able to immunoprecipitate a small proportion of the EGFR extracted from these cells (FIG. 68C). Both techniques showed greater binding of mAb175, however, when compared to mAb528, which binds to the L2 domain, mAb175 and mAb806 only bind a subset of EGFR on the surface of these cells (FIG. 68B and FIG. 68C). Similar observations were seen with a second prostate cell line (LnCap); (data not shown) and a colon line (LIM1215) both of which also contain EGFR autocrine loops (Sizeland, A. M. and Burgess, A. W. (1992) Mol Cell Biol. 3, 1235-1243; Sizeland, A. M. and Burgess, A. W. (1991) Mol Cell Biol. 11, 4005-4014). Clearly, mAb806 and mAb175 can recognize only a small proportion of the EGFR on cells in the presence of an autocrine stimulation loop.

Since mAb175 and mAb806 bind more effectively to the EGFR expressed in DU145 cells than U87MG cells, a study was conducted to analyze the anti-tumor activity of these antibodies in DU145 xenografts grown in nude mice. Xenografts were allowed to establish for 18 days before therapy commenced (3 times a week for 3 weeks on days indicated). At this time the average tumor volume was 90 mm³ (FIG. 68D). Both mAb175 and mAb806 inhibited the growth of DU145 xenografts. The control group was sacrificed on day 67 and had a mean tumor volume of 1145 mm³ compared with 605 and 815 mm³ for the mAb806 and rnAb175 groups respectively (p<0.007 and 0.02 respectively) (FIG. 68D).

3D-Structure of EGFR₂₈₇₋₃₀₂ in Contact with the Fab Fragments of mAb806 and mAb175

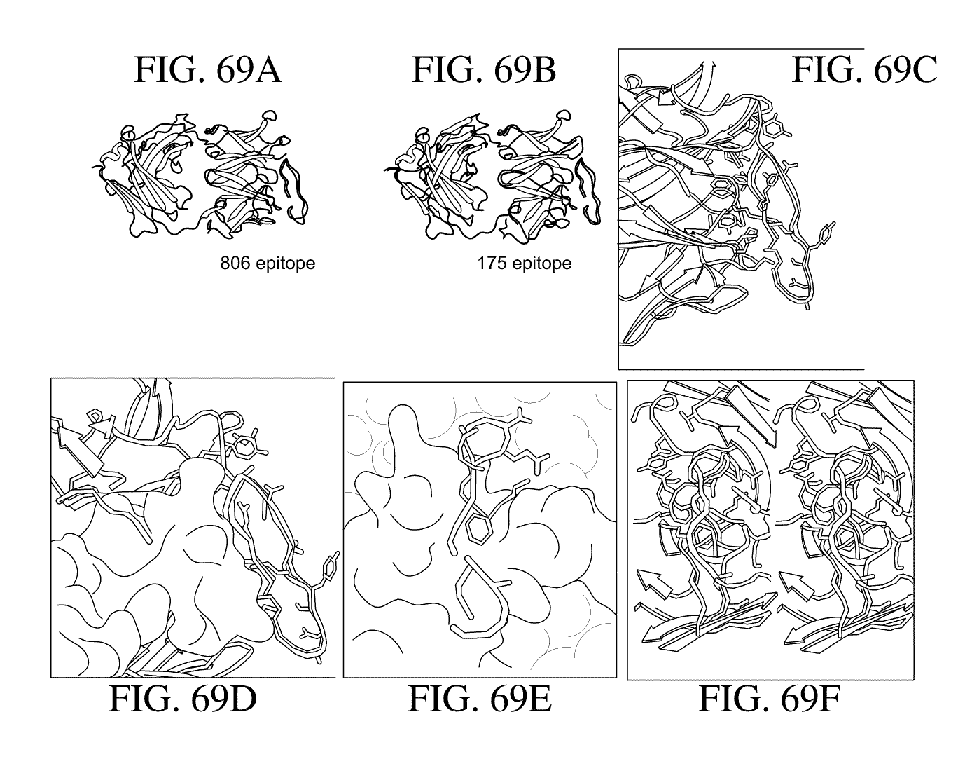

In order to understand the molecular details of how mAb806 and mAb175 could recognize EGFR in some, but not all conformations, the crystal structures of Fab fragments for both antibodies were determined in complex with the oxidized EGER₂₈₇₋₃₀₂ epitope (at 2.0 and 1.59 Å resolution respectively, FIGS. 69A & 69B) and alone (at 2.3 Å and 2.8 Å resolution, respectively). In both cases, the free and complexed Fab structures were essentially the same and the conformations of the peptide and CDR loops of the antibodies were well defined (FIG. 69). The epitope adopts a β-ribbon structure, with one edge of the ribbon pointing towards the Fab and V299 buried at the centre of the antigen-binding site (FIGS. 69C-E). Both ends of the epitope are exposed to solvent, consistent with these antibodies binding much longer polypeptides.

Of the 20 antibody residues in contact withh the epitope, there are only two substitutions between mAb806 and mAb175 (FIG. 65). mAb175 contact residues are: light-chain S30, S31, N32, Y49, HS0, Y91, F94, W96 and heavy-chain D32, Y33, A34, Y51, S53, Y54, S55, N57, R59, A99, G100, R101; the mAb806 contact residues are the same, with sequence differences for the light-chain, N30 and heavy-chain, F33. _{EGFR287-302} binds to the Fab through close contacts between peptide residues 293-302, with most of the contacts being between residues 297 and 302. The only hydrogen bonds between main chain atoms of EGFR₂₈₇₋₃₀₂ and the Fab are for residues 300 and 302 (FIG. 69F). Recognition of the epitope sequence occurs through side-chain hydrogen bonds to residues E293 (to H50 and R101 of the Fab), D297 (to Y51 and N57), R300 (to D32) and K301 (via water molecules to Y51 and W96). Hydrophobic contacts are made at G298, V299 and C302.

The conformation of the epitope backbone between 293 and 302 was essentially identical in the Fab806 and Fab175 crystals (rms deviation=0.4 A, for Cα atoms in these residues). Although constrained by the disulfide bond, the N-terminus of the peptide (287-292) does not make significant contact in either antibody structure and conformations in this region differ. However, this segment in the Fab806 complex appears rather disordered. More interestingly, the conformation of the EGFR₂₈₇₋₃₀₂ peptide in contact with the antibodies is quite closely related to the EGFR₂₈₇₋₃₀₂ conformation observed in the backbone of the tethered or untethered EGFR structures (Li et al., 2005; Garrett et al., 2002). For EGFR₂₈₇₋₃₀₂ from the Fab175 complex, the rms deviations in Cα positions are 0.66 and 0.75 Å, respectively (FIG. 69).

To gain further insight into the recognition of EGFR by mAb806 and mAb175, the conformation of ¹⁵N-labelled oxidized peptide EGFR₂₈₇₋₃₀₂ was studied by NMR spectroscopy in solution, free and in the presence of 806 Fab (see Materials and Methods). For the free peptide, resonances were assigned and compared to those for random coil. Essentially, the free peptide adopted a random coil structure, not the beta ribbon as seen in the native EGFR (Garrett et al. (2002) Cell 20; 110, 763-773).

Upon addition of the Fab, resonance shifts were observed. However, due to the weak signal arising from significant line broadening upon addition of the Fab and successful crystallization of the complexes, the solution structure of the Fab806-epitope complex was not pursued further. Clearly though, when the peptide binds to the Fab fragment of InAb806 (or mAb175) it appears that the Fab selects or induces the conformation of the peptide which matches that peptide in the native receptor.

In order to study why mAb806 and mAb175 recognize only some conformations of EGFR, the Fab fragment of mAb175 was docked onto an extra-cellular domain of EGFR (tethered and untethered monomers) by superimposing EGFR₂₈₇₋₃₀₂. For a Δ2-7-like fragment there were no significant steric clashes with the receptor. In the untethered form there was substantially more accessible surface area of the Fab buried (920 Å² compared with 550^{Å2} in the tethered form). Therefore, this antigen may make additional contacts with non-CDR regions of the antibody, as has been indicated by yeast expression mutants (Chao et al. (2004) J. Mol. Biol. 342, 539-550). Conversely, docking the whole EGFR ectodomain onto the Fab, there is substantial spatial overlap with the part of the CR1 domain preceding the epitope (residues 187-286) and running through the centre of the Fab (FIGS. 69D and 69E). Hence, as the CR1 domain has essentially the same structure in tethered or untethered conformations, mAb806 or mAb175 will be unable to bind to either form of EGFR. Clearly, there must be a difference between the orientation of the epitope with respect to the CR1 domain in either known conformations of the wtEGFR and the orientation that permits epitope binding. Inspection of the CR1 domain indicated that the disulfide bond (271-283) preceding EGFR₂₈₇₋₃₀₂ constrains the polypeptide which blocks access to the epitope; disruption of this disulfide, even though it is not involved in direct binding to the antibodies, would be expected to allow partial unfolding of the CR1 domain so that mAb175 or mAb806 could gain access to the epitope.

Breaking of the EGFR 271-283 Disulfide Bond Increases mAb806 Binding

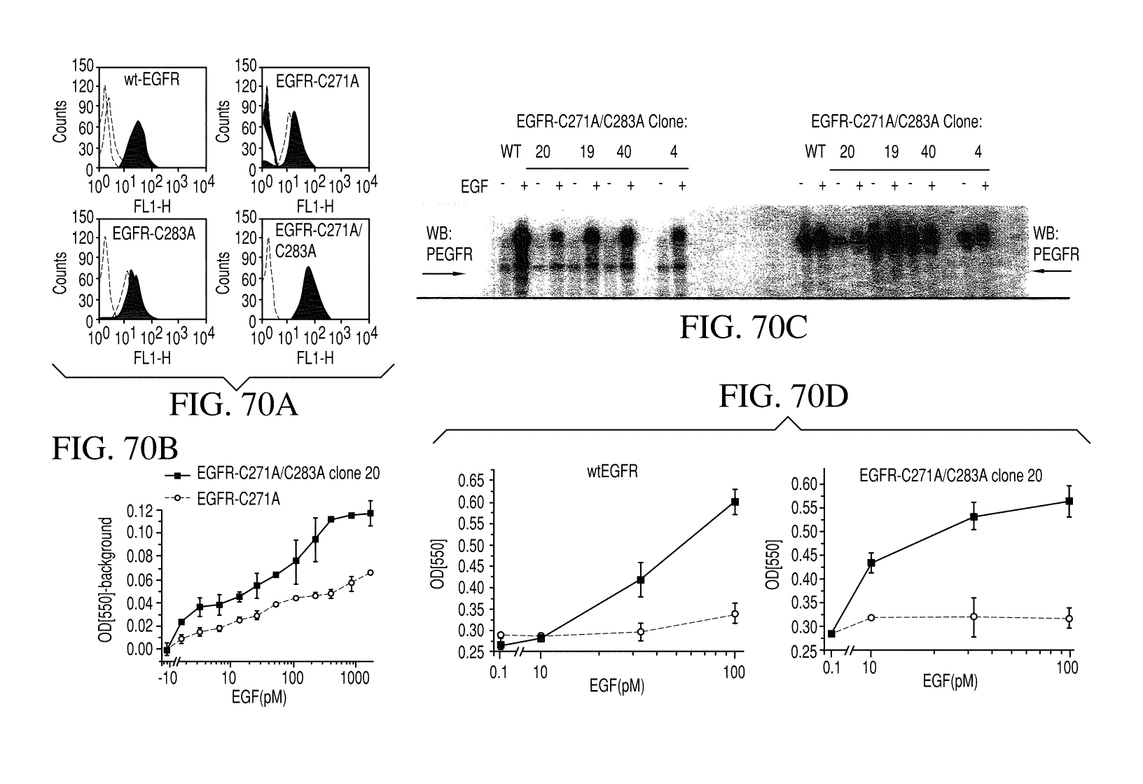

Disulfide bonds in proteins provide increased structural rigidity but in some cell surface receptors, particularly those for cytokines and growth factors, transient breaking of disulfide bonds and disulfide exchange can control the receptor's function (Hogg, P. J. (2003) Trends in biochemical sciences 28, 210-214). As this was one mechanism by which mAb806 and mAb175 could gain access to their binding site, increasing the accessibility of the epitope was attempted by mutating either or both of the cysteine residues at positions 271 and 283 to alanine residues (C271A/C283A). The vectors capable of expressing full length C271A-, C283A- or C271A/C283A-EGFR were transfected into the IL-3 dependent Ba/F3 cell line. Stable Ba/F3 clones, which expressed the C271A- and C271A/C283A-EGFR mutant at levels equivalent to the wtEGFR were selected (FIG. 70A. Ba/F3 cells expressing high levels of mutant C283A-EGFR were not observed. As previously described, the wtEGFR reacts poorly with mAb806; however, the mutant receptors reacted equally strongly with mAb528, mAb806 and the anti-FLAG antibody, suggesting that the receptor is expressed at the cell surface, is folded correctly and that the epitope for tnAb806 is completely accessible in such cases. To confirm that mAb806 recognizes the C271A/C283A mutant more efficiently than the wtEGFR, the ratio of mAb806 binding to the binding of mAb528 was determined Since both the wild-type and C271A/C283A EGFR were N-terminally FLAG-tagged, the ratio of mAb806 and mAb528 binding to the M2 antibody was also determined. As reported previously, mAb806 only recognized a small proportion of the total wtEGFR expressed on the surface of Ba/F3 cells (the mAb806/528 binding ratio is 0.08) (Table 8). In contrast, mAb806 recognized virtually all of the C271A/C283A mutant EGFR expressed on the cell surface (an mAb806/528 binding ratio of 1.01) (FIG. 70A and Table 8).

TABLE 8
mAb806 reactivity with cells expressing
the wild-type or C271A/C283A EGFR

| | Ratios of antibody binding | | |
|---|---|---|---|
| Cell Line | mAb 528/M2 | mAb806/M2 | mAb806/mAb 528 |
| wtEGFR- | 1.37 | 0.11 | 0.08 |
| FLAG | | | |
| wt-EGFR | — | — | 0.07 |
| C271/283* | 1.08 ± 0.10 | 1.09 ± 0.38 | 1.01 ± 0.13 |

*Average for four independent clones

Mutation of the two cysteines did not compromise EGF binding or receptor function. BaF3 cells expressing the C271A/C283A EGFR mutant proliferate in the presence of EGF (FIG. 70B). A left-shift in the dose response curve for EGF in cells expressing the C271A/C283A mutations was reproducibly observed, suggesting either higher affinity for the ligand, or enhanced signaling potential for the mutant receptor. Western blotting analysis confirmed that the C271A/C283A mutant is expressed at similar levels to the wtEGFR and is tyrosine phosphorylated in response to EGF stimulation (FIG. 70C). Consistent with previous studies in other cell lines, mAb806 has no effect on the in vitro EGF-induced proliferation of Ba/F3 cells expressing the wtEGFR, while the ligand blocking mAb528 completely inhibits the EGF-induced proliferation of these cells (FIG. 70D, left panel). In contrast, mAb806 totally ablated the EGF-induced proliferation in BaF3 cells expressing the C271A/C283A mutant (FIG. 70D, right panel). When the 271-283 cysteine loop is disrupted, not only does mAb806 bind more effectively, but once bound, mAb806 prevents ligand induced proliferation.

TABLE 9
Data Collection and Refinement Statistics

| | | | |
|---|---|---|---|
| 806 (native) | 806 (peptide) | 175 (native) | 175 (peptide) |

Data Collection

| | | | | |
|---|---|---|---|---|
| Space Group | P2₁2₁2 | P2₁ | P2₁2₁2₁ | P2₁2₁2 |
| Cell Dimensions (Å) | | | | |
| a | 140.37 | 35.92 | 36.37 | 83.17 |
| b | 74.62 | 83.16 | 94.80 | 69.26 |
| c | 83.87 | 72.21 | 108.90 | 71.47 |
| | | β = 92.43 | | |
| Source | in-house | BNL X29 | in-house | in-house |
| Wavelength (Å) | 1.542 | 1.1 | 1.542 | 1.542 |
| Resolution Range (Å) | 29.7-2.2 | 50-2.0 | 50-2.8 | 14.18-1.59 |
| | (2.27-2.20) | (2.07-2.0) | (2.87-2.8) | (1.65-1.59) |
| R_{merge} (%) | 6.4 (26.7) | 6.6 (28.2) | | 8.6 (30.0) |
| I/σI | 12.2 (3.2) | 22 (3.15) | | 10.2 (2.2) |
| Completeness (%) | 98.3 (91.3) | 96.6 (79.2) | 98.4 (90.5) | 78.8 (11.8) |
| | | | | 98.1 at 1.89 Å |
| Total Reflections | 156497 | 98374 | | 205401 |
| Unique Reflections | 44905 | 27692 | 9171 | 43879 |

Refinement

| | | | | |
|---|---|---|---|---|
| Resolution range (Å) | 20-2.3 | 72.17-2.00 | 50-2.6 | 14.18-1.6 |
| Reflections | 37397 | 26284 | 9171 | 41611 |
| R_{cryst} | 0.225 | 0.226 | 0.210 | 0.203 |
| R_{free} | 0.289 | 0.279 | 0.305 | 0.257 |
| Protein Atoms | 6580 | 3294 | 3276 | 3390 |
| Solvent Atoms | 208 | 199 | 46 | 247 |
| r.m.s.d bond length (Å) | 0.022 | 0.007 | 0.015 | 0.014 |
| r.m.s.d bond length (°) | 1.70 | 1.12 | 1.77 | 1.48 |
| Average B-factor (Å²) | 40.3 | 33.6 | 37.5 | 20.7 |
| Overall anisotrpic | −1.52 | 2.42 | 0.20 | 1.13 |
| B-factors (Å²) B11 | | | | |

### Discussion

Structural studies with the EGFR₂₈₇₋₃₀₂ epitope show that both mAb806 and mAb175 recognized the same 3D-structural motif in the wtEGFR structures, indicating that this backbone conformation also occurs in and is exposed in the Δ2-7EGFR. Critically, however, the orientation of the epitope in these structures would prevent antibody access to the relevant amino acids. This is consistent with the experimental observation that mAb806 does not bind wtEGFR expressed on the cell surface at physiological levels.

The results with the EGFR_{C271A/C283A} mutant indicate that the CR1 domain can open up to allow mAb806 and mAb175 to bind stoichiometrically to this mutant receptor. This mutant receptor can still adopt a native conformation as it is fully responsive to EGF stimulation but, unlike the wtEGFR, is fully inhibited by mAb806. If a misfolded form of the EGFR with this disulfide bond broken were to exist on the surface of cancer cells, the data clearly shows it would be capable of initiating cell signaling and should be inhibited by either mAb806 or mAb175.

Another explanation of the data is that during ligand activation the structural rearrangement of the receptor could induce local unfolding in the vicinity of the epitope, allowing the receptor to adopt a conformation which permits binding. In crystal structures, the epitope lies near the physical centre of the EGFR ectodomain and access to the epitope is blocked by both the folded CR1 domain and the quaternary structure of the EGFR ectodomain. In the tethered and the untethered conformations, the integrity of the CR1 domain is stabilized by additional interactions with either the L1:ligand:L2 domains (untethered) or the L2:CR2 domains (tethered). However, the epitope region has some of the highest thermal parameters found in the ectodomain: the mAb806/175 epitope is structurally labile. During receptor activation, when the receptor undergoes a transition between the tethered and untethered conformations, mAb806 and mAb175 can access the epitope. Thus at the molecular level, these mechanisms could contribute to the negligible binding of mAb806 and mAb175 to normal cells and the substantially higher levels of binding to tumor cells which have overexpressed and/or activated EGFR.

### Example 24

### Monoclonal Antibodies 124 and 1133

As discussed in Example 1 above, mAb 124 and mAb1133 were generated at the same time as mAb806 and found to display similar properties, in particular specificity for the over-expressed wild-type EGFR, to the unique properties of mAb806 discussed herein.

Initial screens were conducted in New York (Jungbluth et al. (2003) A Monoclonal Antibody Recognizing Human Cancers with Amplification/Over-Expression of the Human Epidermal Growth Factor Receptor PNAS. 100, 639-644. ELISA competition assessments and Biacore analyses were conducted to determine whether mAb124 and/or mAb1133 recognize an epitope identical to mAb806 or an alternative EGFR determinant.

### FACS Analysis

Antibody binding to U87MG.Δ2-7, A431 and HN5 cells was assessed by FACS. All antibodies displayed a similar specificity as that of mAb806 with strong binding to the de2-7 EGFR and low binding to over-expressed wild-type EGFR.

### Competition ELISA

A series of competition ELISAs were conducted to determine whether the 124 and 1133 antibodies competed with the mAb806 epitope. Briefly, the denatured soluble domain of the EGFR (sEGFR) was coated on to ELISA plates. The unlabeled 124 or 1133 antibodies were then added across the plate in increasing concentrations. Following washing, biotinylated mAb806 was added to each well to determine if it could still bind the sEGFR. Detection of bound mAb806 was achieved using streptavidin-conjugated HRP. If an antibody binds the same (or overlapping) epitope as mAb806 then mAb806 binding is not expected.

Results are summarized in Table 10. A concentration dependant inhibitory binding effect was observed for mAb124 and mAb1133: mAb806 binding increased as concentration of unlabeled antibody was decreased, suggesting that the 124 and 1133 antibodies recognize an epitope identical to mAb806 or one in close proximity.

TABLE 10
Summary mAb124 and mAb1133 Competition
ELISA binding to sEGFR.

| Unlabeled Blocking Antibody | Binding of biotin-labeled 806 |
|---|---|
| 124 | None |
| 1133 | None |
| 806 (control for inhibition) | None |
| Irrelevant IgG2b | ++++ |

### FACS Analysis: Cell Binding Competition

U87MG.Δ2-7 cells were pre-incubated with unlabeled antibody 124, 1133. Positive control 806 and isotype control were included in the assay. Cells were washed, then stained with Alexa488-conjugated mAb806 and the level of 806 binding was determined by FACS.

Results are summarized in Table 11. The 124 and 1133 antibodies blocked mAb806 binding to the cell surface indicating recognition of an epitope identical to mAb806 or one in close proximity.

TABLE 11
FACS Analysis: U87MG.Δ2-7 Cell Binding Competition

| Unlabeled | Inhibition of |
|---|---|
| Blocking Antibody | Alexa488-labeled 806 |
| 124 | +++ |
| 1133 | +++ |
| 806 | ++++ |
| IgG2b control | none |

BIAcore Analysis: Binding to the mAb806 Peptide Epitope

The EGFR amino acid sequence ₂₈₇CGADSYEMEEDGVRKC₃₀₂ (SEQ ID NO:14) containing the mAb806 epitope was synthesized as a peptide and immobilized onto the biosensor chip. Binding of antibodies 124, 1133 and 806 (200 nM) to this peptide was measured. Maximal binding resonance units (RU) obtained are summarized in Table 12. The 124, 1133 showed clear binding to the peptide confirming recognition of the 806 peptide epitope.

TABLE 12
BIAcore Analysis: Maximal binding
to the mAb806 peptide epitope

| | Binding to mAb806 |
|---|---|
| Antibody | peptide (RU) |
| 806 | 1100 |
| 124 | 1000 |
| 1133 | 800 |

### Discussion

As shown in this Example, mAb124 and mAb1133 bind to the EGFR peptide recognized by mAb806 and block binding of mAb806 to the extracellular domain of EGFR and cells expressing the de2-7 EGFR. Thus, these three antibodies recognize the same determinant on EGFR.

### Example 25

### Clinical Testing of ch806

A clinical study was designed to examine the in-vivo specificity of ch806 in a tumor targeting/biodistribution/pharmacokinetic analysis in patients with diverse tumor types.

### 1. Materials and Methods

### Trial Design

This first-in-man trial was an open label, dose escalation Phase I study. The primary objective was to evaluate the safety of a single infusion of ch806 in patients with advanced tumors expressing the 806 antigen. The secondary study objectives were to determine the biodistribution, pharmacokinetics and tumor uptake of ¹¹¹In-ch806; determine the patient's immune response to ch806; and to assess early evidence of clinical activity of ch806. A single dose was chosen for this study in order to optimally assess the in-vivo specificity of ch806 for EGFR expressed on tumor. The protocol was approved by the Human Research and Ethics Committee of the Austin Hospital prior to study commencement. The trial was performed under the Australian Therapeutic Goods Administration Clinical Trials Exemption (CTX) scheme. All patients gave written informed consent.

Eligibility criteria included: advanced or metastatic tumors positive for 806 antigen expression based on chromogenic in-situ hybridisation or immunohistochemistry of archived tumor samples (tumors were defined as 806 positive if immunohistochemical assessment of archived tumour samples showed any cells positive for 806 expression, see below); histological or cytologically proven malignancy; measurable disease on CT scan with at least one lesion ≧2 cm; expected survival of at least 3 months; Karnofsky performance scale (KPS)≧70; adequate hematologic, hepatic and renal function; age >18 yrs; and able to give informed consent. Exclusion criteria included: active central nervous system metastases (unless adequately treated and stable); chemotherapy, immunotherapy, biologic therapy, or radiation therapy within four weeks prior to study entry; prior antibody exposure [unless no evidence of human anti-chimeric antibodies (HACA)]; failure to fully recover from effects of prior cancer therapy; concurrent use of systemic corticosteroids or immunosuppressive agents; uncontrolled infection or other serious disease; pregnancy or lactation; women of childbearing potential not using medically acceptable means of contraception.

Patients received a single infusion of ch806 trace labelled with Indium-111 (¹¹¹In 200-280 MBq; 5-7 mCi) by intravenous infusion in normal saline/5% human serum albumin over 60 minutes. The planned dose escalation meant patients were enrolled into one of four dose levels: 5, 10, 20 and 40 mg/m². These doses were chosen to allow assessment of the specificity of ch806 to EGFR expressed on tumor, and to determine if any normal tissue compartment binds ch806 (and affects pharmacokinetics or biodistribution) in-vivo. Biodistribution, pharmacokinetics, and immune response were evaluated in all patients.

Whole body gamma camera imaging for assessment of biodistribution and tumour uptake was performed on Day 0, Day 1, Day 2 or 3, Day 4 or 5, and Day 6 or 7 following ¹¹¹In-ch806 infusion. Blood samples for pharmacokinetics were obtained at these time-points, and additionally on Day 14 (±2 days) and Day 21 (±2 days). Blood samples for assessment of HACA levels were obtained at baseline, and weekly until Day 30. Toxicity assessment was performed at each study visit. Physical examination and routine hematology and biochemistry were performed weekly until end of study (Day 30). Restaging was performed on Day 30.

### Dose Escalation Criteria

The first patient at each dose level was observed for four weeks prior to enrollment of any additional patients. If no dose limiting toxicity (DLT) was observed in any of the first 2 patients within 4 weeks of the infusion of ch8063, 4 patients were then to be entered on the next highest dosage tier. If one patient in any cohort of 2 patients experienced a DLT within 4 weeks from the first dose, an additional 4 patients (maximum of 6) were entered at that dosage level. If no more than one patient out of 6 in any dose level experienced ≧Grade 3 toxicity, subsequent patients were entered at the next dose level.

DLT was defined as Grade 3 non-haematological toxicity, or Grade 4 haematological toxicity as defined by the NCl Common Terminology Criteria for Adverse Events (CTCAE v3.0). Maximum tolerated dose (MTD) was defined as the ch806 dose below that where 2 or more patients out of 6 experienced DLT.

### Radiolabeling of Ch806

Clinical grade ch806 was produced in the Biological Production Facility of the Ludwig Institute for Cancer Research, Melbourne, Australia. The antibody ch806 was labelled with ¹¹¹In (MDS Nordion, Kanata, Canada) via the bi-functional metal ion chelate CHX-A″-DTPA according to methods described previously (Scott et al. (2000) Cancer Res 60, 3254-3261; Scott et al. (2001) J. Clin. Oncol. 19(19), 3976-3987).

### Gamma Camera Imaging

Whole body images of ¹¹¹In-ch806 biodistribution were obtained in all patients on Day 0 after infusion of ¹¹¹In-ch806, and on at least 3 further occasions up to Day 7 following infusion. Single photon emission computed tomography (SPECT) images of a region of the body with known tumor were also obtained on at least one occasion during this period. All gamma camera images were acquired on a dual-headed gamma camera (Picker International, Cleveland, Ohio).

### Pharmacokinetics

Blood for pharmacokinetic analysis was collected on Day 0—pre ¹¹¹In-ch806 infusion; then at 5 minutes, 60 minutes, 2 h and 4 h post ¹¹¹In-ch806 infusion, Day 1, Day 2 or 3, Day 4 or 5, and Day 6 or 7. Further blood for pharmacokinetics of ch806 protein was also obtained on Day 14 (±2 days) and Day 21 (±2 days) and Day 30 (±2 days).

Serum samples were aliquoted in duplicate and counted in a gamma scintillation counter (Packard Instruments, Melbourne, Australia), along with appropriate ¹¹¹In standards. The results of the serum were expressed as % injected dose per litre (% ID/L). Measurement of patient serum ch806 protein levels following each infusion was performed using a validated protocol for the immunochemical measurement of ch806 protein in human serum⁴⁰. The limit of quantitation for ch806 in serum samples was 70 ng/mL. All samples were assayed in triplicate and were diluted by a factor of at least 1:2. Measured serum levels of ch806 were expressed as μg/mL.

Pharmacokinetic calculations were performed on serum ¹¹¹In-ch806 measurements following the infusion, and ELISA determined patient sera ch806 protein levels, using a curve fitting program (WinNonlin Pro Node 5.0.1, Pharsight Co., Mountain View, Calif.). Estimates were determined for the following parameters: T½α and T½β (half lives of the initial and terminal phases of disposition); V1, volume of central compartment; Cₘₐₓ (maximum serum concentration); AUC (area under the serum concentration curve extrapolated to infinite time); and CL (total serum clearance).

### Whole Body Clearance and Tumor and Organ Dosimetry of ¹¹¹In-ch806

Whole body and normal organ (liver, lungs, kidney and spleen) dosimetry calculations were performed based on regions of interest in each individual patient ¹¹¹In-ch806 infusion image dataset, allowing calculation of cumulated activity and analysis using OLINDA for final dosimetry results (Stabin et al. (2005) J. Nucl. Med. 46(6), 1023-1027). Regions of interest were also defined for suitable tumors at each time point on ¹¹¹In-ch806 image datasets, corrected for background and attenuation, and dosimetry calculation was performed to derive the concentration of ¹¹¹In-ch806 in tumor/gm (Scott et al. (2005) Clin. Cancer Res. 11(13), 4810-4817). This was converted to μg ch806/gm tumor tissue based on the injected mg ch806 protein dose.

### HACA Analysis

Blood samples for HACA assessment were taken prior to ch806 infusion, then weekly until 30 days after ch806 infusion. Samples were analysed by ELISA, and by surface plasmon resonance technology using a BIAcore2000 instrument, as described previously (Scott et al., 2005; Liu et al. (2003) Hybrid Hybridomics 22(4), 219-28; Ritter et al. (2001) Cancer Res. 61(18), 685-6859).

### Immunohistochemistry Method

Formalin-fixed paraffin embedded tumor tissue from each patient on the trial was immunostained as follows: Briefly, 4 μm sections of paraffin embedded tissue were mounted onto SuperFrost® Plus slides (Menzel-Glaser, Germany), de-paraffinized and rehydrated prior to microwave antigen retrieval in Target Retrieval Solution, pH 6.0 (10 min; Dako, Glostrup, Denmark). Sections were then treated with 3% H₂O₂ for 10 min, to eliminate endogenous peroxidase and incubated at room temperature for 60 min with m806 antibody (4 μg/ml) or with appropriate concentration of isotype-matched negative control antibody (IgG2b; Chemicon, Temecula, Calif.). Antibody binding was detected using the PowerVision Kit (ImmunoVision Technologies, Brisbane, Calif.). To allow visualization of the immunostaining, sections were incubated with the chromogen 3-amino-9-ethylcarbazole (0.4%, Sigma Chemical Co. MO, USA) for 10 min and counterstained with Mayer's haematoxylin. Negative controls for the immunostaining procedure were prepared by omission of the primary antibody. Results were expressed as a percentage of positive tumor cell staining.

Chromogenic In situ Hybridization Method

Formalin fixed paraffin embedded tumor tissue from each patient on the trial was sectioned and mounted on SuperFrost® Plus slides, de-paraffinized and rehydrated prior to pre-treatment with the SpotLight® Tissue Pre-treatment Kit (Zymed Laboratories Inc. South San Francisco, Calif.). Sections were then covered with the SpotLight® EGFR DNA probe, denatured at 95° C. for 10 min and incubated overnight at 37° C. Following hybridization, slides were washed in 0.5×SSC. Detection of the probe was carried out using the SpotLight® CISH™ Polymer Detection Kit. Sections that showed clusters of signals or ≧5 individual signals in >25% of cancer cells were considered to have an amplification of the EGFR gene that correlated with m806 reactivity.

### 2. Results

### Patients

Eight patients (1 female and 7 male; mean age of 61 years (range 44-75)] completed the trial (Table 16). Primary tumor sites, prior therapy history, and sites of disease at study entry are also shown in Table 16. All 8 patients had 806 antigen positivity in archived tumors (Table 16).

All patients fulfilled inclusion criteria and, except for Patient 8 (who had a primary brain tumor), all had metastatic disease at study entry. Sites of disease classified as target lesions included: lung (5 patients), brain (1 patient), lymph nodes (1 patient), supraglottis (1 patient). Other sites of metastatic disease (non-target lesions) included a supra-renal mass, bone and lymph nodes (Table 16). The median Karnofsky performance status was 90 (range 80-100).

TABLE 16
Patient Characteristics

| | Dose | | | | | IHC of | | Disease | Tumor |
|---|---|---|---|---|---|---|---|---|---|
| Pt. | Level | Age | | KPS | Site of Primary | positive | Prior | Sites at | response to |
| No. | (mg/m²) | (yrs) | Sex | (%) | Tumour | cells (%) | Therapies | Study Entry | ch806 |
| 1 | 5 | 71 | M | 10 | NSCLC | 50-75 | RT | Lung, | PD |
| | | | | | | | | Adrenal | |
| 8 | 5 | 44 | M | 90 | Anaplastic | >75* | Surgery, | Brain | SD |
| | | | | | astrocytoma | | RT, CT | | |
| 2 | 10 | 49 | F | 80 | SCC Anus | <10 | Chemo, | LN, Lung, | SD |
| | | | | | | | RT | Bone | |
| 3 | 10 | 75 | M | 90 | NSCLC | 50-75 | Surgery | Lung | SD |
| | | | | | | | RT | | |
| 4 | 20 | 52 | M | 100 | Colon | <10† | Surgery, | Lung, LN | PD |
| | | | | | | | CT | | |
| 5 | 20 | 65 | M | 80 | Mesothelioma | >75 | RT, CT | Lung | SD |
| 6 | 40 | 59 | M | 80 | SCC vocal cord | >75 | Surgery, | Soft Tissue | SD |
| | | | | | | | RT, CT | | |
| 7 | 40 | 71 | M | 90 | SCC skin | 50-75 | Surgery, | Lung, LN | PD |
| | | | | | | | CT | | |

Abbreviations: F = female; M = male; NSCLC = non small cell lung carcinoma; SCC = squamous cell carcinoma; RT = radiotherapy; CT = chemotherapy; LN = lymph nodes; PD = progressive disease; SD = stable disease
*positive for de2-7 EGFR expression
†positive for EGFR gene amplification

### Adverse Events and HACA

Adverse events related to ch806 are listed in Tables 17 and 18. No infusion related adverse events were observed. There was no DLT, and hence MTD was not reached. The principle toxicities that in the investigator's opinion were possibly attributable to ch806 were: transient pruritis, mild nausea, fatigue/lethargy, and possible effects on serum ALP and GGT levels. A CTC grade 2 elevation in GGT level in Patient 5 was observed, however this was on a background of a baseline grade 1 elevation, and was transient in nature. Three serious adverse events (SAEs) were reported but none were attributed to ch806. Overall, ch806 was safe and well tolerated at all dose levels with generally predictable and manageable minor toxicities being observed. Further dose escalation was not performed due to the limited amount of cGMP ch806 available for the trial.

A positive immune response to ch806 (with concordance of both ELISA and BIAcore methodologies) was observed in only one of the eight patients (Patient 1).

TABLE 17
Occurrence of Adverse Events Related to ch806

| | Dose Level (mg/m²)* | | | | Total Number of Episodes |
|---|---|---|---|---|---|
| Adverse Event | 5 | 10 | 20 | 40 | of Each Event |
| Dizziness | 0 | 0 | 0 | 1 | 1 |
| Fatigue | 0 | 0 | 1 | 0 | 1 |
| Lethargy | 0 | 0 | 0 | 1 | 1 |
| Appetite | 0 | 0 | 0 | 1 | 1 |
| suppressed | | | | | |
| Nausea | 0 | 1 | 0 | 1 | 2 |
| Pruritis | 1 | 0 | 0 | 0 | 1 |
| ALP - elevated | 0 | 0 | 1 | 0 | 1 |
| GGT - elevated | 0 | 0 | 1 | 0 | 1 |
| Total | 1 | 1 | 3 | 4 | 9 |

*Numbers represent number of episodes of any event at each dose level

TABLE 18
Distribution of Study Agent Related Adverse Events

| | Maximum CTC Grade Toxicity* | | | |
|---|---|---|---|---|
| Dose Level | | | | 4 = Life- |
| (mg/m²) | 1 = Mild | 2 = Moderate | 3 = Severe | threatening |
| 5 | 1 | 0 | 0 | 0 |
| 10 | 1 | 0 | 0 | 0 |
| 20 | 2 | 1 | 0 | 0 |
| 40 | 4 | 0 | 0 | 0 |
| Overall | 8 | 1 | 0 | 0 |

*Number of patients

Radiolabeling of ch806

There were a total of 8 infusions of ¹¹¹In-ch806 administered during the trial. The mean (±SD) radiochemical purity and immunoreactivity of ¹¹¹In-ch806 was measured to be 99.3±0.1% and 77.4±7.0% respectively.

Biodistribution of ch806

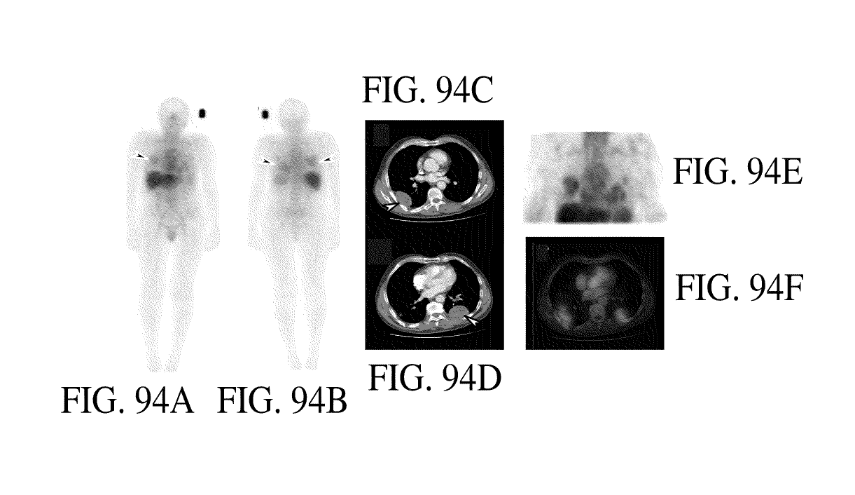

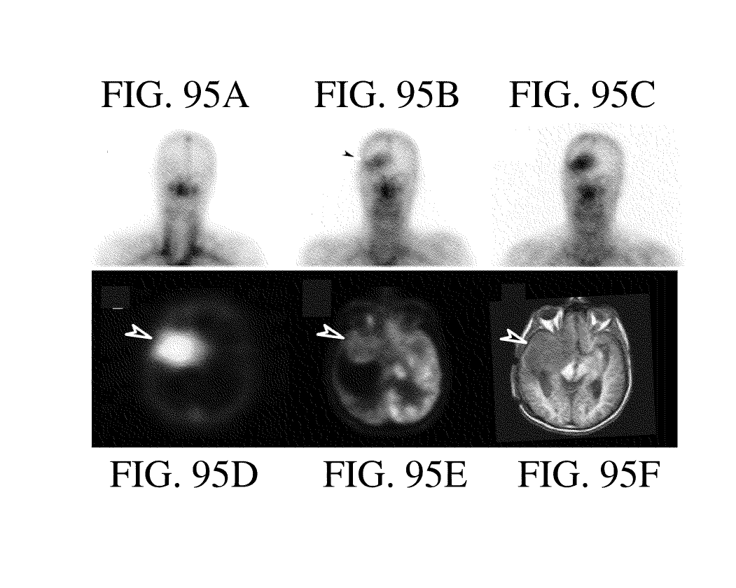

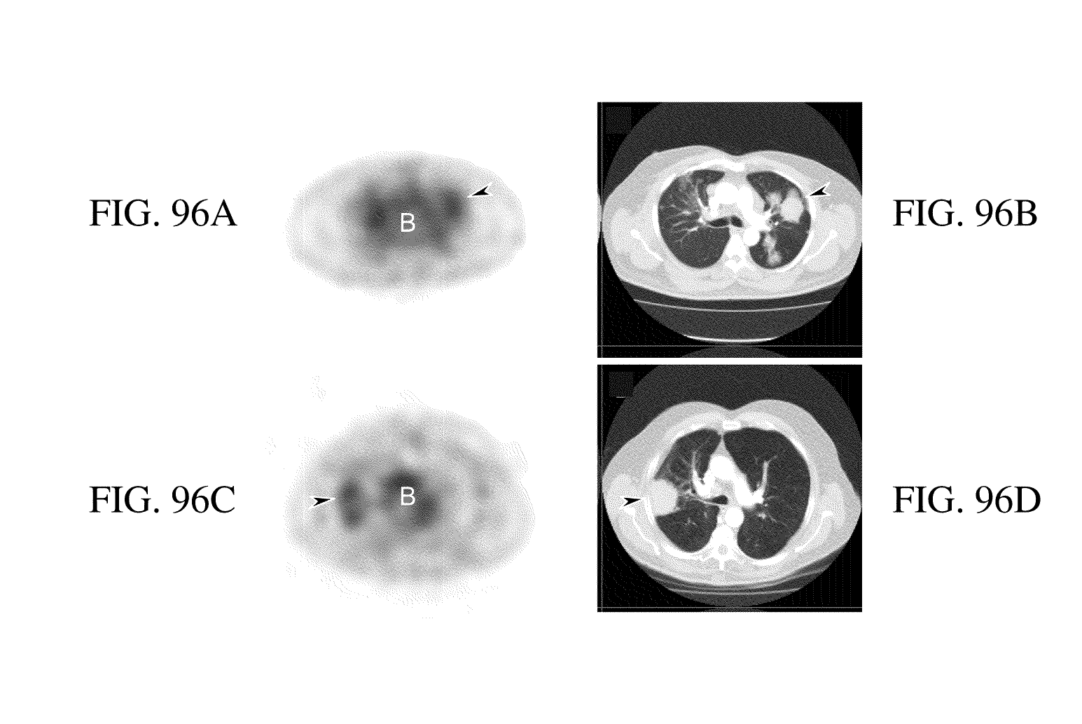

The initial pattern of ¹¹¹In-ch806 biodistribution in patients at all dose levels was consistent with blood pool activity, which cleared gradually with time. Over the one week period post injection the uptake of ¹¹¹In-ch806 in liver and spleen was consistent with the normal clearance of ¹¹¹In-chelate metabolites through the reticuloendothelial system. Specific localization of ¹¹¹In-ch806 was observed in target lesions (≧2 cm) of all patients at all dose levels (FIG. 94), including target lesions located in the lungs (Patients 1, 3, 4, 5, and 7), the abdomen (Patients 1 and 2), and the supraglottic region in the right side of the neck (Patient 6). High uptake of ¹¹¹In-ch806 in a brain tumor (Patient 8) was also demonstrated (FIG. 95) Importantly, uptake of ¹¹¹In-ch806 in tumor was not dependent on a the level of 806 antigen expression. For example, Patient 4 demonstrated high uptake by both lung target lesions, despite <10% positivity by IHC for 806 reactivity in archived tumor (FIG. 96). This degree of uptake of ¹¹¹In-ch806 in target lesions in Patient 4 was comparable to that seen in Patient 3, where 50-75% of tumor cells were positive for 806 antigen staining on archived sample immunohistochemistry (FIG. 96).

### Pharmacokinetics

Individual patient pharmacokinetic parameters T½α and T½β, V1, Cₘₐₓ, AUC and CL for the single infusion of ¹¹¹In-ch806 are shown in Table 19. The Kruskal-Wallis rank sum test was applied to the alpha and beta half lives, V1 and clearance. No significant difference between dose levels was observed (P>0.05).

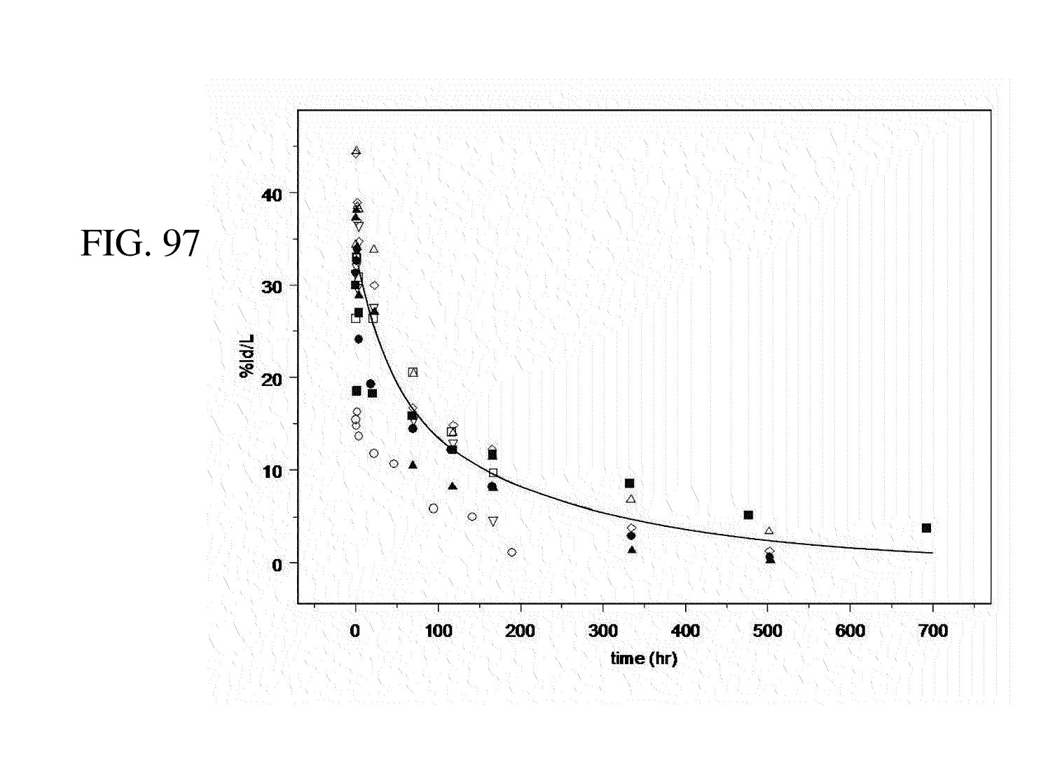

The pharmacokinetic curve fit to the pooled population ELISA data is shown in FIG. 97. The mean±SD pharmacokinetic parameters were T½α 29.16±21.12 hrs, T½β 172.40±90.85 hrs, V1 2984.59±91.91 ml, and CL 19.44±4.05 ml/hr. Measured peak and trough ch806 serum concentrations (Cₘₐₓ and Cₘᵢₙ) data are presented in Table 20 for each patient. As expected, linear relationships were observed for Cₘₐₓ and Cₘᵢₙ with each dose level. The mean±SD values determined for the ch806 ELISA pharmacokinetic data were in good agreement with the values obtained for the ¹¹¹In-ch806 pharmacokinetic data (Table 19).

TABLE 19
Mean ± SD Pharmacokinetic Parameter Estimates for ¹¹¹In-CHX-A″-DTPA-ch806
in each Dose Level and across all Dose Levels.

| Dose | T½ α | | T½ β | | V1 | | CL | | AUC | |
|---|---|---|---|---|---|---|---|---|---|---|
| Level | (hr) | | (hr) | | (mL) | | (mL/hr) | | (hr*mg/mL) | |
| (mg/m²) | Mean | SD | Mean | SD | Mean | SD | Mean | SD | Mean | SD |
| 5 | 10.91 | 3.4 | 183.9 | 110.2 | 2963.06 | 493.23 | 21.97 | 16.59 | 541.17 | 371.75 |
| 10 | 11.75 | 4.4 | 124.5 | 9.25 | 3060.29 | 721.70 | 28.58 | 8.60 | 566.79 | 26.39 |
| 20 | 9.34 | 8.3 | 125.3 | 73.66 | 2902.06 | 1064.77 | 30.98 | 21.65 | 1438.12 | 957.18 |
| 40 | 8.95 | 3.2 | 133.9 | 10.79 | 4742.42 | 169.10 | 37.99 | 6.47 | 2269.04 | 381.68 |
| ALL | 10.24 | 1.32 | 141.90 | 28.30 | 3416.96 | 886.04 | 29.88 | 6.61 | | |

TABLE 20
Cmax and Cmin Serum ch806 Levels
Determined by ELISA Analysis.

| | DOSE LEVEL | Cₘₐₓ* | Cₘᵢₙ* |
|---|---|---|---|
| PT. NO. | (MG/M²) | (μG/ML) | (μG/ML) |
| 1 | 5 | 1.38 ± 0.02 | 0.10 ± 0.05† |
| 8 | 5 | 1.52 ± 0.17 | 0.96 ± 0.08 |
| 2 | 10 | 5.92 ± 0.11 | 1.50 ± 0.01 |
| 3 | 10 | 6.27 ± 0.45 | 1.83 ± 0.20 |
| 4 | 20 | 12.25 ± 0.66 | 4.05 ± 0.05 |
| 5 | 20 | 11.22 ± 0.77 | 1.58 ± 0.04 |
| 6 | 40 | 27.76 ± 2.10 | 6.90 ± 0.38 |
| 7 | 40 | 32.32 ± 0.84 | 6.80 ± 0.13 |

*Cₘₐₓ = 60 min post injection.; Cₘᵢₙ = Day 7
†Day 8 serum level

### Dosimetry of ¹¹¹In-ch806

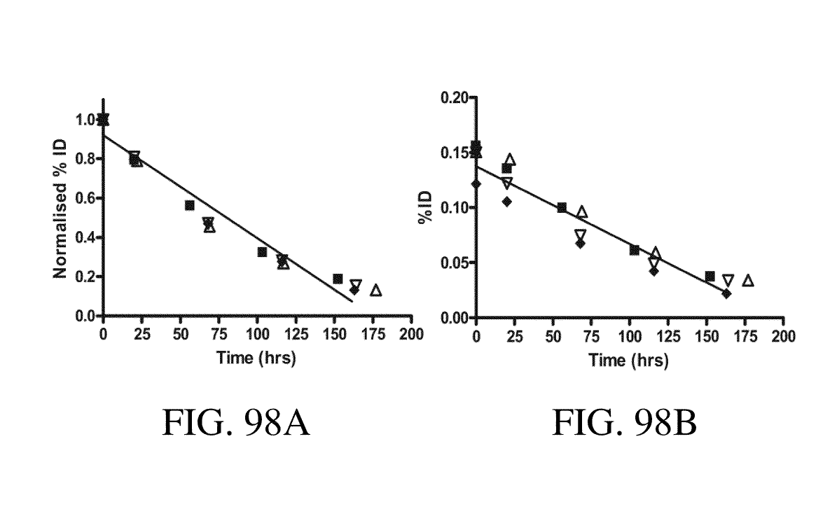

Whole body clearance was similar in all patients across all dose levels, with a T_{1/2} biologic (mean±SD) of 948.6±378.6 hrs. Due to the relatively short physical half-life, calculation of biological halftime was extremely sensitive to small changes in effective halftime. There was no statistical significant difference in whole body clearance between dose levels [Kruskal-Wallis rank sum test: P-value=0.54] (FIG. 98).

The clearance of ¹¹¹In-ch806 from normal organs (liver, lungs, kidney and spleen) showed no difference between dose levels, and the mean T_{1/2} effective was calculated to be 78.3, 48.6, 69.7 and 66.2 hrs respectively. There was no statistically significant difference in clearance between these normal organs. In particular, liver clearance showed no difference between dose levels (FIG. 98), indicating no saturable antigen compartment in the liver for ch806.

Tumor dosimetry analysis was completed for 6 patients. Patients 1 and 2 had target lesions close to the cardiac blood pool, or motion during some image acquisitions, which prevented accurate analysis. The measured peak uptake of ¹¹¹In-ch806 occurred 5-7 days post infusion, and ranged from 5.2−13.7×10⁻³% injected dose/gm tumor tissue.

### Assessment of Clinical Activity

At the completion of this one month study period 5 patients were found to have stable disease, and 3 patients progressive disease (Table 16). Interestingly, one patient (Patient 7, 40 mg/m² dose level) had clinical evidence of transient shrinkage of a palpable auricular lymph node (proven to be metastatic SCC on fine needle aspiration) during the study period, which suggests possible biologic activity of ch806. However, this patient had confirmed progressive disease by RECIST at study completion.

### Additional Data

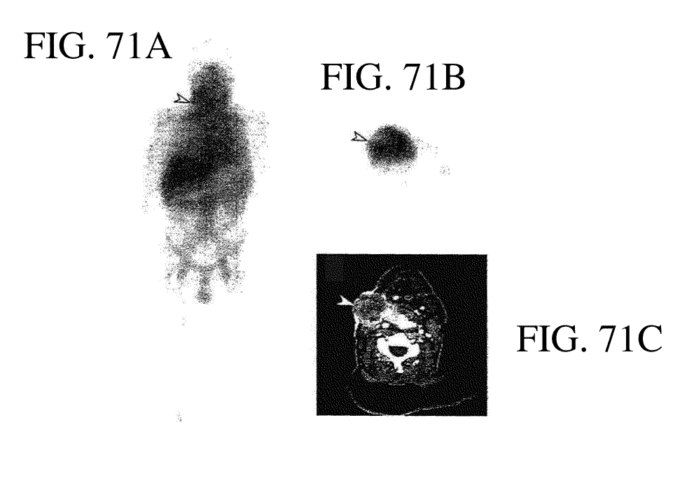

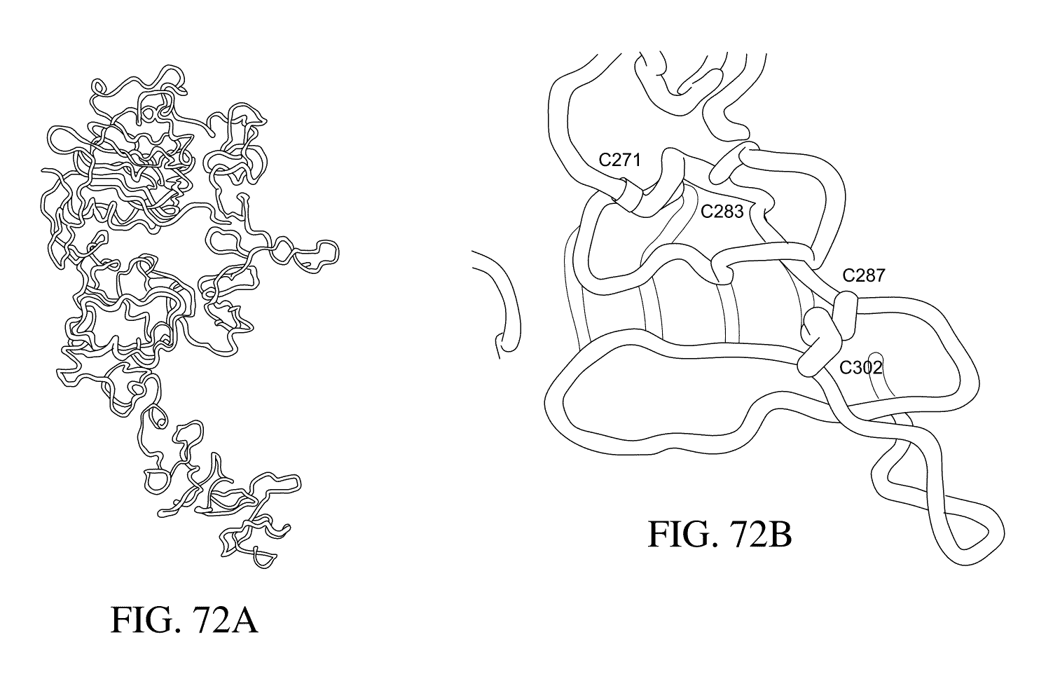

Eight patients [1 female and 7 male; mean age of 61 years (range 44-75)] completed this phase 1 trial as reported (Scott et al. (2007) Proc. Natl. Acad. Sci. U.S.A. 104, 4071-4076). All patients fulfilled inclusion criteria and, except for Patient 8 (who had a primary brain tumor), all had metastatic disease at study entry. Ab uptake by the tumor was seen in all patients, and ¹¹¹In-ch806, the chimerized version of mAb806, demonstrated prompt and high level uptake in tumor (FIG. 71). The clearance of ¹¹¹In-ch806 from normal organs (liver, lungs, kidney and spleen) showed no difference between dose levels (Scott et al., 2007). In particular, liver clearance showed no difference between dose levels, indicating no saturable antigen compartment in the liver for ch806. Total liver uptake was a maximum of 14.45±2.43% ID immediately post infusion, and declined to 8.45±1.63% ID by 72 hours, and 3.18±0.87% ID by one week post infusion. This is in marked contrast to the uptake of antibodies to wtEGFR (e.g. 225), which have been shown to reach over 30% ID in liver (for a 40 mg dose) for over 3 days post infusion (Divgi et al. (1991) J. Natl. Cancer Inst. 83, 97-104). The measured peak tumor uptake of ¹¹¹In-ch806 occurred 5-7 days post infusion. Calculation of quantitative tumor uptake in Patients 1 and 3 could not be accurately performed due to proximity of target lesion to cardiac blood pool and patient movement. Peak ch806 uptake in tumor ranged from 5.21 to 13.73×10⁻³% ID/gm tumor tissue. Calculation of actual ch806 concentration in tumor showed peak values of (mean±SD) 0.85±0 μg/gm (5 mg/^{m2}), 0.92±0 μg/gm (10 mg/^{m2}), 3.80±1.10 μg/gm (20 mg/m²), and 7.05±1.40 μg/gm (40 mg/m²).

### Discussion

As set forth in this Example, this study represents the first reported demonstration of the biodistribution and tumor targeting of a chimeric antibody against an epitope only exposed on overexpressed, mutant or ligand activated forms of the EGFR. Ch806 showed excellent targeting of tumor sites in all patients, no evidence of normal tissue uptake, and no significant toxicity. These in vitro and in vivo characteristics of ch806 distinguish it from all other antibodies targeting EGFR.

At doses up to 40 mg/m², ch806 was well tolerated, no DLT was observed and MTD was not reached. The principle toxicities that were possibly attributable to ch806 were transient pruritis, mild nausea, fatigue/lethargy, and possible effects on serum ALP and GGT levels. The advanced nature of these patient's malignancies meant their disease could also have been contributing factors to these adverse events. Of the adverse events that were possibly related to study drug, all were mild, many were self-limiting, and none required any active treatment. Importantly, no skin rash or gastrointestinal tract disturbances were observed in any patient, even at the highest dose level. The excellent tolerability of ch806 in this single-dose study justifies the next step of testing in repetitive dose trials.

The biodistribution of ch806 in all patients showed gradual clearance of blood pool activity, and no definite normal tissue uptake of ¹¹¹In-ch806. Excellent tumor uptake of ch806 was also evident in all patients, including lung, lymph node, and adrenal metastases, and in mesothelioma and glioma. This was observed at all dose levels including 5 mg/m² (the lowest dose studied), which is one tenth to one twentieth of the dose required to visualise uptake in tumor by other antibodies to wtEGFR³³. This difference in uptake of ch806 compared to antibodies to wtEGFR can be attributed to their substantial normal tissue (liver and skin) uptake due to wtEGFR acting as an antigen sink³³. In addition, the localization of ¹¹¹In-ch806 was high even in patients with low expression of 806 assessed by immunohistochemistry of archived tumor samples (FIG. 96). The uptake of ¹¹¹In-ch806 in glioma was particularly impressive (FIG. 97), and comparable to any published data on antibody targeting of brain tumor following systemic or even locoregional infusion. This data supports the unique selectivity of ch806 to EGFR expressed by a broad range of tumors, and confirms the lack of normal tissue uptake of this antibody in human

Pharmacokinetic analyses showed that ch806 has a terminal half-life of more than a week, and no dose dependence of ¹¹¹In-ch806 serum clearance. Linear relationships also were observed for AUC, Cₘₐₓ and Cₘᵢₙ, with dose levels above 10 mg/m² achieving trough serum concentrations above 1 μg/mL. The V1, C1, T½ α and T½ β values were consistent between dose levels, and in keeping with typical IgG1 human antibodies (Scott et al., 2005; Steffens et al. (1997) J. Clin. Oncol 15, 1529-1537; Scott et al. (2001) J. Clin. Oncol. 19(19), 3976-3987). The clearance of ch806 was also determined to be slower when ELISA ch806 calculations were compared to ¹¹¹In-ch806 measurements. While this difference may be explained by the small number of patients studied, the longer sampling time points for the ch806 ELISA would support this value as being more representative of true ch806 clearance. The pharmacokinetic values for ch806 are comparable to other chimeric antibodies reported to date (Steffens et al., 1997; Scott et al., 2001), and supports a weekly dosing schedule of ch806.

The quantitative dosimetry and pharmacokinetic results indicate that there is no saturable normal tissue compartment for ch806 for the dose levels assessed in this trial Importantly, the lack of dose dependence on pharmacokinetic and whole body and liver organ clearance is in marked contrast to all reported studies of antibodies to wtEGFR (Baselga J. and Artega C. L. (2005) J. Clin. Oncol. 23, 2445-2449; Divgi et al. J. Natl. Cancer Inst. 83(2), 97-104; Baselga J (2001) Eur. J. Cancer 37 Suppl. 4, S16-22; Gibson et al. (2006) Clin. Colorectal Cancer 6(1), 29-31; Rowinsky et al. (2004) J. Clin. Oncol. 22, 3003-3015; Tan et al. (2006) Clin. Cancer Res. 12(21), 6517-6522) supporting the tumour specificity and lack of normal tissue binding of ch806 in humans. These observations provide compelling evidence of the potential for ch806 (or humanized forms) to selectively target EGFR in tumor, avoid the normal toxicity of other EGFR antibodies and kinase inhibitors (particularly skin) (Lacouture A E (2006) Nature Rev. Cancer 6, 803-812; Adams G. P. and Weiner L. M. (2005) Nat. Biotechnol. 23(9), 1147-1157) and potentially achieve greater therapeutic effect. Moreover, the possibility of payload delivery (due to the rapid internalisation of mAb 806 in tumor cells), and combination treatment with other biologics such as EGFR antibodies and tyrosine kinase inhibitors where combined toxicity is likely be minimised, is strongly supported by the data from this trial. This study provides clear evidence of the ability to target an epitope on EGFR that is specific for tumor, and further clinical development of this unique approach to cancer therapy is ongoing.

### Example 26

### Sequence Comparisons

The VH chain and VL chain CDRs for each of mAb806, mAb175, mAb124, mAb1133, and hu806 are set forth and compared herein.

TABLE 13
Murine Antibody Isotype and CDR Sequence Comparisons (Kabat)¹

| | | |
|---|---|---|
| CDR1 | CDR2 | CDR3 |

A. Variable Light Chain

| | | | |
|---|---|---|---|
| 806 | HSSQDINSNIG | HGTNLDD | VQYAQFPWT |
| (IgG2b) | (SEQ ID NO: 18) | (SEQ ID NO: 19) | (SEQ ID NO: 20) |
| 124 | HSSQDINSNIG | HGTNLDD | VQYGQFPWT |
| (IgG2a) | (SEQ ID NO: 28) | (SEQ ID NO: 29) | (SEQ ID NO: 30) |
| 175 | HSSQDISSNIG | HGTNLED | VQYGQFPWT |
| (IgG2a) | (SEQ ID NO: 135) | (SEQ ID NO: 136) | (SEQ ID NO: 137) |
| 1133 | HSSQDINSNIG | HGTNLDD | VQYGQFPWT |
| (IgG2a) | (SEQ ID NO: 38) | (SEQ ID NO: 39) | (SEQ ID NO: 40) |

B. Variable Heavy Chain

| | | | |
|---|---|---|---|
| 806 | SDFAWN | YISYSGNTRYNPSLKS | VTAGRGFPY |
| (IgG2b) | (SEQ ID NO: 15) | (SEQ ID NO: 16) | (SEQ ID NO: 17) |
| 124 | SDYAWN | YISYSANTRYNPSLKS | ATAGRGFPY |
| (IgG2a) | (SEQ ID NO: 23) | (SEQ ID NO: 24) | (SEQ ID NO: 25) |
| 175 | SDYAWN | YISYSANTRYNPSLKS | ATAGRGFPY |
| (IgG2a) | (SEQ ID NO: 130) | (SEQ ID NO: 131) | (SEQ ID NO: 132) |
| 1133 | SDYAWN | YISYSGNTRYNPSLRS | ATAGRGFPY |
| (IgG2a) | (SEQ ID NO: 33) | (SEQ ID NO: 34) | (SEQ ID NO: 35) |

¹differences to the mAb806 CDR sequences are underlined

The CDRs given above for the respective antibody isotypes are based on a Kabat analysis. As will be apparent to those of skill in the art, the CDRs may also be defined based on other analysis, for example a composite of Kabat and Chothia definitions. For example, applying a composite Kabat and Chothia analysis to the above isotypes, the sequences of the VL chain CDRs and VH chains CDRs for the respective isotypes are as set forth in Table 14.

TABLE 14
Murine Antibody Isotype and CDR Sequence Comparisons
(Composite Kabat and Chothia)¹

| | | |
|---|---|---|
| CDR1 | CDR2 | CDR3 |

A. Variable Light Chain

| | | | |
|---|---|---|---|
| 806 | HSSQDINSNIG | HGTNLDD | VQYAQFPWT |
| (IgG2b) | (SEQ ID NO: 18)² | (SEQ ID NO: 139)² | (SEQ ID NO: 20)² |
| 124 | HSSQDINSNIG | HGTNLDD | VQYGQFPWT |
| (IgG2a) | (SEQ ID NO: 28) | (SEQ ID NO: 140) | (SEQ ID NO: 30) |
| 175 | HSSQDISSNIG | HGTNLED | VQYGQFPWT |
| (IgG2a) | (SEQ ID NO: 135) | (SEQ ID NO: 141) | (SEQ ID NO: 137) |
| 1133 | HSSQDINSNIG | HGTNLDD | VQYGQFPWT |
| (IgG2a) | (SEQ ID NO: 38) | (SEQ ID NO: 142) | (SEQ ID NO: 40) |

B. Variable Heavy Chain

| | | | |
|---|---|---|---|
| 806 | GYSITSDFAWN | GYISYSGNTRYNPSLKS | VTAGRGFPY |
| (IgG2b) | (SEQ ID NO: 143)³ | (SEQ ID NO: 144)³ | (SEQ ID NO: 17)³ |
| 124 | GYSITSDYAWN | GYISYSANTRYNPSLKS | ATAGRGFPY |
| (IgG2a) | (SEQ ID NO: 145) | (SEQ ID NO: 146) | (SEQ ID NO: 25) |
| 175 | GYSITSDYAWN | GYISYSANTRYNPSLKS | ATAGRGFPY |
| (IgG2a) | (SEQ ID NO: 147) | (SEQ ID NO: 148) | (SEQ ID NO: 132) |
| 1133 | GYSITSDYAWN | GYISYSGNTRYNPSLRS | ATAGRGFPY |
| (IgG2a) | (SEQ ID NO: 149) | (SEQ ID NO: 150) | (SEQ ID NO: 35) |

¹differences to the mAb806 CDR sequences are underlined
²See FIG. 17 of U.S. patent application No. 10/145,598 (U.S. Pat. No. 7,589,180)
³See FIG. 16 of U.S. patent application No. 10/145,598 (U.S. Pat. No. 7,589,180)

TABLE 15
mAb806 and hu806 CDR Sequence Comparisons(Kabat)¹

| | | |
|---|---|---|
| CDR1 | CDR2 | CDR3 |

A. Variable Light Chain

| | | | |
|---|---|---|---|
| mAb806 | HSSQDINSNIG | HGTNLDD | VQYAQFPWT |
| | (SEQ ID NO: 18) | (SEQ ID NO: 19) | (SEQ ID NO: 20) |
| hu806 | HSSQDINSNIG | HGTNLDD | VQYAQFPWT |
| | (SEQ ID NO: 49) | (SEQ ID NO: 50) | (SEQ ID NO: 51) |

B. Variable Heavy Chain

| | | | |
|---|---|---|---|
| mAb806 | SDFAWN | YISYSGNTRYNPSLKS | VTAGRGFPY |
| | (SEQ ID NO: 15) | (SEQ ID NO: 16) | (SEQ ID NO: 17) |
| hu806 | SDFAWN | YISYSGNTRYQPSLKS | VTAGRGFPY |
| | (SEQ ID NO: 44) | (SEQ ID NO: 45) | (SEQ ID NO: 46) |

¹differences to the mAb806 CDR sequences are underlined

As shown above, the CDR sequences of mAb806, mAb175, mAb124 and mAb1133 isotypes are identical except for highly conservative amino acid changes that would be expected to give rise to homologous protein folding for epitope recognition. This data, cumulatively with the binding and other data provided in the Examples above, shows that these isotypes and the hu806 are closely-related family member variants exhibiting the same unique properties discussed above for mAb806 (e.g., binding to an epitope on the EGFR that is accessible to binding only in overexpressed, mutated or ligand activated forms of the EGFR, resulting in unique specificity for tumor-expressed EGFR, but not wtEGFR in normal tissue) and demonstrating that antibodies of distinct variable region sequences, particularly of varying CDR sequences, have the same characteristics and binding capabilities.

### REFERENCES

“Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985).
Aboud-Pirak, E., Hurwitz, E., Bellot, F., Schlessinger, J., and Sela, M. (1989) Proc. Natl. Acad. Sci. USA. 86, 3778-3781.
Aboud-Pirak, E., Hurwitz, E., Pirak, M. E., Bellot, F., Schlessinger, J., and Sela, M. (1988) J. Natl. Cancer Inst. 80, 1605-1611.
Adams G P, Weiner L M (2005) Nat. Biotechnol. 23(9): 1147-1157.
Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985)
Arteaga, C. l. and Baselga, J. (2004) Cancer Cell. 5, 525-531.
Ashley, D. M., Batra, S. K., and Bigner, D. D. “Monoclonal antibodies to growth factors and growth factor receptors: their diagnostic and therapeutic potential in brain tumors.” J. Neurooncol., 35: 259-273, 1997.
Atlas, I., Mendelsohn, J., Baselga, J., Fair, W. R., Masui, H., and Kumar, R. “Growth regulation of human renal carcinoma cells: role of transforming growth factor a.” Cancer Res., 52: 3335-3339, 1992.
Baselga J (2001) Eur. J. Cancer 37 Suppl 4:S16-22.
Baselga J & Arteaga C L (2005) J. Clin. Oncol. 23, 2445-2459.
Baselga J, Artega C L (2005) J. Clin. Oncol. 23: 2445-2449.
Baselga J, Pfister D, Cooper M R, Cohen R, Burtness B, Bos M, D'Andrea G, Seidman A, Norton L, Gunnett K, et al. (2000) J. Clin. Oncol. 18(4):904-914.
Baselga J, Pfister D, Cooper M R, et al. “Phase I studies of anti-epidermal growth factor receptor chimeric antibody C225 alone and in combination with cisplatin.” J. Clin. Oncol. 2000; 18: 904-14.
Baselga J, Tripathy D, Mendelsohn J, Baughman S, Benz C C, Dantis L, Sklarin N T, Seidman A D, Hudis C A, Moore J, et al. (1996) J. Clin. Oncol. 14: 737-744.
Baselga, 1. (2006) Science. 312, 1175-1178.
Baselga, J. and Arteaga, C. L. (2005) J. Clin. Oncol. 23, 2445-2459.
Baselga, J. Clinical trials of Herceptin (R) (trastuzumab). Eur. J. Cancer, 37: 1824, 2001.
Baselga, J., Norton, L., Albanell, J., Kim, Y. M., and Mendelsohn, J. “Recombinant humanized anti-HER2 antibody (Herceptin) enhances the antitumor activity of paclitaxel and doxorubicin against HER2/neu overexpressing human breast cancer xenografts.” Cancer Res., 58: 2825-2831, 1998.
Baselga, J., Norton, L., Masui, H., Pandiella, A., Coplan, K., Miller, W. H., and Mendelsohn, J. “Antitumor effects of doxorubicin in combination with anti-epidermal growth factor receptor monoclonal antibodies.” J. Natl. Cancer Inst. (Bethesda), 85: 1327-1333, 1993.
Baselga, J., Pfister, D., Cooper, M. R., Cohen, R., Burtness, B., Bos, M., D'Andrea, G., Seidman, A., Norton, L., Gunnett, K., Falcey, J., Anderson, V., Waksal, H., and Mendelsohn, J. “Phase I Studies of Anti-Epidermal Growth Factor Receptor Chimeric Antibody C225 Alone and in Combination With Cisplatin.” J. Clin. Oncol. 18: 904, 2000.

Baselga, J., Tripathy, D., Mendelsohn, J., Baughman, S., Benz, C. C, Dantis, L., Sklarin, N. T., Seidman, A. D., Hudis, C. A., Moore, J., Rosen, P. P., Twaddell, T., Henderson, l. C., and Norton, L. “Phase II study of weekly intravenous recombinant humanized anti-p185HER2 monoclonal antibody in patients with HER2/neu-overexpressing metastatic breast cancer.” J. Clin. Oncol., 14: 737-744, 1996.
Batra S K, Castelino-Prabhu S, Wikstrand C J, et al. “Epidermal growth factor ligand-independent, unregulated, cell-transforming potential of a naturally occurring human mutant EGFRvIII gene.” Cell Growth Differ. 1995; 6: 1251-9.
Bernier, J. (2006) Expert. Rev Anticancer Ther. 6, 1539-1552.
Bhattacharya-Chatterjee, M., S. K. Chatterjee, et al. (2001). “The anti-idiotype vaccines for immunotherapy.” Curr. Opin. Mol. Ther. 3(1): 63-9. Biol. Cell. 13, 4029-4044.
Bouyain, S., Longo, P. A., L1, S., Ferguson, K. M., and Leahy, D. J. (2005) Proc. Natl. Acad. Sci. USA. 102, 15024-15029.
Brady, L. W., Miyamoto, C., Woo, D. V., Rackover, M., Emrich, J., Bender, H., Dadparvar, S., Steplewski, Z., Koprowski, H., Black, P., et al. “Malignant astrocytomas treated with iodine-125 labeled monoclonal antibody 425 against epidermal growth factor receptor: a Phase I trial.” Int. J. Radiat. Oncol. Biol. Phys., 22: 225-230, 1992.
Brown, G. and N. Ling (1988). Murine Monoclonal Antibodies. Antibodies, Volume 1. A Practical Approach. D. Catty. Oxford, England, IRL Press: 81-104.
Burgess A W (2008) Growth Factors 26, 263-274.
Burgess A W, Cho H S, Eigenbrot C, Ferguson K M, Garrett T P, Leahy D J, Lemmon M A, Sliwkowski M X, Ward C W, & Yokoyama S (2003) Mol. Cell. 12: 541-552.
Burgess, A. W., Cho, H. S., Eigenbrot, C., Ferguson, K. M., Garrett, T. P., Leahy, D. J., Lemmon, M. A., Sliwkowski, M. x., Ward, C. W., and Yokoyama, S. (2003) Mol. Cell. 12, 541-552.
Chao G, Cochran J R, & Wittrup K D (2004) J. Mol. Biol. 342, 539-550.
Chao, G., Cochran, 1. R, and Wittrup, K. D. (2004) J. Mol. Biol. 342, 539-550.
Ching K Z, Ramsey E, Pettigrew N, D'Cunha R, Jason M, & Dodd J G (1993) Mol. Cell. Biochem. 126, 151-158.
Cho, H. S, and Leahy, D. J. (2002) Science 297, 1330-1333.
Cho, H. S., Mason, K., Ramyar, K. x., Stanley, A. M., Gabelli, S. B., DelUley, D. W., Jr., and Leahy, D. J. (2003) Nature 421, 756-760.
Clarke, K., et al., “In vivo biodistribution of a humanized anti-Lewis Y monoclonal antibody (hu3S193) in MCF-7 xenografted BALB/c nude mice.” Cancer Res, 2000. 60(17): p. 4804-11.
Clarke, K., Lee, F. T., Brechbiel, M. W., Smyth, F. E., Old, L. J., and Scott, A. M. “Therapeutic efficacy of anti-Lewis (y) humanized 3S193 radioimmunotherapy in a breast cancer model: enhanced activity when combined with Taxol chemotherapy.” Clin. Cancer Res., 6: 3621-3628, 2000.
Clayton, A. H., Walker, F., Orchard, S. G., Henderson, C., Fuchs, D., Rothacker, J., Nice, E. C., and Burgess, A. W. (2005) J. Biol. Chem. 280, 30392-30399.
Daugherty B L, DeMartino J A, Law M F, Kawka D W, Singer I I, Mark G E. “Polymerase chain reaction facilitates the cloning, CDR-grafting, and rapid expression of a murine monoclonal antibody directed against the CD18 component of leukocyte integrins.” Nucleic Acids Res. 1991 19(9):2471-6.
de Larco, J. E. and Todaro, G. J. (1978) J. Cell. Physiol. 94, 335-342.
de Larco, J. E., Reynolds, R., Carlberg, K., Engle, C., and Todaro, G. J. (1980) J. Biol. Chem. 255, 3685-3690.
den Eynde, B. and Scott, A. M. Tumor Antigens. In: P. J. Delves and I. M. Roitt (eds.), Encyclopedia of Immunology, Second Edition, pp. 2424 31. London: Academic Press, 1998.
DeNardo S J, Kroger L A, DeNardo G L. “A new era for radiolabeled antibodies in cancer?” Curr. Opin. Immunol. 1999; 11: 563-9.
Divgi C R, Welt S, Kris M, Real F X, Yeh S D, Gralla R, Merchant B, Schweighart S, Unger M, Larson S M, et al. (1991) J. Natl. Cancer Inst. 83(2):97-104.
Divgi, C. R., Welt, S., Kris, M., Real, F. X., Yeh, S. D., Gralla, R., Merchant, B., Schweighart, S., Unger, M., Larson, S. M., et al. “Phase I and imaging trial of indium 11-labeled anti-epidermal growth factor receptor monoclonal antibody 225 in patients with squamous cell lung carcinoma.” J. Natl. Cancer Inst., 83: 97-104, 1991.
Domagala, T., Konstantopoulos, N., Smyth, F., Jorissen, R. N., Fabri, L., Geleick, D., Lax, I., Schlessinger, J., Sawyer, W., Howlett, G. J., Burgess, A. W., and Nice, E. C. “Stoichiometry, kinetic and binding analysis of the interaction between Epidermal Growth Factor (EGF) and the Extracellular Domain of the EGF receptor.” Growth Factors. 18: 11-29, 2000.
Domagala, T., N. Konstantopoulos, et al. (2000).“Stoichiometry, kinetic and binding analysis of the interaction between epidermal growth factor (EGF) and the extracellular domain of the EGF receptor.” Growth Factors 18 (1):11-29.
Ekstrand A J, Sugawa N, James C D, et al. “Amplified and rearranged epidermal growth factor receptor genes in human glioblastomas reveal deletions of sequences encoding portions of the N- or C-terminal tails.” Proc. Natl. Acad. Sci. USA 1992; 89: 4309-13.
Ekstrand, A. J., James, C. D., Cavenee, W. K., Seliger, B., Pettersson, R. F., and Collins, V. P. (1991) Cancer Res. 51, 2164-2172.
Emsley, P. and Cowtan, K. (2004) Acta crystallographica 60, 2126-2132.
Fullot, T., Magdelenat, H., Mady, E., Stasiecki, P., Fohanno, D., Gropp, P., Poisson, M., and Delattre, J. Y. “A Phase I study of an anti-epidermal growth factor receptor monoclonal antibody for the treatment of malignant gliomas.” Neurosurgery (Baltimore), 39: 478-483, 1996.
Fairlie, W. D., Uboldi, A. D., De Souza, D. P., Hemmings, G. 1, Nicola, N. A., and Baca, M. (2002) Protein expression and purification 26, 171-178.
Fan, Z., and Mendelsohn, J. “Therapeutic application of anti-growth factor receptor antibodies.” Curr. Opin. Oncol., 10: 67-73, 1998.
Fan, Z., Baselga, J., Masui, H., and Mendelsohn, J. “Antitumor effect of antiepidermal growth factor receptor monoclonal antibodies plus cis-diamminedichloroplatinum on well established A431 cell xenografts.” Cancer Res., 53: 4637-4642, 1993.
Fan, Z., Masui, H., Altas, I., and Mendelsohn, J. “Blockade of epidermal growth factor receptor function by bivalent and monovalent fragments of 225 anti-epidermal growth factor receptor monoclonal antibodies.” Cancer Res., 53: 4322-4328, 1993.
Feldkamp, M. M., Lala, P., Lau, N., Roncari, L., and Guha, A. “Expression of activated-epidermal growth factor receptors, Ras-guanosine triphosphate, and mitogenactivated protein kinase in human glioblastoma multiforme specimens.” Neurosurgery (Baltimore), 45: 1442-1453, 1999.
Ferguson K M (2008) Annu. Rev. Biophys. 37, 353-373.
Ferguson K M, Berger M B, Mendrola J M, Cho H S, Leahy D J, & Lemmon M A (2003) Mol. Cell. 11, 507-517.
Ferguson, K. M., Berger, M. B., Mendrola, J. M., Cho, H. S., Leahy, D. J., and Lemmon, M. A. (2003) Mol. Cell. 11, 507-517.
Fernandes H, Cohen S, Bishayee S. “Glycosylation-induced conformational modification positively regulates receptor-receptor association: a study with an aberrant epidermal growth factor receptor (EGFRvIII/deEGFR) expressed in cancer cells.” J. Biol. Chem. 2001; 276: 5375-83.

Filmus J, Pollak M N, Cailleau R, et al. “MDA-468, a human breast cancer cell line with a high number of epidermal growth factor (EGF) receptors, has an amplified EGF receptor gene and is growth inhibited by EGF.” Biochem. Biophys. Res. Commun. 1985; 128: 898-905.
Filmus, J., Trent, J. M., Pollak, M. N., and Buick, R. N. “Epidermal growth factor receptor gene-amplified MDA-468 breast cancer cell line and its nonamplified variants.” Mol. Cell. Biol., 7: 251-257, 1987.
Gadella, T. W. J. and Jovin, T. M. (1995) Journal of Cell Biology 129, 1543-1558.
Gan H. K., Walker F., Burgess A. W., Rigopoulos A., Scott A. M. and Johns T. G. “The Epidermal Growth Factor Receptor (EGFR) Tyrosine Kinase Inhibitor AG1478 Increases the Formation of Inactive Untethered EGFR Dimers: Implications For Combination Therapy With Monoclonal Antibody 806.” J. Biol. Chem. (2007);282(5):2840-50.
Garcia de Palazzo, I. E., Adams, G. P., Sundareshan, P., Wong, A. J., Testa, J. R., Bigner, D. D., and Weiner, L. M. “Expression of mutated epidermal growth factor receptor by non-small cell along carcinomas.” Cancer Res., 53: 3217-3220, 1993.
Garnett (2001) Advanced drug Delivery Reviews 53: 171-216
Garrett T P, McKern N M, Lou M, Elleman T C, Adams T E, Lovrecz G O, Zhu H J, Walker F, Frenkel M J, Hoyne P A, et al. (2002) Cell 110, 763-773.
Garrett, T. P., McKern, N. M., Lou, M., Elleman, T. C., Adams, T. F., Lovrecz, G. 0., Zhu, H. J., Walker, F., Frenkel, M. J., Hoyne, P. A., Jorissen, R. N., Nice, E. C., Burgess, A. W., and Ward, C. W. (2002) Cell, 110, 763-773.
Garrett, T. P., McKern, N. M., Lou, M., Elleman, T. C., Adams, T. E., Lovrecz, G. 0., Kofler, M., Jorissen, R. N., Nice, E. c., Burgess, A. W., and Ward, C. W. (2003) Mol. Cell. 11, 495-505.
Gibson T B, Ranganathan A, Grothey A (2006) Clin. Colorectal Cancer 6(1):29-31.
Gill, G. N., Kawamoto, T., Cochet, C., Le, A., Sato, J. D., Masui, H., McLeod, C., and Mendelsohn, J. (1984) J. Biol. Chem. 259, 7755-7760.
Goldstein N I, Prewett M, Zuklys K, et al. “Biological efficacy of a chimeric antibody to the epidermal growth factor receptor in a human tumor xenograft model.” Clin. Cancer Res. 1995; 1: 1311-8.
Goldstein N I, Prewett M, Zuklys K, Rockwell P, Mendelsohn J (1995) Clin. Cancer Res. 1(11):1311-1318.
Graeven U, Kremer B, Sudhoff T, Kiling B, Rojo F, Weber D, Tillner J, Unal C, Schmiegel W (2006) Br. J. Cancer 94(9):1293-1299.
Grandis, J. R., Melhem, M. F., Gooding, W. E., Day, R., Holst, V. A., Wagener, M. M., Drenning, S. D., and Tweardy, D. J. “Levels of TGP-a and BOFR protein in head and neck squamous cell carcinoma and patient survival.” J. Natl. Cancer Inst., 90: 32CE, 1998.
Green, M. C., Murray, J. L., and Hortobagyi, G. N. “Monoclonal antibody therapy for solid tumors.” Cancer Treat. Rev., 26: 269-286, 2000.
Gunther, N., Betzel, C., and Weber, W. “The secreted form of the epidermal growth factor receptor. Characterization and crystallization of the receptor ligand complex.” J. Biol. Chem. 265: 22082-5, 1990.
Halaisch, M. E., Schmidt, U., Botefur, I. C., Holland, J. P., and Ohnuma, T. “Marked inhibition of glioblastoma target cell tumorigenicity in vitro by retrovirus-mediated transfer of a hairpin ribozyme against deletion-mutant epidermal growth factor receptor messenger RNA.” J. Neurosurg., 92: 297-305, 2000.
Han, Y., Caday, C. G., Nanda, A., Cavenee, W. K., and Huang, H. J. “Tyrphostin AG1478 preferentially inhibits human glioma cells expressing truncated rather than wild-type epidermal growth factor receptors. Cancer Res.” 56: 3859-3861, 1996.
Harari, D., and Yarden, Y. “Molecular mechanisms underlying ErbB2/HER2 action in breast cancer.” Oncogene, 19: 6102-6114, 2000.
Hellstrom et al., “Antibodies For Drug Delivery”, in Controlled Drug Delivery (2nd Ed.), Robinson et al. (eds.), pp. 623-53 (Marcel Dekker, Inc. 1987)
Herbst R S, Kim E S, & Harari P M (2001) Expert Opin. Biol. Ther. 1, 719-732.
Hills D, Rowlinson-Busza D, Gullick W J (1995) Int. J. Cancer 63(4):537-543.
Hills D, Rowlinson-Busza G, Gullick W J. “Specific targeting of a mutant, activated EGF receptor found in glioblastoma using a monoclonal antibody.” Int. J. Cancer 1995; 63: 537-43.
Hogg P J (2003) Trends Biochem. Sci. 28, 210-214.
Hogg, P. J. (2003) Trends in biochemical sciences 28, 210-214.
Holbro, T. and Hynes, N. E. (2004) Annu. Rev. Pharmacal. Toxicol. 44: 195-217., 195-217.
Hoofs, R. W., Vriend, G., Sander, C., and Abola, E. E. (1996) Nature 381,272.
Huang H S, Nagane M, Klingbeil C K, et al. “The enhanced tumorigenic activity of a mutant epidermal growth factor receptor common in human cancers is mediated by threshold levels of constitutive tyrosine phosphorylation and unattenuated signaling.” J. Biol. Chem. 1997; 272: 2927-35.
Humphrey P A, Wong A J, Vogelstein B, Zalutsky M R, Fuller G N, Archer G E, Friedman H S, Kwatra M M, Bigner S H, & Bigner D D (1990) Proc. Natl. Acad. Sci. USA 87, 4207-4211.
Humphrey P A, Wong A J, Vogelstein B, Zalutsky M R, Fuller G N, Archer G E, Friedman H S, Kwatra M M, Bigner S H, Bigner D D (1990) Proc. Natl. Acad. Sci. USA 87(11):4207-4211.
Humphrey, P. A., Wong, A. 1., Vogelstein, B., Zalutsky, M. R., Fuller, G. N., Archer, G. E., Friedman, H. S., Kwatra, M. M., Bigner, S. H., and Bigner, D. D. “Anti-synthetic peptide antibody reacting at the fusion junction of deletion mutant epidermal growth factor receptors in human glioblastoma” (1990) Proc. Natl. Acad. Sci. USA 87, 4207-4211.
Johns T G, Adams T E, Cochran J R, Hall N E, Hoyne P A, Olsen M J, Kim Y S, Rothacker J, Nice E C, Walker F, et al. (2004) J. Biol. Chem. 279, 30375-30384.
Johns T G, Adams T E, Wittrup K D, Hall N E, Hoyne P A, Cochrane J R, Olsen M J, Kim Y S, Rothacker J, Nice E C, et al. (2004) J. Biol. Chem. 279(29):30375-30384.
Johns T G, Luwor R B, Murone C, Walker F, Weinstock J, Vitali A A, Perera R M, Jungbluth A A, Stockert E, Old L J, et al. (2003) Proc. Natl. Acad. Sci. USA 100, 15871-15876.
Johns T G, Luwor R B, Murone C, Walker F, Weinstock J, Vitali A A, Perera R M, Jungbluth A A, Stockert E, Old L J, et al. (2003) Proc. Natl. Acad. Sci. USA 100(26):15871-15876.
Johns T G, Mellman I, Cartwright G A, Ritter G, Old L J, Burgess A W, Scott A M (2005) FASEB J. 19(7):780-782.
Johns T G, Perera R M, Vernes S C, Vitali A A, Cao D X, Cavenee W K, Scott A M and Furnari F B. “The efficacy of EGFR-specific antibodies against glioma xenografts is influenced by receptor levels, activation status and heterodimerization.” Clin. Cancer Res. (2007);13(6): 1911-1925.
Johns T G, Stockert E, Ritter G, Jungbluth A A, H-J. Su Huang, Cavenee W K, Smyth F E, Hall C M, Watson N, Nice E C, Gullick W J, Old L J, Burgess A W, Scott A M. “Novel monoclonal antibody specific for the DE2-7 Epidermal Growth Factor Receptor (EGFR) that also recognizes the EGFR expressed in cells containing amplification of the EGFR gene.” Int. J. Cancer (2002) 98: 398-408
Johns T G, Stockert E, Ritter G, Jungbluth A A, Huang H J, Cavenee W K, Smyth F E, Hall C M, Watson N, Nice E C, et al. (2002) Int. J. Cancer. 98, 398-408.
Johns T G, Stockert E, Ritter G, Jungbluth A A, Huang H J, Cavenee W K, Smyth F E, Hall C M, Watson N, Nice E C, et al. (2002) Int. J. Cancer 98(3):398-408.
Johns, T. G., Adams, T. E., Cochran, J. R., Hall, N. E., Hoyne, P. A., Olsen, M. J., Kim, Y S., Rothacker, J., Nice, E. C., Walker, F., Ritter, G., Jungbluth, A. A., Old, L. J., Ward, C. W., Burgess, A. W., Wittrup, K. D., and Scott, A. M. “Identification of the Epitope for the EGFR-Specific Monoclonal Antibody 806 Reveals that it Preferentially Recognizes an Untethered Form of the Receptor.” J. Biol. Chem. (2004) 279: 30375-30384
Johns, T. G., et al., “Novel monoclonal antibody specific for the de2-7 epidermal growth factor receptor (EGFR) that also recognizes the EGFR expressed in cells containing amplification of the EGFR gene.” Int. J. Cancer, 2002. 98(3): p. 398-408.
Johns, T. G., Luwor, R. B., Murone, C., Walker, F., Weinstock, J., Vitali, A. A., Perera, R. M., Old, L. J., Nice, E. C., Burgess, A. W. and Scott, A. M. “Anti-tumor efficacy of cytotoxic drugs and the monoclonal antibody 806 is enhanced by the epidermal growth factor receptor (EGFR) inhibitor AG1478.” PNAS (2003) 100: 15871-15876
Johns, T. G., Mellman I., Cartwright G. A., Ritter G., Old L. J., Burgess A. W. and Scott A. M. “The anti-tumor monoclonal antibody 806 recognizes a high-mannose form of the EGF receptor that reaches the cell surface when cells over-express the receptor.” FASEB J. (2005) 19(7):780-2.
Jorissen, R. N., Walker, F. W., Pouliot, N., Garrett, T. P. J., Ward, C. W., and Burgess, A. W. “Epidermal growth factor receptor: mechanisms of activation and signaling.” Exp. Cell Res. 284,31-53, 2003.
Jungbluth A A, Stockert E, Huang H J, Collins V P, Coplan K, Iversen K, Kolb D, Johns T J, Scott A M, Gullick W J, et al. (2003) Proc. Natl. Acad. Sci. USA 100, 639-644.
Jungbluth A A, Stockert E, Huang H J, Collins V P, Coplan K, Iversen K, Kolb D, Johns T G, Scott A M, Gullick W J, et al. (2003) Proc. Natl. Acad. Sci. USA. 100(2): 639-644.
Jungbluth, A. A., Stockert, E., Huang, H-J. S., Collins, V P, Coplan, K., Iversen, K., Kolb, D., Johns T. G., Scott A. M., Gullick W. J., Ritter, G., Cohen L., Cavanee W. K., Old, L. J. A “Monoclonal Antibody Recognizing Human Cancers with Amplification/Over-Expression of the Human Epidermal Growth Factor Receptor.” PNAS (2003) 100: 639-644.
Korshunov, A., Golanov, A., Sycheva, R., and Pronin, I. “Prognostic value of tumor associated antigen immunoreactivity and apoptosis in cerebral glioblastomas: an analysis of 163 cases.” J. Clin. Pathol., 52,-574-580, 1999.
Kwok T T, Sutherland R M. “Differences in EGF related radiosensitisation of human squamous carcinoma cells with high and low numbers of EGF receptors.” Br. J. Cancer 1991; 64: 251-4.
Lacouture A E (2006) Nature Rev. Cancer. 6: 803-812.
Laskowski, R. A., MacArthur, M. W., Moss, D. S., and Thornton, J. M. (1993) J. Appl. Cryst. 26, 283-291.
Lee F. T., Mountain A. J., O'Keefe G. J., Sagona J., Rigopoulos A., Smyth F. E., Govindan S. V., Goldenberg D. M., Old L. J. and Scott A M “ImmunoPET detection of xenografts expressing de2-7 EGFR using Iodine-124 labelled ch806 via residualising ligand IMPR4.” J. Nucl. Med. (2006) 47 (5) suppl 1: 429P.
Li D., Ji H., Zaghlul S., McNamara K., Liang M. C., Shimamura T., Kubo S., Takahashi M., Chirieac L. R., Padera R. F., Scott A. M., Jungbluth, A. A., Cavenee W. K., Old L. J., Demetri G. D.,
Wong K K. “Therapeutic anti-EGFR antibody 806 generates responses in murine de novo EGFR mutant-dependent lung carcinomas.” J. Clin. Invest. (2007); 117(2): 346-352.
Li S, Kussie P, & Ferguson K M (2008) Structure 16, 216-227.
Li S, Schmitz K R, Jeffrey P D, Wiltzius J J, Kussie P, & Ferguson K M (2005) Cancer Cell. 7, 301-311.
Lindmo, T., et al., “Determination of the immunoreactive fraction of radiolabeled monoclonal antibodies by linear extrapolation to binding at infinite antigen excess.” J. Immunol. Methods, 1984. 72(1): p. 77-89.
Liu Z, Panousis C, Smyth F E, Murphy R, Wirth V, Cartwright G, Johns T G, Scott A M (2003) Hybrid Hybridomics 22(4):219-28.
Liu, Z., Panousis, C., Smyth, F. E., Murphy, R., Wirth, V., Cartwright, G., Johns, T. G., and Scott, A. M. “Generation of Anti-Idiotype Antibodies for Application in Clinical Immunotherapy Laboratory Analyses.” Hybridoma and Hybridomics, (2003) 22 (4): 219-228.
Luwor R B, Johns T G, Murone C, H-J. Su Huang, Cavenee W K, Ritter G, Old L J, Burgess A W, Scott A M. “Monoclonal Antibody 806 Inhibits the Growth of Tumor Xenografts Expressing Either the DE2-7 or Amplified Epidermal Growth Factor Receptor (EGFR) but not Wild-Type EGFR.” Cancer Research (2001) 61: 5355-5361.
Luwor R B, Johns T G, Murone C, Huang H J, Cavenee W K, Ritter G, Old L J, Burgess A W, & Scott A M (2001) Cancer Res. 61, 5355-5361.
Luwor R B, Johns T G, Murone C, Huang H J, Cavenee W K, Ritter G, Old L J, Burgess A W, Scott A M (2001) Cancer Res. 61(14): p. 5355-5361.
Luwor, R. B., Zhu, H-J., Walker, F., Vitali A. A., Perera, R. M., Burgess, A. W., Scott, A. M. and Johns, T. G. “The Tumor Specific de2-7 Epidermal Growth Factor Receptor (EGFR) confers Increased survival in BaF/3 Cells Via a PI-3 Kinase Dependent Mechanism.” Oncogene (2004) 23: 6095-6104
Lynch D H & Yang X D (2002) Semin Oncol. 29, 47-50.
MacDonald, A., Chisholm, G. D., and Habib, F. K. (1990) Br. J. Cancer. 62, 579-584.
Maloney D G, Grillo-Lopez A J, White C A, Bodkin D, Schilder R J, Neidhart J A, Janakiraman N, Foon K A, Liles T M, Dallaire B K, et al. (1997) Blood 90(6):2188-2195.
Masui, H., Kawamoto, T., Sato, J. D., Wolf, B., Sato, G., and Mendelsohn, J. “Growth inhibition of human tumor cells in athymic mice by anti-epidermal growth factor receptor monoclonal antibodies.” Cancer Res., 44: 1002-1007, 1984.
Mellinghoff, I. K., Cloughesy, T. F., and Mischel, P. S. (2007) Clin. Cancer Res. 13, 378-381.
Mendelsohn J & Baselga J (2006) Semin Oncol. 33, 369-385.
Mendelsohn, J. “Epidermal growth factor receptor inhibition by a monoclonal antibody as anticancer therapy. Clin. Cancer Res.” 3: 2703-2707, 1997.
Mickey. D. D., Stone, K. R., Wunderli, H., Mickey, G. H., Vollmer, R. T., and Paulson, D. F. (1977) Cancer Res. 37, 4049-4058.
Milano G, Spano J P, & Leyland-Jones B (2008) Br. J. Cancer 99, 1-5.
Mineo C, Gill G N, Anderson R G. “Regulated migration of epidermal growth factor receptor from caveolae.” J. Biol. Chem. 1999; 274: 30636-43.
Mishima K, Johns T G, Luwor R B, Scott A M, Stockert E, Jungbluth A A, Ji X, Suvarna P, Voland J R, Old L J, H-J. Su Huang, Cavenee W K. “Growth Suppression of Intracranial Xenografted Glioblastomas Overexpressing Mutant Epidermal Growth Factor Receptors by Systemic Administration of Monoclonal Antibody (mAb) 806, a Novel Monoclonal Antibody Directed to the Receptor.” Cancer Research (2001) 61: 5349-5354.
Mishima K, Johns T G, Luwor R B, Scott A M, Stockert E, Jungbluth A A, Ji X D, Suvarna P, Voland J R, Old L J, et al. (2001) Cancer Res. 61(14):5349-5354.
Mishima, K. Nagane, M., Lin, H., Cavenee, W. K., and Huang, H-J. S. “Expression of a tumor-specific mutant epidermal growth factor receptor mediates glioma cell invasion in vivo.” Proc. Am. Assoc. Cancer Res., 40: 519, 1999.
Mishima, K., Mazar, A. P., Gown, A., Skelly, M., Ji, X. D., Wang, X. D., Jones, T. R., Cavenee, W. K., and Huang, H-J. S. “A peptide derived from the non-receptor-binding region of urokinase plasminogen activator inhibits glioblastoma growth and angiogenesis in vivo in combination with cisplatin.” Proc. Natl. Acad. Sci. USA, 97: 8484-8489, 2000.
Moscatello, D. K., Holgado-Madruga, M., Godwin, A. K., Ramirez, G., Gunn, G., Zoltick, P. W., Biegel, J. A., Hayes, R. L., and Wong, A. J. “Frequent expression of a mutant epidermal growth factor receptor in multiple human tumors.” Cancer Res., 55: 5536-5539, 1995.
Murshudov G N, Vagin A A, & Dodson E J (1997) Acta Crystallogr. D. Biol. Crystallogr. 53, 240-255.
Murshudov, G. N., Vagin, A. A., and Dodson, E. J. (1997) Acta crystallographica 53, 240-255.
Nagane, M., Coufal, F., Lin, H., Bogler, 0., Cavenee, W. K., and Huang, H. J. “A common mutant epidermal growth factor receptor confers enhanced tumorigenicity on human glioblastoma cells by increasing proliferation and reducing apoptosis.” Cancer Res. 56: 5079-86, 1996.
Nagane, M., Levitzki, A., Gazit, A., Cavenee, W. K., and Huang, H-J. S. “Drug resistance of human glioblastoma cells conferred by a tumor-specific mutant epidermal growth factor receptor through modulation of Bcl-XL and caspase-3-like proteases.” Proc. Natl. Acad. Sci. USA, 95: 5724-5729, 1998.
Nagane, M., Lin, H., Cavenee, W. K., and Huang, H-J. S. “Aberrant receptor signaling in human malignant gliomas: mechanisms and therapeutic implications.” Cancer Lett., 162 (Suppl. 1):517-S21, 2001.
Neidhardt, F. C., Bloch, P. L., and Smith, D. F. (1974) Journal of bacteriology 119, 736-747.
Nishikawa R, Ji X D, Harmon R C, Lazar C S, Gill G N, Cavenee W K, Huang H J (1994) Proc. Natl. Acad. Sci. USA 91(16):7727-7731.
Nishikawa R, Ji X D, Harmon R C, Lazar C S, Gill G N, Cavenee W K, & Huang H J (1994) Proc. Natl. Acad. Sci. USA. 91, 7727-7731.
Nishikawa, R., Ji, X. D., Harmon, R. C., Lazar, C. S., Gill, G. N., Cavenee, W. K., and Huang, H. J. A mutant epidermal growth factor receptor common in human glioma confers enhanced tumorigenicity. Proc. Natl. Acad. Sci. USA, 91: 7727-7731, 1994.
Ogiso, H., Ishitani, R, Nureki, 0., Fukai, S., Yamanaka, M., Kim, l. H., Saito, K., Sakamoto, A., Inoue, M., Shirouzu, M., and Vokoyama, S. (2002) Cell 20: 110775-787.
Okamoto S, Yoshikawa K, Obata Y, et al. “Monoclonal antibody against the fusion junction of a deletion-mutant epidermal growth factor receptor.” Br. J. Cancer 1996; 73: 1366-72.
Olapade-Olaopa, E. 0., Moscatello, D. K., MacKay, E. H., Horsburgh, T., Sandhu, D. P., Terry, T. R., Wong, A. J., and Habib, F. K. “Evidence for the differential expression of a variant EGF receptor protein in human prostate cancer.” Br. J. Cancer. 82: 186-94, 2000.
Old, L. J. “Immunotherapy for cancer.” Sci. Am., 275: 102-109, 1996.
Otwinowski, Z. and Minor, W. (1997) “Processing of X-ray diffraction data collected in oscillation mode.” Academic Press (New York).
Padlan E A. “A possible procedure for reducing the immunogenicity of antibody variable domains while preserving their ligand-binding properties.” Mol. Immunol. 1991 28(4-5):489-98.
Padlan et al., EP 519596, Merck/NIH
Palacios, R, Henson, G., Steinmetz, M., and McKeam, J. P. (1984) Nature. 309, 126-131.
Panousis C, Rayzman V M, Johns T G, Renner C, Liu Z, Cartwright G, Lee F-T, Wang D, Kypridis A, Smyth F E, et al. (2005) Br. J. Cancer. 92(6):1069-1077.
Panousis, C., Rayzman, V. M., Johns, T. G., Renner C., Liu Z., Cartwright, G., Lee F-T., Wang, D., Gan, H., Cao, D., Kypridis, A., Smyth, F. E., Brechbiel, M. W., Burgess, A. W., Old, L. J. and Scott, A. M. “Engineering and characterization of chimeric monoclonal antibody 806 (ch806) for targeted immunotherapy of tumours expressing de2-7 EGFR or amplified EGFR.” Br. J. Cancer (2005) 92: 1069-1077.
PCT Patent Publication No. WO 02/092771.
PCT Patent Publication No. WO 2009/017394.
PCT Patent Publication No. WO 2009/023265.
PCT Patent Publication No. WO 2010/096434.
Perera R. M., Narita Y., Furnari, F. B., Luwor, R. B., Burgess, A. W., Old, L. J., Cavenee, W. K., Scott, A. M. and Johns, T. G. “A novel EGFR antibody that displays synergistic anti-tumor activity when combined with conventional EGFR therapeutics.” Clinical Cancer Research (2005) 11: 6390-6399.
Perera R. M., Zoncu R., Johns T. G., Pypaert M., Lee F. T., Mellman I., Old L. J., Toomre D. K., and Scott A. M. “Internalization, intracellular trafficking, and biodistribution of monoclonal antibody 806: a novel anti-epidermal growth factor receptor antibody.” Neoplasia. (2007); 9(12):1099-110
Perera R M, Narita Y, Furnari F B, Gan H K, Murone C, Ahlkvist M, Luwor R B, Burgess A W, Stockert E, Jungbluth A A, et al. (2005) Clin. Cancer. Res. 11, 6390-6399.
Perera R M, Narita Y, Furnari F B, Tavernasi M L, Luwor R B, Burgess A W, Stockert E, Jungbluth A A, Old L J, Cavenee W K, et al. (2005) Clin. Cancer Res. 11(17):6390-6399.
Perez-Soler, R., Donato, N. J., Shin, D. M., Rosenblum, M. G., Zhang, H. Z., Tomos, C., Brewer, H., Chan, J. C., Lee, J. S., Hong, W. K., et al. “Tumor epidermal growth factor receptor studies in patients with non-small-cell lung cancer or head and neck cancer treated with monoclonal antibody RG 83852.” J. Clin. Oncol., 12: 730-739, 1994.
Pietras, R. J., Pegam, M. D-, Finn, R—S., Maneval, D. A., and Slmon, D. J. “Remission of human breast cancer xenografts on therapy with humanized monoclonal antibody to HER-2 receptor and DNA-reactive drugs.” Oncogene, 17: 2235-2249, 1998.
Ponten J, Macintyre E H. “Long term culture of normal and neoplastic human glia.” Acta Pathol. Microbiol. Scand. 1968; 74: 465-86.
Press, O. W., DeSantes, K., Anderson, S. K., and Geissler, F. “Inhibition of catabolism of radiolabeled antibodies by tumor cells using lysosomotropic amines and carboxylic ionophores.” Cancer Res. 50: 1243-50, 1990.
Ramos T C, Figueredo J, Catala M, Gonzales S, Selva J C, Cruz T M, Toldeo C, Silva S, Pestano Y, Ramos M, et al. (2006) Cancer Biol. Ther. 5(4):375-379.
Reist C J, Batra S K, Pegram C N, et al. “In vitro and in vivo behavior of radiolabeled chimeric anti-EGFRvIII monoclonal antibody: comparison with its murine parent.” Nucl. Med. Biol. 1997; 24: 639-47.
Reist, C. J., Archer, G. E., Kurpad, S, N., Wikstrand, C. J., Vaidyanathan, G., Willingham, M. C., Moscatello, D. K., Wong, A. J., Bigner, D. D., and Zalutsky, M. R. “Tumor-specific anti-epidermal growth factor receptor variant III monoclonal antibodies: use of the tyramine-cellobiose radioiodination method enhances cellular retention and uptake in tumor xenografts.” Cancer Res., 55: 4375-4382, 1995.
Reist, C. J., Archer, G. E., Wikstrand, C. J., Bigner, D. D., and Zalutsky, M R “Improved targeting of an anti-epidermal growth factor receptor variant III monoclonal antibody in tumor xenografts after labeling using N-succinimidyl 5-iodo-3-pyridinecarboxylate.” Cancer Res. 57: 1510-5, 1997.
Reist, C. J., Batra, S. K., Pegram, C. N., Bigner, D. D., and Zalutsky, M. R. “In vitro and in vivo behavior of radiolabeled chimeric anti-EGFRvIII monoclonal antibody: comparison with its murine parent.” Nucl. Med. Biol. 24: 63947, 1997.
Reist, C. J., Garg, P. K., Alston, K. L., Bigner, D. D., and Zalutsky, M. R. “Radioiodination of internalizing monoclonal antibodies using N-succinimidyl 5-iodo-3-pyridinecarboxylate.” Cancer Res. 56: 4970-7, 1996.
Rettig W J, Old L J (1989) Annu. Rev. Immunol. 7: 481-511.
Ritter G, Cohen L S, Williams C Jr, Richards E C, Old L J, Welt S (2001) Cancer Res. 61(18):685-6859.
Rodeck, U., Herlyn, M., Herlyn, D., Molthoff, C., Atkinson, B., Varello, M., Steplewski, Z., and Koprowski, H. “Tumor growth modulation by a monoclonal antibody to the epidermal growth factor receptor: immunologically mediated and effector cell-independent effects.” Cancer Res., 47: 3692-3696, 1987.
Rowinsky E K, Schwartz G H, Gollob J A, Thompson J A, Vogelzang N J, Figlin R, Bukowski R, Haas N, Lockbaum P, Li Y P, et al. (2004) J. Clin. Oncol. 22: 3003-3015.
Safa, M. M. and K. A. Foon (2001). “Adjuvant immunotherapy for melanoma and colorectal cancers.” Semin. Oncol. 28 (1): 68-92.
Salomon, D. S., Brandt, R., C₁₋ardiello, F., and Normanno, N. “Epidermal growth factor-related peptides and their receptors in human malignancies.” Crit. Rev. Oncol. Hematol., 19: 183-232, 1995.
Sampson J H, Crotty L E, Lee S, Archer G E, Ashley D M, Wikstrand C J, Hale L P, Small C, Dranoff G, Friedman A H, et al. (2000) Proc. Natl. Acad. Sci. USA 97, 7503-7508.
Sampson, J. H, Crotty, L. E., Lee, S., Archer, G. E., Ashley, D. M., Wikstrand, C. J., Hale, L. P., Small, C., Dranoff, G.,: Friedman, A. H., Friedman, H. S., and Bigner, D. D, “Unarmed, tumor-specific monoclonal antibody effectively treats brain tumors.” Proc. Natl. Acad. Sci. USA, 97: 7503-7508, 2000.
Sandler A B (2006) Oncology (Williston Park) 20, 35-40.
Santon, J. B., Cronin, M. T., MacLeod, C. L., Mendelsohn, J., Masui, H., and Gill, G. N. “Effects of epidermal growth factor receptor concentration on tumorigenicity of A431 cells in nude mice.” Cancer Res. 46: 4701-5, 1986.
Sato, J. D., Le, A. D., and Kawamoto, T. “Derivation and assay of biological effects of monoclonal antibodies to epidermal growth factor receptors.” Methods Enzymol. 146: 63-81, 1987.
Schlessinger, J. (2002) Cell 20; 110, 669-672.
Schmiedel J, Blaukat A, Li S, Knochel T, & Ferguson K M (2008) Cancer Cell 13, 365-373.
Scott A. M., Gill S. S., Lee F., Liu Z., Skrinos E., Murone C., Saunder T., Chappell B., Papenfuss A., Old L. J. “A Phase I single dose escalation trial of ch806 in patients with advanced tumors expressing the 806 antigen.” Journal of Clinical Oncology, 2006 ASCO Annual Meeting Proceedings Part I. Vol. 24, No. 18S (June 20 Supplement), (2006): 13028.
Scott A. M., Lee F T., Tebbutt N., Herbertson R., Gill S. S., Liu Z., Skrinos E., Murone C., Saunder T. H., Chappell B., Papenfuss A. T., Poon A. M. T., Hopkins W., Smyth F. E., MacGregor D., Chem. L. M., Jungbluth A. A., Ritter, G., Brechbiel M. W., Murphy R., Burgess A W, Hoffman E. W., Johns T. J., Old L. J. “A Phase I clinical trial with monoclonal antibody ch806 targeting transitional state and mutant epidermal growth factor receptors.” Proc. Natl. Acad. Sci. USA, (2007) 104 (10): 4071-6. Epub 2007 February
Scott A M, Geleick D, Rubira M, Clarke K, Nice E C, Smyth F E, Stockert E, Richards E C, Carr F J, Harris W J, et al. (2000) Cancer Res 60: 3254-3261.
Scott A M, Lee F-T, Hopkins W, Cebon J S, Wheatley J M, Liu Z, Smyth F E, Murone C, Sturrock S, MacGregor D, et al. (2001) J. Clin. Oncol. 19(19):3976-3987.
Scott A M, Lee F T, Jones R, Hopkins W, MacGregor D, Cebon J, Hannah A, U P, Rigopolous A, Sturrock S, et al. (2005) Clin. Cancer Res. 11(13):4810-4817.
Scott A M, Lee F T, Tebbutt N, Herbertson R, Gill S S, Liu Z, Skrinos E, Murone C, Saunder T H, Chappell B, et al. (2007) Proc. Natl. Acad. Sci. USA 104, 4071-4076.
Scott A M, Wiseman G, Welt S, Adjei A, Lee F T, Hopkins W, Divgi C R, Hanson L H, Mitchell P, Gansen D N, et al. (2003) Clin. Cancer Res. 9: 1639-47.
Scott, A. M., and Welt, S. Antibody-based immunological therapy. Curr. Opin. Immunol., 9: 717-722, 1997.
Seymour L. “Novel anti-cancer agents in development: exciting prospects and new challenges.” Cancer Treat. Rev. 1999; 25: 301-12.
Sivasubramanian A, Chao G, Pressler H M, Wittrup K D, & Gray J J (2006) Structure 14, 401-414.
Sizeland A M & Burgess A W (1991) Mol. Cell. Biol. 11, 4005-4014.
Sizeland, A. M. and Burgess, A. W. (1991) Mol. Cell. Bio, 11, 4005-4014.
Sizeland, A. M. and Burgess, A. W. (1992) Mol. Biol. Cell 3, 1235-1243.
Slamon, D. J., Godolphin, W., Jones, L. A., Holt, J. A., Wong, S. G., Keith, D. E., Levin, W. J., Stuart, S. G., Udove, J., Ullrich, A., and Press, M. F. “Studies of the HER-2/neu proto-oncogene in human breast and ovarian cancer.” Science (Wash. DC). 244: 707-712, 1989.
Sliwkowski, M. X., Lofgren, J. A., Lewis, G. D., Hotaling, T. E., Fendly, B. M., and Fox, J. A. “Nonclinical studies addressing the mechanism of action of trastuzumab (Herceptin).” Semin Oncol., 26 (Suppl. 12): 60-70, 1999.
Sok, J. c., Coppelli, F. M., Thomas, S. M., Lango, M. N., Xi, S., Hunt, J. 1., Freilino, M. l., Graner, M. W., Wikstrand, C. J., Bigner, D. D., Gooding, W. E., Furnari, F. B., and Grandis, J. R. (2006) Clin. Cancer Res. 12, 5064-5073.
Solomon B M & Jatoi A (2008) Curr. Oncol. Rep. 10, 304-308.
Sridhar S S, Seymour L, Shepherd F A (2003) Lancet Oncol. 4(7):397-406.
Stabin M G, Sparks R B, Crowe E (2005) J. Nucl. Med. 46(6):1023-1027.
Stamos, J., Sliwkowski, M. X., and Eigenbrot, C. (2002) J. Biol. Chem. 277, 46265-46272.
Steffens M G, Boerman O C, Oosterwijk-Wakka J C, Oosterhof G O, Witjes J A, Koenders E B, Oyen W J, Buijs W C, Debruyne F M, Corstens F H, et al. (1997) J. Clin. Oncol. 15: 1529-1537.
Sturgis, E. M., Sacks, P. G., Masui, H., Mendelsohn, J., and Schantz, S. P. “Effects of antiepidermal growth factor receptor antibody 528 on the proliferation and differentiation of head and neck cancer.” Otolaryngol. Head Neck Surg. 111: 633-43, 1994.
Sugawa N, Ekstrand A J, James C D, Collins V P (1990) Proc. Natl. Acad. Sci. USA. 87(21):8602-8606.
Sugawa, N., Ekstrand, A. J., James, C. D., and Collins, V. P. “Identical splicing of aberrant epidermal growth factor receptor transcripts from amplified rearranged genes in human glioblastomas.” Proc. Natl. Acad. Sci. USA, 87: 8602-8606, 1990.
Tan A R, Moore D F, Hidalgo M, Doroshow J H, Polpin E A, Goodin S, Mauro D, Rubin E H (2006) Clin. Cancer Res. 12(21): 6517-6522.
Tang, C. K., Gong, X. Q., Moscatello, D. K., Wong, A. J., and Lippman, M. E. “Epidermal growth factor receptor in enhances tumorigenicity in human breast cancer.” Cancer Res., 60: 3081-3087, 2000.
Teramoto, T., Onda, M., Tokunaga, A., and Asano, G “Inhibitory effect of antiepidermal growth factor receptor antibody on a human gastric cancer.” Cancer (Phila.), 77: 1639-1645, 1996.
Thorpe et al., “The Preparation And Cytotoxic Properties Of Antibody-Toxin Conjugates”, Immunol. Rev., 62: 119-58 (1982)
Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985)
Todaro, G. J., Delarco, J. E., and Cohen, S. (1976) Nature 264, 26-31.
Trail, P. A., and Bianchi, A. B. “Monoclonal antibody drug conjugates in the treatment of cancer.” Curr. Opin. Immunol., 11: 584-588, 1999.
U.S. Pat. No. 5,208,020.
U.S. patent application Ser. No. 10/145,598, filed May 13, 2002.
U.S. patent application Ser. No. 12/317,683, filed Dec. 23, 2008.
U.S. patent application Ser. No. 12/388,504, filed Feb. 18, 2009.
U.S. patent application Ser. No. 12/733,146, filed Feb. 12, 2010.
U.S. Pat. No. 4,137,230.
U.S. Pat. No. 4,151,042.
U.S. Pat. No. 4,162,940.
U.S. Pat. No. 4,190,580.
U.S. Pat. No. 4,225,494.
U.S. Pat. No. 4,228,239.
U.S. Pat. No. 4,248,870.
U.S. Pat. No. 4,256,746.
U.S. Pat. No. 4,260,608.
U.S. Pat. No. 4,263,294.
U.S. Pat. No. 4,264,596.
U.S. Pat. No. 4,265,814.
U.S. Pat. No. 4,294,757.
U.S. Pat. No. 4,307,016.
U.S. Pat. No. 4,308,268.
U.S. Pat. No. 4,308,269.
U.S. Pat. No. 4,309,428.
U.S. Pat. No. 4,317,821.
U.S. Pat. No. 4,320,200.
U.S. Pat. No. 4,322,348.
U.S. Pat. No. 4,331,598.
U.S. Pat. No. 4,360,462.
U.S. Pat. No. 4,361,650.
U.S. Pat. No. 4,362,663.
U.S. Pat. No. 4,364,866.
U.S. Pat. No. 4,371,533.
U.S. Pat. No. 4,424,219.
U.S. Pat. No. 4,450,234.
U.S. Pat. No. 4,563,304.
U.S. Pat. No. 4,675,187.
U.S. Pat. No. 4,676,980.
U.S. Pat. No. 5,141,736.
U.S. Pat. No. 5,217,713.
U.S. Pat. No. 5,475,092.
U.S. Pat. No. 5,585,499.
U.S. Pat. No. 5,635,483.
U.S. Pat. No. 5,846,545.
U.S. Pat. No. 6,214,345.
U.S. Pat. No. 6,534,660.
U.S. Pat. No. 6,586,618.
U.S. Pat. No. 6,756,397.
U.S. Pat. No. 6,884,869.
U.S. Pat. No. 7,049,316.
U.S. Pat. No. 7,090,843.
U.S. Pat. No. 7,091,186.
U.S. Pat. No. 7,098,308.
U.S. Pat. No. 7,214,685.
U.S. Pat. No. 7,223,837.
U.S. Pat. No. 7,256,257.
U.S. Pat. No. 7,276,497.
U.S. Pat. No. 7,329,760.
U.S. Pat. No. 7,388,026.
U.S. Pat. No. 7,423,116.
U.S. Pat. No. 7,498,298.
U.S. Pat. No. 7,553,816.
U.S. Pat. No. 7,589,180.
U.S. Pat. No. 7,655,660.
U.S. Pat. No. 7,655,661.
U.S. Pat. No. 7,659,241.
U.S. Patent Publication No. 2003/0083263.
U.S. Patent Publication No. 2004/0018194.
U.S. Patent Publication No. 2004/0121940.
U.S. Patent Publication No. 2004/0157782.
U.S. Patent Publication No. 2005/0009751.
U.S. Patent Publication No. 2005/0113308.
U.S. Patent Publication No. 2006/0116422.
U.S. Patent Publication No. 2006/0229253.
U.S. Patent Publication No. 2007/0135346.
U.S. Patent Publication No. 2007/0258987.
U.S. Patent Publication No. 2008/0213289.
U.S. Patent Publication No. 2008/0226657.
U.S. Patent Publication No. 2008/0241128.
U.S. Patent Publication No. 2008/0248051.
U.S. Patent Publication No. 2008/0248053.
U.S. Patent Publication No. 2008/0260685.
U.S. Patent Publication No. 2008/0300192.
U.S. Patent Publication No. 2008/0311136.
U.S. Patent Publication No. 2008/0317747.
U.S. Patent Publication No. 2009/0010945.
U.S. Patent Publication No. 2009/0018086.
U.S. Patent Publication No. 2009/0047296.
U.S. Patent Publication No. 2009/0111756.
U.S. Patent Publication No. 2009/0220510.
U.S. Patent Publication No. 2009/0281158.
U.S. Patent Publication No. 2009/0318668.
U.S. Patent Publication No. 2010/0056762.
U.S. Provisional Patent Application No. 60/290,410, filed May 11, 2001.
U.S. Provisional Patent Application No. 60/326,019, filed Sep. 28, 2001.
U.S. Provisional Patent Application No. 60/342,258, filed Dec. 21, 2001.
U.S. Provisional Patent Application No. 60/964,715, filed Aug. 14, 2007.
Uemura, H., E. Okajima, et al. (1994).“Internal image anti-idiotype antibodies related to renal-cell carcinoma-associated antigen G250.” Int. J. Cancer 56 (4): 609-14.
Ullrich A, Coussens L, Hayflick J S, Dull T J, Gray A, Tam A W, Lee J, Yarden Y, Libermann T A, & Schlessinger J (1984) Nature. 309, 418-425.
Ulrich, A., Coussens, L., Hayflick, J. S., Dull, T. J., Gray, A., Tam, A. W., Lee, J., Yarden, Y., Libermann, T. A., Schlessinger, J., and (1984) Nature. 309, 418-425.
Vagin A A & Isupov M N (2001) Acta Crystallogr. D. Biol. Crystallogr. 57, 1451-1456.
Vagin, A. and Teplyakov, A. (1997) J. Appl. Cryst. 30, 1022-1025.
van de Loosdrecht, A. A., Beelen, R. H., Ossenkoppele, G. J., Broekhoven, M. G., and Langenhuijsen, M. M. (1994) J. Immunol. Methods. 174, 311-320.
Van den Eynde B J, Scott A M (1998) in Encyclopedia of Immunology, eds Roitt D P J, Roitt I M (Academic Press: London), pp 2424-2431.
Voldborg B R, Damstrup L, Spang-Thomsen M, Poulsen H S (1997) Ann Oncol. 8(12): 1197-206.
Voldborg, B. R., Damstrup, L., Spang-Thomsen, M., and Poulsen, H. S. “Epidermal growth factor receptor (EGFR) and EGFR mutations, function and possible role in clinical trials.” Ann. Oncol., 8: 1197-1206, 1997.
Wade J D, Hojo K, Kawasaki K, Johns T G, Catimel B, Rothacker J, & Nice E C (2006) Anal. Biochem. 348, 315-317.
Wade, J. D., Hojo, K., Kawasaki, K., Johns, T. G., Catimel, B., Rothacker, J., and Nice, E. C. (2006) Anal. Biochem. 348, 315-317.
Waksal, H. W. “Role of an anti-epidermal growth factor receptor in treating cancer.” Cancer Metastasis Rev., 18: 427-436, 1999.
Walker F, Hibbs M L, Zhang H H, Gonez L J, & Burgess A W (1998) Growth Factors 16, 53-67.
Walker F, Orchard S G, Jorissen R N, Hall N E, Zhang H H, Hoyne P A, Adams T E, Johns T G, Ward C, Garrett T P, et al. (2004) J. Biol. Chem. 279, 22387-22398.
Walker, F., Orchard, S. G., Jorissen, R L N, Hall, N. E., Zhang, H. H., Hoyne, P. A., Adams, T. E., Johns, T. G., Ward, C., Garrett, T. P., Zhu, l., Nerrie, M., Scott, A. M., Nice, E. C., and Burgess, A. W. (2004) J. Biol. Chem. 79, 22387-22398.
Weiner, L. M. “An overview of monoclonal antibody therapy of cancer.” Semin Oncol., 26 (Suppl. 12): 41-50, 1999.
Welt S, Divgi C R, Real F X, Yeh S D, Garin-Chesa P, Finstad C L, Sakamoto J, Cohen A, Sigurdson E R, Kemeny N, et al. (1990) J. Clin. Oncol. 8(11):1894-906.
Welt S, Divgi C R, Scott A M, Garin-Chesa P, Finn R D, Graham M, Carswell E A, Cohen A, Larson S M, Old L J (1994) J. Clin. Oncol. 12: 1193-1203.
Wersall, P., Ohlsson, I., Biberfeld, P., Collins, V. P., von Krusenstjerna, S., Larsson, S., Mellstedt, H., and Boethius, J. “Intratumoral infusion of the monoclonal antibody, mAb 425, against the epidermal-growth-factor receptor in patients with advanced malignant glioma.” Cancer Immunol. Immunother., 44: 157-164, 1997.
Whitson K. B., Red M. L., Whitson S. R., McCoy A., Vitali A. A., Walker F., Johns T. G., Beth A. H. and Staros J. A. “Functional Effects of Selective Glycosylation at Asn-579 of the Epidermal Growth Factor Receptor.” Biochemistry (2005) 44: 14920-14931
Wikstrand, C. J., Cokgor, I., Sampson, J. H., and Bigner, D. D. “Monoclonal antibody therapy of human gliomas: current status and future approaches.” Cancer Metastasis Rev., 18: 451-464, 1999.
Wikstrand, C. J., Hale, L. P., Batra, S. K., Hill, M. L., Humphrey, P. A., Kurpad, S, N., McLendon, R. E., Moscatello, D., Pegram, C. N., Reist, C. J., et al. “Monoclonal antibodies against EGFRvIII are tumor specific and react with breast and lung carcinomas and malignant gliomas.” Cancer Res.55: 3140-3148, 1995.
Wikstrand, C. J., McLendon, R. E., Friedman, A. H., and Bigner, D. D. “Cell surface localization and density of the tumor-associated variant of the epidermal growth factor receptor, EGFRvIII.” Cancer Res. 57: 4130-40, 1997.
Wikstrand, C. J., Reist, C. J., Archer, G. E., Zalutsky, M. R., and Bier, D. D. “The class III variant of the epidermal growth factor receptor (EGFRvIII): characterization and utilization as an immunotherapeutic target.” J. Neurovirol., 4: 148-158, 1998.
Wong A J, Bigner S H, Bigner D D, Kinzler K W, Hamilton S R, Vogelstein B (1987) Proc. Natl. Acad. Sci. USA 84(19):6899-6903.
Wong, A. J., Ruppert, J. M., Bigner, S. H., Grzeschik, C. H., Humphrey, P. A., Bigner, D. S., and Vogelstein, B. “Structural alterations of the epidermal growth factor receptor gene in human gliomas.” Proc. Natl. Acad. Sci. USA, 89: 2965-2969, 1992.
Yamazaki H, Fukui Y, Ueyama Y, et al. “Amplification of the structurally and functionally altered epidermal growth factor receptor gene (c-erbB) in human brain tumors.” Mol. Cell. Biol. 1988; 8: 1816-20.
Yamazaki H, Ohba Y, Tamaoki N, et al. “A deletion mutation within the ligand binding domain is responsible for activation of epidermal growth factor receptor gene in human brain tumors.” Jpn. J. Cancer Res. 1990; 81: 773-9.
Yarden, Y. and Schlessinger, J. (1987) Biochemistry. 26, 1443-1451.
Yarden, Y. and Sliwkowski, M. X. (2001) Nat. Rev. Mol. Cell. Biol. 2, 127-137.
Yen, L., Benlimame, N., Nie, Z. R., Xiao, D., Wang, T., Al Moustafa, A. E., Esurni, H., Milanini, J., Hynes, N. E., Pages, G., and Alaoui-Jamali, M. A. (2002) Mol. Biol. Cell. 13(11):4029-44.
Ymer, S., Tucker, W. Q., Sanderson, C. l., Hapel, A. J., Campbell, H. D., and Young, l. G. (1985) Nature. 19-25; 317,255-258.
Yoshitake et al. (1979) Eur. J. Biochem., 101, 395-399
Zhang, x., Gureasko, J., Shen, K., Cole, P. A., and Kuriyan, J. (2006) Cell. 125, 1137-1149.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout the Specification and provided in a list of references above, each of which is incorporated herein by reference in its entirety.

What is claimed is:

1. An isolated antibody capable of binding EGFR on tumors containing amplifications of the EGFR gene, wherein cells of said tumors contain multiple copies of the EGFR gene, and on tumors that express the truncated version of the EGFR receptor de2-7, wherein said antibody does not bind to the de2-7 EGFR junctional peptide consisting of the amino acid sequence of SEQ ID NO:13, wherein said antibody binds to an epitope within the sequence of residues 287-302 (SEQ ID NO:14) of human wild-type EGFR, and wherein said antibody does not comprise a heavy chain variable region sequence having the amino acid sequence set forth in SEQ ID NO:11 and does not comprise a light chain variable region sequence having the amino acid sequence set forth in SEQ ID NO:12.

2. An isolated antibody according to claim 1, wherein said antibody comprises a heavy chain and a light chain, said heavy chain having the amino acid sequence set forth in SEQ ID NO:42, and said light chain having the amino acid sequence set forth in SEQ ID NO:47.

3. An isolated antibody according to claim 1, wherein said antibody comprises a heavy chain and a light chain, wherein the variable region of said heavy chain comprises polypeptide binding domain regions having amino acid sequences highly homologous to the amino acid sequences set forth in SEQ ID NOS:44, 45, and 46.

4. An isolated antibody according to claim 1, wherein said antibody comprises a heavy chain and a light chain, wherein the variable region of said light chain comprises polypeptide binding domain regions having amino acid sequences highly homologous to the amino acid sequences set forth in SEQ ID NOS:49, 50, and 51.

5. An isolated antibody according to one or more of claims 1, 2, 3, 4, 18 and 19, wherein said isolated antibody is the form of an antibody F(ab′)₂, scFv fragment, diabody, triabody or tetrabody.

6. An isolated antibody according to one or more of claims 1, 2, 3, 4, 18 and 19, further comprising a detectable or functional label.

7. An isolated antibody according to claim 6, wherein said detectable or functional label is a covalently attached drug.

8. An isolated antibody according to claim 6, wherein said label is a radiolabel.

9. An isolated antibody according to one or more of claims 1, 2, 3, 4, 18 and 19, wherein said isolated antibody is peglyated.

10. Use of an isolated antibody according to one or more of claims 1, 2, 3, 4, 18 and 19 in the manufacture of a medicament for the treatment of a tumor in a human patient.

11. A pharmaceutical composition comprising an isolated antibody according to one or more of claims 1, 2, 3, 4, 18 and 19.

12. A pharmaceutical composition according to claim 11, further comprising a pharmaceutically acceptable vehicle, carrier or diluent.

13. A pharmaceutical composition according to claim 11, further comprising an anti-cancer agent selected from the group consisting of chemotherapeutic agents, anti-EGFR antibodies, radioimmunotherapeutic agents, chemical ablation agents, toxins, immunomodulators, cytokines, cytotoxic agents, drugs and combinations thereof.

14. A pharmaceutical composition according to claim 13, wherein said anti-cancer agent is conjugated to said antibody.

15. A pharmaceutical composition according to claim 14, wherein said anti-cancer agent is conjugated to said antibody using one or more linker, spacer and stretcher compounds.

16. A pharmaceutical composition according to claim 13, wherein said chemotherapeutic agents are selected from the group consisting of tyrosine kinase inhibitors, phosphorylation cascade inhibitors, post-translational modulators, cell growth or division inhibitors (e.g. anti-mitotics), signal transduction inhibitors, and combinations thereof.

17. Use of a therapeutically effective amount of a pharmaceutical composition according to claim 11 in the manufacture of a medicament for the prevention and/or treatment of cancer in a mammal.

18. An isolated antibody capable of binding EGFR on tumors containing amplifications of the EGFR gene, wherein cells of said tumors contain multiple copies of the EGFR gene, and on tumors that express the truncated version of the EGFR receptor de2-7, wherein said antibody comprises a heavy chain and a light chain, said heavy chain having an amino acid sequence that is substantially homologous to the amino acid sequence set forth in SEQ ID NO:42, and said light chain having an amino acid sequence that is substantially homologous to the amino acid sequence set forth in SEQ ID NO:47.

19. An isolated antibody capable of binding EGFR on tumors containing amplifications of the EGFR gene, wherein cells of said tumors contain multiple copies of the EGFR gene, and on tumors that express the truncated version of the EGFR receptor de2-7, wherein said antibody does not bind to the de2-7 EGFR junctional peptide consisting of the amino acid sequence of SEQ ID NO:13, wherein said antibody binds to an epitope within the sequence of residues 287-302 (SEQ ID NO:14) of human wild-type EGFR,
said antibody comprising a light chain and a heavy chain, wherein the variable region of said light chain comprises a first polypeptide binding domain region having an amino acid sequence corresponding to the amino acid sequence set forth in Formula I:
HSSQDIXₐₐ₁SNIG (I),
wherein Xₐₐ₁ is an amino acid residue having an uncharged polar R group;
a second polypeptide binding domain region having an amino acid sequence corresponding to the amino acid sequence set forth in Formula II:
HGTNLXₐₐ₂D (II),
wherein Xₐₐ₂ is an amino acid residue having a charged polar R group;
and a third polypeptide binding domain region having an amino acid sequence corresponding to the amino acid sequence set forth in Formula III:
VQYXₐₐ₃QFPWT (III),
wherein Xₐₐ₃ is selected from the group consisting of A, G, and an amino acid residue which is conservatively substituted for A or G; and
wherein the variable region of said heavy chain comprises a first polypeptide binding domain region having an amino acid sequence corresponding to the amino acid sequence set forth in Formula IV:
SDXₐₐ₄AWN (IV),
wherein Xₐₐ₄ is selected from the group consisting of F, Y, and an amino acid residue which is conservatively substituted for F or Y;
a second polypeptide binding domain region having an amino acid sequence corresponding to the amino acid sequence set forth in Formula V, Formula VI, or Formula VII:
YISYSGNTRYXₐₐ₅PSLKS (V),
wherein Xₐₐ₅ is an amino acid residue having an uncharged polar R group,
YISYSXₐₐ₆NTRYNPSLKS (VI),
wherein Xₐₐ₆ is selected from the group consisting of G, A, and an amino acid residue which is conservatively substituted for G or A,
YISYSGNTRYNPSLXₐₐ₇S (VII),
and Xₐₐ₇ is a basic amino acid residue; and
a third polypeptide binding domain region having an amino acid sequence corresponding to the amino acid sequence set forth in Formula VIII:
Xₐₐ₈TAGRGFPY (VIII),
wherein Xₐₐ₈ is selected from the group consisting of V, A, and an amino acid residue which is conservatively substituted for V or A,
and wherein said antibody does not comprise a heavy chain variable region sequence having the amino acid sequence set forth in SEQ ID NO:2 and does not comprise a light chain variable region sequence having the amino acid sequence set forth in SEQ ID NO:4.

20. An isolated antibody according to claim 19, wherein Xₐₐ₁ is N; Xₐₐ₂ is D; Xₐₐ₃ is A; Xₐₐ₄ is F; Xₐₐ₅ is an amino acid residue having an uncharged polar R group; Xₐₐ₆ is G; Xₐₐ₇ is K; and Xₐₐ₈ is V.

21. An isolated antibody according to claim 20, wherein Xₐₐ₅ is N or Q.
